(19) United States
(12) Patent Application Publication
(10) Pub. No.: US 2005/0038035 A1
(43) Pub. Date: Feb. 17, 2005

(54) Heterocyclic amide compounds as apolipoprotein b inhibitors

(76) Inventors: Hisashi Takasugi, Osaka (JP); Yoshikazu Inoue, Osaka (JP); Takeshi Terasawa, Osaka (JP); Akira Nagayoshi, Osaka (JP); Yoshiro Furukawa, Osaka (JP); Masafumi Mikami, Osaka (JP); Kazumasa Hinoue, Osaka (JP); Makoto Ohtsubo, Osaka (JP); Daisuke Fukumoto, Osaka (JP)

Correspondence Address: OBLON, SPIVAK, MCCLELLAND, MAIER & NEUSTADT, P.C., 1940 DUKE STREET, ALEXANDRIA, VA, 22314, US

(21) Appl. No.: 10/496,967

(22) PCT Filed: Oct. 24, 2002
(86) PCT No.: PCT/JP02/11034

(30) Foreign Application Priority Data
Nov. 28, 2001 (AU) PR 9164
Feb. 11, 2002 (AU) PS 0443
Apr. 4, 2002 (TW) 91106855
Apr. 9, 2002 (WO) PCT/JP02/03529

(51) Int. Cl. A61K 31/496; C07D 43/02

(52) U.S. Cl. 514/253.01; 514/254.01; 514/255.03; 544/360; 544/373

(57) ABSTRACT

The present invention relates to a compound of the formula (I) wherein R¹ is optionally substituted aryl; R² is optionally substituted aryl, optionally substituted heteroaryl, optionally substituted lower cycloalkyl, optionally substituted aryloxy, optionally substituted arylsulfonyl, vinyl, carbamoyl, protected carboxy or protected amino; ring A is bivalent residue derived from optionally substituted aryl or optionally substituted heteroaryl; X is bivalent residue derived from the group consisting of cycloalkene, naphthalene, unsaturated 5 or 6-membered heteromonocyclic group, each of which is optionally substituted, and substituted benzene; Y is -(A¹)ₘ₁-(A²)ₘ₂-; and Z is direct bond or piperazine, or a salt thereof. The compound of the present invention and a salt thereof inhibit apolipoprotein B (Apo B) secretion and are useful as a medicament for prophylactic and treatment of diseases or conditions resulting from elevated circulating levels of Apo B.

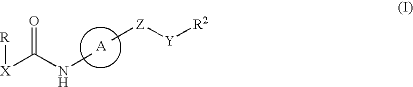

### TECHNICAL FIELD

This invention relates to new amide compounds and salts thereof which inhibit apolipoprotein B (Apo B) secretion and are useful as a medicament.

### BACKGROUND ART

Apo B is the main component of lipoprotein such as VLDL (very low density lipoprotein), IDL (intermediate density lipoprotein) and LDL (low density lipoprotein). Compounds that inhibit Apo B secretion are useful for the treatment of diseases or conditions resulting from elevated circulating levels of Apo B, such as hyperlipemia, hyperlipidemia, hyperlipoproteinemia, hypercholesterolemia, hypertriglyceridemia, atherosclerosis, pancreatitis, non-insulin dependent diabetes mellitus (NIDDM), obesity and coronary heart diseases. Compounds that inhibit Apo B secretion have been described in WO96/40640, WO98/23593, WO98/56790 and WO00/32582. Compounds that inhibit Apo B secretion are also useful in reducing intestinal fat absorption, reducing food intake and treating obesity in combination with a known anti-obesity agent (EP 1 099 438, EP 1 099 439 and EP 1 099 441).

### DISCLOSURE OF INVENTION

This invention relates to new amide compounds.

One object of this invention is to provide new and useful amide compounds and salts thereof that inhibit Apo B secretion.

A further object of this invention is to provide a pharmaceutical composition comprising said amide compound or a pharmaceutically acceptable salt thereof.

Still further object of this invention is to provide a use of said amide compounds or pharmaceutically acceptable salts thereof as a medicament for prophylactic and therapeutic treatment of diseases or conditions resulting from elevated circulating levels of Apo B, such as hyperlipemia, hyperlipidemia, hyperlipoproteinemia, hypercholesterolemia, hypertriglyceridemia, atherosclerosis, pancreatitis, non-insulin dependent diabetes mellitus (NIDDM), obesity, coronary heart diseases, myocardial infarction, stroke, restenosis and Syndrome X.

Another object of this invention is to provide a method for inhibiting or decreasing Apo B secretion in a mammal, which comprises administering an Apo B secretion inhibiting or decreasing amount of said amide compound or a pharmaceutically acceptable salt thereof to the mammal.

Still further object of this invention is to provide a method for preventing or treating a disease or condition resulting from elevated circulating levels of Apo B in a mammal, such as hyperlipemia, hyperlipidemia, hyperlipoproteinemia, hypercholesterolemia, hypertriglyceridemia, atherosclerosis, pancreatitis, NIDDM, obesity, coronary heart diseases, myocardial infarction, stroke, restenosis and Syndrome X, which method comprises administering an effective amount of said amide compound or a pharmaceutically acceptable salt thereof to the mammal.

The object amide compounds of the present invention are novel and can be represented by the following general formula (I) 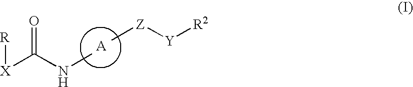
wherein
R¹ is aryl optionally substituted by substituent(s);
R² is aryl, heteroaryl, lower cycloalkyl, aryloxy, arylsulfonyl, vinyl, carbamoyl, protected carboxy or protected amino, each of said aryl, heteroaryl, lower cycloalkyl, aryloxy and arylsulfonyl is optionally substituted by substituent(s); 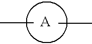
   is bivalent residue derived from aryl or heteroaryl, each of which is optionally substituted by nitro, oxo or optionally protected amino;
X is bivalent residue derived from the group consisting of cycloalkene, naphthalene, unsaturated 5 or 6-membered heteromonocyclic group, each of which is optionally substituted by substituent(s), and benzene which is substituted by substituent(s);
Y is -(A¹)ₘ₁-(A²)ₘ₂-
   wherein A¹ is —NH—, —N(R³)—, —CO—, —NH—CO—, —CO—NH—, —CO—CH═CH—, —O—, —CH₂—O—, —CH₂—NH—CO—, —CH₂—CO—NH— or —CH(OH)—, wherein R³ is amino protective group,
   A² is lower alkylene optionally substituted by aryl, and
   m1 and m2 are independently 0 or 1; and
Z is direct bond or bivalent residue derived from piperazine or piperazine substituted by lower alkyl;
provided that when Z is direct bond, then R² is aryl, heteroaryl, lower cycloalkyl, aryloxy, arylsulfonyl or protected amino, each of said aryl, heteroaryl, lower cycloalkyl, aryloxy and arylsulfonyl is optionally substituted by substituent(s),
or a salt thereof.

The preferred embodiments of the amide compound of the present invention represented by the general formula (I) are as follows.

(1) The compound of the general formula (I), wherein
R¹ is aryl optionally substituted by substituent(s);
R² is aryl, heteroaryl, lower cycloalkyl, aryloxy, arylsulfonyl, vinyl, carbamoyl, protected carboxy or protected amino, each of said aryl, heteroaryl, lower cycloalkyl, aryloxy and arylsulfonyl is optionally substituted by substituent(s) selected from the group consisting of lower alkyl, trihalo(lower)alkyl, optionally protected amino, optionally substituted heteroaryl, cyano, lower alkoxy, halogen, aryloxy, lower alkylenedioxy, oxo, lower alkanoylamino and amino protective group; 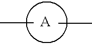
   is bivalent residue derived from aryl or heteroaryl, each of which is optionally substituted by nitro, oxo or optionally protected amino;
X is bivalent residue derived from the group consisting of cycloalkene, naphthalene, unsaturated 5 or 6-membered heteromonocyclic group, each of which is optionally substituted by substituent(s), and benzene which is substituted by substituent(s);
Y is -(A¹)ₘ₁-(A²)ₘ₂-
   wherein A¹ is —NH—, —N(R³)—, —CO—, —NH—CO—, —CO—NH—, —CO—CH═CH—, —O—, —CH₂—O—, —CH₂—NH—CO—, —CH₂—CO—NH— or —CH(OH)—, wherein R³ is amino protective group,
   A² is lower alkylene optionally substituted by aryl, and
   m1 and m2 are independently 0 or 1; and
Z is direct bond or bivalent residue derived from piperazine or piperazine substituted by lower alkyl;
provided that when Z is direct bond, then R² is aryl, heteroaryl, lower cycloalkyl, aryloxy, arylsulfonyl or protected amino, each of said aryl, heteroaryl, lower cycloalkyl, aryloxy and arylsulfonyl is optionally substituted by substituent(s) selected from the group consisting of lower alkyl, trihalo(lower)alkyl, optionally protected amino, optionally substituted heteroaryl, cyano, lower alkoxy, halogen, aryloxy, lower alkylenedioxy, oxo, lower alkanoylamino and amino protective group,
or a salt thereof.

(2) The compound of (1) above, wherein
R¹ is phenyl optionally substituted by substituent(s) selected from the group consisting of lower alkyl, lower alkoxy, halogen, trihalo(lower)alkyl, trihalo(lower)alkoxy, lower alkanoyl, di(lower)alkylamino and lower alkylthio;
R² is phenyl, naphthyl, indanyl, pyridinyl, pyrimidinyl, pyrazinyl, thiazolyl, pyrrolyl, imidazolyl, triazolyl, thienyl, indolyl, lower cycloalkyl, phenoxy, naphthyloxy, phenylsulfonyl or protected amino, each of said phenyl, naphthyl, indanyl, pyridinyl, pyrimidinyl, pyrazinyl, thiazolyl, pyrrolyl, imidazolyl, triazolyl, thienyl, indolyl, lower cycloalkyl, phenoxy, naphthyloxy and phenylsulfonyl is optionally substituted by substituent(s) selected from the group consisting of lower alkyl, trihalo(lower)alkyl, optionally protected amino, optionally substituted pyrrolyl, cyano, lower alkoxy, halogen, aryloxy, lower alkylenedioxy, oxo, lower alkanoylamino and amino protective group; 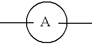
   is bivalent residue derived from phenyl optionally substituted by nitro or optionally protected amino, indanyl, pyridinyl, indolinyl, tetrahydroisoquinolyl or isoindolinyl each of which is optionally substituted by oxo or amino;
X is bivalent residue derived from the group consisting of cycloalkene, naphthalene, unsaturated 5 or 6-membered heteromonocyclic group, each of which is optionally substituted by substituent(s), and benzene which is substituted by substituent(s), wherein the substituent is selected from the group consisting of lower alkyl, lower alkoxy, halogen, lower alkanoyl, lower alkoxy(lower)alkyl and hydroxy(lower)alkyl;
Y is -(A¹)ₘ₁-(A²)ₘ₂-
   wherein A¹ is —NH—, —N(R³)—, —CO—, —NH—CO—, —CO—NH—, —CO—CH═CH—, —O—, —CH₂—O—, —CH₂—NH—CO—, —CH₂—CO—NH— or —CH(OH)—, wherein R³ is amino protective group,
   A² is lower alkylene optionally substituted by aryl, and
   m1 and m2 are independently 0 or 1; and
Z is direct bond,
or a salt thereof.

(3) The compound of (2) above, wherein
R¹ is phenyl optionally substituted by substituent(s) selected from the group consisting of methyl, ethyl, isopropyl, methoxy, chloro, fluoro, bromo, trifluoromethyl, trifluoromethoxy, acetyl, dimethylamino and methylthio;
R¹ is pyridinyl, pyrimidinyl, pyrazinyl or thiazolyl, each of said pyridinyl, pyrimidinyl, pyrazinyl and thiazolyl is optionally substituted by substituent(s) selected from the group consisting of methyl, amino, acetylamino or tert-butoxycarbonylamino, optionally substituted pyrrolyl, cyano and methoxy; 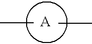
   is bivalent residue derived from phenyl or pyridinyl;
X is 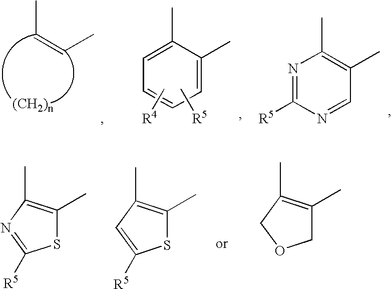
   wherein R⁴ is lower alkyl, lower alkoxy, lower alkanoyl, hydroxy(lower)alkyl, lower alkoxy(lower)alkyl or halogen,
   R⁵ is hydrogen or lower alkyl, and
   n is 3, 4, 5 or 6;
Y is direct bond or bivalent residue selected from the group consisting of 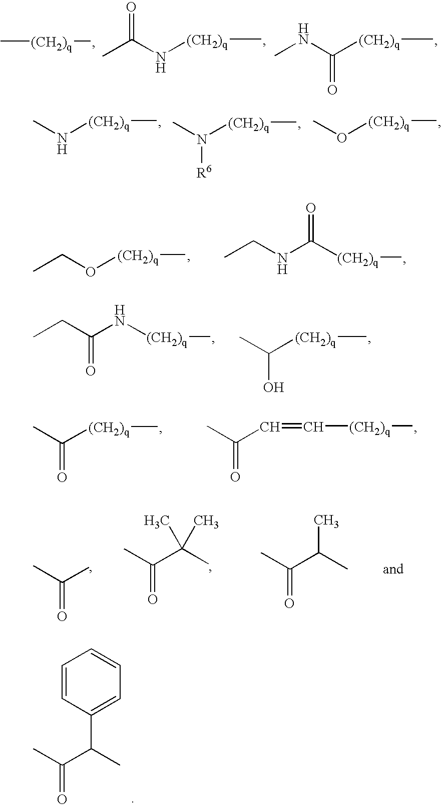
   wherein q is an integer of 0 to 3, and R⁶ is amino protective group,
or a salt thereof.

(4) The compound of the formula (I), wherein X is 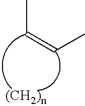
wherein n is 3, 4, 5 or 6,
or a salt thereof.

(5) The compound of (4) above, wherein
R¹ is phenyl optionally substituted by substituent(s) selected from the group consisting of lower alkyl, lower alkoxy, halogen, trihalo(lower)alkyl, trihalo(lower)alkoxy, lower alkanoyl, di(lower)alkylamino and lower alkylthio;
R² is aryl, heteroaryl, lower cycloalkyl, aryloxy, arylsulfonyl, vinyl, carbamoyl, protected carboxy or protected amino, each of said aryl, heteroaryl, lower cycloalkyl, aryloxy and arylsulfonyl is optionally substituted by substituent(s) selected from the group consisting of lower alkyl, trihalo(lower)alkyl, optionally protected amino, optionally substituted heteroaryl, cyano, lower alkoxy, halogen, aryloxy, lower alkylenedioxy, oxo, lower alkanoylamino and amino protective group; 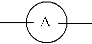
   is bivalent residue derived from aryl or heteroaryl;
X is 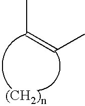
   wherein n is 3, 4, 5 or 6;
Y is -(A¹)ₘ₁-(A²)ₘ₂-
   wherein A¹ is —NH—, —N(R³)—, —CO—, —NH—CO—, —CO—NH—, —CO—CH═CH—, —O—, —CH₂—O—, —CH₂—NH—CO—, —CH₂—CO—NH— or —CH(OH)—, wherein R³ is amino protective group,
   A² is lower alkylene optionally substituted by aryl, and
   m1 and m2 are independently 0 or 1; and
Z is direct bond or bivalent residue derived from piperazine or piperazine substituted by lower alkyl;
provided that when Z is direct bond, then R² is aryl, heteroaryl, lower cycloalkyl, aryloxy, arylsulfonyl or protected amino, each of said aryl, heteroaryl, lower cycloalkyl, aryloxy and arylsulfonyl is optionally substituted by substituent(s) selected from the group consisting of lower alkyl, trihalo(lower)alkyl, optionally protected amino, optionally substituted heteroaryl, cyano, lower alkoxy, halogen, aryloxy, lower alkylenedioxy, oxo, lower alkanoylamino and amino protective group,
or a salt thereof.

(6) The compound of (5) above, wherein
R² is phenyl, naphthyl, indanyl, pyridinyl, pyrimidinyl, thiazolyl, pyrrolyl, imidazolyl, triazolyl, thienyl, indolyl, lower cycloalkyl, phenoxy, naphthyloxy, phenylsulfonyl, vinyl, carbamoyl, protected carboxy or protected amino, each of said phenyl, naphthyl, indanyl, pyridinyl, pyrimidinyl, thiazolyl, pyrrolyl, imidazolyl, triazolyl, thienyl, indolyl, lower cycloalkyl, phenoxy, naphthyloxy and phenylsulfonyl is optionally substituted by substituent(s) selected from the group consisting of lower alkyl, trihalo(lower)alkyl, optionally protected amino, optionally substituted pyrrolyl, cyano, lower alkoxy, halogen, aryloxy, lower alkylenedioxy, oxo, lower alkanoylamino and amino protective group; 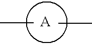
   is bivalent residue derived from phenyl, indanyl, pyridinyl, indolinyl, isoindolinyl or 1,2,3,4-tetrahydroisoquinolinyl;
Y is direct bond or bivalent residue selected from the group consisting of 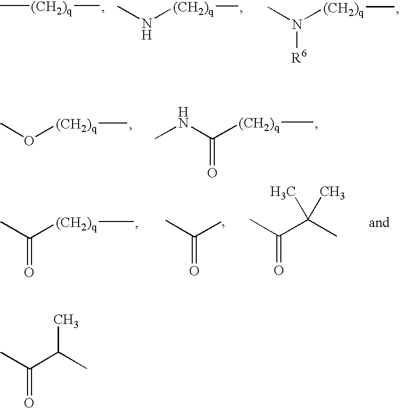
   wherein q is an integer of 0 to 3, and R⁶ is amino protective group;
provided that when Z is direct bond, then R² is phenyl, naphthyl, indanyl, pyridinyl, pyrimidinyl, thiazolyl, pyrrolyl, imidazolyl, triazolyl, thienyl, indolyl; lower cycloalkyl, phenoxy, naphthyloxy, phenylsulfonyl or protected amino, each of said phenyl, naphthyl, indanyl, pyridinyl, pyrimidinyl, thiazolyl, pyrrolyl, imidazolyl, triazolyl, thienyl, indolyl, lower cycloalkyl, phenoxy, naphthyloxy and phenylsulfonyl is optionally substituted by substituent(s) selected from the group consisting of lower alkyl, trihalo(lower)alkyl, optionally protected amino, optionally substituted pyrrolyl, cyano, lower alkoxy, halogen, aryloxy, lower alkylenedioxy, oxo, lower alkanoylamino and amino protective group, or a salt thereof.

(7) The compound of (4) above, having the following formula: 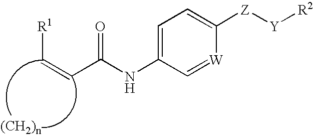
wherein
R¹is phenyl optionally substituted by substituent(s) selected from the group consisting of lower alkyl, lower alkoxy, halogen, trihalo(lower)alkyl, trihalo(lower)alkoxy, lower alkanoyl and di(lower)alkylamino;
R² is aryl or heteroaryl, each of said aryl and heteroaryl is optionally substituted by substituent(s) selected from the group consisting of lower alkyl, trihalo(lower)alkyl, optionally protected amino, optionally substituted heteroaryl, cyano, lower alkoxy, lower alkanoylamino and amino protective group;
W is CH or N;
Y is -(A¹)ₘ₁-(A²)ₘ₂-
   wherein A¹ is —NH—, —N(R³)—, —CO—, —NH—CO—, —CO—NH—, —CO—CH═CH—, —O—, —CH₂—O—, —CH₂—NH—CO—, —CH₂—CO—NH— or —CH(OH)—, wherein R³ is amino protective group,
   A² is lower alkylene optionally substituted by aryl, and
   m1 and m2 are independently 0 or 1;
Z is direct bond; and
n is 3, 4, 5 or 6,
or a salt thereof.

(8) The compound of (7) above, having the following formula: 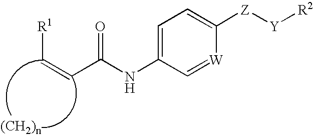
wherein
R¹ is phenyl optionally substituted by substituent(s) selected from the group consisting of lower alkyl and trihalo(lower)alkyl;
R² is pyridinyl or thiazolyl, each of said pyridinyl and thiazolyl is optionally substituted by optionally protected amino;
W is CH or N;
Y is -(A¹)ₘ₁-(A²)ₘ₂-
   wherein A¹ is —NH—, —N(R³)— or —O—, wherein R³ is amino protective group,
   A² is lower alkylene optionally substituted by aryl, and
   m1 and m2 are independently 0 or 1;
Z is direct bond; and
n is 4,
or a salt thereof.

(9) The compound of (4) above, having the following formula: 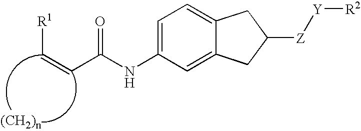
wherein
R¹ is phenyl optionally substituted by substituent(s) selected from the group consisting of lower alkyl, lower alkoxy, halogen, trihalo(lower)alkyl, trihalo(lower)alkoxy, lower alkanoyl, di(lower)alkylamino and lower alkylthio;
R² is aryl, heteroaryl or protected amino, each of said aryl and heteroaryl is optionally substituted by substituent(s) selected from the group consisting of lower alkyl, trihalo(lower)alkyl, optionally protected amino, optionally substituted heteroaryl, cyano, lower alkoxy, halogen, aryloxy, lower alkylenedioxy, lower alkanoylamino and amino protective group;
Y is -(A¹)ₘ₁-(A²)ₘ₂-
   wherein A¹ is —NH—, —N(R³)—, —CO—, —NH—CO—, —CO—CH═CH— or —O—, wherein R³ is amino protective group,
   A² is lower alkylene optionally substituted by aryl, and
   m1 and m2 are independently 0 or 1;
Z is direct bond; and
n is 3, 4, 5 or 6,
or a salt thereof.

(10) The compound of (4) above, having the following formula: 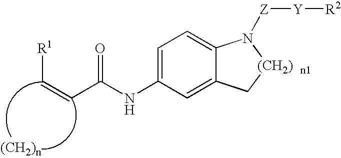
wherein
R¹ is phenyl optionally substituted by substituent(s) selected from the group consisting of lower alkyl, lower alkoxy, halogen, trihalo(lower)alkyl, trihalo(lower)alkoxy, lower alkanoyl, di(lower)alkylamino and lower alkylthio;
R² is aryl or heteroaryl, each of said aryl and heteroaryl is optionally substituted by substituent(s) selected from the group consisting of lower alkyl, trihalo(lower)alkyl, optionally protected amino, optionally substituted heteroaryl, cyano, lower alkoxy, halogen, aryloxy, lower alkylenedioxy, oxo, lower alkanoylamino and amino protective group;
Y is -(A¹)ₘ₁-(A²)ₘ₂-
   wherein A¹ is —NH—, —N(R³)—, —CO—, —NH—CO—, —CO—CH═CH— or —O—, wherein R³ is amino protective group,
   A² is lower alkylene optionally substituted by aryl, and
   m1 and m2 are independently 0 or 1;
Z is direct bond;
n is 3, 4, 5 or 6; and
n1 is 1 or 2,
or a salt thereof.

(11) The compound of (4) above, having the following formula: 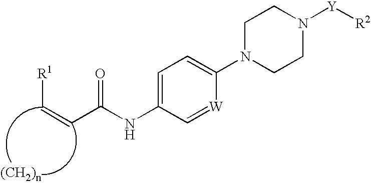
wherein
R¹ is phenyl optionally substituted by substituent(s) selected from the group consisting of lower alkyl, lower alkoxy, halogen, trihalo(lower)alkyl, trihalo(lower)alkoxy, lower alkanoyl, di(lower)alkylamino and lower alkylthio;
R² is aryl, heteroaryl, vinyl, carbamoyl, protected carboxy or protected amino, each of said aryl and heteroaryl is optionally substituted by substituent(s) selected from the group consisting of lower alkyl, trihalo(lower)alkyl, optionally protected amino, optionally substituted heteroaryl, cyano, lower alkoxy, halogen, aryloxy, lower alkylenedioxy, oxo, lower alkanoylamino and amino protective group;
W is CH or N;
Y is -(A¹)ₘ₁-(A²)ₘ₂-
   wherein A¹ is —NH—, —N(R³)—, —CO—, —NH—CO—, —CO—CH═CH— or —O—, wherein R³ is amino protective group,
   A² is lower alkylene optionally substituted by aryl, and
   m1 and m2 are independently 0 or 1;
n is 3, 4, 5 or 6,
or a salt thereof.

(12) The compound of (11) above, having the following formula: 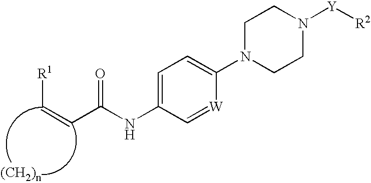
wherein
R¹ is phenyl optionally substituted by substituent(s) selected from the group consisting of lower alkyl and trihalo(lower)alkyl;
R² is aryl optionally substituted by cyano;
W is CH or N;
Y is -(A²)ₘ₂-
   wherein A² is lower alkylene, and
   m2 is 1;
n is 4,
or a salt thereof.

(13) The compound of (4) above, having the following formula: 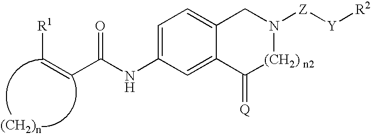
wherein
R¹ is phenyl optionally substituted by substituent(s) selected from the group consisting of lower alkyl, lower alkoxy, halogen, trihalo(lower)alkyl, trihalo(lower)alkoxy, lower alkanoyl, di(lower)alkylamino and lower alkylthio;
R² is aryl, heteroaryl or protected amino, each of said aryl and heteroaryl is optionally substituted by substituent(s) selected from the group consisting of lower alkyl, trihalo(lower)alkyl, optionally protected amino, optionally substituted heteroaryl, cyano, lower alkoxy, halogen, aryloxy, lower alkylenedioxy, oxo, lower alkanoylamino and amino protective group;
Y is -(A¹)ₘ₁-(A²)ₘ₂-
   wherein A¹ is —NH—, —N(R³)—, —CO—, —NH—CO—, —CO—CH═CH— or —O—, wherein R³ is amino protective group,
   A² is lower alkylene optionally substituted by aryl, and
   m1 and m2 are independently 0 or 1;
Z is direct bond;
Q is O or a pair of hydrogen atoms;
n is 3, 4, 5 or 6; and
n2 is 0 or 1,
or a salt thereof.

(14) The compound of the formula (I), wherein X is 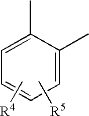
wherein R⁴ is lower alkyl, lower alkoxy, lower alkanoyl, hydroxy(lower)alkyl, lower alkoxy(lower)alkyl or halogen, and
R⁵ is hydrogen or lower alkyl,
or a salt thereof.

(15) The compound of (14) above, wherein
R¹ is phenyl optionally substituted by substituent(s) selected from the group consisting of lower alkyl and trihalo(lower)alkyl;
R² is heteroaryl optionally substituted by optionally protected amino; 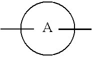
   is bivalent residue derived from aryl or pyridinyl;
X is 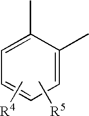
   wherein R⁴ is lower alkyl, and R⁵ is hydrogen;
Y is -(A¹)ₘ₁-(A²)ₘ₂-
   wherein A¹ is —NH—, —N(R³)—, —O—, wherein R³ is amino protective group,
   A² is lower alkylene optionally substituted by aryl, and
   m1 and m2 are independently 0 or 1; and
Z is direct bond,
or a salt thereof.

(16) The compound of the formula (I) selected from the group consisting of
4′,5-dimethyl-N-(4-{[2-(2-pyridinyl)ethyl]amino}phenyl)-1,1′-biphenyl-2-carboxamide (Example 43),
5-methyl-N-(4-{[2-(2-pyridinyl)ethyl]amino}phenyl)-4′-(trifluoromethyl)-1,1′-biphenyl-2-carboxamide (Example 44),
N-{4-[2-(6-amino-2-pyridinyl)ethoxy]phenyl}-5-methyl-4′-(trifluoromethyl)-1,1′-biphenyl-2-carboxamide (Example 106),
2-(4-methylphenyl)-N-(4-{[2-(2-pyridinyl)ethyl]amino}phenyl)-1-cyclohexene-1-carboxamide (Example 115),
N-(4-{[2-(2-pyridinyl)ethyl]amino}phenyl)-2-[4-(trifluoromethyl)phenyl]-1-cyclohexene-1-carboxamide (Example 116),
N-(4-{[2-(6-amino-2-pyridinyl)ethyl]amino}phenyl)-2-[4-(trifluoromethyl)phenyl]-1-cyclohexene-1-carboxamide (Example 145),
N-(4-{[2-(2-amino-1,3-thiazol-4-yl)ethyl]amino}phenyl)-2-(4-methylphenyl)-1-cyclohexene-1-carboxamide (Example 169),
N-{4-[4-(3-cyanobenzyl)-1-piperazinyl]phenyl}-2-[4-(trifluoromethyl)phenyl]-1-cyclohexene-1-carboxamide (Example 190),
N-{6-[4-(3-cyanobenzyl)-1-piperazinyl]-3-pyridinyl}-2-[4-(trifluoromethyl)phenyl]-1-cyclohexene-1-carboxamide (Example 212),
N-(6-{[2-(6-amino-2-pyridinyl)ethyl]amino}-3-pyridinyl)-2-[4-(trifluoromethyl)phenyl]-1-cyclohexene-1-carboxamide (Example 388),
or a salt thereof.

(17) A compound of the following formula: 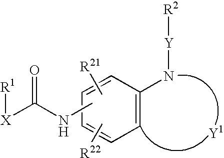
wherein
R¹ is 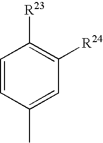
   wherein R²³ and R²⁴ are independently hydrogen or a substituent;
R²¹ and R²² are independently hydrogen or a substituent;
R² is unsaturated 5 to 6-membered heteromonocyclic group, which is optionally substituted by one or more substituent(s);
X is cycloalkenylene optionally substituted by one or more substituent(s);
Y¹ is bivalent group selected from the group consisting of ethylene, trimethylene and vinylene, wherein CH₂ is optionally replaced by NH or O, and CH is optionally replaced by N, and said bivalent group is optionally substituted by one or more substituent(s);
   and
Y is —(CH₂)ᵣ—, —CO—(CH₂)ₛ— or —CO—NH—, wherein r is 1, 2 or 3 and s is 1 or 2,
or a salt thereof.

(18) A compound of the formula: 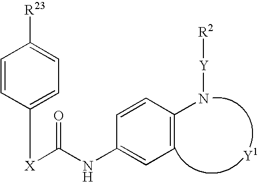
wherein
R²³ is hydrogen, lower alkyl, lower alkoxy, halogen, trihalo(lower)alkyl or di(lower)alkylamino;
R² is 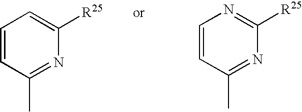
   wherein R²⁵ is hydrogen, amino or 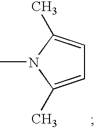
X is 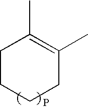
   wherein p is 1 or 2;
Y¹ is —CH₂—CH₂—; and
Y is —CO—CH₂—,
or a salt thereof.

Examples of a preferable group represented by Y include the following. 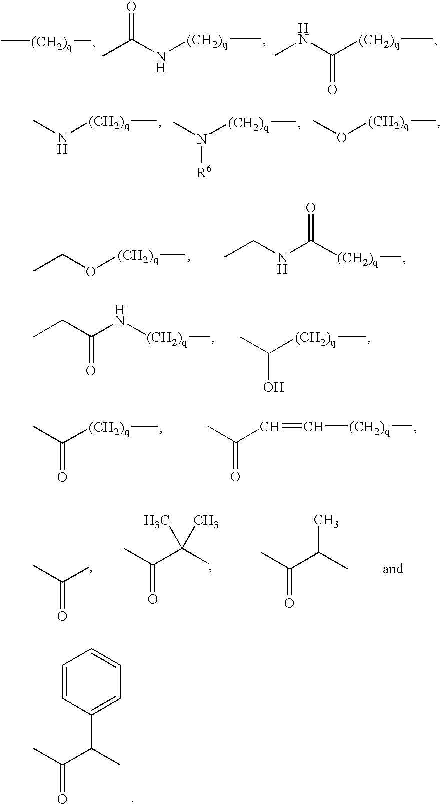
wherein q is an integer of 0 to 3, and R⁶ is amino protective group.

Examples of a preferable group represented by the formula: -Z-Y—R² include -Z-(CH₂)_{q}—R², -Z-CONH—(CH₂)_{q}—R², -Z-NHCO—(CH₂)_{q}—R², -Z-NH—(CH₂)_{q}—R², -Z-N(R³)—(CH₂)_{q}—R², -Z-O—(CH₂)_{q}—R², -Z-CH₂O—(CH₂)_{q}—R², -Z-CO—(CH₂)_{q}—R², -Z-CH(OH)—(CH₂)_{q}—R² and -Z-CO—CH═CH—(CH₂)_{q}—R² wherein R², R³, Z and q are as defined above.

Suitable salts of the object compound (I) may be pharmaceutically acceptable salts such as conventional non-toxic salts and include, for example, a salt with a base or an acid addition salt such as a salt with an inorganic base, for example, an alkali metal salt (e.g., sodium salt, potassium salt, etc.), an alkaline earth metal salt (e.g., calcium salt, magnesium salt, etc.), an ammonium salt; a salt with an organic base, for example, an organic amine salt (e.g., triethylamine salt, pyridine salt, picoline salt, ethanolamine salt, triethanolamine salt, dicyclohexylamine salt, N,N′-dibenzylethylenediamine salt, etc.); an inorganic acid addition salt (e.g., hydrochloride, hydrobromide, sulfate, phosphate, etc.); an organic carboxylic or sulfonic acid addition salt (e.g., formate, acetate, trifluoroacetate, maleate, tartrate, citrate, fumarate, methanesulfonate, benzenesulfonate, toluenesulfonate, etc.); and a salt with a basic or acidic amino acid (e.g., arginine, aspartic acid, glutamic acid, etc.).

In the above and subsequent descriptions of the present specification, suitable examples and illustration of the various definitions which the present invention intends to include within the scope thereof are explained in detail as follows.

The term “lower” is used to intend a group having 1 to 6, preferably 1 to 4, carbon atom(s), unless otherwise provided.

Suitable “lower alkyl” and “lower alkyl” moiety in the terms “trihalo(lower)alkyl”, “di(lower)alkylamino”, “lower alkylthio”, “hydroxy(lower)alkyl”, “lower alkoxy(lower)alkyl”, “mono(or di or tri)aryl(lower)alkyl”, “mono or di or tri)phenyl(lower)alkyl”, “lower alkylsulfonyl”, “aryl(lower)alkylsulfonyl”, “lower alkylsulfonylamino”, “aryl(lower)alkylsulfonylamino”, “bis[(lower)alkylsulfonyl]amino” and “bis[aryl(lower)alkylsulfonyl]amino”, include straight or branched alkyl having 1 to 6 carbon atom(s), such as methyl, ethyl, propyl, isopropyl, butyl, isobutyl, sec-butyl, tert-butyl, pentyl, tert-pentyl and hexyl, in which more preferred one is C₁-C₄ alkyl.

Suitable “lower alkoxy” and “lower alkoxy” moiety in the terms “trihalo(lower)alkoxy”, “lower alkoxy(lower)alkyl”, “(lower)alkoxycarbonyl”, “mono(or di or tri)phenyl(lower)alkoxycarbonyl” and “(lower)alkoxycarbonylamino” include straight or branched alkoxy having 1 to 6 carbon atom(s), such as methoxy, ethoxy, propoxy, isopropoxy, butoxy, isobutoxy, sec-butoxy, tert-butoxy, pentyloxy, tert-pentyloxy and hexyloxy, in which more preferred one is C₁-C₄ alkoxy.

Suitable “halogen” and “halogen” moiety in the terms “trihalo(lower)alkyl” and “trihalo(lower)alkoxy” may be fluorine, bromine, chlorine and iodine.

Suitable “trihalo(lower)alkyl” includes trihalo(C₁-C₆)alkyl such as trifluoromethyl, trichloromethyl and tribromomethyl, in which more preferred one is trihalo(C₁-C₄)alkyl, and the particularly preferred one is trifluoromethyl.

Suitable “trihalo(lower)alkoxy” includes trihalo(C₁-C₆)alkoxy such as trifluoromethoxy, trichloromethoxy and tribromomethoxy, in which more preferred one is trihalo(C₁-C₄)alkoxy, and the particularly preferred one is trifluoromethoxy.

Suitable “lower alkanoyl” includes straight or branched alkanoyl having 1 to 6 carbon atom(s), such as formyl, acetyl, propanoyl, butanoyl, 2-methylpropanoyl, pentanoyl, 3-methylbutanoyl, 2,2-dimethylpropanoyl and hexanoyl, in which more preferred one is C₁-C₄ alkanoyl, and the particularly preferred one is acetyl.

Suitable “di(lower)alkylamino” includes di(C₁-C₆)alkylamino such as dimethylamino, diethylamino, dipropylamino, diisopropylamino, dibutylamino, dipentylamino, dihexylamino, ethylmethylamino, methylpropylamino and ethylpropylamino, in which more preferred one is di(C₁-C₄)alkylamino, and the particularly preferred one is dimethylamino.

Suitable “lower alkylthio” includes (C₁-C₆)alkylthio such as methylthio, ethylthio, propylthio, isopropylthio, butylthio, isobutylthio, sec-butylthio, tert-butylthio, pentylthio, tert-pentylthio and hexylthio, in which more preferred one is C₁-C₄ alkylthio, and the particularly preferred one is methylthio.

Suitable “lower alkylene” includes straight or branched alkylene having 1 to 6 carbon atoms, such as methylene, ethylene, trimethylene, tetramethylene, propylene, ethylidene and propylidene, in which more preferred one is C₁-C₃ alkylene.

Suitable “lower alkylenedioxy” includes straight or branched alkylenedioxy having 1 to 6 carbon atoms, such as methylenedioxy, ethylenedioxy, trimethylenedioxy, tetramethylenedioxy, propylenedioxy, ethylidenedioxy and propylidenedioxy, in which more preferred one is C₁-C₃ alkylenedioxy, and most preferred one is methylenedioxy.

Suitable “hydroxy(lower) alkyl” includes hydroxy(C₁-C₆)alkyl such as hydroxymethyl, 2-hydroxyethyl, 1-hydroxyethyl, 3-hydroxypropyl, 2-hydroxypropyl, 1-hydroxypropyl and 4-hydroxybutyl, in which more preferred one is hydroxymethyl.

Suitable “lower alkoxy(lower)alkyl” includes (C₁-C₆)alkoxy(C₁-C₆)alkyl such as methoxymethyl, 2-methoxyethyl, 3-methoxypropyl, 1-methoxy-1-methylethyl, 4-methoxybutyl and ethoxymethyl, 2-ethoxyethyl, 3-ethoxypropyl and 4-ethoxybutyl, in which more preferred ones are methoxymethyl and 1-methoxy-1-methylethyl.

Suitable “cycloalkene” includes cycloalkene having 3 to 8 carbon atoms, preferably 5 to 8 carbon atoms, more preferably 5 or 6 carbon atoms, and having 1 or 2 double bonds, preferably 1 double bond in the ring. Suitable examples of “cycloalkene” include cyclopropene, cyclobutene, cyclopentene, cyclohexene, cycloheptene, cyclooctene, cyclohexadiene, cycloheptadiene and cyclooctadiene, in which more preferred one is cyclohexene.

“Cycloalkene” at X is optionally substituted by 1 to 4 substituent(s). Suitable examples of such substituent include lower alkyl, lower alkoxy, hydroxy(lower)alkyl, lower alkoxy(lower)alkyl and halogen.

Suitable “unsaturated 5 or 6-membered heteromonocyclic group” includes 5 or 6-membered aromatic heteromonocyclic group containing 1 to 4 heteroatom(s) selected from sulfur, oxygen and nitrogen such as pyridinyl, N-oxidopyridinyl, pyrimidinyl, pyrazinyl, pyridazinyl, thiazolyl, isothiazolyl, thiadiazolyl, oxazolyl, isoxazolyl, imidazolyl, pyrazolyl, furyl, thienyl, pyrrolyl and dihydrofuranyl, in which more preferred ones are pyrimidinyl, thiazolyl, thienyl and dihydrofuranyl.

“Unsaturated 5 or 6-membered heteromonocyclic group” at X is optionally substituted by 1 to 4 substituent(s). Suitable examples of such substituent include lower alkyl, lower alkoxy, hydroxy(lower)alkyl, lower alkoxy(lower)alkyl and halogen, in which more preferred one is lower alkyl.

“Benzene” at X is substituted by 1 to 4 substituent(s). Suitable examples of such substituent include lower alkyl, lower alkoxy, hydroxy(lower)alkyl, lower alkoxy(lower)alkyl and halogen.

“Bivalent residue derived from the group consisting of cycloalkene, naphthalene, unsaturated 5 or 6-membered heteromonocyclic group, each of which is optionally substituted by substituent(s), and benzene which is substituted by substituent(s)” means a bivalent residue derived from the ring selected from “cycloalkene, naphthalene, unsaturated 5 or 6-membered heteromonocyclic group, each of which is optionally substituted by substituent(s), and benzene which is substituted by substituent(s)” by removal of two hydrogen atoms. Preferably, X is 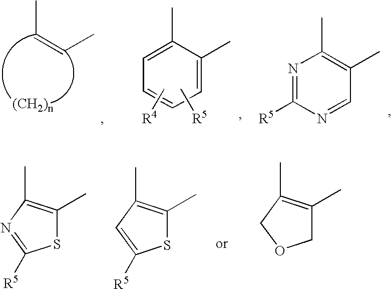
wherein R⁴ is lower alkyl, lower alkoxy, hydroxy(lower)alkyl, lower alkoxy(lower)alkyl or halogen,
R⁵ is hydrogen or lower alkyl, and
n is 3, 4, 5 or 6.

Suitable examples of “amino protective group” include acyl such as lower alkanoyl (e.g., formyl, acetyl, etc.), lower alkoxycarbonyl (e.g., tert-butoxycarbonyl, etc.), mono (or di or tri) phenyl(lower)alkoxycarbonyl (e. g., benzyloxycarbonyl, etc.), and a conventional protective group such as mono(or di or tri)aryl(lower)alkyl, for example, mono(or di or tri)phenyl(lower)alkyl (e.g., benzyl, trityl, etc.), lower alkylsulfonyl (e.g., methylsulfonyl, etc.), aryl(lower)alkylsulfonyl (e.g., benzylsulfonyl, etc.) and

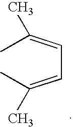

“Optionally protected amino” include amino and protected amino. Suitable examples of protected amino include lower alkanoylamino, lower alkylsulfonylamino, aryl(lower)alkylsulfonylamino, (lower)alkoxycarbonylamino, bis[(lower)alkylsulfonyl]amino, bis[aryl(lower)alkylsulfonyl]amino and

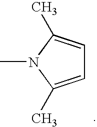

Suitable “lower alkanoyl” and “lower alkanoyl” moiety in the term “lower alkanoylamino” includes alkanoyl having 1 to 6 carbon atom(s) such as formyl, acetyl, propionyl, butyryl, isobutyryl, valeryl, isovaleryl, pivaloyl and hexanoyl, in which more preferred one is C₁-C₄ alkanoyl.

Suitable “(lower)alkoxycarbonyl” includes methoxycarbonyl, ethoxycarbonyl, propoxycarbonyl, isopropoxycarbonyl, butoxycarbonyl, isobutoxycarbonyl, sec-butoxycarbonyl, tert-butoxycarbonyl, pentyloxycarbonyl, tert-pentyloxycarbonyl and hexyloxycarbonyl, in which more preferred ones are methoxycarbonyl and tert-butoxycarbonyl.

Suitable “mono (or di or tri)phenyl(lower)alkoxycarbonyl” includes benzyloxycarbonyl and phenethyloxycarbonyl.

Suitable “aryl” and “aryl” moiety in the term “aryloxy” includes aryl having 6 to 10 carbon atoms which is optionally substituted by suitable substituent such as lower alkyl. “Aryl” includes fused carbocyclic group wherein benzene ring is fused with a saturated or unsaturated carbon ring. Suitable examples of aryl include phenyl, tolyl, naphthyl, indenyl and indanyl, in which more preferred ones are phenyl, tolyl and naphthyl.

Suitable “aryl” moiety in the terms “mono(or di or tri)aryl(lower)alkyl”, “aryl(lower)alkylsulfonyl”, “aryl(lower)alkylsulfonylamino”, “bis[aryl(lower)alkylsulfonyl]amino” and “arylsulfonyl” includes aryl having 6 to 10 carbon atoms which is optionally substituted by suitable substituent such as lower alkyl. Suitable examples of aryl moiety include phenyl, tolyl and naphthyl, in which more preferred ones are phenyl and tolyl.

Suitable “mono(or di or tri)aryl(lower)alkyl” include mono(or di or tri)phenyl(lower)alkyl such as benzyl, benzhydryl and trityl.

Suitable “lower alkylsulfonyl” include methylsulfonyl, ethylsulfonyl, propylsulfonyl, isopropylsulfonyl, butylsulfonyl, isobutylsulfonyl, sec-butylsulfonyl, tert-butylsulfonyl, pentylsulfonyl and hexylsulfonyl, in which more preferred one is methylsulfonyl.

Suitable “aryl(lower)alkylsulfonyl” include phenyl(lower)alkylsulfonyl such as benzylsulfonyl, phenethylsulfonyl and 1-phenylethylsulfonyl.

Suitable “lower alkanoylamino” includes formylamino, acetylamino, propionylamino, butyrylamino, isobutyrylamino, valerylamino, isovalerylamino, pivaloylamino and hexanoylamino, in which more preferred ones are formylamino and acetylamino.

Suitable “lower alkylsulfonylamino” includes methylsulfonylamino, ethylsulfonylamino, propylsulfonylamino, isopropylsulfonylamino, butylsulfonylamino, isobutylsulfonylamino, sec-butylsulfonylamino, tert-butylsulfonylamino, pentylsulfonylamino and hexylsulfonylamino, in which more preferred one is methylsulfonylamino.

Suitable “aryl(lower)alkylsulfonylamino” includes benzylsulfonylamino, phenylethylsulfonylamino and phenylpropylsulfonylamino, in which more preferred one is benzylsulfonylamino.

Suitable “(lower)alkoxycarbonylamino” includes methoxycarbonylamino, ethoxycarbonylamino, propoxycarbonylamino, isopropoxycarbonylamino, butoxycarbonylamino, isobutoxycarbonylamino, sec-butoxycarbonylamino, tert-butoxycarbonylamino, pentyloxycarbonylamino, tert-pentyloxycarbonylamino and hexyloxycarbonylamino, in which more preferred ones are methoxycarbonylamino and tert-butoxycarbonylamino.

Suitable “bis[(lower)alkylsulfonyl]amino” includes bis(methylsulfonyl)amino, bis(ethylsulfonyl)amino, bis(propylsulfonyl)amino, bis(isopropylsulfonyl)amino, bis(butylsulfonyl)amino, bis(isobutylsulfonyl)amino, bis(sec-butylsulfonyl)amino, bis(tert-butylsulfonyl)amino, bis(pentylsulfonyl)amino and bis(hexylsulfonyl)amino, in which more preferred one is bis(methylsulfonyl)amino.

Suitable “bis[aryl(lower)alkylsulfonyl]amino” includes bis(benzylsulfonyl)amino, bis(phenylethylsulfonyl)amino and bis(phenylpropylsulfonyl)amino, in which more preferred one is bis(benzylsulfonyl)amino.

Suitable “heteroaryl” includes 5 to 10-membered aromatic heteromonocyclic or fused heterocyclic group containing 1 to 4 heteroatom(s) selected from sulfur atom, oxygen atom and nitrogen atom. “Heteroaryl” includes fused heterocyclic group wherein benzene ring is fused with a saturated or unsaturated heterocyclic ring.

Suitable examples of “heteroaryl” include pyridinyl, pyrimidinyl, pyrazinyl, pyridazinyl, pyrrolyl, imidazolyl, pyrazolyl, triazolyl, tetrazolyl, thiazolyl, isothiazolyl, thiadiazolyl, oxazolyl, isoxazolyl, furyl, thienyl, indolyl, isoindolyl, indolizinyl, indazolyl, benzimidazolyl, benzotriazolyl, quinolyl, isoquinolyl, phthalazinyl, quinoxalinyl, quinazolinyl, cinnolinyl, benzofuranyl, benzothienyl, benzoxazolyl, benzothiazolyl, benzimidazolyl, indolinyl, isoindolinyl, tetrahydroquinolinyl and tetrahydroisoquinolinyl.

Suitable examples of “heteroaryl” at R² include pyridinyl, thiazolyl, pyrimidinyl, imidazolyl, pyrrolyl, triazolyl and indolyl, in which more preferred ones are pyridinyl and thiazolyl, and the most preferred one is pyridinyl.

Suitable “bivalent residue derived from aryl” includes C₆-C₁₀ arylene. “Bivalent residue derived from aryl” include bivalent fused carbocyclic group wherein benzene ring is fused with a saturated or unsaturated carbon ring.

Suitable examples of “bivalent residue derived from aryl” include phenylene, naphthylene, indenediyl and indandiyl, in which more preferred one is phenylene.

Suitable “bivalent residue derived from heteroaryl” includes bivalent 5 to 10-membered aromatic heteromonocyclic or fused heterocyclic group containing 1 to 4 heteroatom(s) selected from sulfur atom, oxygen atom and nitrogen atom. “Bivalent residue derived from heteroaryl” includes bivalent fused heterocyclic group wherein benzene ring is fused with a saturated or unsaturated heterocyclic ring.

Suitable examples of “bivalent residue derived from heteroaryl” include pyridinediyl, pyrimidinediyl, pyrazinediyl, pyridazinediyl, pyrrolediyl, imidazolediyl, pyrazolediyl, triazolediyl, tetrazolediyl, thiazolediyl, isothiazolediyl, thiadiazolediyl, oxazolediyl, isoxazolediyl, furandiyl, thiophenediyl, indolediyl, isoindolediyl, indolizinediyl, indazolediyl, benzimidazolediyl, benzotriazolediyl, quinolinediyl, isoquinolinediyl, phthalazinediyl, quinoxalinediyl, quinazolinediyl, cinnolinediyl; benzofurandiyl, benzothiophenediyl, benzoxazolediyl, benzothiazolediyl, benzimidazolediyl, indolinediyl, isoindolinediyl, tetrahydroquinolinediyl and tetrahydroisoquinolinediyl.

Suitable examples of “bivalent residue derived from heteroaryl” at ring A include pyridinediyl, indolinediyl, 1,2,3,4-tetrahydroisoquinolinediyl and isoindolinediyl.

Suitable “lower cycloalkyl” includes cycloalkyl having 3 to 8 carbon atoms, preferably 3 to 6 carbon atoms, more preferably 5 or 6 carbon atoms. Suitable examples of “lower cycloalkyl” include cyclopropyl, cyclobutyl, cyclopentyl, cyclohexyl, cycloheptyl and cyclooctyl, in which more preferred one is cyclopentyl and cyclohexyl.

“Bivalent residue derived from piperazine” means bivalent residue derived from piperazine by removal two hydrogen atoms, such as piperazine-1,4-diyl, piperazine-1,3-diyl, piperazine-1,2-diyl, piperazine-2,3-diyl and piperazine-2,5-diyl, in which more preferred one is piperazine-1,4-diyl.

Suitable “bivalent residue derived from piperazine substituted by lower alkyl” includes 3-methylpiperazine-1,4-diyl.

Suitable “optionally substituted heteroaryl” for substituent(s) at R² includes optionally substituted pyrrolyl, preferably pyrrolyl optionally substituted by 1 to 3 lower alkyl, in which more preferred one is 2,5-dimethyl-1H-pyrrol-1-yl.

Suitable examples of “carboxy protective group” include lower alkyl (e.g., methyl, ethyl, tert-butyl, etc.) and mono(or di or tri)phenyl(lower)alkyl optionally substituted by nitro (e.g., benzyl, 4-nitrobenzyl, benzhydryl, trityl, etc.).

“Optionally protected carboxy” include carboxy and protected carboxy. Suitable examples of protected carboxy include lower alkoxycarbonyl (e.g., methoxycarbonyl, ethoxycarbonyl, propoxycarbonyl, isopropoxycarbonyl, butoxycarbonyl, isobutoxycarbonyl, tert-butoxycarbonyl, etc.) and mono (or di or tri)phenyl(lower)alkoxycarbonyl optionally substituted by nitro (e.g., benzyloxycarbonyl, 4-nitrobenzyloxycarbonyl, benzhydryloxycarbonyl, trityloxycarbonyl, etc.).

The object compound (I) of the present invention can be prepared by the following processes. 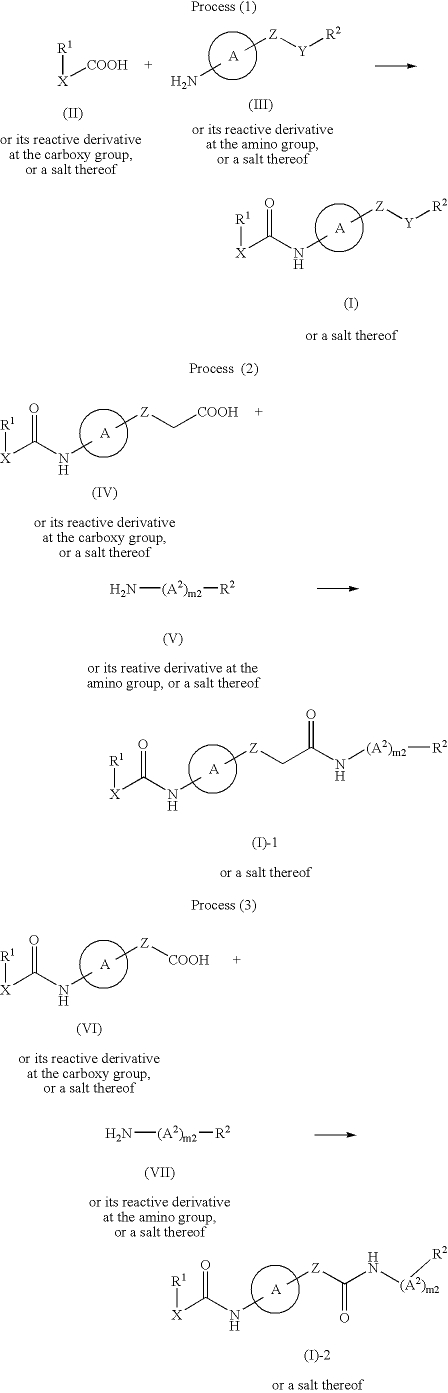 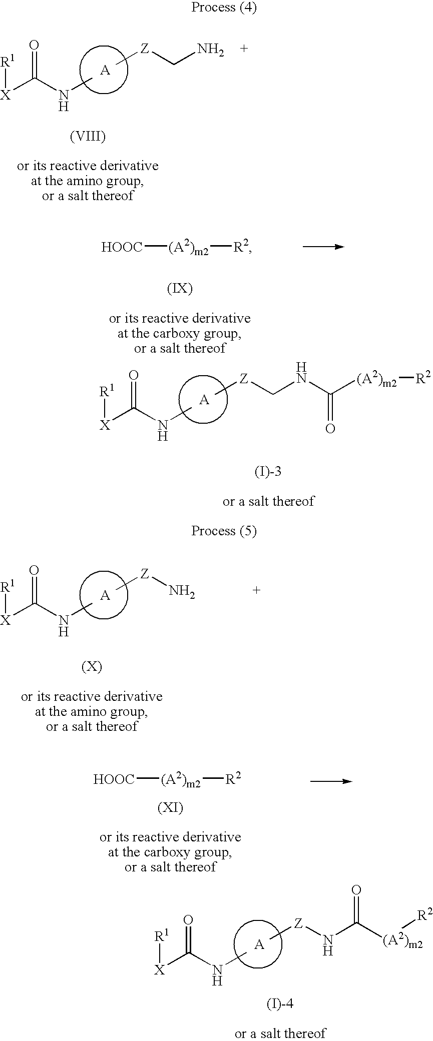 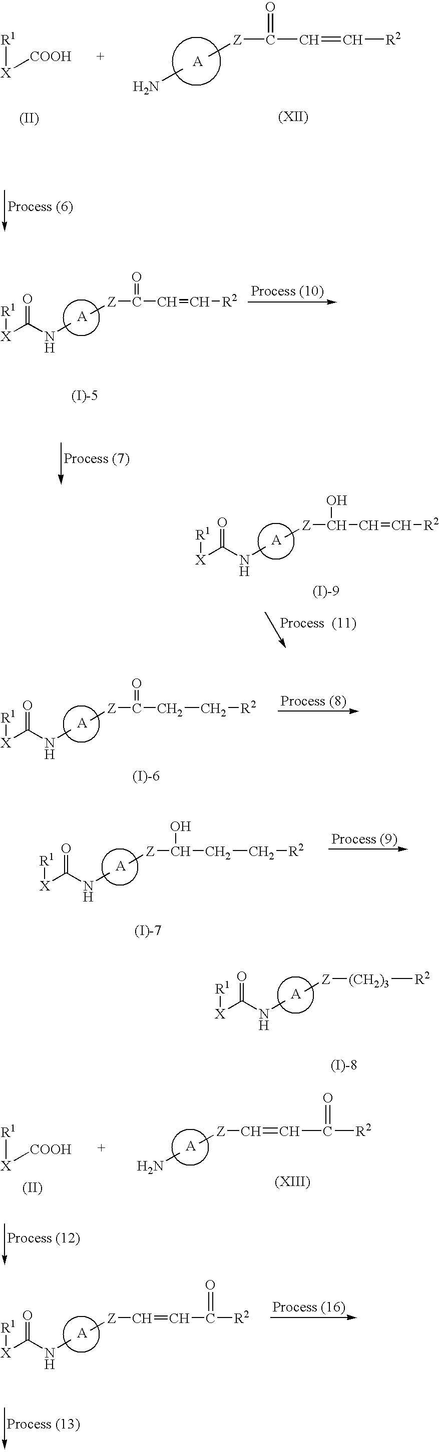 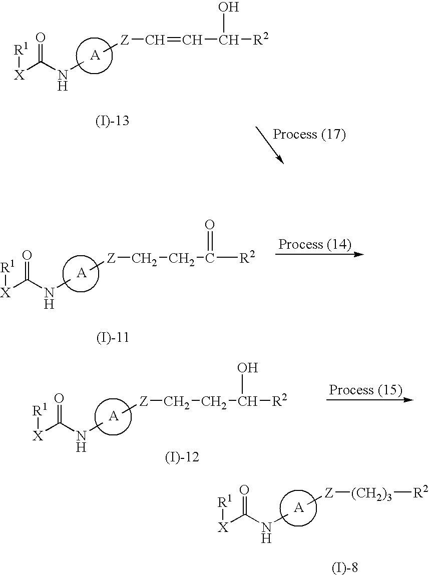 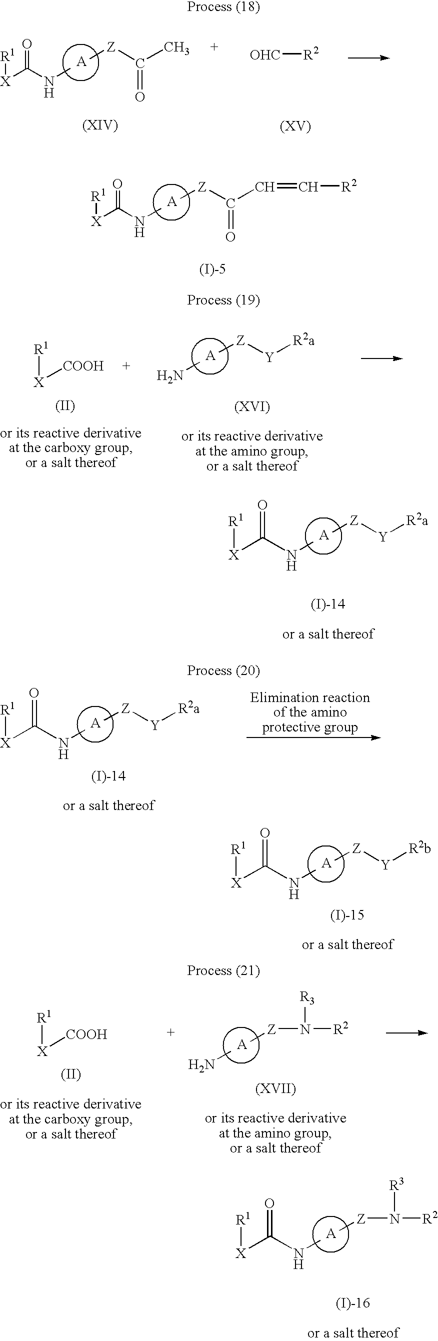 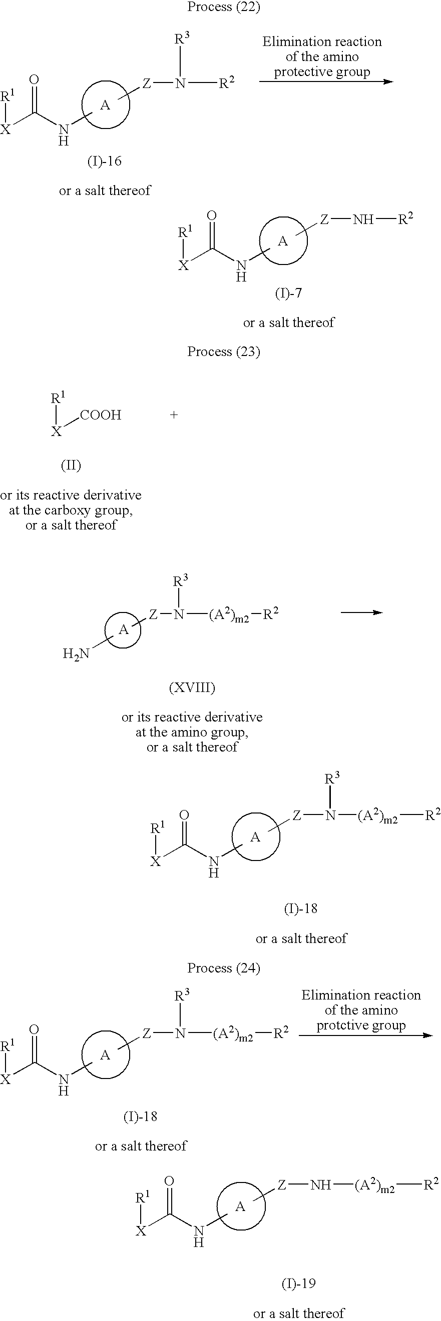 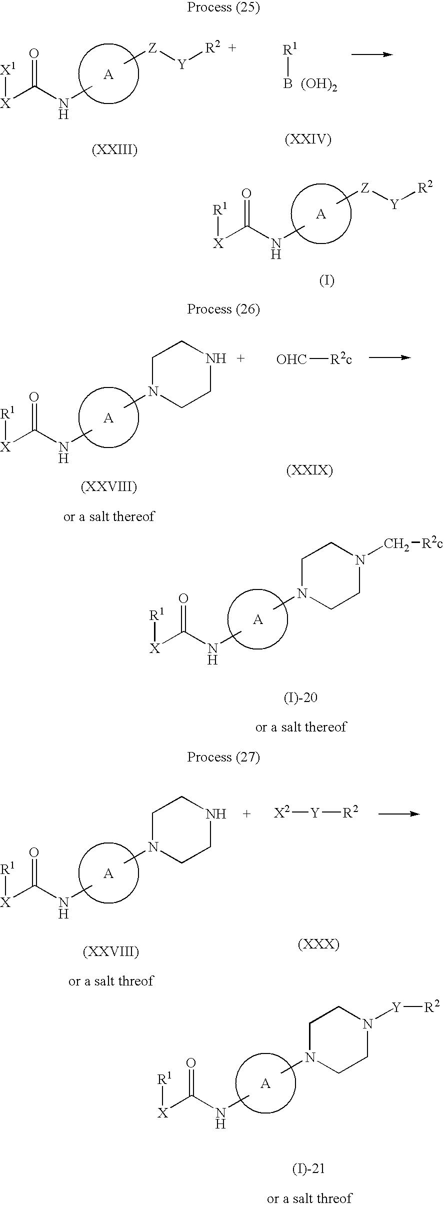
wherein R¹, R², R³, X, Y, Z, ring A, A² and m2 are as defined above,
R¹⁶ is amino protective group,
R²a is aryl, heteroaryl, lower cycloalkyl, aryloxy or arylsulfonyl, each of which is substituted by protected amino,
R²b is aryl, heteroaryl, lower cycloalkyl, aryloxy or arylsulfonyl, each of which is substituted by amino,
R²c is aryl, heteroaryl or lower cycloalkyl, each of which is optionally substituted by substituent(s), and
X¹ and X² are each leaving group.

The starting compounds can be prepared by the following processes or by the method of Preparation mentioned below or by a process known in the art for preparing their structurally analogous compounds. 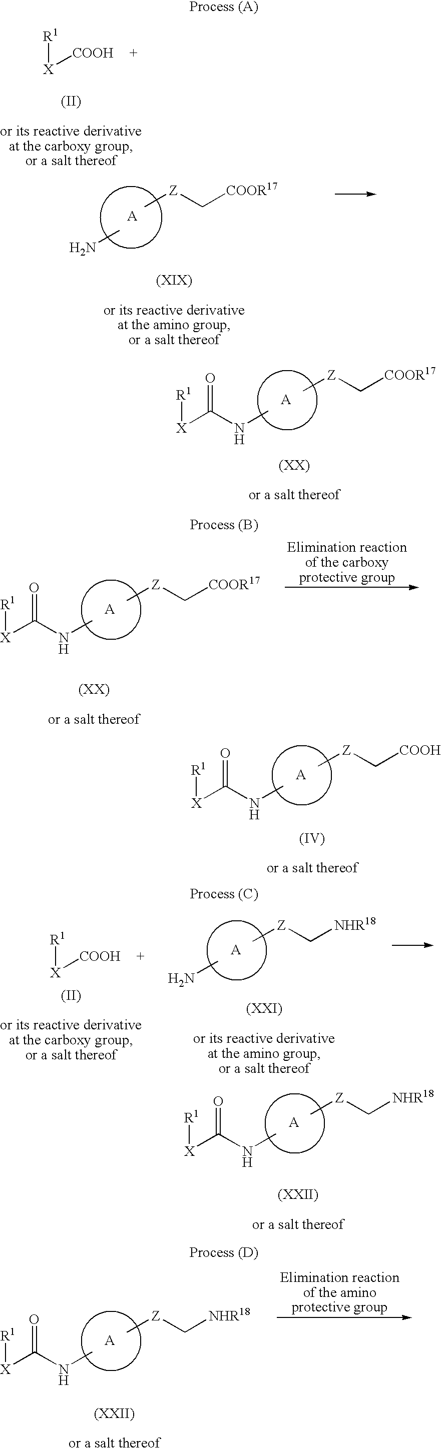 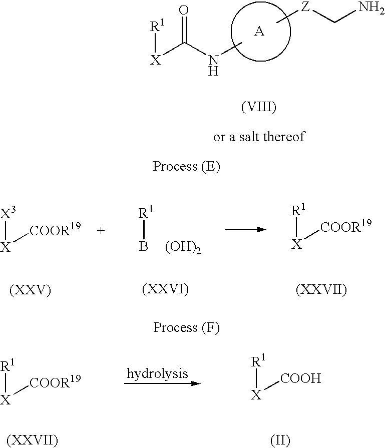
wherein R¹, X, Z and ring A are as defined above,
R¹⁷ and R¹⁹ are each carboxy protective group,
R¹⁸ is amino protective group, and
X³ is leaving group.

Suitable examples of a leaving group represented by X¹, X² and X³ include halogen (e.g., fluorine, bromine, chlorine and iodine), alkylsulfonyloxy group (e.g., trifluoromethanesulfonyloxy and methanesulfonyloxy) and arylsulfonyloxy group (e.g., p-toluenesulfonyloxy).

The processes for preparing the object and starting compounds are explained in detail in the following.

Process (1)

The compound (I) or a salt thereof can be prepared by reacting the compound (II) or its reactive derivative at the carboxy group, or a salt thereof with the compound (III) or its reactive derivative at the amino group, or a salt thereof.

Suitable reactive derivative of the compound (III) includes Schiff's base type imino or its tautomeric enamine type isomer formed by the reaction of the compound (III) with a carbonyl compound such as aldehyde, ketone or the like; a silyl derivative formed by the reaction of the compound (III) with a silyl compound such as N,O-bis(trimethylsilyl)acetamide, N-trimethylsilylacetamide or the like; a derivative formed by the reaction of the compound (III) with phosphorus trichloride or phosgene.

Suitable reactive derivative of the compound (II) includes an acid halide, an acid anhydride and an activated ester. The suitable example may be an acid chloride; an acid azide; a mixed acid anhydride with an acid such as substituted phosphoric acid (e.g., dialkylphosphoric acid, phenylphosphoric acid, diphenylphosphoric acid, dibenzylphosphoric acid, halogenated phosphoric acid, etc.), dialkylphosphorous acid, sulfurous acid, thiosulfuric acid, alkanesulfonic acid (e.g., methanesulfonic acid, ethanesulfonic acid, etc.), sulfuric acid, alkylcarbonic acid, aliphatic carboxylic acid (e.g., pivalic acid, pentanoic acid, isopentanoic acid, 2-ethylbutyric acid, trichloroacetic acid, etc.); aromatic carboxylic acid (e.g., benzoic acid, etc.); a symmetrical acid anhydride; an activated amide with imidazole, 4-substituted imidazole, dimethylpyrazole, triazole or tetrazole; an activated ester (e.g., cyanomethyl ester, methoxymethyl ester, dimethyliminomethyl [(CH₃)₂N⁺═CH—] ester, vinyl ester, propargyl ester, p-nitrophenyl ester, 2,4-dinitrophenyl ester, trichlorophenyl ester, pentachlorophenyl ester, mesylphenyl ester, phenylazophenyl ester, phenyl thioester, p-nitrophenyl thioester, p-cresyl thioester, carboxymethyl thioester, pyranyl ester, pyridinyl ester, piperidyl ester, 8-quinolyl thioester, etc.); or an ester with an N-hydroxy compound (e.g., N,N-dimethylhydroxylamine, 1-hydroxy-2-(1H)-pyridone, N-hydroxysuccinimide, N-hydroxybenzotriazole, N-hydroxyphthalimide, 1-hydroxy-6-chloro-1H-benzotriazole, etc.). These reactive derivatives can optionally be selected from them according to the kind of the compound (II) to be used.

The reaction is usually carried out in a conventional solvent such as water, acetone, dioxane, acetonitrile, chloroform, methylene chloride, ethylene dichloride, tetrahydrofuran, ethyl acetate, N,N-dimethylformamide, pyridine or any other organic solvents which do not adversely affect the reaction, or a mixture thereof.

When the compound (II) is used in free acid form or its salt form in the reaction, the reaction is preferably carried out in the presence of a conventional condensing agent such as N,N′-dicyclohexylcarbodiimide; N-cyclohexyl-N′-morpholinoethylcarbodiimide; N-cyclohexyl-N′-(4-diethylaminocyclohexyl)carbodiimide; N,N′-diisopropylcarbodiimide; N-ethyl-N′-(3-dimethylaminopropyl)carbodiimide; N,N-carbonyl-bis-(2-methylimidazole); pentamethyleneketene-N-cyclohexylimine; diphenylketene-N-cyclohexylimine; ethoxyacetylene; 1-alkoxy-1-chloroethylene; trialkyl phosphite; isopropyl polyphosphate; phosphorus oxychloride (phosphoryl chloride); phosphorus trichloride; thionyl chloride; oxalyl chloride; triphenylphosphine; 2-ethyl-7-hydroxybenzisoxazolium salt; 2-ethyl-5-(m-sulfophenyl)isoxazolium hydroxide intramolecular salt; 1-(p-chlorobenzenesulfonyloxy)-6-chloro-1H-benzotriazole; so-called Vilsmeier reagent prepared by the reaction of N,N-dimethylformamide with thionyl chloride, phosgene, phosphorus oxychloride, etc.; or the like.

The reaction may also be carried out in the presence of an organic or inorganic base such as an alkali metal bicarbonate, tri(lower) alkylamine, pyridine, N-(lower)alkylmorpholine, N,N-di(lower)alkylbenzylamine, or the like.

The reaction temperature is not critical, and the reaction is usually carried out under cooling to heating.

Process (2)

The compound (I)-1 or a salt thereof can be prepared by reacting the compound (IV) or its reactive derivative at the carboxy group, or a salt thereof with the compound (V) or its reactive derivative at the amino group, or a salt thereof.

This reaction can be carried out in the same manner as in the aforementioned Process (1), and therefore the reagents to be used and the reaction conditions (e.g., solvent, reaction temperature, etc.) can be referred to those of Process (1).

Process (3)

The compound (I)-2 or a salt thereof can be prepared by reacting the compound (VI) or its reactive derivative at the carboxy group, or a salt thereof with the compound (VII) or its reactive derivative at the amino group, or a salt thereof.

This reaction can be carried out in the same manner as in the aforementioned Process (1), and therefore the reagents to be used and the reaction conditions (e.g., solvent, reaction temperature, etc.) can be referred to those of Process (1).

Process (4)

The compound (I)-3 or a salt thereof can be prepared by reacting the compound (VIII) or its reactive derivative at the amino group, or a salt thereof with the compound (IX) or its reactive derivative at the carboxy group, or a salt thereof.

This reaction can be carried out in the same manner as in the aforementioned Process (1), and therefore the reagents to be used and the reaction conditions (e.g., solvent, reaction temperature, etc.) can be referred to those of Process (1).

Process (5)

The compound (I)-4 or a salt thereof can be prepared by reacting the compound (X) or its reactive derivative at the amino group, or a salt thereof with the compound (XI) or its reactive derivative at the carboxy group, or a salt thereof.

This reaction can be carried out in the same manner as in the aforementioned Process (1), and therefore the reagents to be used and the reaction conditions (e.g., solvent, reaction temperature, etc.) can be referred to those of Process (1).

Process (6)

The compound (I)-5 or a salt thereof can be prepared by reacting the compound (II) or its reactive derivative at the carboxy group, or a salt thereof with the compound (XII) or its reactive derivative at the amino group, or a salt thereof.

This reaction can be carried out in the same manner as in the aforementioned Process (1), and therefore the reagents to be used and the reaction conditions (e.g., solvent, reaction temperature, ,etc.) can be referred to those of Process (1).

Process (7)

The compound (I)-6 can be prepared by subjecting the compound (I)-5 to catalytic hydrogenation.

Suitable catalysts to be used in the catalytic hydrogenation are conventional ones such as platinum catalysts (e.g., platinum plate, spongy platinum, platinum black, colloidal platinum, platinum oxide, platinum wire, etc.), palladium catalysts (e.g., spongy palladium, palladium black, palladium oxide, palladium on carbon, palladium hydroxide on carbon, colloidal palladium, palladium on barium sulfate, palladium on barium carbonate, etc.), and the like.

The hydrogenation is usually carried out in a conventional solvent such as water, alcohol (e.g., methanol, ethanol, isopropyl alcohol, etc.), tetrahydrofuran, dioxane, toluene, methylene chloride, ethylene dichloride, chloroform, N,N-dimethylformamide, N,N-dimethylacetamide or any other organic solvents which do not adversely affect the reaction, or a mixture thereof.

The reaction temperature is not critical, and the reaction is usually carried out under cooling to warming.

Process (8)

The compound (I)-7 can be prepared by subjecting the compound (I)-6 to reduction using a suitable reducing agent.

Suitable reducing agents to be used in the reduction are hydrides (e.g., sodium borohydride, sodium cyanoborohydride, lithium aluminum hydride, etc.).

The reduction is usually carried out in a conventional solvent such as water, alcohol (e.g., methanol, ethanol, isopropyl alcohol, etc.), tetrahydrofuran, dioxane, toluene, methylene chloride, ethylene dichloride, chloroform, N,N-dimethylformamide, N,N-dimethylacetamide or any other organic solvents which do not adversely affect the reaction, or a mixture thereof.

The reaction temperature is not critical, and the reaction is usually carried out under cooling to warming.

Process (9)

The compound (I)-8 can be prepared by subjecting the compound (I)-7 to catalytic hydrogenation in the presence of an acid.

Suitable catalysts to be used in the catalytic hydrogenation are conventional ones such as platinum catalysts (e.g., platinum plate, spongy platinum, platinum black, colloidal platinum, platinum oxide, platinum wire, etc.), palladium catalysts (e.g., spongy palladium, palladium black, palladium oxide, palladium on carbon, palladium hydroxide on carbon, colloidal palladium, palladium on barium sulfate, palladium on barium carbonate, etc.), and the like.

Suitable acid to be used in the catalytic hydrogenation includes hydrochloric acid, hydrogen chloride, and the like.

The hydrogenation is usually carried out in a conventional solvent such as water, alcohol (e.g., methanol, ethanol, isopropyl alcohol, etc.), tetrahydrofuran, dioxane, toluene, methylene chloride, ethylene dichloride, chloroform, N,N-dimethylformamide, N,N-dimethylacetamide or any other organic solvents which do not adversely affect the reaction, or a mixture thereof.

The reaction temperature is not critical, and the reaction is usually carried out under cooling to warming.

Process (10)

The compound (I)-9 can be prepared by subjecting the compound (I)-5 to reduction using a suitable reducing agent.

This reaction can be carried out in the same manner as in the aforementioned Process (8), and therefore the reagents to be used and the reaction conditions (e.g., solvent, reaction temperature, etc.) can be referred to those of Process (8).

Process (11)

The compound (I)-8 can be prepared by subjecting the compound (I)-9 to catalytic hydrogenation in the presence of an acid.

This reaction can be carried out in the same manner as in the aforementioned Process (9), and therefore the reagents to be used and the reaction conditions (e.g., solvent, reaction temperature, etc.) can be referred to those of Process (9).

Process (12).

The compound (I)-10 or a salt thereof can be prepared by reacting the compound (II) or its reactive derivative at the carboxy group, or a salt thereof with the compound (XIII) or its reactive derivative at the amino group, or a salt thereof.

This reaction can be carried out in the same manner as in the aforementioned Process (1), and therefore the reagents to be used and the reaction conditions (e.g., solvent, reaction temperature, etc.) can be referred to those of Process (1).

Process (13)

The compound (I)-11 can be prepared by subjecting the compound (I)-10 to catalytic hydrogenation.

This reaction can be carried out in the same manner as in the aforementioned Process (7), and therefore the reagents to be used and the reaction conditions (e.g., solvent, reaction temperature, etc.) can be referred to those of Process (7).

Process (14)

The compound (I)-12 can be prepared by subjecting the compound (I)-11 to reduction using a suitable reducing agent.

This reaction can be carried out in the same manner as in the aforementioned Process (8), and therefore the reagents to be used and the reaction conditions (e.g., solvent, reaction temperature, etc.) can be referred to those of Process (8).

Process (15)

The compound (I)-8 can be prepared by subjecting the compound (I)-12 to catalytic hydrogenation in the presence of an acid.

This reaction can be carried out in the same manner as in the aforementioned Process (9), and therefore the reagents to be used and the reaction conditions (e.g., solvent, reaction temperature, etc.) can be referred to those of Process (9).

Process (16)

The compound (I)-13 can be prepared by subjecting the compound (I)-10 to reduction using a suitable reducing agent.

This reaction can be carried out in the same manner as in the aforementioned Process (8), and therefore the reagents to be used and the reaction conditions (e.g., solvent, reaction temperature, etc.) can be referred to those of Process (8).

Process (17)

The compound (I)-8 can be prepared by subjecting the compound (I)-13 to catalytic hydrogenation in the presence of an acid.

This reaction can be carried out in the same manner as in the aforementioned Process (9), and therefore the reagents to be used and the reaction conditions (e.g., solvent, reaction temperature, etc.) can be referred to those of Process (9).

Process (18)

The compound (I)-5 can be prepared by reacting the compound (XIV) with the compound (XV) in the presence of a base or an acid.

Suitable base to be used in the reaction includes an inorganic base and an organic base such as alkali metal hydroxide (e.g., sodium hydroxide, potassium hydroxide, etc.), alkaline earth metal hydroxide (e.g., magnesium hydroxide, calcium hydroxide, barium hydroxide, etc.), alkali metal carbonate (e.g., sodium carbonate, potassium carbonate, etc.), alkaline earth metal carbonate (e.g., magnesium carbonate, calcium carbonate, barium carbonate, etc.), alkoxide (e.g., sodium methoxide, sodium ethoxide, etc.), trialkylamine (e.g., trimethylamine, triethylamine, etc.), and the like.

Suitable acid to be used in the reaction includes hydrochloric acid, hydrobromic acid, hydrogen chloride, hydrogen bromide, and the like.

This reaction is usually carried out in a conventional solvent such as water, alcohol (e.g., methanol, ethanol, isopropyl alcohol, etc.), tetrahydrofuran, dioxane, toluene, methylene chloride, ethylene dichloride, chloroform, or any other organic solvents which do not adversely affect the reaction, or a mixture thereof.

The reaction temperature is not critical, and the reaction is usually carried out under cooling to heating.

Process (19)

The compound (I)-14 or a salt thereof can be prepared by reacting the compound (II) or its reactive derivative at the carboxy group, or a salt thereof with the compound (XVI) or its reactive derivative at the amino group, or a salt thereof.

This reaction can be carried out in the same manner as in the aforementioned Process (1), and therefore the reagents to be used and the reaction conditions (e.g., solvent, reaction temperature, etc.) can be referred to those of Process (1).

Process (20)

The compound (I)-15 or a salt thereof can be prepared by subjecting the compound (I)-14 or a salt thereof to elimination reaction of the amino protective group.

Suitable method of this elimination reaction includes conventional one such as hydrolysis, reduction and the like.

(i) For Hydrolysis:

The hydrolysis is preferably carried out in the presence of a base or an acid including Lewis acid.

Suitable base includes an inorganic base and an organic base such as an alkali metal [e.g., sodium, potassium, etc.], an alkaline earth metal [e.g., magnesium, calcium, etc.], the hydroxide or carbonate or hydrogencarbonate thereof, trialkylamine [e.g., trimethylamine, triethylamine, etc.], picoline, 1,5-diazabicyclo[4.3.0]non-5-ene, or the like.

Suitable acid includes an organic acid [e.g., formic acid, acetic acid, propionic acid, trichloroacetic acid, trifluoroacetic acid, etc.], and an inorganic acid [e.g., hydrochloric acid, hydrobromic acid, sulfuric acid, hydrogen chloride, hydrogen bromide, etc.].

The elimination using Lewis acid such as trihaloacetic acid [e.g., trichloroacetic acid, trifluoroacetic acid, etc.], or the like is preferably carried out in the presence of cation trapping agents [e.g., anisole, phenol, etc.]. This reaction is usually carried out without solvent.

The reaction may be carried out in a conventional solvent such as water, alcohol (e.g., methanol, ethanol, isopropyl alcohol, etc.), tetrahydrofuran, dioxane, toluene, methylene chloride, ethylene dichloride, chloroform, N,N-dimethylformamide, N,N-dimethylacetamide or any other organic solvents which do not adversely affect the reaction, or a mixture thereof.

The reaction temperature is not critical and the reaction is usually carried out under cooling to warming.

(ii) For Reduction:

Reduction is carried out in a conventional manner, including chemical reduction and catalytic reduction.

Suitable reducing reagent to be used in chemical reduction are hydrides (e.g., hydrogen iodide, hydrogen sulfide, lithium aluminum hydride, sodium borohydride, sodium cyanoborohydride, etc.), or a combination of a metal (e.g., tin, zinc, iron, etc.) or metallic compound (e.g., chromium chloride, chromium acetate, etc.) and an organic acid or inorganic acid (e.g., formic acid, acetic acid, propionic acid, trifluoroacetic acid, p-toluenesulfonic acid, hydrochloric acid, hydrobromic acid, etc.).

Suitable catalysts to be used in catalytic reduction are conventional ones such as platinum catalysts (e.g., platinum plate, spongy platinum, platinum black, colloidal platinum, platinum oxides platinum wire, etc.), palladium catalysts (e.g., spongy palladium, palladium black, palladium oxide, palladium on carbon, palladium hydroxide on carbon, colloidal palladium, palladium on barium sulfate, palladium on barium carbonate, etc.), nickel catalysts (e.g., reduced nickel, nickel oxide, Raney nickel, etc.), cobalt catalysts (e.g., reduced cobalt, Raney cobalt, etc.), iron catalysts (e.g., reduced iron, Raney iron, Ullman iron, etc.), and the like.

The reduction is usually carried out in a conventional solvent such as water, alcohol (e.g., methanol, ethanol, isopropyl alcohol, etc.), tetrahydrofuran, dioxane, toluene, methylene chloride, ethylene dichloride, chloroform, N,N-dimethylformamide, N,N-dimethylacetamide or any other organic solvents which do not adversely affect the reaction, or a mixture thereof.

Additionally, in case that the above-mentioned acids to be used in chemical reduction are in a liquid state, they can also be used as a solvent.

The reaction temperature of this reduction is not critical and the reaction is usually carried out under cooling to warming.

Process (21)

The compound (I)-16 or a salt thereof can be prepared by reacting the compound (II) or its reactive derivative at the carboxy group, or a salt thereof with the compound (XVII) or its reactive derivative at the amino group, or a salt thereof.

This reaction can be carried out in the same manner as in the aforementioned Process (1), and therefore the reagents to be used and the reaction conditions (e.g., solvent, reaction temperature, etc.) can be referred to those of Process (1).

Process (22)

The compound (I)-17 or a salt thereof can be prepared by subjecting the compound (I)-16 or a salt thereof to elimination reaction of the amino protective group.

This reaction can be carried out in the same manner as in the aforementioned Process (20), and therefore the reagents to be used and the reaction conditions (e.g., solvent, reaction temperature, etc.) can be referred to those of Process (20).

Process (23)

The compound (I)-18 or a salt thereof can be prepared by reacting the compound (II) or its reactive derivative at the carboxy group, or a salt thereof with the compound (XVIII) or its reactive derivative at the amino group, or a salt thereof.

This reaction can be carried out in the same manner as in the aforementioned Process (1), and therefore the reagents to be used and the reaction conditions (e.g., solvent, reaction temperature, etc.) can be referred to those of Process (1).

Process (24)

The compound (I)-19 or a salt thereof can be prepared by subjecting the compound (I)-18 or a salt thereof to elimination reaction of the amino protective group.

This reaction can be carried out in the same manner as in the aforementioned Process (20), and therefore the reagents to be used and the reaction conditions (e.g., solvent, reaction temperature, etc.) can be referred to those of Process (20).

Process (25)

The compound (I) can be prepared by reacting the compound (XXIII) and the compound (XXIV) in the presence of tetrakis(triphenylphosphine)palladium and a base such as triethylamine.

This reaction can be carried out in a solvent such as N,N-dimethylformamide which does not adversely affect the reaction. The reaction temperature is not critical and the reaction is usually carried out under cooling to heating.

Process (26)

The compound (I)-20 or a salt thereof can be prepared by reacting the compound (XXVIII) or a salt thereof with the compound (XXIX) in the presence of a reducing agent. Suitable reducing agent to be used in the reaction includes sodium triacetoxyborohydride, and the like.

This reaction is usually carried out in a conventional solvent such as methylene chloride, ethylene dichloride, chloroform, tetrahydrofuran, dioxane or any other organic solvents which do not adversely affect the reaction, or a mixture thereof.

The reaction temperature is not critical, and the reaction is usually carried out under cooling to heating.

Process (27)

The compound (I)-21 or a salt thereof can be prepared by reacting the compound (XXVIII) or a salt thereof and the compound (XXX) in the presence of a base.

Suitable base to be used in the reaction includes an inorganic base and an organic base such as alkali metal hydroxide (e.g., sodium hydroxide, potassium hydroxide, etc.), alkaline earth metal hydroxide (e.g., magnesium hydroxide, calcium hydroxide, barium hydroxide, etc.), alkali metal carbonate (e.g., sodium carbonate, potassium carbonate, cesium carbonate, etc.), alkaline earth metal carbonate (e.g., magnesium carbonate, calcium carbonate, barium carbonate, etc.), alkoxide (e.g., sodium methoxide, sodium ethoxide, etc.), trialkylamine (e.g., trimethylamine, triethylamine, etc.), and the like.

This reaction is usually carried out in a conventional solvent such as water, alcohol (e.g., methanol, ethanol, isopropyl alcohol, etc.), acetone, tetrahydrofuran, dioxane, toluene, methylene chloride, ethylene dichloride, chloroform, or any other organic solvents which do not adversely affect. the reaction, or a mixture thereof.

The reaction temperature is not critical and the reaction is usually carried out under cooling to heating.

Process (A)

The compound (XX) or a salt thereof can be prepared by reacting the compound (II) or its reactive derivative at the carboxy group, or a salt thereof with the compound (XIX) or its reactive derivative at the amino group, or a salt thereof.

This reaction can be carried out in the same manner as in the aforementioned Process (I), and therefore the reagents to be used and the reaction conditions (e.g., solvent, reaction temperature, etc.) can be referred to those of Process (1).

Process (B)

The compound (IV) or a salt thereof can be prepared by subjecting the compound (XX) or a salt thereof to elimination reaction of the carboxy protective group.

Suitable method of this elimination reaction includes conventional one such as hydrolysis.

The hydrolysis can be carried out in the same manner as in the aforementioned Process (20), and therefore the reagents to be used and the reaction conditions (e.g., solvent, reaction temperature, etc.) can be referred to those of Process (20).

Process (C)

The compound (XXII) or a salt thereof can be prepared by reacting the compound (II) or its reactive derivative at the carboxy group, or a salt thereof with the compound (XXI) or its reactive derivative at the amino group, or a salt thereof.

This reaction can be carried out in the same manner as in the aforementioned Process (1), and therefore the reagents to be used and the reaction conditions (e.g., solvent, reaction temperature, etc.) can be referred to those of Process (1).

Process (D)

The compound (VIII) or a salt thereof can be prepared by subjecting the compound (XXII) or a salt thereof to elimination reaction of the amino protective group.

This reaction can be carried out in the same manner as in the aforementioned Process (20), and therefore the reagents to be used and the reaction conditions (e.g., solvent, reaction temperature, etc.) can be referred to those of Process (20).

Process (E)

The compound (XXVII) can be prepared by reacting the compound (XXV) and the compound (XXVI) in the presence of lithium chloride, tetrakis(triphenylphosphine)palladium(0) and a base such as sodium carbonate.

This reaction can be carried out in a solvent such as a mixture of toluene and water. The reaction temperature is not critical and the reaction is usually carried out under cooling to heating.

This reaction can be carried out in a similar manner as in Preparation 18 mentioned below.

Process (F)

The compound (II) can be prepared by subjecting the compound (XXVII) to elimination reaction of the carboxy protective group.

Suitable method of this elimination reaction includes conventional one such as hydrolysis.

The hydrolysis can be carried out in the same manner as in the aforementioned Process (20), and therefore the reagents to be used and the reaction conditions (e.g., solvent, reaction temperature, etc.) can be referred to those of Process (20).

Suitable salts of the starting compounds and their reactive derivatives in Processes (1) to (27) and (A) to (F) can be referred to the ones as exemplified for the compound (I).

The compounds obtained by the above processes can be isolated and purified by a conventional method such as pulverization, recrystallization, column chromatography, reprecipitation, or the like.

It is to be noted that the compound (I) and the other compounds may include one or more stereoisomer(s) such as optical isomer(s) and geometrical isomer(s) due to asymmetric carbon atom(s) and double bond(s), and all of such isomers and mixtures thereof are included within the scope of this invention.

The object compounds (I) and pharmaceutically acceptable salts thereof include solvates [e.g., enclosure compounds (e.g., hydrate, etc.)].

The object compounds (I) and pharmaceutically acceptable salts thereof possess a strong inhibitory activity on the secretion of Apo B.

Accordingly, the object compounds (I) and pharmaceutically acceptable salts thereof are useful as an Apo B secretion inhibitor.

The object compounds (I) and pharmaceutically acceptable salts thereof are useful as a medicament for the prophylaxis or treatment of diseases or conditions resulting from elevated circulating levels of Apo B such as hyperlipemia, hyperlipidemia, hyperlipoproteinemia, hypoalphalipoproteinemia, hypercholesterolemia, hypertriglyceridemia, atherosclerosis, pancreatitis, non-insulin dependent diabetes mellitus (NIDDM), obesity, coronary heart diseases, myocardial infarction, stroke, restenosis and Syndrome X.

The present invention therefore provides a method for inhibiting or decreasing Apo B secretion in a mammal, in particular in human, which comprises administering an Apo B secretion inhibiting or decreasing amount of a compound of formula (I) or a pharmaceutically acceptable salt thereof to the mammal.

The present invention also provides a method for preventing or treating diseases or conditions resulting from elevated circulating levels of Apo B in a mammal, in particular in human, which comprises administering an effective amount of a compound of formula (I) or a pharmaceutically acceptable salt thereof to the mammal.

The object compounds (I) and pharmaceutical acceptable salts thereof are also useful in reducing intestinal fat absorption and reducing food intake for the prophylaxis or treatment of obesity. Furthermore, the object compounds (I) and pharmaceutical acceptable salts thereof possess an inhibitory activity on the lipid transfer of microsomal triglyceride transfer protein (MTP).

In order to illustrate the usefulness of the object compound (I), the pharmacological test result of the compound (I) is shown in the following.

Test Compounds:

4′-chloro-4-methyl-N-(4-{[2-(2-pyridinyl)ethyl]amino}phenyl)-1,1′-biphenyl-2-carboxamide (Example 31)
5-methyl-N-(4-{[2-(2-pyridinyl)ethyl]amino}phenyl)-4′-(trifluoromethyl)-1,1′-biphenyl-2-carboxamide (Example 44)
4′-fluoro-5-methyl-N-(4-{[2-(2-pyridinyl)ethyl]amino}phenyl)-1,1′-biphenyl-2-carboxamide (Example 46)
N-(4-{[2-(6-amino-2-pyridinyl)ethyl]amino}phenyl)-4,4′-dimethyl-1,1′-biphenyl-2-carboxamide (Example 53)
N-(4-{[2-(6-amino-2-pyridinyl)ethyl]amino}phenyl)-4′-chloro-4-methyl-1,1′-biphenyl-2-carboxamide (Example-55)
N-(4-{[2-(6-amino-2-pyridinyl)ethyl]amino}phenyl)-4′-fluoro-4-methyl-1,1′-biphenyl-2-carboxamide (Example 56)
N-{6-[4-(3-cyanobenzyl)-1-piperazinyl]-3-pyridinyl}-2-[4-(trifluoromethyl)phenyl]-1-cyclohexene-1-carboxamide (Example 212)
N-(4-{[2-(2-pyridinyl)ethyl]amino}phenyl)-2-[4-(trifluoromethyl)phenyl]-1-cycloheptene-1-carboxamide (Example 232)
   Test 1: Measurement of Inhibition of Apo B Secretion

HepG2 cells were seeded in Eagles medium containing 10% fetal calf serum (FCS) at a density of 30000 cells/well in 96-well plates and allowed to grow for 3 days before treatment. At this time, the medium was replaced with fresh medium containing 0.1% dimethyl sulfoxide (DMSO) and the indicated concentrations of a test compound. After 15-hour incubation, the amount of Apo B and Apo AI accumulated in the media was determined by ELISA.

The assay was carried out at ambient temperature. A flat bottomed micro ELISA plate (manufactured by Nunc) was coated with an anti Apo B monoclonal antibody solution (5 mg/ml in 0.05% carbonate buffer, pH 9.6).by adding the antibody solution at a volume of 100 μl per well. After 1-hour incubation on a plate mixer, the unbound materials were removed by washing the well 3 times with a washing buffer (phosphate buffered saline, pH 7.2 containing 0.1% bovine serum albumin and 0.05% Tween-20). Then 20 μl of a solution of the test compound (dissolved in the culture medium) and 100 μl of a solution of peroxidase coupled anti Apo B antibody were added. After 1-hour incubation on a plate mixer, washing was performed 3 times to remove the unbound materials. A freshly prepared substrate solution (2.5 mg/ml ortho-phenylene diamine and 0.018% H₂O₂ in 0.11 M Na₂HPO₄-0.044 M sodium citrate buffer, pH 5.4) at a volume of 200 μl was then added to each well. After 20-minute incubation, the enzyme reaction was terminated by adding 50 μl of 0.5 M sulfuric acid. Absorbance of each well was determined at 490 nm using a microplate reader. Apo B concentration was calculated from a standard curve generated from purified Apo B standard that was run in parallel in the same plate. Inhibition of Apo B secretion by the test compound is calculated taking 0.1% DMSO treated cells as controls.

Measurement of Apo AI was performed similar to that of Apo B, except for diluting the sample 11-fold with a dilution buffer (phosphate buffered saline, pH 7.2 containing 0.5% bovine serum albumin and 0.05% Tween-20).

Apo B secretion inhibitors are identified as compounds that decrease Apo B secretion without affecting the secretion of Apo AI.

Test Results:

TABLE 1

| | Inhibition of Apo B |
|---|---|
| Test compound | secretion at 10⁻⁸ M |
| (Example No.) | (%) |
| 31 | 96 |
| 44 | 83 |
| 46 | 94 |
| 53 | 84 |
| 55 | 86 |
| 56 | 93 |
| 212 | 84 |
| 232 | 93 |

Test 2: Lipids Lowering Effect on ddY-Mice

Male ddY-mice were housed in temperature- and humidity-controlled rooms and fed with laboratory chow. The animals were randomized according to their body weight and deprived of food just before the experiment. A blood sample (baseline blood sample) was collected from the retro orbital venous plexus before administration of the test drug, and then the animals were orally dosed with the test drug in a vehicle (aqueous solution of 0.5% methylcellulose). Blood samples were drawn at 2 hours after drug administration for the measurement of cholesterol and triglyceride.

Plasma total-cholesterol and plasma triglyceride were determined by conventional enzyme methods using commercially available kits. The cholesterol CII-Test Wako (Wako Pure Chemical Industries, Ltd.) was used for the measurement of cholesterol, and the triglyceride E-test Wako (Wako Pure Chemical Industries, Ltd.) was used for the measurement of triglyceride.

Lipids lowering effects were shown in percent relative to the baseline level (level at 0 hr).

Test Results:

TABLE 2

| | | Cholesterol | Triglyceride |
|---|---|---|---|
| Test compound | Dose | (% of 0 hr) | (% of 0 hr) |
| (Example No.) | (mg/kg) | 2 hr | 2 hr |
| 31 | 3.2 | 86 | 36 |
| 44 | 3.2 | 80 | 25 |
| 46 | 3.2 | 79 | 19 |
| 53 | 3.2 | 71 | 17 |
| 55 | 3.2 | 75 | 16 |
| 56 | 3.2 | 81 | 31 |

Test 3: Lipid Lowering Effect on ddY-Mice

Male ddY-mice were housed in temperature- and humidity-controlled rooms and fed with laboratory chow. The animals were randomized according to their body weight and food was deprived about 16 hours before experiment. Baseline blood sample was collected from the retro orbital venous plexus then the animals were orally dosed with drugs in olive oil (10 ml/kg). For control group, 10 ml/kg of olive oil was loaded orally. Blood samples were drawn at 2 hours after drug administration for the measurement of triglyceride (TG) elevation. Plasma TG was determined by conventional enzyme method (The triglyceride E-test Wako).

Lipid lowering effects were shown in percent of the TG increase in drug treated group, relative to the TG increase in control group.
Lipid lowering effect (%)=(TG increase in drug treated group/TG increase in control group)×100

TABLE 3

| Test compound | Dose | Lipid lowering |
|---|---|---|
| (Example No.) | (mg/kg) | effect (%) |
| 212 | 0.32 | 55 |
| 232 | 0.32 | 29 |

For therapeutic administration, the object compound (I) of the present invention and pharmaceutically acceptable salts thereof are used in the form of a conventional pharmaceutical preparation in admixture with a conventional pharmaceutically acceptable carrier such as an organic or inorganic solid or liquid excipient which is suitable for oral, parenteral or external administration. The pharmaceutical preparation may be compounded in a solid form such as granule, capsule, tablet, dragee, suppository or ointment, or in a liquid form such as solution, suspension or emulsion for injection, intravenous drip, ingestion, eye drop, endermism, inhalation, etc. If needed, there may be included in the above preparation auxiliary substance such as stabilizing agent, wetting or emulsifying agent, buffer or any other commonly used additives.

The effective ingredient may usually be administered in a unit dose of 0.01 mg/kg to 100 mg/kg, preferably 0.1 mg/kg to 10 mg/kg, 1 to 4 times a day. However, the above dosage may be increased or decreased according to age, body weight and conditions of the patient or administering method.

Suitable mammal to which the object compounds (I) and pharmaceutical acceptable salts thereof or above preparations are applied, includes a human being, a companion animal such as a dog and a cat, livestock such as a cow and a pig, and the like.

The object compounds (I) and pharmaceutical acceptable salts thereof may, if desired, be administered with one or more therapeutic agents and formulated for administration by any convenient route in a conventional manner. Appropriate doses will be readily appreciated by those skilled in the art. For example, the object compounds (I) and pharmaceutical acceptable salts thereof may be administered in combination with an HMG CoA reductase inhibitor. The object compounds (I) and pharmaceutical acceptable salts thereof may be also administered in combination with a known anti-obesity agent, for example, β₃-adrenergic receptor agonist, a cholecystokinin-A agonist, a monoamine reuptake inhibitor, a sympathomimetic agent, a serotoninergic agent, a dopamine agonist, a melanocyte-stimulating hormone receptor agonist or mimetic, a melanocyte-stimulating hormone receptor analog, a cannabinoid receptor antagonist, a melanin concentrating hormone antagonist, leptin, a leptin analog, a leptin receptor agonist, a galanin antagonist, a lipase inhibitor, a bombesin agonist, a Neuropeptide-Y antagonist, a thyromimetic agent, dehydroepiandrosterone or an analog thereof, a glucocorticoid receptor agonist or antagonist, an orexin receptor antagonist, a urocortin binding protein antagonist, a glucagon-like peptide-1 receptor agonist, a ciliary neurotrophic factor, a human agouti-related protein antagonist, and the like, for the prophylaxis or treatment of obesity.

The following Preparations and Examples are given for the purpose of illustrating the present invention in detail.

Preparation 1

To a solution of 4-fluoronitrobenzene (12.71 g) and 2-(2-pyridinyl)ethylamine (12.22 g) in N,N-dimethylformamide (70 ml) was added triethylamine (10.12 g) at ambient temperature and the mixture was stirred at 60° C. for 16 hours. The mixture was cooled to 5° C. and poured into a mixture of ethyl acetate and water. The separated organic layer was washed with water and brine, dried over magnesium sulfate and evaporated in vacuo. The residue was triturated with diisopropyl ether, collected by filtration, washed with diisopropyl ether and dried in vacuo to give 2-[2-(4-nitroanilino)ethyl]pyridine (21.21 g) as a yellow solid.

¹H-NMR(DMSO-d₆): δ 3.02 (2H, t, J=7.0 Hz), 3.55 (2H, td, J=7.0 Hz, 5.6 Hz), 6.65 (2H, d, J=9.3 Hz), 7.24 (1H, dd, J=7.8 Hz, 4.9 Hz), 7.31 (1H, d, J=7.8 Hz), 7.39 (1H, t, J=5.6 Hz), 7.65-7.8 (1H, m), 7.98 (1H, d, J=9.3 Hz), 8.52 (1H, d, J=4.0 Hz)

APCI-MS(m/z): 244(M⁺+1)

Preparation 2

To a solution of 2-[2-(4-nitroanilino)ethyl]pyridine (17.87 g) in tetrahydrofuran (150 ml) were added di-tert-butyl dicarbonate (19.25 g) and triethylamine (8.92 g) at ambient temperature and the mixture was refluxed for 16 hours. The mixture was evaporated in vacuo and the residue was purified by column chromatography on silica gel eluting with hexane:ethyl acetate (2:1) to give tert-butyl 4-nitrophenyl[2-(2-pyridinyl)ethyl]carbamate (18.21 g) as a yellow solid.

¹H-NMR(DMSO-d₆): δ 1.37 (9H, s), 2.95 (2H, t, J=8.0 Hz), 4.09 (2H, t, J=8.0 Hz), 7.2-7.3 (2H, m), 7.52 (2H, d, J=9.1 Hz), 7.65-7.75 (1H, m), 8.17 (2H, d, J=9.1 Hz), 8.23 (1H, d, J=4.8 Hz)

APCI-MS (m/z): 344 (M⁺+1)

Preparation 3

To a suspension of tert-butyl 4-nitrophenyl[2-(2-pyridinyl)ethyl]carbamate (20.03 g) in ethanol (400 ml) were added iron(III) chloride (anhydrous) (189 mg) and active-charcoal (20 g) and the mixture was heated to 80° C. To the mixture was added dropwise hydrazine hydrate (11.67 g) and the mixture was stirred at 80° C. for 4 hours. The active-charcoal was filtered off by celite and washed with ethanol. The filtrate was evaporated in vacuo and the residue was purified by column chromatography on silica gel eluting with ethyl acetate to give tert-butyl 4-aminophenyl[2-(2-pyridinyl)ethyl]carbamate (15.03 g) as a light brown solid.

¹H-NMR(DMSO-d₆): δ 1.29 (9H, s), 2.86 (2H, t, J=7.0 Hz), 3.78 (2H, t, J=7.0 Hz), 5.04 (2H, br s), 6.52 (2H, d, J=8.5 Hz), 6.80 (2H, d, J=8.5 Hz), 7.15-7.3 (2H, m), 7.65-7.75 (1H, m), 8.45 (1H, d, J=4.2 Hz)

APCI-MS(m/z): 314(M+H)⁺

### EXAMPLE 1

A mixture of 2-isopropyl-4-[4-(trifluoromethyl)phenyl]-5-pyrimidinecarboxylic acid (495 mg), tert-butyl 4-aminophenyl[2-(2-pyridinyl)ethyl]carbamate (470 mg) and 1-hydroxybenzotriazole hydrate (223 mg) and 1-[3-(dimethylamino)propyl]-3-ethylcarbodiimide hydrochloride (315 mg) in N,N-dimethylformamide (20 ml) was stirred at ambient temperature for 15 hours. The reaction mixture was poured into a mixture of ethyl acetate and water. The organic layer was washed with brine and dried over magnesium sulfate and the solvent was evaporated in vacuo. A mixture of the residue and trifluoroacetic acid (8 ml) was stirred at ambient temperature for 2 hours. The reaction mixture was poured into a mixture of ethyl acetate and water and adjusted to pH 8.0 with 20% aqueous potassium carbonate solution. The organic layer was washed with brine and dried over magnesium sulfate. The solvent was evaporated in vacuo and the residue was chromatographed on silica gel eluting with ethyl acetate:n-hexane (7:3). The fractions containing the desired product were collected and evaporated in vacuo and the residue was recrystallized from ethyl acetate and diisopropyl ether to give 2-isopropyl-N-(4-{[2-(2-pyridinyl)ethyl]amino}phenyl)-4-[4-(trifluoromethyl)phenyl]-5-pyrimidinecarboxamide (0.366 g).

¹H-NMR(DMSO-d₆): δ 1.36 (6H, d, J=6.88 Hz), 2.97 (2H, d, J=7.42 Hz), 3.21-3.41 (3H, m), 5.64 (1H, t, J=5.76 Hz), 6.56 (2H, d, J=8.86 Hz), 7.19-7.32 (4H, m), 7.64-7.74 (1H, m), 7.88 (2H, d, J=8.20 Hz), 7.98 (2H, d, J=8.20 Hz), 8.51 (1H, d, J=4.80 Hz), 8.97 (1H, s), 10.28 (1H, s)

### EXAMPLE 2

A mixture of 2-methyl-4-[4-(trifluoromethyl)phenyl]-5-pyrimidinecarboxylic acid (423 mg), tert-butyl 4-aminophenyl[2-(2-pyridinyl)ethyl]carbamate (470 mg) and 1-hydroxybenzotriazole hydrate (223 mg) and 1-[3-(dimethylamino)propyl]-3-ethylcarbodiimide hydrochloride (315 mg) in N,N-dimethylformamide (20 ml) was stirred at ambient temperature for 15 hours. The reaction mixture was poured into a mixture of ethyl acetate and water. The organic layer was washed with 5% aqueous potassium carbonate solution and brine, and dried over magnesium sulfate. The solvent was evaporated in vacuo and the residue was chromatographed on silica gel eluting with ethyl acetate:n-hexane (8:2→10:0). The fractions containing the desired product were collected and evaporated in vacuo to give tert-butyl 4-[({2-methyl-4-[4-(trifluoromethyl)phenyl]-5-pyrimidinyl}carbonyl)amino]phenyl-[2-(2-pyridinyl)ethyl]carbamate (0.65 g).

¹H-NMR(DMSO-d₆): δ 1.32 (9H, s), 2.73 (3H, s), 2.89 (2H, t, J=6.78 Hz), 3.91 (2H, t, J=6.78 Hz), 7.15-7.25 (4H, m), 7.54 (2H, d, J=8.80 Hz), 7.65-7.69 (1H, m), 7.86-7.98 (4H, m), 8.44-8.47 (1H, m), 9.01 (1H, s), 10.71 (1H, s)

### EXAMPLE 3

A mixture of tert-butyl 4-[({2-methyl-4-[4-(trifluoromethyl)phenyl]-5-pyrimidinyl}carbonyl)amino]phenyl-[2-(2-pyridinyl)ethyl]carbamate (0.65 g) and trifluoroacetic acid (6 ml) was stirred at ambient temperature for 2 hours. The reaction mixture was poured into a mixture of ethyl acetate and water and adjusted to pH 8.0 with 20% aqueous potassium carbonate solution. The organic layer was washed with brine and dried over magnesium sulfate. The solvent was evaporated in vacuo and the residue was recrystallized from ethyl acetate and diisopropyl ether to give 2-methyl-N-(4-{[2-(2-pyridinyl)ethyl]amino}phenyl)-4-[4-(trifluoromethyl)-phenyl]-5-pyrimidinecarboxamide (0.52 g).

¹H-NMR(DMSO-d₆): δ 2.76 (3H, s), 2.97 (2H, t, J=7.44 Hz), 3.31-3.42 (2H, m), 5.63 (1H, t, J=5.70 Hz), 6.55 (2H, d, J=8.88 Hz), 7.19-7.31 (4H, m), 7.66-7.71 (1H, m), 7.85-7.97 (4H, m), 8.49-8.53 (1H, m), 8.93 (1H, s), 10.24 (1H, s)

APCI-MS(m/z): 478(M+H)⁺

### EXAMPLE 4

tert-Butyl 4-({[4-(4-chlorophenyl)-2-methyl-5-pyrimidinyl]carbonyl}amino)phenyl[2-(2-pyridinyl)ethyl]carbamate was obtained from 4-(4-chlorophenyl)-2-methyl-5-pyrimidinecarboxylic acid and tert-butyl 4-aminophenyl[2-(2-pyridinyl)ethyl]carbamate in the same manner as in Example 2.

¹H-NMR(DMSO-d₆): δ 1.33 (9H, s), 2.73 (3H, s), 2.86-2.94 (2H, m), 3.87-3.95 (2H, m),7.15-7.26 (4H, m), 7.52-7.81 (5H, m), 7.79 (2H, d, J=8.61 Hz), 8.46 (1H, d, J=3.98 Hz), 8.94 (1H, s), 10.67 (1H, s)

### EXAMPLE 5

4-(4-Chlorophenyl)-2-methyl-N-(4-{[2-(2-pyridinyl)ethyl]amino}phenyl)-5-pyrimidinecarboxamide was obtained from tert-butyl 4-({[4-(4-chlorophenyl)-2-methyl-5-pyrimidinyl]carbonyl)amino)phenyl[2-(2-pyridinyl)ethyl]-carbamate in the same manner as in Example 3.

¹H-NMR(DMSO-d₆): δ 2.74 (3H, s), 2.98 (2H, t, J=7.26 Hz), 3.32-3.41 (2H, m), 5.60-5.66 (1H, m), 6.56 (2H,. d, J=8.74 Hz), 7.19-7.32 (2H, m), 7.28 (2H, d, J=8.74 Hz), 7.56 (2H, d, J=8.44 Hz), 7.70-7.74 (1H, m), 7.79 (2H, d, J=8.44 Hz), 8.51 (1H, d, J=4.80 Hz), 8.86 (1H, s), 10.21 (1H, s)

Preparation 4

A mixture of methyl 3-(4-chlorophenyl)-3-oxopropanoate (3.2 g), 25% hydrobromide-acetic acid (2 ml) and pyridinium tribromide (5.9 g) in acetic acid (10 ml) was stirred at ambient temperature for 3 hours. The reaction mixture was poured into a mixture of ethyl acetate and water. The organic layer was washed with 5% aqueous potassium carbonate solution and brine, dried over magnesium sulfate, and evaporated in vacuo. A mixture of the residue and thioacetamide (1.7 g) in ethanol (10 ml) was refluxed under stirring for 1.5 hours. The reaction mixture was poured into a mixture of ethyl acetate and water. The organic layer was washed with 5% aqueous potassium carbonate solution and brine, and dried over magnesium sulfate. The solvent was evaporated in vacuo and the residue was chromatographed on silica gel eluting with ethyl acetate:n-hexane (8:2). The fractions containing the desired product were collected and evaporated in vacuo to give methyl 4-(4-chlorophenyl)-2-methyl-1,3-thiazole-5-carboxylate (1.35 g).

¹H-NMR(DMSO-d₆): δ 2.73 (3H, s), 4.29 (3H, s), 7.21 (2H, d, J=7.56 Hz), 7.95 (2H, d, J=7.56 Hz)

Preparation 5

A solution of methyl 4-(4-chlorophenyl)-2-methyl-1,3-thiazole-5-carboxylate (1.2 g) in 4N sodium hydroxide solution (1.6 ml), methanol (20 ml) and tetrahydrofuran (10 ml) was refluxed under stirring for 1.5 hours. The reaction mixture was evaporated in vacuo and the residue was dissolved in a mixture of ethyl acetate and water. The aqueous layer was acidified to pH 1.0 with 10% hydrochloric acid and extracted with ethyl acetate. The extract was washed with brine and dried over magnesium sulfate. The solvent was evaporated in vacuo and the residue was washed with n-hexane to give 4-(4-chlorophenyl)-2-methyl-1,3-thiazole-5-carboxylic acid.

¹H-NMR(DMSO-d₆): δ 2.70 (3H, s), 7.49 (2H, d, J=8.58 Hz), 7.77 (2H, d, J=8.58 Hz)

### EXAMPLE 6

A mixture of 4-(4-chlorophenyl)-2-methyl-1,3-thiazole-5-carboxylic acid (251 mg) and 1-hydroxybenzotriazole hydrate (149 mg) and 1-[3-(dimethylamino)propyl]-3-ethylcarbodiimide hydrochloride (210 mg) in N,N-dimethylformamide (15 ml) was stirred at ambient temperature for 1 hour. 4-(2-Pyridinylmethyl)phenylamine (193 mg) was added to the mixture and the resultant mixture was stirred at ambient temperature for 8 hours. The resultant mixture was poured into a mixture of ethyl acetate and water. The organic layer was washed with brine and dried over magnesium sulfate. The solvent was evaporated in vacuo and the residue was chromatographed on silica gel eluting with ethyl acetate:n-hexane (8:2). The fractions containing the desired product were collected and evaporated in vacuo and the residue was recrystallized from ethyl acetate and diisopropyl ether to give 4-(4-chlorophenyl)-2-methyl-N-[4-(2-pyridinylmethyl)phenyl]-1,3-thiazole-5-carboxamide (0.241 g).

¹H-NMR(DMSO-d₆): δ 2.74 (3H, s), 4.04 (2H, s), 7.17-7.27 (4H, m), 7.45-7.57 (4H, m), 7.67-7.99 (3H, m), 8.41 (1H, d, J=3.96 Hz), 10.45 (1H, s)

### EXAMPLE 7

4-(4-Chlorophenyl)-2-methyl-N-[4-(2-pyridinylmethyl)-phenyl]-5-pyrimidinecarboxamide was obtained from 4-(4-chlorophenyl)-2-methyl-5-pyrimidinecarboxylic acid and 4-(2-pyridinylmethyl)phenylamine in the same manner as in Example 6.

¹H-NMR(DMSO-d₆): δ 2.74 (3H, s), 4.04 (2H, s), 7.17-7.27 (4H, m), 7.46-7.57 (4H, m), 7.65-7.79 (3H, m), 8.41 (1H, d, J=4.82 Hz), 8.89 (1H, s), 10.58 (1H, s)

### EXAMPLE 8

4-(4-Chlorophenyl)-N-[4-(2-pyridinylmethyl)phenyl]-5-pyrimidinecarboxamide was obtained from 4-(4-chlorophenyl)-5-pyrimidinecarboxylic acid and 4-(2-pyridinylmethyl)phenylamine in the same manner as in Example 6.

¹H-NMR(DMSO-d₆): δ 4.04 (2H, s), 7.17-7.24 (4H, m), 7.47-7.82 (7H, m), 8.46 (1H, d, J=3.97 Hz), 9.02 (1H, s), 9.37 (1H, s), 10.65 (1H, s)

### EXAMPLE 9

A mixture of 4′-chloro-5-methyl-1,1′-biphenyl-2-carboxylic acid (370 mg), 4-[2-(2-pyridinyl)ethoxy]aniline (321 mg) and 1-hydroxybenzotriazole hydrate (213 mg) and 1-[3-(dimethylamino)propyl]-3-ethylcarbodiimide hydrochloride (301 mg) in N,N-dimethylformamide (10 ml) was stirred at ambient temperature for 15 hours. The reaction mixture was poured into a mixture of ethyl acetate and water. The organic layer was washed with 5% aqueous potassium carbonate solution and brine and dried over magnesium sulfate. The solvent was evaporated in vacuo and the residue was chromatographed on silica gel eluting with ethyl acetate:n-hexane (7:3). The fractions containing the desired product were collected and evaporated in vacuo and the residue was recrystallized from ethyl acetate and diisopropyl ether to give 4′-chloro-5-methyl-N-{4-[2-(2-pyridinyl)ethoxy]phenyl}-1,1′-biphenyl-2-carboxamide (354 mg).

¹H-NMR(DMSO-d₆): δ 2.40 (3H, s), 3.16 (2H, t, J=6.64 Hz), 4.30, (2H, t, J=6.64 Hz), 6.84 (2H, d, J=9.00 Hz), 7.22-7.48 (11H, m), 7.68-7.72 (1H, m), 8.51 (1H, d, J=4.32 Hz), 10.01 (1H, s)

APCI-MS(m/z): 443(M+H)⁺

### EXAMPLE 10

4′,5-Dimethyl-N-{4-[2-(2-pyridinyl)ethoxy]phenyl}-1,1′-biphenyl-2-carboxamide was obtained from 4′,5-dimethyl-1,1′-biphenyl-2-carboxylic acid and 4-[2-(2-pyridinyl)ethoxy]-aniline in the same manner as in Example 9.

¹H-NMR(DMSO-d₆): δ 2.28 (3H, s), 2.39 (3H, s), 3.15 (2H, t, J=6.62 Hz), 4.29 (2H, t, J=6.62 Hz), 6.83 (2H, d, J=8.96 Hz), 7.14-7.43 (11H, m), 7.68-7.76 (1H, m), 8.51 (1H, d, J=4.30 Hz), 9.95 (1H, s)

APCI-MS(m/z): 423(M+H)⁺

### EXAMPLE 11

4′-Chloro-4-methyl-N-{4-[2-(2-pyridinyl)ethoxy]phenyl}-1,1′-biphenyl-2-carboxamide was obtained from 4′-chloro-4-methyl-1,1′-biphenyl-2-carboxylic acid and 4-[2-(2-pyridinyl)ethoxy]aniline in the same manner as in Example 9.

¹H-NMR(DMSO-d₆): δ 2.40 (3H, s), 3.16 (2H, t, J=6.62 Hz), 4.31 (2H, t, J=6.62 Hz), 6.85 (2H, d, J=8.94 Hz), 7.20-7.45 (11H, m), 7.68-7.76 (1H, m), 8.51 (1H, d, J=4.44 Hz), 10.11 (1H, s)

APCI-MS(m/z): 443(M+H)⁺

### EXAMPLE 12

4,4′-Dimethyl-N-{4-[2-(2-pyridinyl)ethoxy]phenyl}-1,1′-biphenyl-2-carboxamide was obtained from 4,4′-dimethyl-1,1′-biphenyl-2-carboxylic acid and 4-[2-(2-pyridinyl)ethoxy]-aniline in the same manner as in Example 9.

¹H-NMR(DMSO-d₆): δ 2.28 (3H, s), 2.39 (3H, s), 3.16 (2H, t, J=6.66 Hz), 4.30 (2H, t, J=6.66 Hz), 6.84 (2H, d, J=8.98 Hz), 7.13-7.44 (11H, m), 7.68-7.76 (1H, m), 8.51 (1H, d, J=4.40 Hz), 10.04 (1H, s)

APCI-MS(m/z): 423(M+H)⁺

### EXAMPLE 13

4-Methoxy-N-{4-[2-(2-pyridinyl)ethoxy]phenyl}-4′-(trifluoromethyl)-1,1′-biphenyl-2-carboxamide was obtained from 4-methoxy-4′-(trifluoromethyl)-1,1′-biphenyl-2-carboxylic acid and 4-[2-(2-pyridinyl)ethoxy]aniline in the same manner as in Example 9.

¹H-NMR(DMSO-d₆): δ 3.16 (2H, t, J=6.62 Hz), 3.87(3H, s), 4.31 (2H, t, J=6.62 Hz), 6.85 (2H, d, J=8.94 Hz), 7.15-7.45 (11H, m), 7.68-7.46 (3H, m), 7.59 (2H, d, J=8.18 Hz), 8.51 (1H, d, J=4.26 Hz), 10.19 (1H, s)

APCI-MS(m/z): 493(M+H)⁺

Preparation 6

A mixture of 2-chloro-5-nitropyridine (3.13 g), 2-(2-aminoethyl)pyridine (2.93 g) and triethylamine (3.03 g) in N,N-dimethylformamide (20 ml) was stirred at ambient temperature for 20 hours. The reaction mixture was poured into water and the precipitate was collected by filtration. The precipitate was dissolved in a mixture of ethyl acetate and tetrahydrofuran, washed with brine, and dried over magnesium sulfate. The solvent was concentrated in vacuo and the precipitate was collected by filtration to give 5-nitro-N-[2-(2-pyridinyl)ethyl]-2-pyridinamine (4.42 g).

¹H-NMR(DMSO-d₆): δ 3.04 (2H, t, J=7.39 Hz), 3.98-4.09 (2H, m), 6.56 (1H, d, J=9.38 Hz), 7.20-7.27 (2H, m), 7.67-7.75 (1H, m), 8.09 (1H, d, J=7.55 Hz), 8.20-8.25 (1H, m), 8.51-8.53 (1H, m), 8.93 (1H, d, J=2.72 Hz)

Preparation 7

A mixture of 5-nitro-N-[2-(2-pyridinyl)ethyl]-2-pyridinamine (710 mg) in methanol (40 ml) and tetrahydrofuran (10 ml) was hydrogenated over 10% palladium on carbon (230 mg) under an atmospheric pressure of hydrogen at ambient temperature under stirring for 3 hours. After removal of the catalyst, the solvent was evaporated in vacuo to give N²-[2-(2-pyridinyl)ethyl]-2,5-pyridinediamine (621 mg).

### EXAMPLE 14

A mixture of 4-methoxy-4′-(trifluoromethyl)-1,1′-biphenyl-2-carboxylic acid (445 mg), N²-[2-(2-pyridinyl)ethyl]-2,5-pyridinediamine (354 mg) and 1-hydroxybenzotriazole hydrate (213 mg) and 1-[3-(dimethylamino)propyl]-3-ethylcarbodiimide hydrochloride (301 mg) in N,N-dimethylformamide (10 ml) was stirred at ambient temperature for 15 hours. The reaction mixture was poured into a mixture of ethyl acetate and water. The organic layer was washed with 5% aqueous potassium carbonate solution and brine and dried over magnesium sulfate. The solvent was evaporated in vacuo and the residue was chromatographed on silica gel eluting with ethyl acetate:methanol (94:6). The fractions containing the desired product were collected and evaporated in vacuo and the residue was recrystallized from ethyl acetate and diisopropyl ether to give 4-methoxy-N-(6-{[2-(2-pyridinyl)ethyl]amino}-3-pyridinyl)-4′-(trifluoromethyl)-1,1′-biphenyl-2-carboxamide (507 mg).

¹H-NMR(DMSO-d₆): δ 2.97 (2H, t, J=7.38 Hz), 3.51-3.61(2H, m), 3.87 (3H, s), 6.41 (1H, d, J=8.90 Hz), 6.46 (1H, t, J=5.64 Hz), 7.15-7.28 (4H, m), 7.42-7.76 (7H, m), 8.07 (1H, d, J=2.43 Hz), 8.50 (1H, d, J=4.42 Hz), 10.00 (1H, s)

APCI-MS(m/z): 493(M+H)⁺

### EXAMPLE 15

4′-Chloro-5-methyl-N-(6-{[2-(2-pyridinyl)ethyl]amino}-3-pyridinyl)-1,1′-biphenyl-2-carboxamide was obtained from 4′-chloro-5-methyl-1,1′-biphenyl-2-carboxylic acid and N²-[2-(2-pyridinyl)ethyl]-2,5-pyridinediamine in the same manner as in Example 14.

¹H-NMR(DMSO-d₆): δ 2.40 (3H, s), 2.97 (2H, t, J=7.40 Hz), 3.51-3.61 (2H, m), 6.40-6.47 (2H, m), 7.17-7.31 (3H, m), 7.42-7.51 (7H, m), 7.64-7.69 (1H, m), 8.04 (1H, d, J=2.45 Hz), 8.50 (1H, d, J=4.20 Hz), 9.84 (1H, s)

APCI-MS(m/z): 443(M+H)⁺

### EXAMPLE 16

5-Methyl-N-(6-{[2-(2-pyridinyl)ethyl]amino}-3-pyridinyl)-4′-(trifluoromethyl)-1,1′-biphenyl-2-carboxamide was obtained from 5-methyl-4′-(trifluoromethyl)-1,1′-biphenyl-2-carboxylic acid and N²-[2-(2-pyridinyl)ethyl]-2,5-pyridinediamine in the same manner as in Example 14.

¹H-NMR(DMSO-d₆): δ 2.42 (3H, s), 2.97 (2H, t, J=7.40 Hz), 3.53-3.60 (2H, m), 6.39-6.47 (2H, m), 7.17-7.31 (4H, m), 7.44-7.78 (7H, m), 8.05 (1H, d, J=2.43 Hz), 8.50 (1H, d, J=4.12 Hz), 9.92 (1H, s)

APCI-MS(m/z): 477(M+H)⁺

Preparation 8

2-Chloro-5-nitropyridine (4.76 g) was added portionwise to a solution of 2-hydroxyethylpyridine (4.43 g) and potassium tert-butoxide (4.04 g) in tetrahydrofuran (60 ml). The mixture was stirred at a temperature between 5 to 20° C. under ice-cooling and the resultant mixture was stirred at ambient temperature for 3 hours. The reaction mixture was poured into a mixture of ethyl acetate and water. The organic layer was washed with 5% aqueous potassium carbonate solution and brine and dried over magnesium sulfate. The solvent was evaporated in vacuo and the residue was chromatographed on silica gel eluting with ethyl acetate:n-hexane (5:5). The fractions containing the desired product were collected and concentrated in vacuo and the precipitate was collected by filtration to give 5-nitro-2-[2-(2-pyridinyl)ethoxy]pyridine (2.42 g).

¹H-NMR(DMSO-d₆): δ 3.24 (2H, t, J=6.68 Hz), 4.80 (2H, t, J=6.68 Hz), 6.98 (1H, d, J=9.16 Hz), 7.24-7.28 (1H, m), 7.35 (1H, d, J=7.78 Hz), 7.69-7.77 (1H, m), 8.42-8.52 (2H, m), 9.09 (1H, d, J=2.86 Hz).

Preparation 9

A mixture of 5-nitro-2-[2-(2-pyridinyl)ethoxy]pyridine (736 mg), iron powder (900 mg) and ammonium chloride (101 mg) in ethanol (40 ml) and water (8 ml) was refluxed under stirring for 2.5 hours. After removal of the insoluble materials by filtration, the solvent was evaporated in vacuo and the residue was dissolved in ethyl acetate and water. The organic layer was washed with brine and dried over magnesium sulfate. The solvent was evaporated in vacuo to give 6-[2-(2-pyridinyl)ethoxy]-3-pyridinamine (664 mg).

### EXAMPLE 17

A mixture of 5-methyl-4′-(trifluoromethyl)-1,1′-biphenyl-2-carboxylic acid (420 mg), 6-[2-(2-pyridinyl)ethoxy]-3-pyridinamine (339 mg) and 1-hydroxybenzotriazole hydrate (213 mg) and 1-[3-(dimethylamino)propyl]-3-ethylcarbodiimide hydrochloride (301 mg) in N,N-dimethylformamide (10 ml) was stirred at ambient temperature for 14 hours. The reaction mixture was poured into a mixture of ethyl acetate and water. The organic layer was washed with 5% aqueous potassium carbonate solution and brine and dried over magnesium sulfate. The solvent was evaporated in vacuo and the residue was chromatographed on silica gel eluting with ethyl acetate:n-hexane (8:2). The fractions containing the desired product were collected and evaporated in vacuo and the residue was recrystallized from ethyl acetate and diisopropyl ether to give 5-methyl-N-{6-[2-(2-pyridinyl)ethoxy]-3-pyridinyl}-4′-(trifluoromethyl)-1,1′-biphenyl-2-carboxamide (567 mg).

¹H-NMR(DMSO-d₆): δ 2.43 (3H, s), 3.17 (2H, t, J=6.72 Hz), 4.58 (2H, t, J=6.72 Hz), 6.72 (1H, d, J=8.86 Hz), 7.23-7.38.(4H, m), 7.55-7.74 (7H, m), 8.28 (1H, d, J=2.46 Hz), 8.51 (1H, d, J=4.26 Hz), 10.27 (1H, s)

APCI-MS(m/z): 478(M+H)⁺

### EXAMPLE 18

4′-Chloro-5-methyl-N-{6-[2-(2-pyridinyl)ethoxy]-3-pyridinyl}-1,1′-biphenyl-2-carboxamide was obtained from 4′-chloro-5-methyl-1,1′-biphenyl-2-carboxylic acid and 6-[2-(2-pyridinyl)ethoxy]-3-pyridinamine in the same manner as in Example 17.

¹H-NMR(DMSO-d₆): δ 2.41 (3H, s), 3.17 (2H, t, J=6.84 Hz), 4.58 (2H, t, J=6.84 Hz), 6.72 (1H, d, J=8.84 Hz), 7.20-7.67 (9H, m), 7.70-7.82 (2H, m), 8.28 (1H, d, J=2.54 Hz), 8.51 (1H, d, J=4.24 Hz), 10.19 (1H, s)

APCI-MS(m/z): 444(M+H)⁺

### EXAMPLE 19

4-Methoxy-N-{6-[2-(2-pyridinyl)ethoxy]-3-pyridinyl}-4′-(trifluoromethyl)-1,1′-biphenyl-2-carboxamide was obtained from 4-methoxy-4′-(trifluoromethyl)-1,1′-biphenyl-2-carboxylic acid and 6-[2-(2-pyridinyl)ethoxy]-3-pyridinamine in the same manner as in Example 17.

¹H-NMR(DMSO-d₆): δ 3.17 (2H, t, J=6.78 Hz), 4.58 (2H, t, J=6.78 Hz), 6.73 (1H, d, J=8.80 Hz), 7.17-7.26 (3H, m), 7.32 (1H, d, J=7.84 Hz), 7.46 (1H, d, J=8.20 Hz), 7.59 (2H, d, J=8.16 Hz), 7.68-7.83 (4H, m), 8.30 (1H, d, J=2.46 Hz), 8.51 (1H, d, J=4.40 Hz), 10.36 (1H, s)

APCI-MS(m/z): 494(M+H)⁺

Preparation 10

A solution of 5-methyl-4′-(trifluoromethyl)-1,1′-biphenyl-2-carboxylic acid (1.4 g), thionyl chloride (0.55 ml) and N,N-dimethylformamide (11 mg) in toluene (14 ml) was stirred at 55-60° C. for 2 hours. The reaction mixture was evaporated in vacuo and the residue was dissolved in toluene and evaporated in vacuo to give 5-methyl-4′-(trifluoromethyl)-1,1′-biphenyl-2-carbonyl chloride (1.5 g).

Preparation 11

An aqueous solution of 4N NaOH (12 ml) was added to a solution of 4′-aminoacetophenone (5.4 g) and 2-pyridinecarboxaldehyde (4.5 g) in ethanol (50 ml) at ambient temperature under stirring and the resultant mixture was stirred at ambient temperature for 2 hours. The reaction mixture was adjusted to pH 8.0 with 6N hydrochloric acid and concentrated in vacuo to about ½ volume. Water (150 ml) was added to the above resultant mixture and the mixture was stirred at ambient temperature for 0.5 hour. The precipitate was collected by filtration, washed with water and dried to give (2E)-1-(4-aminophenyl)-3-(2-pyridinyl)-2-propen-1-one (7.0 g).

¹H-NMR(DMSO-d₆): δ 6.22 (2H, s), 6.47 (2H, d, J=8.64 Hz), 7.32-7.48 (1H, m), 7.64 (1H, d, J=15.35 Hz), 7.79-8.00 (4H, m), 8.15 (1H, d, J=15.35 Hz), 8.68 (1H, d, J=4.67 Hz)

Preparation 12

A solution of (2E)-1-(4-aminophenyl)-3-(2-pyridinyl)-2-propen-1-one (2.52 g) in methanol (100 ml) was hydrogenated over 10% palladium on carbon (1.25 g) under an atmospheric pressure of hydrogen at ambient temperature under stirring for 2.5 hours. After removal of the catalyst, the solvent was evaporated in vacuo and the residue was triturated with a mixture of ethyl acetate and diisopropyl ether to give 1-(4-aminophenyl)-3-(2-pyridinyl)-1-propanone (3.52 g).

¹H-NMR(DMSO-d₆): δ 3.05 (2H, t, J=7.01 Hz), 3.29 (2H, t, J=7.01 Hz), 6.03(2H, s), 6.57 (2H, d, J=8.62 Hz), 7.14 (1H, m), 7.31 (1H, d, J=7.76 Hz), 7.71 (2H, d, J=8.62 Hz), 7.63-7.69 (1H, m), 8.45 (1H, d, J=4.51 Hz)

### EXAMPLE 20

A solution of 5-methyl-4′-(trifluoromethyl)-1,1′-biphenyl-2-carbonyl chloride (598 mg) in tetrahydrofuran (5 ml) was added to a mixture of 1-(4-aminophenyl)-3-(2-pyridinyl)-1-propanone (453 mg) and triethylamine (405 mg) in tetrahydrofuran (15 ml) at ambient temperature. The mixture was stirred at ambient temperature for 3 hours. The resultant mixture was poured into a mixture of ethyl acetate and water and the organic layer was washed with 5% aqueous potassium carbonate solution and brine and dried over magnesium sulfate. The solvent was evaporated in vacuo and the residue was chromatographed on silica gel eluting with ethyl acetate and n-hexane (8:2). The fractions containing the desired product were collected and evaporated and the residue was recrystallized from ethyl acetate and diisopropyl ether to give 5-methyl-N-{4-[3-(2-pyridinyl)propanoyl]phenyl}-4′-(trifluoromethyl)-1,1′-biphenyl-2-carboxamide (718 mg).

¹H-NMR(DMSO-d₆): δ 2.43 (3H, s), 3.08 (2H, t, J=6.88 Hz), 3.43 (2H, t, J=6.88 Hz), 7.15-7.18 (1H, m), 7.30-7.40 (3H, m), 7.56-7.76 (8H, m), 7.93 (2H, d, J=8.76 Hz), 8.44 (1H, d, J=4.20 Hz), 10.61 (1H, s)

APCI-MS(m/z): 530(M+H)⁺

### EXAMPLE 21

Sodium borohydride (70 mg) was added to a solution of 5-methyl-N-{4-[3-(2-pyridinyl)propanoyl]phenyl}-4′-(trifluoromethyl)-1,1′-biphenyl-2-carboxamide (600 mg) in methanol (15 ml) at ambient temperature under stirring. The mixture was stirred at ambient temperature for 3 hours. The resultant solution was evaporated in vacuo and residue was dissolved in a mixture of ethyl acetate and water. The organic layer was washed with 5% aqueous potassium carbonate solution and brine and dried over magnesium sulfate. The solvent was evaporated in vacuo to give N-{4-[1-hydroxy-3-(2-pyridinyl)propyl]phenyl}-5-methyl-4′-(trifluoromethyl)-1,1′-biphenyl-2-carboxamide (470 mg).

¹H-NMR(DMSO-d₆): δ 1.90-1.97 (2H, m), 2.42 (3H, s), 2.64-2.78 (2H, m), 4.47 (1H, m), 5.23 (1H, d, J=4.36 Hz), 7.16-7.25 (4H, m), 7.35 (2H, d, J=9.02 Hz), 7.44-7.65 (6H, m), 7.74 (2H, d, J=8.32 Hz), 8.45 (1H, d, J=4.34 Hz), 10.23 (1H, s)

### EXAMPLE 22

A solution of N-{4-[1-hydroxy-3-(2-pyridinyl)propyl]phenyl}-5-methyl-4′-(trifluoromethyl)-1,1′-biphenyl-2-carboxamide (450 mg) in methanol (15 ml) and 4N hydrogen chloride in dioxane (1 ml) was hydrogenated over 10% palladium on carbon (200 mg) under an atmospheric pressure of hydrogen at ambient temperature under stirring for 6 hours. After removal of the catalyst, the solvent was evaporated in vacuo and the residue was dissolved in a mixture of water and ethyl acetate. The solution was adjusted to pH 8.0 with 5% aqueous potassium carbonate solution. The organic layer was washed with brine and dried over magnesium sulfate. The solvent was evaporated in vacuo and the residue was chromatographed on silica gel eluting with ethyl acetate and n-hexane (5:5). The fractions containing the desired product. were collected and evaporated and the residue was crystallized from ethyl acetate and diisopropyl ether to give 5-methyl-N-{4-[3-(2-pyridinyl)propyl]phenyl}-4′-(trifluoromethyl)-1,1′-biphenyl-2-carboxamide (236 mg).

¹H-NMR(DMSO-d₆): δ 1.86-1.97 (2H, m), 2.43 (3H, s), 2.49-2.59 (2H, m), 2.71 (2H, t, J=7.34 Hz), 7.10 (1H, d, J=8.36 Hz), 7.18-7.76 (13H, m), 8.47 (1H, d, J=4.16 Hz), 10.20 (1H, s)

APCI-MS(m/z): 475(M+H)⁺

Preparation 13

4-Nitrobenzoyl chloride (3.71 g) was added to a mixture of 2-(2-aminoethyl)pyridine (2.93 g) and triethylamine (4.04 g) in tetrahydrofuran (80 ml) under cooling. The mixture was stirred at ambient temperature for 3 hours. The resultant mixture was poured into a mixture of ethyl acetate and water and the organic layer was washed with 5% aqueous potassium carbonate solution and brine and dried over magnesium sulfate. The solvent was evaporated in vacuo and the residue was recrystallized from ethyl acetate and diisopropyl ether to give 4-nitro-N-[2-(2-pyridinyl)ethyl]benzamide (4.7 g).

¹H-NMR(DMSO-d₆): δ 3.04 (2H, t, J=7.56 Hz), 3.66-3.73 (2H, m), 7.20-7.28 (2H, m), 7.68-7.76 (1H, m), 8.04-8.99 (2H, m), 8.29-8.34 (2H, m), 8.52-8.54 (1H, m), 8.94 (1H, t, J=5.35 Hz)

Preparation 14

A solution of 4-nitro-N-[2-(2-pyridinyl)ethyl]benzamide (1.0 g) in methanol (30 ml) and tetrahydrofuran (30 ml) was hydrogenated over 10% palladium on carbon (500 mg) under an atmospheric pressure of hydrogen at ambient temperature under stirring for 5 hours. After removal of the catalyst, the solvent was evaporated in vacuo to give 4-amino-N-[2-(2-pyridinyl)ethyl]benzamide (750 mg) as a white solid.

¹H-NMR(DMSO-d₆): δ 2.96 (2H, t, J=7.78 Hz), 3.50-3.60 (2H, m), 5.57 (2H, s), 6.50 (2H, d, J=8.60 Hz), 7.18-7.24 (2H, m), 7.54 (2H, d, J=8.60 Hz), 7.65-7.74 (1H, m), 8.08 (1H, t, J=5.46 Hz), 8.50 (1H, d, J=4.48 Hz)

### EXAMPLE 23

A solution of 5-methyl-4′-(trifluoromethyl)-1,1′-biphenyl-2-carbonyl chloride (298 mg) in tetrahydrofuran (5 ml) was added to a mixture of 4-amino-N-[2-(2-pyridinyl)ethyl]benzamide (241 mg) and triethylamine (202 mg) in tetrahydrofuran (10 ml) at ambient temperature. The mixture was stirred at ambient temperature for 3 hours. The resultant mixture was poured into a mixture of ethyl acetate and water and the organic layer was washed with 5% aqueous potassium carbonate solution and brine and dried over magnesium sulfate. The solvent was evaporated in vacuo and the residue was chromatographed on silica gel eluting with ethyl acetate and n-hexane (5:5-7:3). The fractions containing the desired product were collected and evaporated and the residue was recrystallized from ethyl acetate and diisopropyl ether to give 5-methyl-N-[4-({[2-(2-pyridinyl)ethyl]amino}carbonyl)phenyl]-4′-(trifluoromethyl)-1,1′-biphenyl-2-carboxamide (417 mg).

¹H-NMR(DMSO-d₆): δ 2.43 (3H, s), 2.98 (2H, t, J=7.72 Hz), 3.51-3.65 (2H, m), 7.22-7.39 (3H, m), 7.76-7.78 (11H, m), 8.46-8.52 (2H, m), 10.49 (1H, s)

APCI-MS(m/z): 504(M+H)⁺

Preparation 15

A mixture of (4-nitrophenyl)acetic acid (3.62 g), 2-pyridinamine (2.26 g), 1-hydroxybenzotriazole hydrate (2.97 g) and 1-[3-(dimethylamino)propyl]-3-ethylcarbodiimide hydrochloride (4.2 g) in N,N-dimethylformamide (10 ml) was stirred at ambient temperature for 14 hours. The reaction mixture was poured into a mixture of ethyl acetate and water. The organic layer was washed with 5% aqueous potassium carbonate solution and brine, and dried over magnesium sulfate. The solvent was concentrated in vacuo and the resulting precipitate was collected by filtration to give 2-(4-nitrophenyl)-N-(2-pyridinyl)acetamide (3.23 g).

¹H-NMR(DMSO-d₆): δ 3.92 (2H, s), 7.08-7.14 (1H, m), 7.63 (2H, d, J=8.76 Hz), 7.76-7.81 (1H, m), 8.04 (1H, d, J=8.39 Hz), 8.18 (2H, d, J=8.76 Hz), 8.31-8.34 (1H, m), 10.82 (1H, s)

Preparation 16

2-(4-Aminophenyl)-N-(2-pyridinyl)acetamide was obtained from 2-(4-nitrophenyl)-N-(2-pyridinyl)acetamide in the same manner as in Preparation 14.

¹H-NMR(DMSO-d₆): δ 3.53 (2H, s), 5.09 (2H, br s), 6.51-6.65 (2H, m), 6.93-7.09 (3H, m), 7.69-7.78 (1H, m), 8.07 (1H, d, J=8.36 Hz), 8.28-8.31 (1H, m), 10.47 (1H, s)

### EXAMPLE 24

A mixture of 5-methyl-4′-(trifluoromethyl)-1,1′-biphenyl-2-carboxylic acid (420 mg), 2-(4-aminophenyl)-N-(2-pyridinyl)acetamide (358 mg), 1-hydroxybenzotriazole hydrate (213 mg) and 1-[3-(dimethylamino)propyl]-3-ethylcarbodiimide hydrochloride (301 mg) in N,N-dimethylformamide (10 ml) was stirred at ambient temperature for 14 hours. The reaction mixture was poured into a mixture of ethyl acetate and water. The organic layer was washed with 5% aqueous potassium carbonate solution and brine, and dried over magnesium sulfate. The solvent was evaporated in vacuo and the residue was chromatographed on silica gel eluting with ethyl acetate and n-hexane (5:5-7:3). The fractions containing the desired product were collected and evaporated and the residue was recrystallized from ethyl acetate and diisopropyl ether to give 5-methyl-N-{4-[2-oxo-2-(2-pyridinylamino)ethyl]phenyl}-4′-(trifluoromethyl)-1,1′-biphenyl-2-carboxamide.

¹H-NMR(DMSO-d₆): δ 2.42 (3H, s), 3.65 (2H, s), 7.08-7.09 (1H, m), 7.24 (2H, d, J=8.44 Hz), 7.35 (2H, d, J=8.52 Hz), 7.44-7.76 (8H, m), 8.04 (1H, d, J=8.24 Hz), 8.29-8.32 (1H, m), 10.26 (1H, s), 10.63 (1H, s)

Preparation 17

A solution of 5-methyl-4′-(trifluoromethyl)-1,1′-biphenyl-2-carbonyl chloride (598 mg) in tetrahydrofuran (5 ml) was added to a mixture of 4′-aminoacetophenone (270 mg) and triethylamine (405 mg) in tetrahydrofuran (15 ml) at ambient temperature. The mixture was stirred at ambient temperature for 3 hours. The resultant mixture was poured into a mixture of ethyl acetate and water and the organic layer was washed with 5% aqueous potassium carbonate solution and brine and dried over magnesium sulfate. The solvent was evaporated in vacuo and the residue was recrystallized from ethyl acetate and diisopropyl ether to give N-(4-acetylphenyl)-5-methyl-4′-(trifluoromethyl)-1,1′-biphenyl-2-carboxamide (676 mg).

¹H-NMR(DMSO-d₆): δ 2.44 (3H, s), 2.52 (3H, m), 7.36-7.40 (2H, m), 7.57-7.76 (7H, m), 7.90 (2H, d, J=8.70 Hz), 10.62 (1H, s)

### EXAMPLE 25

An aqueous solution of 4N NaOH (0.5 ml) was added to a solution of N-(4-acetylphenyl)-5-methyl-4′-(trifluoromethyl)-1,1′-biphenyl-2-carboxamide (650 mg) and 2-acetoaminopyridine-6-carboxaldehyde (292 mg) in ethanol (15 ml) at ambient temperature under stirring and the resultant mixture was stirred at ambient temperature for 4 hours. The reaction mixture was poured into a mixture of water and ethyl acetate and extracted with ethyl acetate. The extract was washed with brine and dried over magnesium sulfate. The solvent was evaporated in vacuo to give N-(4-{(2E)-3-[6-(acetylamino)-2-pyridinyl]-2-propenoyl}phenyl)-5-methyl-4′-(trifluoromethyl)-1,1′-biphenyl-2-carboxamide (920 mg) as a yellow powder.

¹H-NMR(DMSO-d₆): δ 2.14 (3H, s), 2.44 (3H, m), 7.36-7.41 (2H, m), 7.57-7.81 (11H, m), 7.91-8.14 (3H, dm), 10.54 (1H, s), 10.71 (1H, s)

### EXAMPLE 26

A solution of N-(4-{(2E)-3-[6-(acetylamino)-2-pyridinyl]-2-propenoyl}phenyl)-5-methyl-4′-(trifluoromethyl)-1,1′-biphenyl-2-carboxamide (920 mg) in methanol (30 ml) was hydrogenated over 10% palladium on carbon (350 mg) under an atmospheric pressure of hydrogen at ambient temperature under stirring for 5 hours. After removal of the catalyst, the solvent was evaporated in vacuo and the residue was chromatographed on silica gel eluting with ethyl acetate and n-hexane (5:5-7:3). The fractions containing the desired product were collected and evaporated to give N-(4-{3-[6-(acetylamino)-2-pyridinyl]-1-hydroxypropyl}phenyl)-5-methyl-4′-(trifluoromethyl)-1,1′-biphenyl-2-carboxamide.

¹H-NMR(DMSO-d₆): δ 1.87-1.95 (2H, m), 2.09 (3H, s), 2.43 (3H, m), 2.58-2.70 (2H, m), 4.50-4.52 (1H, m), 5.19 (1H, d, J=4.28 Hz), 6.91 (1H, d, J=7.44 Hz), 7.23 (2H, d, J=8.46 Hz), 7.33-7.67 (8H, m), 7.25 (2H, d, J=8.30 Hz), 7.87 (1H, d, J=8.18 Hz), 10.22 (1H, s), 10.33 (1H, s)

### EXAMPLE 27

N-(4-{3-[6-(Acetylamino)-2-pyridinyl]propyl}phenyl)-5-methyl-4′-(trifluoromethyl)-1,1′-biphenyl-2-carboxamide was obtained from N-(4-{3-[6-(acetylamino)-2-pyridinyl]-1-hydroxypropyl}phenyl)-5-methyl-4′-(trifluoromethyl)-1,1′-biphenyl-2-carboxamide in the same manner as in Example 22.

¹H-NMR(DMSO-d₆): δ 1.83-1.94 (2H, m), 2.06 (3H, s), 2.42 (3H, m), 2.51-2.67 (4H, m), 6.93 (1H, d, J=7.26 Hz), 7.10 (2H, d, J=8.38 Hz), 7.33-7.76 (10H, m), 7.88 (1H, d, J=8.20 Hz), 10.19 (1H, s), 10.35 (1H, s)

(−)APCI-MS(m/z): 530(M−H)⁻

### EXAMPLE 28

A solution of N-(4-{3-[6-(acetylamino)-2-pyridinyl]propyl}phenyl)-5-methyl-4′-(trifluoromethyl)-1,1′-biphenyl-2-carboxamide (150 mg) and 6N hydrochloric acid (5 ml) in methanol (10 ml) was refluxed under stirring for 4 hours. The resultant mixture was evaporated in vacuo and the residue was dissolved in a mixture of ethyl acetate and water. The mixture was adjusted to pH 9.0 with 20% aqueous potassium carbonate solution and the organic layer was washed with brine and dried over magnesium sulfate. The solvent was evaporated in vacuo and the residue was chromatographed on silica gel eluting with ethyl acetate and n-hexane (6:4). The fractions containing the desired product were collected and evaporated and the residue was recrystallized from ethyl acetate and diisopropyl ether to give N-{4-[3-(6-amino-2-pyridinyl)propyl]phenyl}-5-methyl-4′-(trifluoromethyl)-1,1′-biphenyl-2-carboxamide.

¹H-NMR(DMSO-d₆): δ 1.77-1.99 (2H, m), 2.42 (3H, m), 2.46-2.57 (4H, m), 5.75 (2H, s), 6.24 (1H, d, J=8.18 Hz), 6.32 (1H, d, J=7.20 Hz), 7.09 (2H, d, J=8.36 Hz), 7.23 (1H, d J=7.50 Hz), 7.29-7.45 (4H, m), 7.53 (1H, d, J=7.64 Hz), 7.62 (2H, d, J=8.20 Hz), 10.19 (1H, s)

APCI-MS(m/z): 490(M+H)⁺

Preparation 18

To a mixture of methyl 5-methyl-2-{[(trifluoromethyl)sulfonyl]oxy}benzoate (16.0 g), lithium chloride (6.8 g) and tetrakis(triphenylphosphine)palladium(0) (3.1 g) in toluene (192 ml) was added a solution of sodium carbonate (14.8 g) in water (74 ml) under stirring and followed by addition of 4-methylbenzeneboronic acid (8.0 g). The mixture was stirred at 100° C. for 6 hours. The mixture was poured into a mixture of ethyl acetate and water. The separated organic layer was washed with water and evaporated in vacuo. To the residue was added a mixture of sodium hydroxide (5.3 g) in water (53 ml) and ethanol (64 ml) and the mixture was stirred under reflux for 8 hours. The solvent was evaporated. To the residue was added a mixture of ethyl acetate and water and the mixture was adjusted to pH 2 with 6N hydrochloric acid. The separated organic layer was washed with water, dried over magnesium sulfate and evaporated in vacuo. The residue was triturated with hexane and collected by filtration. The precipitate was recrystallized from a mixture of toluene and n-hexane to give 4,4′-dimethyl-1,1′-biphenyl-2-carboxylic acid (7.83 g).

¹H-NMR(DMSO-d₆): δ 2.33 (3H, s), 2.36 (3H, s), 7.19 (4H, s), 7.24 (1H, d, J=7.8 Hz), 7.35 (1H, dd, J=1.3 Hz, 7.8 Hz), 7.50 (1H, d, J=1.3 Hz), 12.66 (1H, s)

### EXAMPLE 29

1-[3-(Dimethylamino)propyl]-3-ethylcarbodiimide (0.17 g) was added to a solution of tert-butyl 4-aminophenyl[2-(2-pyridinyl)ethyl]carbamate (0.31 g), 4,4′-dimethyl-1,1′-biphenyl-2-carboxylic acid (0.25 g), 1-hydroxybenzotriazole hydrate (0.17 g) and 4-dimethylaminopyridine (2.4 mg) in dichloromethane (3 ml) under ice-cooling and the mixture was stirred at ambient temperature for 18 hours. To the reaction mixture was added a solution of 10% hydrogen chloride in methanol (9 ml) and the mixture was stirred at ambient temperature for 24 hours. The reaction mixture was poured into a mixture of ethyl acetate, tetrahydrofuran and water and the mixture was adjusted to pH 9 with 20%. aqueous potassium carbonate solution. The separated organic layer was washed with water, dried over magnesium sulfate and evaporated in vacuo. The residue was triturated with a mixture of ethyl acetate and diethyl ether to give 4,4′-dimethyl-N-(4-{[2-(2-pyridinyl)ethyl]amino}phenyl)-1,1′-biphenyl-2-carboxamide (0.15 g).

¹H-NMR(DMSO-d₆): δ 2.28 (3H, s), 2.38 (3H, s), 2.96 (2H, t, J=7.1 Hz), 3.30-3.39 (2H, m), 5.50 (1H, t, J=5.5 Hz), 6.50 (2H, d, J=8.7 Hz), 7.12-7.32 (11H, m), 7.70 (1H, dt, J=1.8 Hz, 7.5 Hz), 8.48-8.52 (1H, m), 9.78 (1H, s)

(+)APCI-MS: 422(M+H)⁺

Preparation 19

4-Methyl-4′-(trifluoromethyl)-1,1′-biphenyl-2-carboxylic acid was obtained in the same manner as in Preparation 18.

### EXAMPLE 30

4-Methyl-N-(4-{[2-(2-pyridinyl)ethyl]amino}phenyl)-4′-(trifluoromethyl)-1,1′-biphenyl-2-carboxamide was obtained from 4-methyl-4′-(trifluoromethyl)-1,1′-biphenyl-2-carboxylic acid and tert-butyl 4-aminophenyl[2-(2-pyridinyl)ethyl]-carbamate in the same manner as in Example 29.

¹H-NMR(DMSO-d₆): δ 2.42(3H, s), 2.96(2H, t, J=7.2 Hz), 3.28-3.40(2H, m), 5.53(1H, t, J=5.7 Hz), 6.51(2H, d, J=8.8 Hz), 7.18-7.42(7H, m), 7.57-7.77(5H, m), 8.50(1H, dt, J=0.8 Hz, 4.9 Hz), 9.91(1H, s)

(+)APCI-MS: 476(M+H)⁺

Preparation 20

4′-Chloro-4-methyl-1,1′-biphenyl-2-carboxylic acid was obtained in the same manner as in Preparation 18. ¹H-NMR(DMSO-d₆): δ 2.38 (3H, s), 7.22-7.47 (6H, m), 7.61 (1H, s), 12.78 (1H, s)

### EXAMPLE 31

4′-Chloro-4-methyl-N-(4-{[2-(2-pyridinyl)ethyl]amino}-phenyl)-1,1′-biphenyl-2-carboxamide was obtained from 4′-chloro-4-methyl-1,1′-biphenyl-2-carboxylic acid and tert-butyl 4-aminophenyl[2-(2-pyridinyl)ethyl]carbamate in the same manner as in Example 29.

¹H-NMR (DMSO-d₆): δ 2.39 (3H, s), 2.96 (2H, t, J=7.2 Hz), 3.28-3.40 (2H, m), 5.53 (1H, t, J=5.7 Hz), 6.51 (2H, d, J=8.8 Hz), 7.18-7.38 .(7H, m), 7.42 (4H, s), 7.70 (1H, dt, J=1.8 Hz, 7.6 Hz), 8.51 (1H, dd, J=0.8 Hz,. 4.8 Hz), 9.84 (1H, s)

(+)APCI-MS: 442(M+H)⁺

Preparation 21

4′-Fluoro-4-methyl-1,1′-biphenyl-2-carboxylic acid was obtained in the same manner as in Preparation 18.

¹H-NMR(DMSO-d₆): δ 7.15-7.41 (6H, m), 7.56 (1H, s), 12.74 (1H, s)

### EXAMPLE 32

4′-Fluoro-4-methyl-N-(4-{[2-(2-pyridinyl)ethyl]amino}-phenyl)-1,1′-biphenyl-2-carboxamide was obtained from 4′-fluoro-4-methyl-1,1′-biphenyl-2-carboxylic acid and tert-butyl 4-aminophenyl[2-(2-pyridinyl)ethyl]carbamate in the same manner as in Example 29.

¹H-NMR(DMSO-d₆): δ 2.39 (3H, s), 2.96 (2H, t, J=7.2 Hz), 3.27-3.3,9 (2H, m), 5.51 (1H, t, J=5.7 Hz), 6.51 (2H, d, J=8.8 Hz), 7.13-7.47 (1H, m), 7.70 (1H, dt, J=1.8 Hz, 7.6 Hz), 8.51 (1H, d, J=4.1 Hz), 9.79 (1H, s)

(+)ESI-MS: 426(M+H)⁺, 448(M+Na)⁺

Preparation 22

4-Methyl-1,1′-biphenyl-2-carboxylic acid was obtained in the same manner as in Preparation 18.

¹H-NMR(DMSO-d₆): δ 2.38(3H, s), 7.23-7.45(7H, m), 7.53(1H, s), 12.69(1H, s)

### EXAMPLE 33

4-Methyl-N-(4-{[2-(2-pyridinyl)ethyl]amino}phenyl)-1,1′-biphenyl-2-carboxamide was obtained from 4-methyl-1,1′-biphenyl-2-carboxylic acid and tert-butyl 4-aminophenyl[2-(2-pyridinyl)ethyl]carbamate in the same manner as in Example 29.

¹H-NMR(DMSO-d₆): δ 2.39 (3H, s), 2.96 (2H, t, J=7.2 Hz), 3.27-3.38 (2H, m), 5.50 (1H, t, J=5.6 Hz), 6.50 (2H, d, J=8.7 Hz), 7.17-7.45 (12H, m), 7.70 (1H, dt, J=1.6 Hz, 7.7 Hz), 8.50 (1H, d, J=4.5 Hz), 9.76 (1H, s)

(+)ESI-MS: 408(M+H)⁺, 430(M+Na)⁺

Preparation 23

4-Ethyl-4′-methyl-1,1′-biphenyl-2-carboxylic acid was obtained in the same manner as in Preparation 18.

¹H-NMR(DMSO-d₆): δ 1.22 (3H, t, J=7.6 Hz), 2.33 (3H, s), 2.67 (2H, q, J=7.6 Hz), 7.20 (4H, s), 7.26 (1H, d, J=7.8 Hz), 7.39 (1H, dd, J=1.7 Hz, 7.8 Hz), 7.52 (1H, d, J=1.7 Hz), 12.67 (1H, s)

### EXAMPLE 34

4-Ethyl-4′-methyl-N-(4-{[2-(2-pyridinyl)ethyl]amino}-phenyl)-1,1′-biphenyl-2-carboxamide was obtained from 4-ethyl-4′-methyl-1,1′-biphenyl-2-carboxylic acid and tert-butyl 4-aminophenyl[2-(2-pyridinyl)ethyl]carbamate in the same manner as in Example 29.

¹H-NMR(DMSO-d₆): δ 1.24 (3H, t, J=7.5 Hz), 2.29 (3H, s), 2.69 (2H, q, J=7.5 Hz), 2.96 (2H, t, J=7.2 Hz), 2.28-3.44 (2H, m), 5.50 (1H, t, J=5.7 Hz), 6.50 (2H, d, J=8.8 Hz),: 7.13-7.38 (11H, m), 7.70 (1H, dt, J=1.8 Hz, 7.7 Hz), 8.50 (1H, d, J=4.5 Hz), 9.76 (1H, s)

(+)ESI-MS: 436(M+H)⁺, 458(M+Na)⁺

Preparation 24

4-Ethyl-4′-(trifluoromethyl)-1,1′-biphenyl-2-carboxylic acid was obtained in the same manner as in Preparation 18.

¹H-NMR(DMSO-d₆): δ 1.23 (3H, t, J=7.6 Hz), 2.71(2H, q, J=7.6 Hz), 7.32 (1H, d, J=7.9 Hz), 7.47 (1H, dd, J=1.6 Hz, 7.9 Hz), 7.52 (2H, d, J=8.2 Hz), 7.66 (1H, d, J=1.6 Hz), 7.75 (2H, d, J=8.2 Hz), 12.82 (1H, s)

### EXAMPLE 35

4-Ethyl-N-(4-{[2-(2-pyridinyl)ethyl]amino}phenyl)-4′-(trifluoromethyl)-1,1′-biphenyl-2-carboxamide was obtained from 4-ethyl-4′-(trifluoromethyl)-1,1′-biphenyl-2-carboxylic acid and tert-butyl 4-aminophenyl[2-(2-pyridinyl)ethyl]carbamate in the same manner as in Example 29.

¹H-NMR(DMSO-d₆): δ 1.26 (3H, t, J=7.5 Hz), 2.72 (2H, q, J=7.5 Hz), 2.96 (2H, t, J=7.4 Hz), 3.28-3.39 (2H, m), 5.52 (1H, t, J=5.7 Hz), 6.51 (2H, d, J=8.8 Hz), 7.17-7.25 (3H, m), 7.30 (1H, d, J=7.8 Hz), 7.37-7.45 (3H, m), 7.58-7.77 (5H, m), 8.49-8.53 (1H, m), 9.88 (1H, s)

(+)ESI-MS: 490(M+H)⁺, 512(M+Na)⁺

Preparation 25

4′-Chloro-4-ethyl-1,1′-biphenyl-2-carboxylic acid was obtained in the same manner as in Preparation 18.

¹H-NMR(DMSO-d₆): δ 1.22 (3H, t, J=7.5 Hz), 2.69 (2H, q, J=7.5 Hz), 7.25-7.41 (3H, m), 7.41-7.48 (3H, m), 7.57-7.62 (1H, m), 12.77 (1H, s)

### EXAMPLE 36

4′-Chloro-4-ethyl-N-(4-{[2-(2-pyridinyl)ethyl]amino}-phenyl)-1,1′-biphenyl-2-carboxamide was obtained from 4′-chloro-4-ethyl-1,1′-biphenyl-2-carboxylic acid and tert-butyl 4-aminophenyl[2-(2-pyridinyl)ethyl]carbamate in the same manner as in Example 29;

¹H-NMR(DMSO-d₆): δ 1.24 (3H, t, J=7.5 Hz), 2.70 (2H, q, J=7.5 Hz), 2.96 (2H, t, J=7.0 Hz), 3.28-3.40 (2H, m), 5.52 (1H, t, J=5.7 Hz), 6.51 (2H, d, J=8.8 Hz), 7.18-7.38 (7H, m), 7.42 (4H, s), 7.70 (1H, dt, J=1.8 Hz, 7.6 Hz), 8.49-8.53 (1H, m), 9.82 (1H, s)

(+)ESI-MS: 456(M+H)⁺, 478(M+Na)⁺

Preparation 26

4-Ethyl-4′-fluoro-1,1′-biphenyl-2-carboxylic acid was obtained in the same manner as in Preparation 18.

¹H-NMR(DMSO-d₆): δ 1.22 (3H, t, J=7.6 Hz), 2.68 (2H, q, J=7.6 Hz), 7.16-7.45 (6H, m), 7.58 (1H, d, J=1.6 Hz), 12.74 (1H, s)

### EXAMPLE 37

4-Ethyl-4′-fluoro-N-(4-{[2-(2-pyridinyl)ethyl]amino}-phenyl)-1,1′-biphenyl-2-carboxamide was obtained from 4-ethyl-4′-fluoro-1,1′-biphenyl-2-carboxylic acid and tert-butyl 4-aminophenyl[2-(2-pyridinyl)ethyl]carbamate in the same manner as in Example 29.

¹H-NMR(DMSO-d₆): δ 1.24 (3H, t, J=7.5 Hz), 2.70 (2H, q, J=7.5 Hz), 2.96 (2H, t, J=7.2 Hz), 3.28-3.39 (2H, m), 5.51 (1H, t, J=5.8 Hz), 6.50 (2H, d, J=8.8 Hz), 7.14-7.48 (11H, m), 7.70 (1H, dt, J=1.8 Hz, 7.6 Hz), 8.45-8.52 (1H, m), 9.77 (1H, s)

(+)ESI-MS: 440(M+H)⁺, 462(M+Na)⁺

Preparation 27

4-Ethyl-1,1′-biphenyl-2-carboxylic acid was obtained in the same manner as in Preparation 18.

¹H-NMR(DMSO-d₆): δ 1.22 (3H, t, J=7.6 Hz), 2.68 (2H, q, J=7.6 Hz), 7.26-7.58 (8H, m), 12.71 (1H, s)

### EXAMPLE 38

4-Ethyl-N-(4-{[2-(2-pyridinyl)ethyl]amino}phenyl)-1,1′-biphenyl-2-carboxamide was obtained according from 4-ethyl-1,1′-biphenyl-2-carboxylic acid and tert-butyl 4-aminophenyl[2-(2-pyridinyl)ethyl]carbamate in the same manner as in Example 29.

¹H-NMR(DMSO-d₆): δ 1.25 (3H, t, J=7.5 Hz), 2.70 (2H, q, J=7.5 Hz), 2.96 (2H, t, J=7,.2 Hz), 3.28-3.44 (2H, m), 5.50 (1H, t, J=5.7 Hz), 6.50 (2H, d, J=8.8 Hz), 7.17-7.46 (12H, m), 7.70 (1H, dt, J=1.7 Hz, 7.6 Hz), 8.48-8.52 (1H, m), 9.75 (1H, s)

(+)ESI-MS: 422(M+H)⁺, 444(M+Na)⁺

Preparation 28

4-Fluoro-4′-methyl-1,1′-biphenyl-2-carboxylic acid was obtained in the same manner as in Preparation 18.

¹H-NMR(DMSO-d₆): δ 2.34 (3H, s), 7.21 (4 Hz s), 7.37-7.43 (2H, m), 7.46-7.53 (1H, m), 13.01 (1H, s)

### EXAMPLE 39

4-Fluoro-4′-methyl-N-(4-{[2-(2-pyridinyl)ethyl]amino}-phenyl)-1,1′-biphenyl-2-carboxamide was obtained from 4-fluoro-4′-methyl-1,1′-biphenyl-2-carboxylic acid and tert-butyl 4-aminophenyl[2-(2-pyridinyl)ethyl]carbamate in the same manner as in Example 29.

¹H-NMR(DMSO-d₆): δ 2.29 (3H, s), 2.96 (2H, t, J=7.2 Hz), 3.28-3.40 (2H, m), 5.53 (1H, t, J=5.7 Hz), 6.51 (2H, d, J=8.8 Hz), 7.14-7.49 (11H, m), 7.70 (1H, dt, J=1.8 Hz, 7.6 Hz), 8.51 (1H, d, J=4.1 Hz), 9.87 (1H, s)

(+)ESI-MS: 426(M+H)⁺, 448(M+Na)⁺

Preparation 29

4-Fluoro-4′-(trifluoromethyl)-1,1′-biphenyl-2-carboxylic acid was obtained in the same manner as in Preparation 18.

### EXAMPLE 40

4-Fluoro-N-(4-{[2-(2-pyridinyl)ethyl]amino}phenyl)-4′-(trifluoromethyl)-1,1′-biphenyl-2-carboxamide was obtained from 4-fluoro-4′-(trifluoromethyl)-1,1′-biphenyl-2-carboxylic acid and tert-butyl 4-aminophenyl[2-(2-pyridinyl)ethyl]-carbamate in the same manner as in Example 29.

¹H-NMR(DMSO-d₆): δ 2.96 (2H, t, J=7.2 Hz), 3.30-3.39 (2H, m), 5.56 (1H, t, J=5.6 Hz), 6.51 (2H, d, J=8.7 Hz), 7.20 (2H, d, J=8.7 Hz), 7.21-7.33 (2H, m), 7.48-7.79 (8H, m), 8.50 (1H, d, J=4.5 Hz), 9.99 (1H, s)

(+)ESI-MS: 480(M+H)⁺, 502(M+Na)⁺

Preparation 30

4′-Chloro-4-fluoro-1,1′-biphenyl-2-carboxylic acid was obtained in the same manner as in Preparation 18.

¹H-NMR(DMSO-d₆): δ 7.29-7.63 (7H, m), 13.13 (1H, s)

### EXAMPLE 41

4′-Chloro-4-fluoro-N-(4-{[2-(2-pyridinyl)ethyl]amino}-phenyl)-1,1′-biphenyl-2-carboxamide was obtained from 4′-chloro-4-fluoro-1,1′-biphenyl-2-carboxylic acid and tert-butyl 4-aminophenyl[2-(2-pyridinyl)ethyl]carbamate in the same manner as in Example 29.

¹H-NMR(DMSO-d₆): δ 2.96 (2H, t, J=7.2 Hz), 3.28-3.41 (2H, m), 5.55 (1H, t, J=5.7 Hz), 6.52 (2H, d, J=8.8 Hz), 7.18-7.36 (2H, m), 7.21 (2H, d, J=8.8 Hz), 7.27-7.53 (7H, m), 7.70 (1H, dt, J=1.8 Hz, 7.6 Hz), 8.51 (1H, d, J=4.1 Hz), 9.93 (1H, s)

(+)ESI-MS: 446(M+H)⁺, 468(M+Na)⁺

Preparation 31

4,4′-Difluoro-1,1′-biphenyl-2-carboxylic acid was obtained in the same manner as in Preparation 18.

¹H-NMR(DMSO-d₆): δ 7.17-7.46 (6H, m), 7.51-7.58 (1H, m), 13.09 (1H, m)

### EXAMPLE 42

4,4′-Difluoro-N-(4-{[2-(2-pyridinyl)ethyl]amino}phenyl)-1,1′-biphenyl-2-carboxamide was obtained from 4,4′-difluoro-1,1′-biphenyl-2-carboxylic acid and tert-butyl 4-aminophenyl[2-(2-pyridinyl)ethyl]carbamate in the same manner as in Example 29.

¹H-NMR(DMSO-d₆): δ 2.96 (2H, t, J=7.2 Hz), 3.28-3.40 (2H, m), 5.54 (1H, t, J=5.7 Hz), 6.51 (2H, d, J=8.8 Hz), 7.16-7.48 (11H, m), 7.68 (1H, dt, J=1.8 Hz, 7.7 Hz), 8.51 (1H, d, J=4.1 Hz), 9.88 (1H, s)

(+)ESI-MS: 430(M+H)⁺, 452(M+Na)⁺

Preparation 32

4-Chloro-4′-methyl-1,1′-biphenyl-2-carboxylic acid was obtained in the same manner as in Preparation 18.

¹H-NMR(DMSO-d₆): δ 7.22 (4H, s), 7.39 (2H, d, J=8.3 Hz), 7.61 (1H, dd, J=2.2 Hz, 8.3 Hz), 7.71 (1H, d, J=2.2 Hz), 13.70 (1H, s)

Preparation 33

4,4′-Dichloro-1,1′-biphenyl-2-carboxylic acid was obtained in the same manner as in Preparation 18.

¹H-NMR(DMSO-d₆): δ 7.31-7.51 (5H, m), 7.66 (1H, dd, J=2.3 Hz, 8.4 Hz), 7.78 (1H, d, J=2.3 Hz), 13.16 (1H, s)

Preparation 34

4-Chloro-4′-fluoro-1,1′-biphenyl-2-carboxylic acid was obtained in the same manner as in Preparation 18.

¹H-NMR(DMSO-d₆): δ 7.14-7.45 (5H, m), 7.64 (1H, dd, J=2.3 Hz, 8.3 Hz), 7.76 (1H, d, J=2.3 Hz), 13.14 (1H, s)

Preparation 35

4-Methoxy-4′-methyl-1,1′-biphenyl-2-carboxylic acid was obtained in the same manner as in Preparation 18.

¹H-NMR(DMSO-d₆): δ 2.33 (3H, s), 3.82 (3H, s), 7.11 (1H, dd, J=2.7 Hz, 8.4 Hz), 7.15-7.22 (5H, m), 7.28 (1H, d, J=8.4 Hz), 12.77 (1H, s)

Preparation 36

4′-Chloro-4-methoxy-1,1′-biphenyl-2-carboxylic acid was obtained in the same manner as in Preparation 18.

¹H-NMR(DMSO-d₆): δ 3.83 (3H, s), 7.15 (1H, dd, J=2.7 Hz, 8.5 Hz), 7.25-7.34 (4H, m), 7.43 (2H, d, J=8.5 Hz), 12.88 (1H, s)

Preparation 37

4′-Fluoro-4-methoxy-1,1′-biphenyl-2-carboxylic acid was obtained in the same manner as in Preparation 18.

¹H-NMR(DMSO-d₆): δ 3.83 (3H, s), 7.07-7.36 (7H, m), 12.84 (1H, s)

### EXAMPLE 43

4′,5-Dimethyl-N-(4-{[2-(2-pyridinyl)ethyl]amino}phenyl)-1,1′-biphenyl-2-carboxamide was obtained from 4′,5-dimethyl-1,1′-biphenyl-2-carboxylic acid and tert-butyl 4-aminophenyl[2-(2-pyridinyl)ethyl]carbamate in the same manner as in Example 29.

¹H-NMR(DMSO-d₆): δ 2.29 (3H, s), 2.38 (3H, s), 2.96(2H, t, J=7.2 Hz), 3.28-3.39 (2H, m), 5.49 (1H, t, J=5.7 Hz), 6.50 (2H, d, J=8.8 Hz), 7.14-7.42 (11H, m), 7.69 (1H, dt, J=1.8 Hz, 7.6 Hz), 8.50 (1H, d, J=4.8 Hz), 9.69 (1H, s)

(+)APCI-MS: 422(M+H)⁺

### EXAMPLE 44

5-Methyl-N-(4-{[2-(2-pyridinyl)ethyl]amino}phenyl)-4′-(trifluoromethyl)-1,1′-biphenyl-2-carboxamide was obtained from 5-methyl-4′-(trifluoromethyl)-1,1′-biphenyl-2-carboxylic acid and tert-butyl 4-aminophenyl[2-(2-pyridinyl)ethyl]-carbamate in the same manner as in Example 29.

¹H-NMR(DMSO-d₆): δ 2.41 (3H, s), 2.96 (2H, t, J=7.1 Hz), 3.25-3.38 (2H, m), 5.54 (1H, s), 6.50 (2H, d, J=8.7 Hz), 7.16-7.36 (6H, m), 7.49 (1H, d, J=7.6 Hz), 7.58-7.78 (5H, m), 8.51 (1H, d, J=4.1 Hz), 9.83 (1H, s)

(+)ESI-MS: 476(M+H)⁺, 498(M+Na)⁺

### EXAMPLE 45

4′-Chloro-5-methyl-N-(4-{[2-(2-pyridinyl)ethyl]amino}-phenyl)-1,1′-biphenyl-2-carboxamide was obtained from 4′-chloro-5-methyl-1,1′-biphenyl-2-carboxylic acid and tert-butyl 4-aminophenyl[2-(2-pyridinyl)ethyl]carbamate in the same manner as in Example 29.

¹H-NMR(DMSO-d₆): δ 2.39 (3H, s), 2.96 (2H, t, J=7.2 Hz), 3.28-3.39 (2H, m), 5.52 (1H, t, J=5.7 Hz), 6.51 (2H, d, J=8.8 Hz), 7.18-7.33 (6H, m), 7.40-7.47 (5H, m), 7.70 (1H, dt, J=1.7 Hz, 7.6 Hz), 8.51 (1H, d, J=4.7 Hz), 9.76 (1H, s)

(+)APCI-MS: 442(M+H)⁺

### EXAMPLE 46

4′-Fluoro-5-methyl-N-(4-{[2-(2-pyridinyl)ethyl]amino}-phenyl)-1,1′-biphenyl-2-carboxamide was obtained from 4′-fluoro-5-methyl-1,1′-biphenyl-2-carboxylic acid and tert-butyl 4-aminophenyl[2-(2-pyridinyl)ethyl]carbamate in the same manner as in Example 29.

¹H-NMR(DMSO-d₆): δ 2.39 (3H, s), 2.96 (2H, t, J=7.2 Hz), 3.28-3.39 (2H, m), 5.51 (1H, t, J=5.7 Hz), 6.50 (2H, d, J=8.8 Hz), 7.14-7.33 (8H, m), 7.37-7.49 (3H, m), 7.70 (1H, dt, J=1.8 Hz, 7.6 Hz), 8.51 (1H, dd, J=0.8 Hz, 4.8 Hz), 9.71 (1H, s)

(−)APCI-MS: 424(M+H)⁻

### EXAMPLE 47

4′-Methoxy-5-methyl-N-(4-{[2-(2-pyridinyl)ethyl]amino}-phenyl)-1,1′-biphenyl-2-carboxamide was obtained from 4′-methoxy-5-methyl-1,1′-biphenyl-2-carboxylic acid and tert-butyl 4-aminophenyl[2-(2-pyridinyl)ethyl]carbamate in the same manner as in Example 29.

¹H-NMR(DMSO-d₆): δ 2.38 (3H, s), 2.96 (2H, t, J=7.2 Hz), 3.24-3.39 (2H, m), 3.75 (3H, s), 5.50 (1H, t, J=5.6 Hz), 6.50 (2H, d, J=8.9 Hz), 6.92 (2H, d, J=8.8 Hz), 7.18-7.40 (9H, m), 7.70 (1H, dt, J=1.9 Hz, 7.6 Hz), 8.50 (1H, d, J=4.8 Hz), 9.67 (1H, s)

(−)APCI-MS: 438(M+H)⁻

Preparation 38

5-Methyl-1,1′-biphenyl-2-carboxylic acid was obtained in the same manner as in Preparation 18.

¹H-NMR(DMSO-d₆): δ 2.38 (3H, s), 7.18 (1H, s), 7.23-7.43 (6H, m), 7.66 (1H, d, J=7.8 Hz), 12.56 (1H, s)

### EXAMPLE 48

5-Methyl-N-(4-{[2-(2-pyridinyl)ethyl]amino}phenyl)-1,1′-biphenyl-2-carboxamide was obtained from 5-methyl-1,1′-biphenyl-2-carboxylic acid and tert-butyl 4-aminophenyl[2-(2-pyridinyl)ethyl]carbamate in the same manner as in Example 29.

¹H-NMR(DMSO-d₆): δ 2.40 (3H, s), 2.95 (2H, t, J=7.1 Hz), 3.27-3.40 (2H, m), 5.49 (1H, t, J=5.7 Hz), 6.49 (2H, d, J=8.7 Hz), 7.14-7.46 (12H, m), 7.70 (1H, dt, J=1.6 Hz, 7.6 Hz), 8.50 (1H, d, J=4.1 Hz), 9.66 (1H, s)

(+)ESI-MS: 408(M+H)⁺, 430(M+Na)⁺

### EXAMPLE 49

5-(Methoxymethyl)-N-(4-{[2-(2-pyridinyl)ethyl]amino}-phenyl)-4′-(trifluoromethyl)-1,1′-biphenyl-2-carboxamide was obtained from 5-(methoxymethyl)-4′-(trifluoromethyl)-1,1′-biphenyl-2-carboxylic acid and tert-butyl 4-aminophenyl[2-(2-pyridinyl)ethyl]carbamate in the same manner as in Example 29.

¹H-NMR(DMSO-d₆): δ 2.96 (2H, t, J=7.2 Hz), 3.28-3.38 (2H, m), 3.34 (3H, s), 4.53 (2H, s), 5.54 (1H, t, J=5.7 Hz), 6.50 (2H, d, J=8.8 Hz), 7.17-7.32 (2H, m), 7.20 (2H, d, J=8.8 Hz), 7.40-7.49 (2H, m), 7.55-7.79 (6H, m), 8.48-8.53 (1H, m), 9.90 (1H, s)

(+)APCI-MS: 506(M+H)⁺

### EXAMPLE 50

5-(Hydroxymethyl)-N-(4-{[2-(2-pyridinyl)ethyl]amino}-phenyl)-4′-(trifluoromethyl)-1,1′-biphenyl-2-carboxamide was obtained from 5-(acetoxymethyl)-4′-(trifluoromethyl)-1,1′-biphenyl-2-carboxylic acid and tert-butyl 4-aminophenyl[2-(2-pyridinyl)ethyl]carbamate in the same manner as in Example 29.

¹H-NMR(DMSO-d₆): δ 2.96 (2H, t, J=7.2 Hz), 3.28-3.39 (2H, m), 4.61 (2H, d, J=5.6 Hz), 5.36 (1H, t, J=5.6 Hz), 5.53 (1H, t, J=5.6 Hz), 6.51 (2H, d, J=8.7 Hz), 7.18-7.32 (2H, m), 7.20 (2H, d, J=8.7 Hz), 7.40-7.48 (2H, m), 7.53-7.79 (6H, in), 8.51 (1H, d, J=4.1 Hz), 9.86 (1H, s)

(+)APCI-MS: 492(M+H)⁺

### EXAMPLE 51

4′,6-Dimethyl-N-(4-{[2-(2-pyridinyl)ethyl]amino}phenyl)-1,1′-biphenyl-2-carboxamide was obtained from 4′,6-dimethyl-1,1′-biphenyl-2-carboxylic acid and tert-butyl 4-aminophenyl[2-(2-pyridinyl)ethyl]carbamate in the same manner as in Example 29.

¹H-NMR(DMSO-d₆): δ 2.08 (3H, s), 2.29 (3H, s),2.942 (2H, t, J=7.2 Hz), 3.25-3.44 (2H, m), 5.47 (1H, t, J=5.8 Hz), 6.45 (2H, d, J=8.8 Hz), 7.11 (2H, d, J=8.8 Hz), 7.15 (4H, s), 7.17-7.25 (1H, m), 7.26-7.40 (4H, m), 7.69 (1H, dt, J=1.8 Hz, 7.6 Hz), 8.50 (1H, dd, J=0.8 Hz, 4.8 Hz), 9.56 (1H, s)

(+)ESI-MS: 422(M+H)⁺, 444(M+Na)⁺

### EXAMPLE 52

6-Methyl-N-(4-{[2-(2-pyridinyl)ethyl]amino}phenyl)-4′-(trifluoromethyl)-1,1′-biphenyl-2-carboxamide was obtained from 6-methyl-4′-(trifluoromethyl)-1,1′-biphenyl-2-carboxylic acid and tert-butyl 4-aminophenyl[2-(2-pyridinyl)ethyl]carbamate in the same manner as in Example 29.

¹H-NMR(DMSO-d₆): δ 2.08 (3H, s), 2.94 (2H, t, J=7.2 Hz), 3.26-3.37 (2H, m), 5.50 (1H, t, J=5.7 Hz), 6.46 (2H, d, J=8.8 Hz), 7.07 (2H, d, J=8.8 Hz), 7.21 (1H, dd, J=5.0 Hz, 7.4 Hz), 7.29 (1H, d, J=7.8 Hz), 7.37-7.51 (5H, m), 7.64-7.77 (3H, m), 8.50 (1H, d, J=4.1 Hz), 9.71(1H, s)

(+)APCI-MS: 476(M+H)⁺

Preparation 39

To a solution of ethyl {6-[(tert-butoxycarbonyl)amino]-2-pyridinyl}acetate (0.835 g) in tetrahydrofuran (20 ml) was added lithium borohydride (0.097 g) at ambient temperature. The reaction mixture was refluxed for 4 hours, cooled to ambient temperature, quenched with water and extracted with ethyl acetate. The organic layer was washed with brine, dried over magnesium sulfate, filtered and concentrated in vacuo. The residue was purified by column chromatography on silica gel eluting with hexane:ethyl acetate (2:1) to give tert-butyl 6-(2-hydroxyethyl)-2-pyridinylcarbamate (0.627 g) as a white solid.

¹H-NMR(CDCl₃): δ 1.51 (9H, s), 2.92 (2H, t, J=5.4 Hz), 3.99(2H, t, J=5.4 Hz), 6.80 (1H, d, J=6.9 Hz), 7.58 (1H, dd, J=8.2 Hz, 6.9 Hz), 7.79(1H, d, J=8.2 Hz)

Preparation 40

To a solution of tert-butyl 6-(2-hydroxyethyl)-2-pyridinylcarbamate (0.606 g), triethylamine (0.7 ml) and 4-dimethylaminopyridine (15 mg) in 1,2-dichloroethane (25 ml) was added p-toluenesulfonyl chloride (0.582 g) portionwise at ambient temperature. The reaction mixture was stirred for 15 hours, quenched with water and extracted with 1,2-dichloroethane. The organic layer was washed with brine, dried over magnesium sulfate, filtered and concentrated in vacuo. The residue was purified by column chromatography on silica gel eluting with hexane:ethyl acetate (4:1) to give 2-(6-[(tert-butoxycarbonyl)amino]-2-pyridinyl )ethyl 4-methylbenzenesulfonate (0.785 g) as a clear oil.

¹H-NMR(CDCl₃): δ 1.52 (9H, s), 2.43 (3H, s), 2.96 (2H, t, J=6.6 Hz), 4.37 (2H, t, J=6.6 Hz), 6.76 (1H, d, J=7.2 Hz), 7.00 (1H, br s), 7.26 (2H, d, J=7.9 Hz), 7.52 (1H, dd, J=8.2 Hz, 7.2 Hz), 7.65 (2H, d, J=7.9 Hz), 7.73 (1H, d, J=8.2 Hz)

Preparation 41

A mixture of 2-(6-[(tert-butoxycarbonyl)amino]-2-pyridinyl}ethyl 4-methylbenzenesulfonate (1.342 g) and sodium azide (0.444 g) in N,N-dimethylformamide (20 ml) was stirred at ambient temperature for 15 hours. The solvent was evaporated. The residue was dissolved in a mixture of ethyl acetate and water, and extracted with ethyl acetate. The organic layer was washed with water and brine, dried over magnesium sulfate, filtered and concentrated in vacuo to give tert-butyl 6-(2-azidoethyl)-2-pyridinylcarbamate (0.880 g) as a yellow oil. The product was used for the next step without further purification.

¹H-NMR(CDCl₃): δ 1.52 (9H; s), 2.93 (2H, t, J=6.9 Hz), 3.64 (2H, t, J=6.9 Hz), 6.84 (1H, d, J=6.6 Hz), 7.59 (1H, dd, J=8.2 Hz, 6.6 Hz), 7.78 (1H, d, J=8.2 Hz)

Preparation 42

A solution of tert-butyl 6-(2-azidoethyl)-2-pyridinylcarbamate (0.88 g) in methanol (35 ml) was hydrogenated over 10% palladium on carbon at ambient temperature under atmospheric pressure of hydrogen for 1 hour. The reaction mixture was filtered through a pad of celite, and the filtrate was concentrated in vacuo to give tert-butyl 6-(2-aminoethyl)-2-pyridinylcarbamate (0.776 g) as a yellow oil. The product was used for the next step without further purification.

¹H-NMR(CDCl₃): δ 1.51 (9H, s), 2.79 (2H, t, J=6.3 Hz), 3,05 (2H, t, J=6.3 Hz), 6.81 (1H, d, J=7.3 Hz), 7.57 (1H, dd, J=8.2 Hz, 7.3 Hz)

Preparation 43

A mixture of tert-butyl 6-(2-aminoethyl)-2-pyridinylcarbamate (0.776 g), 1-fluoro-4-nitrobenzene (0.462 g) and triethylamine (0.69 ml) in 1,3-dimethyl-2-imidazolidinone (10 ml) was heated to 50° C. for 3.5 hours. The reaction mixture was cooled to ambient temperature, poured into water and extracted with ethyl acetate. The organic layer was washed with brine, dried over magnesium sulfate, filtered and concentrated in vacuo. The residue was purified by column chromatography on silica gel eluting with hexane:ethyl acetate (3:2) to give tert-butyl 6-[2-(4-nitroanilino)ethyl]-2-pyridinylcarbamate (0.666 g) as a yellow oil.

¹H-NMR(CDCl₃): δ 1.53 (9H, s), 2.99 (2H, t, J=6.6 Hz), 3.57 (2H, dd, J=12.2 Hz, 6.2 Hz), 5.21 (1H, br s), 6.53 (2H, d, J=9.2 Hz), 6.82 (1H, dd, J=7.6 Hz, 0.7 Hz), 7.30 (1H, br s), 7.59 (1H, d, J=7.8 Hz), 7.95 (1H, d, J=7.9 Hz), 8.05 (2H, d, J=8.9 Hz)

Preparation 44

To a solution of tert-butyl 6-[2-(4-nitroanilino)ethyl]-2-pyridinylcarbamate (0.666 g) and 4-dimethylaminopyridine (23 mg) in tetrahydrofuran (25 ml) was added di-tert-butyl dicarbonate (0.608 g) and the mixture was heated at 50° C. for 1 hour. The reaction mixture was cooled to ambient temperature and concentrated in vacuo. The residue was dissolved in ethyl acetate and water, and extracted with ethyl acetate. The organic layer was washed with brine, dried over magnesium sulfate, filtered and concentrated in vacuo to give tert-butyl 2-{6-[(tert-butoxycarbonyl)amino]-2-pyridinyl}ethyl(4-nitrophenyl)carbamate (0.851 g). The product was used for the next step without further purification.

Preparation 45

A solution of tert-butyl 2-{6-[(tert-butoxycarbonyl)amino]-2-pyridinyl}ethyl(4-nitrophenyl)carbamate (0.851 g) in methanol (30 ml) was hydrogenated over 10% palladium on carbon at ambient temperature under atmospheric pressure of hydrogen for 1 hour. The reaction mixture was filtered through a pad of celite, and the filtrate was concentrated in vacuo. The residue was purified by column chromatography on silica gel eluting with hexane:ethyl acetate (3:2) to give tert-butyl 6-{2-[4-amino(tert-butoxycarbonyl)anilino]ethyl}-2-pyridinylcarbamate (0.701 g) as a yellow oil.

### EXAMPLE 53

N-(4-{[2-(6-Amino-2-pyridinyl)ethyl]amino}phenyl)-4,4′-dimethyl-1,1′-biphenyl-2-carboxamide was obtained from 4,4′-dimethyl-1,1′-biphenyl-2-carboxylic acid and tert-butyl 6-{2-[4-amino(tert-butoxycarbonyl)anilino]ethyl}-2-pyridinylcarbamate in the same manner as in Example 29.

¹H-NMR(DMSO-d₆): δ 2.29 (3H, s), 2.38 (3H, s), 2.71 (2H, t, J=7.2 Hz), 3.18-3.33 (2H, m), 5.49 (1H, t, J=5.6 Hz), 5.82 (2H, s), 6.27 (1H, d, J=8.1 Hz), 6.39 (1H, d, J=7.1 Hz), 6.50 (2H, d, J=8.7 Hz), 7.12-7.35 (10H, m), 9.77 (1H, s)

(+)ESI-MS: 437(M+H)⁺, 459(M+Na)⁺

### EXAMPLE 54

N-(4-{[2-(6-Amino-2-pyridinyl)ethyl]amino}phenyl)-4-methyl-4′-(trifluoromethyl)-1,1′-biphenyl-2-carboxamide was obtained from 4-methyl-4′-(trifluoromethyl)-1,1′-biphenyl-2-carboxylic acid and tert-butyl 6-{2-[4-amino(tert-butoxycarbonyl)anilino]ethyl}-2-pyridinylcarbamate in the same manner as in Example 29.

¹H-NMR(DMSO-d₆): δ 2.41 (3H, s), 2.71 (2H, t, J=7.2 Hz), 3.22-3.32 (2H, m), 5.51 (1H, t, J=5.6 Hz), 5.81 (2H, s), 6.27 (1H, d, J=8.1 Hz), 6.39 (1H, d, J=7.2 Hz), 6.50 (2H, d, J=8.7 Hz), 7.21 (2H, d, J=8.7 Hz), 7.22-7.31 (1H, m), 7.37-7.41 (3H, m), 7.61 (2H, d, J=8.2 Hz), 7.74 (2H, d, J=8.2 Hz), 9.89 (1H, s)

(+)ESI-MS: 491(M+H)⁺, 513(M+Na)⁺

### EXAMPLE 55

N-(4-{[2-(6-Amino-2-pyridinyl)ethyl]amino}phenyl)-4′-chloro-4-methyl-1,1′-biphenyl-2-carboxamide was obtained from 4′-chloro-4-methyl-1,1′-biphenyl-2-carboxylic acid and tert-butyl 6-{2-[4-amino(tert-butoxycarbonyl)anilino]ethyl}-2-pyridinylcarbamate in the same manner as in Example 29.

¹H-NMR(DMSO-d₆): δ 2.39 (3H, s), 2.71 (2H, t, J=7.2 Hz), 3.19-3.31 (2H, m), 5.51 (1H, t, J=5.6 Hz), 5.82 (2H, s), 6.27 (1H, d, J=8.1 Hz), 6.39 (1H, d, J=7.1 Hz), 6.51 (2H, d, J=8.8 Hz), 7.19-7.43 (10H, m), 9.83 (1H, s)

(+)ESI-MS: 457(M+H)⁺, 479(M+Na)⁺

### EXAMPLE 56

N-(4-{[2-(6-Amino-2-pyridinyl)ethyl]amino}phenyl)-4′-fluoro-4-methyl-1,1′-biphenyl-2-carboxamide was obtained from 4′-fluoro-4-methyl-1,1′-biphenyl-2-carboxylic acid and tert-butyl 6-{2-[4-amino(tert-butoxycarbonyl)anilino]ethyl}-2-pyridinylcarbamate in the same manner as in Example 29.

¹H-NMR(DMSO-d₆): δ 2.39 (3H, s), 2.71 (2H, t, J=7.1 Hz), 3.25 (2H, t, J=7.1 Hz), 5.52 (1H, s), 5.81 (2H, s), 6.28 (1H, d, J=8.2 Hz), 6.39 (1H, d, J=7.1 Hz), 6.50 (2H, d, J=8.6 Hz), 7.16-7.35 (8H, m), 7.40-7.46 (2H, m), 9.77 (1H, s)

(+)ESI-MS: 441(M+H)⁺, 463(M+Na)⁺

### EXAMPLE 57

N-(4-{[2-(6-Amino-2-pyridinyl)ethyl]amino}phenyl)-4-methyl-1,1′-biphenyl-2-carboxamide was obtained from 4-methyl-1,1′-biphenyl-2-carboxylic acid and tert-butyl 6-{2-[4-amino(tert-butoxycarbonyl)anilino]ethyl}-2-pyridinylcarbamate in the same manner as in Example 29.

¹H-NMR(DMSO-d₆): δ 2.39 (3H, s), 2.71(2H, t, J=7.2 Hz), 3.18-3.30(2H, m), 5.49(H, t, J=5.6 Hz), 5.81(2H, s), 6.27(1H, d, J=8.1 Hz), 6.39(1H, d, J=7.2 Hz), 6.49(2H, d, J=8.8 Hz), 7.20(2H, d, J=8.8 Hz), 7.22-7.46(9H, m), 9.75(1H, s)

(+)ESI-MS: 423(M+H)⁺, 445(M+Na)⁺

### EXAMPLE 58

N-(4-{[2-(6-Amino-2-pyridinyl)ethyl]amino}phenyl)-4-chloro-4′-(trifluoromethyl)-1,1′-biphenyl-2-carboxamide was obtained from 4-chloro-4′-(trifluoromethyl)-1,1′-biphenyl-2-carboxylic acid and tert-butyl 6-{2-[4-amino(tert-butoxycarbonyl)anilino]ethyl}-2-pyridinylcarbamate in the same manner as in Example 29.

¹H-NMR(DMSO-d₆): δ 2.71 (2H, t, J=7.2 Hz), 3.18-3.30 (2H, m), 5.55 (1H, t, J=5.6 Hz), 5.82 (2H, s), 6.27 (1H, d, J=8.1 Hz), 6.39 (1H, d, J=7.2 Hz), 6.51 (2H, d, J=8.8 Hz), 7.20 (2H, d, J=8.8 Hz), 7.22-7.31 (1H, m), 7.48-7.55 (1H, m), 7.59-7.68.(4H, m), 7.77 (2H, d, J=8.4 Hz), 10.04 (1H, s)

(+)ESI-MS: 511(M+H)⁺, 533(M+Na)⁺

### EXAMPLE 59

N-(4-{[2-(6-Amino-2-pyridinyl)ethyl]amino}phenyl)-4-fluoro-4′-(trifluoromethyl)-1,1′-biphenyl-2-carboxamide was obtained from 4-fluoro-4′-(trifluoromethyl)-1,1′-biphenyl-2-carboxylic acid and tert-butyl 6-{2-[4-amino(tert-butoxycarbonyl)anilino]ethyl}-2-pyridinylcarbamate in the same manner as in Example 29.

¹H-NMR(DMSO-d₆): δ 2.71 (2H, t, J=7.2 Hz), 3.18-3.30 (2H, m), 5.55 (1H, t, J=5.6 Hz), 5.81 (2H, s), 6.27 (1H, d, J=8.0 Hz), 6.38 (1H, d, J=6.9 Hz), 6.50 (2H, d, J=8.8 Hz), 7.19 (2H, d, J=8.8 Hz), 7.22-7.31 (1H, m), 7.42-7.59 (3H, m), 7.61 (2H, d, J=8.3 Hz), 7.76 (2H, d, J=8.3 Hz), 9.98 (1H, s)

(+)ESI-MS: 495(M+H)⁺, 517(M+Na)⁺

### EXAMPLE 60

N-(4-{[2-(6-Amino-2-pyridinyl)ethyl]amino}phenyl)-4-methoxy-4′-(trifluoromethyl)-1,1′-biphenyl-2-carboxamide was obtained from 4-methoxy-4′-(trifluoromethyl)-1,1′-biphenyl-2-carboxylic acid and tert-butyl 6-{2-[4-amino(tert-butoxycarbonyl)anilino]ethyl}-2-pyridinylcarbamate in the same manner as in Example 29.

¹H-NMR(DMSO-d₆): δ 2.71 (2H, t, J=7.2 Hz), 3.19-3.30 (2H, m), 3.86 (3H, s), 5.52 (1H, t, J=5.6 Hz), 5.82 (2H, s), 6.27 (1H, d, J=8.1 Hz), 6.39 (1H, d, J=7.1 Hz), 6.51 (2H, d, J=8.8 Hz), 7.11-7.31 (5H, m), 7.39-7.46 (1H, m), 7.59 (2H, d, J=8.2 Hz), 7.72 (2H, d, J=8.2 Hz), 9.91 (1H, s)

(+)ESI-MS: 507(M+H)⁺, 529(M+Na)⁺

### EXAMPLE 61

5-Chloro-4′-methyl-N-(4-{[2-(2-pyridinyl)ethyl]amino}-phenyl)-1,1′-biphenyl-2-carboxamide was obtained from 5-chloro-4′-methyl-1,1′-biphenyl-2-carboxylic acid and tert-butyl 4-aminophenyl[2-(2-pyridinyl)ethyl]carbamate in the same manner as in Example 29.

¹H-NMR(DMSO-d₆): δ 2.30 (3H, s), 2.96 (2H, t, J=7.2 Hz), 3.29-3.39 (2H, m), 5.53 (1H, t, J=5.8 Hz), 6.51 (2H, d, J=8.8 Hz), 7.17-7.52 (11H, m), 7.70 (1H, dt, J=1.9 Hz, 7.6 Hz), 8.48-8.53 (1H, m), 9.84 (1H, s)

(+)APCI-MS: 442(M+H)⁺

### EXAMPLE 62

5-Chloro-N-(4-{[2-(2-pyridinyl)ethyl]amino}phenyl)-4′-(trifluoromethyl)-1,1′-biphenyl-2-carboxamide was obtained from 5-chloro-4′-(trifluoromethyl)-1,1′-biphenyl-2-carboxylic acid and tert-butyl 4-aminophenyl[2-(2-pyridinyl)ethyl]carbamate in the same manner as in Example 29.

¹H-NMR(DMSO-d₆): δ 2.95 (2H, t, J=7.4 Hz), 3.27-3.40 (2H, m), 5.56 (1H, t, J=5.6 Hz), 6.51 (2H, d, J=8.8 Hz), 7.15-7.32 (2H, m), 7.18 (2H, d, J=8.8 Hz), 7.56-7.98 (8H, m), 8.48-8.52 (1H, m), 9.95 (1H, s)

(+)APCI-MS: 496(M+H)⁺

### EXAMPLE 63

4′,5-Dichloro-N-(4-{[2-(2-pyridinyl)ethyl]amino}phenyl)-1,1′-biphenyl-2-carboxamide was obtained from 4′,5-dichloro-1,1′-biphenyl-2-carboxylic acid and tert-butyl 4-aminophenyl[2-(2-pyridinyl)ethyl]carbamate in the same manner as in Example 29.

¹H-NMR(DMSO-d₆): δ 2.96 (2H, t, J=7.2 Hz), 3.28-3.40 (2H, m), 5.55 (1H, t, J=5.7 Hz), 6.51 (2H, d, J=8.8 Hz), 7.17-7.25 (1H, m), 7.2 (2H, d, J=8.8 Hz), 7.30 (1H, d, J=7.8 Hz), 7.52 (4H, s), 7.50-7.60 (3H, m), 7.70 (1H, dt, J=1.8 Hz, 7.6 Hz), 8.48-8.53 (1H, m), 9.89 (1H, s)

(+)APCI-MS: 463(M+H)⁺

### EXAMPLE 64

6-Methoxy-4′-methyl-N-(4-{[2-(2-pyridinyl)ethyl]amino}-phenyl)-1,1′-biphenyl-2-carboxamide was obtained from 6-methoxy-4′-methyl-1,1′-biphenyl-2-carboxylic acid and tert-butyl 4-aminophenyl[2-(2-pyridinyl)ethyl]carbamate in the same manner as in Example 29.

¹H-NMR(DMSO-d₆): δ 2.27 (3H, s), 2.94 (2H, t, J=7.2 Hz), 3.25-3.37 (2H, m), 3.7 (3H, s), 5.47 (1H, t, J=5.7 Hz), 6.46 (2H, d, J=8.8 Hz), 7.04-7.25 (9H, m), 7.29 (1H, d, J=7.8 Hz), 7.40 (1H, t, J=7.8 Hz), 7.69 (1H, dt, J=1.7 Hz, 7.6 Hz), 8.50 (1H, d, J=4.6 Hz), 9.57 (1H, s)

(+)APCI-Ms: 438(M+H)⁺

### EXAMPLE 65

6-Methoxy-N-(4-{[2-(2-pyridinyl)ethyl]amino}phenyl)-4′-(trifluoromethyl)-1,1′-biphenyl-2-carboxamide was obtained from 6-methoxy-4′-(trifluoromethyl)-1,1′-biphenyl-2-carboxylic acid and tert-butyl 4-aminophenyl[2-(2-pyridinyl)ethyl]carbamate in the same manner as in Example 29.

¹H-NMR(DMSO-d₆): δ 2.95 (2H, t, J=7.2 Hz), 3.27-3.37 (2H, m), 3.74 (3H, s), 5.60 (1H, s), 6.46 (2H, d, J=8.7 Hz), 7.05-7.32 (4H, m), 7.08 (2H, d, J=8.7 Hz), 7.44-7.53 (3H, m), 7.65-7.75 (3H, m), 8.50 (1H, d, J=4.3 Hz), 9.71 (1H, s)

(+)APCI-MS: 492(M+H)⁺

### EXAMPLE 66

1-[3-(Dimethylamino)propyl]-3-ethylcarbodiimide (0.17 g) was added to a solution of N-(4-aminophenyl)-2-(2-pyridinyl)acetamide (0.23 g), 6-methyl-4′-(trifluoromethyl)-1,1′-biphenyl-2-carboxylic acid (0.31 g), 1-hydroxybenzotriazole (0.15 g) and 4-dimethylaminopyridine (2.4 mg) in dichloromethane (3 ml) under ice-cooling and the mixture was stirred at ambient temperature for 18 hours. The reaction mixture was poured into a mixture of ethyl acetate and tetrahydrofuran, and the mixture was washed with saturated aqueous sodium hydrogencarbonate solution and water. The organic layer was dried over magnesium sulfate and evaporated in vacuo. The residue was purified by column chromatography on silica gel using a mixture of ethyl acetate and diisopropyl ether (1:1) as an eluant. The eluted fractions containing the desired product were collected and evaporated in vacuo. The residue was recrystallized from a mixture of ethyl acetate and diisopropyl ether to give 6-methyl-N-{4-[(2-pyridinylacetyl)amino]phenyl}-4′-(trifluoromethyl)-1,1′-biphenyl-2-carboxamide (0.1 g).

¹H-NMR(DMSO-d₆): δ 2.09 (3H, s), 3.80 (2H, s), 7.22-7.50 (11H, m), 7.70-7.79 (3H, m), 8.47-8.51 (1H, m), 10.08 (1H, s), 10.16 (1H, s)

(+)APCI-MS: 490(M+H)⁺

### EXAMPLE 67

6-Methoxy-N-{4-[(2-pyridinylacetyl)amino]phenyl}-4′-(trifluoromethyl)-1,1′-biphenyl-2-carboxamide was obtained from 6-methoxy-4′-(trifluoromethyl)-1,1′-biphenyl-2-carboxylic acid and N-(4-aminophenyl)-2-(2-pyridinyl)acetamide in the same manner as in Example 66.

¹H-NMR(DMSO-d₆): δ 3.75 (3H, s), 3.80 (2H, s), 7.16-7.56 (11H, m), 7.66-7.79 (1H, m), 7.68 (2H, d, J=8.2 Hz), 8.47-8.51 (1H, m), 10.09 (1H, s), 10.17 (1H, s)

(+)APCI-MS: 506(M+H)⁺

### EXAMPLE 68

5-Methyl-N-{4-[2-(2-pyridinyl)ethoxy]phenyl}-4′-(trifluoromethyl)-1,1′-biphenyl-2-carboxamide was obtained from 5-methyl-4′-(trifluoromethyl)-1,1′-biphenyl-2-carboxylic acid and 4-[2-(2-pyridinyl)ethoxy]aniline in the same manner as in Example 66.

¹H-NMR(DMSO-d₆): δ 2.42 (3H, s), 3.16 (2H, t, J=6.5 Hz), 6.84 (2H, d, J=8.8 Hz), 7.20-7.77 (12H, m), 8.51 (1H, d, J=3.9 Hz), 10.11 (1H, s)

(+)APCI-MS: 477(M+H)⁺

### EXAMPLE 69

6-Methyl-N-{4-[2-(2-pyridinyl)ethoxy]phenyl}-4′-(trifluoromethyl)-1,1′-biphenyl-2-carboxamide was obtained from 6-methyl-4′-(trifluoromethyl)-1,1′-biphenyl-2-carboxylic acid and 4-[2-(2-pyridinyl)ethoxy]aniline in the same manner as in Example 66.

¹H-NMR(DMSO-d₆): δ 2.09 (3H, s), 3.14 (2H, t, J=6.6 Hz), 4.28 (2H, t, J=6.6 Hz), 6.79 (2H, d, J=8.9 Hz), 7.19-7.50 (9H, m), 7.66-7.75 (3H, m), 8.50 (1H, d, J=4.8 Hz), 9.99 (1H, s)

(+)APCI-MS: 477(M+H)⁺

Preparation 46

A mixture of 4-nitrobenzyl bromide (25.0 g), 2-pyridinemethanol (11.2 ml), and 1N sodium hydroxide (116 ml) in tetrahydrofuran (375 ml) was stirred at ambient temperature for 24 hours. The solvent was removed by concentration and to the residue was added a mixture of ethyl acetate and water. The mixture was adjusted to pH 1 with 6N hydrochloric acid. The separated aqueous layer was adjusted to pH 8 with 20% aqueous potassium carbonate solution and extracted with ethyl acetate. The extract was washed with water, dried over magnesium sulfate and evaporated in vacuo to give 2-{[(4-nitrobenzyl)oxy]methyl}pyridine (9.55 g) as an oil.

¹H-NMR(DMSO-d₆): δ 4.68 (2H, s), 4.78 (2H, s), 7.29-7.36 (1H, m), 7.51 (1H, d, J=7.8 Hz), 7.67 (2H, d, J=8.8 Hz), 7.83 (1H, dt, J=1.7 Hz, 7.8 Hz), 8.24 (2H, d, J=8.8 Hz), 8.52-8.55 (1H, m)

Preparation 47

4-[(2-Pyridinylmethoxy)methyl]aniline was obtained from 2-{[(4-nitrobenzyl)oxy]methyl}pyridine in the same manner as in Preparation 3.

¹H-NMR(DMSO-d₆): δ 4.39 (2H, s), 4.52 (2H, s), 5.07 (2H, s), 6.54 (2H, d, J=8.3 Hz), 7.02 (2H, d, J=8.3 Hz), 7.27-7.31 (1H, m), 7.43 (1H, d, J=7.8 Hz), 7.79 (1H, dt, J=1.7 Hz, 7.8 Hz), 8.48-8.53 (1H, m)

### EXAMPLE 70

4′,5-Dimethyl-N-{4-[(2-pyridinylmethoxy)methyl]phenyl}-1,1′-biphenyl-2-carboxamide was obtained from 4′,5-dimethyl-1,1′-biphenyl-2-carboxylic acid and 4-[(2-pyridinylmethoxy)-methyl]aniline in the same manner as in Example 66.

¹H-NMR(DMSO-d₆): δ 2.28 (3H, s), 2.40 (3H, s), 4.53(2H, s), 4.57 (2H, s), 7.16 (2H, d, J=8.0 Hz), 7.24-7.35 (7H, m), 7.41-7.56 (4H, m), 7.80 (1H, dt, J=1.8 Hz, 7.6 Hz), 8.51 (1H, d, J=4.1 Hz), 10.16 (1H, s)

(−)APCI-MS: 421(M+H)⁻

### EXAMPLE 71

4-Methoxy-N-(4-{[2-(2-pyridinyl)ethyl]amino}phenyl)-4′-(trifluoromethyl)-1,1′-biphenyl-2-carboxamide was obtained from 4-methoxy-4′-(trifluoromethyl)-1,1′-biphenyl-2-carboxylic acid and tert-butyl 4-aminophenyl[2-(2-pyridinyl)ethyl]carbamate in the same manner as in Example 29 as white crystals.

¹H-NMR(DMSO-d₆): δ 2.96 (2H, t, J=7.4 Hz), 3.33 (2H, td, J=7.4, 5.8 Hz), 3.86 (3H, s), 5.53 (1H, t, J=5.8 Hz), 6.51 (2H, d, J=8.9 Hz), 7.1-7.8 (8H, m), 8.45-8.55 (1H, m), 9.91 (1H, s)

ESI-MS(m/z): 514(M+Na)⁺, 492(M+H)⁺

### EXAMPLE 72

4-Methoxy-4′-methyl-N-(4-{[2-(2-pyridinyl)ethyl]amino}-phenyl)-1,1′-biphenyl-2-carboxamide was obtained from 4-methoxy-4′-methyl-1,1′-biphenyl-2-carboxylic acid and tert-butyl 4-aminophenyl[2-(2-pyridinyl)ethyl]carbamate in the same manner as in Example 29 as white crystals.

¹H-NMR(DMSO-d₆): δ 2.28 (3H, s), 2.96 (2H, t, J=7.0 Hz), 3.33 (2H, td, J=7.0, 5.7 Hz), 3.83 (3H, s), 5.50 (1H, t, J=5.7 Hz), 6.51 (2H, d, J=8.8 Hz), 7.0-7.4 (12H, m), 7.65-7.75 (1H, m), 8.50 (1H, d, J=4.1 Hz), 9.78 (1H, s)

ESI-MS(m/z): 460(M+Na)⁺, 438(M+H)⁺

### EXAMPLE 73

4′-Chloro-4-methoxy-N-(4-{[2-(2-pyridinyl)ethyl]amino}-phenyl)-1,1′-biphenyl-2-carboxamide was obtained from 4′-chloro-4-methoxy-1,1′-biphenyl-2-carboxylic acid and tert-butyl 4-aminophenyl[2-(2-pyridinyl)ethyl]carbamate in the same manner as in Example 29 as white crystals.

¹H-NMR(DMSO-d₆): δ 2.96 (2H, t, J=7.1 Hz), 3.34 (2H, td, J=7.1, 5.7 Hz), 3.85 (3H, s), 5.52 (1H, t, J=5.7 Hz), 6.51 (2H, d, J=8.8 Hz), 7.1-7.4 (11H, m), 7.7-7.85 (1H, m), 8.55-8.65 (1H, m), 9.84 (1H, s)

ESI-MS(m/z) 480(M+Na)⁺, 458 (M+H)⁺

### EXAMPLE 74

4′-Fluoro-4-methoxy-N-(4-{[2-(2-pyridinyl)ethyl]amino}-phenyl)-1,1′-biphenyl-2-carboxamide was obtained from 4′-fluoro-4-methoxy-1,1′-biphenyl-2-carboxylic acid and tert-butyl 4-aminophenyl[2-(2-pyridinyl)ethyl]carbamate in the same manner as in Example 29 as white crystals.

¹H-NMR(DMSO-d₆): δ 2.96 (2H, t, J=7.0 Hz), 3.33 (2H, td, J=7.0, 5.7 Hz), 3.84 (3H, s), 5.52 (1H, t, J=5.7 Hz), 6.51 (2H, d, J=8.8 Hz), 7.0-7.5 (11H, m), 7.65-7.8 (1H, m), 8.50 (1H, d, J=4.8 Hz), 9.80 (1H, s)

ESI-MS(m/z): 464(M+Na)⁺, 442(M+H)⁺

### EXAMPLE 75

To a suspension of tert-butyl 4-aminophenyl[2-(2-pyridinyl)ethyl]carbamate (1.567 g), 2-(4-fluorophenyl)-1-cyclohexene-1-carboxylic acid (1.1 g) and 1-hydroxybenzotriazole hydrate(766 mg) in tetrahydrofuran (40 ml) was added 1-[3-(dimethylamino)propyl]-3-ethylcarbodiimide (776 mg) at ambient temperature. The resulting solution was stirred at ambient temperature for 20 hours and poured into water. The separated organic layer was washed with brine, dried over magnesium sulfate and evaporated in vacuo. The residue was purified by column chromatography on silica gel eluting with hexane:ethyl acetate (1:2) to give tert-butyl 4-({[2-(4-fluorophenyl)-1-cyclohexen-1-yl]carbonyl}amino)-phenyl[2-(2-pyridinyl)ethyl]carbamate (1.59 g) as a light yellow solid.

¹H-NMR(DMSO-d₆): δ 1.29 (9H, s), 1.7-1.9 (4H, m), 2.35-2.5 (4H, m), 2.85 (2H, t, J=7.5 Hz), 3.84 (2H, t, J=7.5 Hz), 7.01 (2H, d, J=8.9 Hz), 7.10 (2H, d, J=8.9 Hz), 7.15-7.35 (6H, m), 7.6-7.75 (1H, m), 8.43 (1H, d, J=4.8 Hz), 9.58 (1H, s)

APCI-MS(m/z): 516(M+H)⁺

### EXAMPLE 76

To a solution of tert-butyl 4-({[2-(4-fluorophenyl)-1-cyclohexen-1-yl]carbonyl}amino)phenyl[2-(2-pyridinyl)ethyl]carbamate (1.58 g) in dichloromethane (10 ml) was added trifluoroacetic acid (2.8 g) at ambient temperature and the mixture was stirred at ambient temperature for 19 hours. The mixture was evaporated in vacuo and a mixture of dichloromethane and aqueous sodium hydrogencarbonate solution was added to the residue. The separated organic layer was washed with brine, dried over magnesium sulfate and evaporated in vacuo. The residue was purified by column chromatography on silica gel eluting with ethyl acetate and crystallized from ethyl acetate to give 2-(4-fluorophenyl)-N-(4-{[2-(2-pyridinyl)ethyl]amino}phenyl)-1-cyclohexene-1-carboxamide (796 mg) as white crystals.

¹H-NMR(DMSO-d₆): δ 1.7-1.9 (4H, m), 2.35-2.5 (4H, m), 2.96 (2H, t, J=7.4 Hz), 3.34 (2H, td, J=7.4, 5.8 Hz), 5.51 (1H, t, J=5.8 Hz), 6.50 (2H, d, J=8.9 Hz), 7.2-7.6 (15H, m), 7.65-7.8 (1H, m), 8.52 (1H, d, J=4.9 Hz), 9.80 (1H, s)

APCI-MS(m/z): 416(M+H)⁺

Preparation 48

A mixture of tert-butyl 4-(2-aminoethyl)-1,3-thiazol-2-ylcarbamate (0.882 g), 1-fluoro-4-nitrobenzene (0.511 g) and triethylamine (0.76 ml) in 1,3-dimethyl-2-imidazolidinone (10 ml) was heated at 50° C. for 3 hours. The reaction mixture was cooled to ambient temperature, poured into water and extracted with ethyl acetate. The organic layer was washed with brine, dried over magnesium sulfate, filtered and concentrated in vacuo. The residue was purified by column chromatography on silica gel eluting with hexane:ethyl acetate (2:1) to give tert-butyl 4-[2-(4-nitroanilino)ethyl]-1,3-thiazol-2-ylcarbamate (0.763 g) as a yellow oil.

¹H-NMR(CDCl₃): δ 1.54 (9H, s), 2.97 (2H, t, J=6.3 Hz), 3.47 (2H, q, J=6.3 Hz), 5.04 (1H, br s), 6.48 (2H, d, J=9.2 Hz), 6.59 (1H, s), 8.04 (2H, d, J=9.2 Hz)

Preparation 49

To a solution of tert-butyl 4-[2-(4-nitroanilino)ethyl]-1,3-thiazol-2-ylcarbamate (0.749 g) and 4-dimethylaminopyridine (25 mg) in tetrahydrofuran (30 ml) was added di-tert-butyl dicarbonate (0.673 g) and the mixture was heated at 50° C. for an hour. The reaction mixture was cooled to ambient temperature and concentrated in vacuo to give tert-butyl 2-{2-[(tert-butoxycarbonyl)amino]-1,3-thiazol-4-yl}ethyl(4-nitrophenyl)carbamate (0.955 g) as a yellow oil. The product was used for the next step without further purification.

Preparation 50

A solution of tert-butyl 2-{2-[(tert-butoxycarbonyl)amino]-1,3-thiazol-4-yl}ethyl(4-nitrophenyl)carbamate (0.955 g) in methanol (30 ml) was hydrogenated over 10% palladium on carbon at ambient temperature under atmospheric pressure of hydrogen for an hour. The reaction mixture was filtered through a pad of celite, and the filtrate was concentrated in vacuo. The residue was purified by column chromatography on silica gel eluting with hexane:ethyl acetate (2:1) to give tert-butyl 4-{2-[N-(4-aminophenyl)-N-(tert-butoxycarbonyl)amino]ethyl}-1,3-thiazol-2-ylcarbamate (0.709 g) as a yellow oil.

¹H-NMR(CDCl₃): δ 1.51 (18H, s), 2.94 (2H, t, J=6.6 Hz), 3.38 (2H, t, J=6.6 Hz), 6.52 (2H, d, J=8.6 Hz), 6.60 (2H, d, J=8.9 Hz), 6.76 (1H, s)

### EXAMPLE 77

To a solution of tert-butyl 4-{2-[N-(4-aminophenyl)-N-(tert-butoxycarbonyl)amino]ethyl}-1,3-thiazol-2-ylcarbamate (0.329 g), 5-methyl-4′(trifluoromethyl)-1,1′-biphenyl-2-carboxylic acid (0.212 g) and 1-hydroxybenzotriazole (0.123 g) in N,N-dimethylformamide (15 ml) was added 1-[3-(dimethylamino)propyl]-3-ethylcarbodiimide hydrochloride (0.174 g), followed by addition of triethylamine (0.16 ml) at ambient temperature. The reaction mixture was stirred at 50° C. for 12 hours and concentrated in vacuo. The residue was dissolved in ethyl acetate and water, and extracted with ethyl acetate. The organic layer was washed with water and brine, dried over magnesium sulfate, filtered and concentrated in vacuo. The residue was purified by column chromatography on silica gel eluting with hexane:ethyl acetate (1:1) to give tert-butyl 2-{2-[(tert-butoxycarbonyl)amino]-1,3-thiazol-4-yl}ethyl[4-({[5-methyl-4′-(trifluoromethyl)-1,1′-biphenyl-2-yl]carbonyl}amino)phenyl]carbamate (0.387 g) as a pale yellow foam.

### EXAMPLE 78

To a solution of tert-butyl 2-{2-[(tert-butoxycarbonyl)amino]-1,3-thiazol-4-yl}ethyl[4-({[5-methyl-4′-(trifluoromethyl)-1,1′-biphenyl-2-yl]carbonyl}amino)phenyl]-carbamate (0.387 g) in dichloromethane (15 ml) was added trifluoroacetic acid (1.3 ml). The reaction mixture was stirred for 15 hours, quenched with 10% aqueous potassium carbonate solution and extracted with dichloromethane. The organic layer was washed with brine, dried over magnesium sulfate, filtered and concentrated in vacuo. The residue was recrystallized from ethyl acetate-diisopropyl ether to give N-(4-{[2-(2-amino-1,3-thiazol-4-yl)ethyl]amino}phenyl)-5-methyl-4′-(trifluoromethyl)-1,1′-biphenyl-2-carboxamide (0.163 g) as a white solid.

¹H-NMR(DMSO-d₆): δ 2.41 (3H, s), 2.63 (2H, t, J=7.3 Hz), 3.19 (2H, q, J=6.9 Hz), 5.46 (1H, t, J=5.7 Hz), 6.20 (1H, s), 6.47 (2H, d, J=8.9 Hz), 6.85 (2H, s), 7.19 (1H, d, J=8.9 Hz), 7.32 (2H, d, J=10.2 Hz), 7.48 (1H, d, J=7.6 Hz), 7.61 (2H, d, J=7.9 Hz), 7.74 (2H, d, J=8.2 Hz), 9.84 (1H, s)

ESI-MS(m/z): 497(M+H)⁺

### EXAMPLE 79

tert-Butyl 2-{2-[(tert-butoxycarbonyl)amino]-1,3-thiazol-4-yl}ethyl(4-{[(4′,5-dimethyl-1,1′-biphenyl-2-yl)carbonyl]amino}phenyl)carbamate was obtained from 4′,5-dimethyl-1,1′-biphenyl-2-carboxylic acid and tert-butyl 4-{2-[N-(4-aminophenyl)-N-(tert-butoxycarbonyl)amino]ethyl}-1,3-thiazol-2-ylcarbamate in the same manner as in Example 77 as a pale yellow foam.

### EXAMPLE 80

N-(4-{[2-(2-Amino-1,3-thiazol-4-yl)ethyl]amino}phenyl)-4′,5-dimethyl-1,1′-biphenyl-2-carboxamide was obtained from tert-butyl 2-{2-[(tert-butoxycarbonyl)amino]-1,3-thiazol-4-yl}ethyl(4-{[(4′,5-dimethyl-1,1′-biphenyl-2-yl)carbonyl]amino}phenyl)carbamate in the same manner as in Example 78 as an orange foam.

¹H-NM(DMSO-d₆): δ 2.29 (3H, s), 2.38 (3H, s), 2.63 (2H, t, J=7.3 Hz), 3.19 (2H, q, J=6.9 Hz), 5.43 (1H, t, J=5.7 Hz), 6.20 (1H, s), 6.47 (2H, d, J=8.9 Hz), 6.85 (2H, s), 7.14-7.24 (6H, m), 7.32 (2H, d, J=8.2 Hz), 7.37 (2H, d, J=8.2 Hz), 9.69 (1H, s)

ESI-MS(m/z): 443(M+H)⁺

### EXAMPLE 81

To a solution of 6-methyl-4′-(trifluoromethyl)-1,1′-biphenyl-2-carboxylic acid (232 mg) in toluene (5 ml) were added thionyl chloride (0.1 ml) and N,N-dimethylformamide (1 drop) and the mixture was stirred at 100° C. for 3 hours. The mixture was evaporated in vacuo and the residue was dissolved in tetrahydrofuran (2 ml) . The obtained acid chloride solution in tetrahydrofuran was added to a solution of tert-butyl 4-aminophenyl (2-{2-[(tert-butoxycarbonyl)amino]-1,3-thiazol-4-yl}ethyl)carbamate (300 mg) and triethylamine (0.19 ml) in tetrahydrofuran (5 ml) at ambient temperature and the mixture was stirred at ambient temperature for 2 hours. The mixture was poured into water and extracted with ethyl acetate. The organic layer was washed with brine, dried over magnesium sulfate and evaporated in vacuo. The residue was purified by column chromatography on silica gel eluting with hexane:ethyl acetate (3:1) to give tert-butyl 2-{2-[(tert-butoxycarbonyl)amino]-1,3-thiazol-4-yl}ethyl[4-({[6-methyl-4′-(trifluoromethyl)-1,1′-biphenyl-2-yl]carbonyl}amino)phenyl]-carbamate (387 mg) as a yellow foam.

### EXAMPLE 82

To a solution of tert-butyl 2-{2-[(tert-butoxycarbonyl)amino]-1,3-thiazol-4-yl}ethyl[4-({[6-methyl-4′-(trifluoromethyl)-1,1′-biphenyl-2-yl]carbonyl}amino)phenyl]-carbamate (387 mg) in dichloromethane (15 ml) was added trifluoroacetic acid (1.7 ml). The reaction mixture was stirred for 15 hours, quenched with 10% aqueous potassium carbonate solution and extracted with dichloromethane. The organic layer was washed with brine, dried over magnesium sulfate, filtered and concentrated in vacuo. The residue was purified by column chromatography on silica gel eluting with hexane:ethyl acetate (3:1) to give N-(4-{[2-(2-amino-1,3-thiazol-4-yl)ethyl]amino}phenyl)-6-methyl-4′-(trifluoromethyl)-1,1′-biphenyl-2-carboxamide (179 mg) as an orange foam.

¹H-NMR(DMSO-d₆): δ 2.08 (3H, s), 2.61 (2H, t, J=7.1 Hz), 3.16 (2H, t, J=7.1 Hz), 5.41 (1H, t, J=5.6 Hz), 6.18 (1H, s), 6.42 (2H, d, J=8.6 Hz), 6.83 (2H, br s), 7.05 (2H, d, J=8.6 Hz), 7.37-7.48 (5H, m), 7.73 (2H, d, J=8.2 Hz), 9.68 (1H, s)

ESI-MS(m/z): 497(M+H)⁺

### EXAMPLE 83

tert-Butyl 2-{2-[(tert-butoxycarbonyl)amino]-1,3-thiazol-4-yl}ethyl[4-({[4-methyl-4′-(trifluoromethyl)-1,1′-biphenyl-2-yl]carbonyl}amino)phenyl]carbamate was obtained from 4-methyl-4′-(trifluoromethyl)-1,1′-biphenyl-2-carboxylic acid and tert-butyl 4-aminophenyl(2-{2-[(tert-butoxycarbonyl)amino]-1,3-thiazol-4-yl}ethyl)carbamate in the same manner as in Example 81 as a pale yellow oil.

### EXAMPLE 84

N-(4-{[2-(2-Amino-1,3-thiazol-4-yl)ethyl]amino}phenyl)-4-methyl-4′-(trifluoromethyl)-1,1′-biphenyl-2-carboxamide was obtained from tert-butyl 2-{2-[(tert-butoxycarbonyl)amino]-1,3-thiazol-4-yl}ethyl[4-({[4-methyl-4′-(trifluoromethyl)-1,1′-biphenyl-2-yl]carbonyl}amino)phenyl]carbamate in the same manner as in Example 82 as a white solid.

¹H-NMR(DMSO-d₆): δ 2.41 (3H, s), 2.62 (2H, t, J=7.1 Hz), 3.21 (2H, t, J=7.1 Hz), 5.47 (1H, br s), 6.20 (1H, s), 6.48 (2H, d, J=8.9 Hz), 6.84 (2H, s), 7.20 (2H, d, J=8.9 Hz), 7.38(1H, s), 7.39 (2H, d, J=7.9 Hz), 7.60 (2H, d, J=7.9 Hz), 7.73 (2H, d, J=8.9 Hz), 9.90 (1H, s)

ESI-MS(m/z): 497(M+H)⁺

### EXAMPLE 85

tert-Butyl 2-{2-[(tert-butoxycarbonyl)amino]-1,3-thiazol-4-yl}ethyl(4-{[(4′,6-dimethyl-1,1′-biphenyl-2-yl)carbonyl]amino}phenyl)carbamate was obtained from 4′,6-dimethyl-1,1′-biphenyl-2-carboxylic acid and tert-butyl 4-aminophenyl(2-{2-[(tert-butoxycarbonyl)amino]-1,3-thiazol-4-yl}ethyl)carbamate in the same manner as in Example 81 as a pale yellow oil.

### EXAMPLE 86

N-(4-{[2-(2-Amino-1,3-thiazol-4-yl)ethyl]amino}phenyl)-4′,6-dimethyl-1,1′-biphenyl-2-carboxamide was obtained from tert-butyl 2-{2-[(tert-butoxycarbonyl)amino]-1,3-thiazol-4-yl}ethyl(4-{[(4′,6-dimethyl-1,1′-biphenyl-2-yl)carbonyl]amino}phenyl)carbamate in the same manner as in Example 82 as a yellow foam.

¹H-NMR(DMSO-d₆): δ 2.07 (3H, s), 2.29 (3H, s), 2.60 (2H, t, J=7.1 Hz), 3.17 (2H, t, J=7.1 Hz), 5.38 (1H, t, J=5.7 Hz), 6.19 (1H, s), 6.42 (2H, d, J=8.9 Hz), 6.83 (2H, s), 7.08-7.14 (6H, m), 7.28-7.37 (3H, m), 9.54 (1H, s)

ESI-MS(m/z): 443(M+H)⁺

### EXAMPLE 87

tert-Butyl 2-{2-[(tert-butoxycarbonyl)amino]-1,3-thiazol-4-yl}ethyl(4-{[(4,4′-dimethyl-1,1′-biphenyl-2-yl)carbonyl]amino}phenyl)carbamate was obtained from 4,4′-dimethyl-1,1′-biphenyl-2-carboxylic acid and tert-butyl 4-aminophenyl(2-{2-[(tert-butoxycarbonyl)amino]-1,3-thiazol-4-yl}ethyl)carbamate in the same manner as in Example 81 as a pale yellow oil.

### EXAMPLE 88

N-(4-{[2-(2-Amino-1,3-thiazol-4-yl)ethyl]amino}phenyl)-4,4′-dimethyl-1,1′-biphenyl-2-carboxamide was obtained from tert-butyl 2-{2-[(tert-butoxycarbonyl)amino]-1,3-thiazol-4-yl}ethyl(4-{[(4,4′-dimethyl-1,1′-biphenyl-2-yl)carbonyl]amino}phenyl)carbamate in the same manner as in Example 82 as a pale brown foam.

¹H-NMR(DMSO-d₆): δ 2.07 (3H, s), 2.29 (3H, s), 2.60 (2H, t, J=7.1 Hz), 3.17 (2H, t, J=7.1 Hz), 5.38 (1H, t, J=5.7 Hz), 6.19 (1H, s), 6.42 (2H, d, J=8.9 Hz), 6.83 (2H, s), 7.08-7.14 (6H, m), 7.28-7.37 (3H, m), 9.54 (1H, s)

ESI-MS(m/z): 443(M+H)⁺

### EXAMPLE 89

tert-Butyl 2-{2-[(tert-butoxycarbonyl)amino]-1,3-thiazol-4-yl}ethyl(4-{[(4′-chloro-5-methyl-1,1′-biphenyl-2-yl)carbonyl]amino}phenyl)carbamate was obtained from 4′-chloro-5-methyl-1,1′-biphenyl-2-carboxylic acid and tert-butyl 4-aminophenyl(2-{2-[(tert-butoxycarbonyl)amino]-1,3-thiazol-4-yl}ethyl)carbamate in the same manner as in Example 81 as a pale yellow oil.

### EXAMPLE 90

N-(4-{[2-(2-Amino-1,3-thiazol-4-yl)ethyl]amino}phenyl)-4′-chloro-5-methyl-1,1′-biphenyl-2-carboxamide was obtained from tert-butyl 2-{2-[(tert-butoxycarbonyl)amino]-1,3-thiazol-4-yl}ethyl(4-{[(4′-chloro-5-methyl-1,1′-biphenyl-2-yl)carbonyl]amino}phenyl)carbamate in the same manner as in Example 82 as a brown foam.

¹H-NMR(DMSO-d₆): δ 2.40 (3H, s), 2.63 (2H, t, J=7.2 Hz), 3.19 (2H, dd, J=12.8, 6.9 Hz), 5.44 (1H, t, J=5.7 Hz), 6.20 (1H, s), 6.48 (2H, d, J=8.9 Hz), 6.84 (2H, s), 7.18-7.29 (4H, m), 7.42-7.44 (5H, m), 9.75 (1H, s)

ESI-MS(m/z): 485(M+Na)⁺

### EXAMPLE 91

tert-Butyl 2-{2-[(tert-butoxycarbonyl)amino]-1,3-thiazol-4-yl}ethyl(4-{[(4′-chloro-4-methyl-1,1′-biphenyl-2-yl)carbonyl]amino}phenyl)carbamate was obtained from 4′-chloro-4-methyl-1,1′-biphenyl-2-carboxylic acid and tert-butyl 4-aminophenyl(2-{2-[(tert-butoxycarbonyl)amino]-1,3-thiazol-4-yl}ethyl)carbamate in the same manner as in Example 81 as a pale yellow oil.

### EXAMPLE 92

N-(4-{[2-(2-Amino-1,3-thiazol-4-yl) ethyl]amino}phenyl)-4′-chloro-4-methyl-1,1′-biphenyl-2-carboxamide was obtained from tert-butyl 2-{2-[(tert-butoxycarbonyl)amino]-1,3-thiazol-4-yl}ethyl(4-{[(4′-chloro-4-methyl-1,1′-biphenyl-2-yl)carbonyl]amino}phenyl)carbamate in the same manner as in Example 82 as a pale brown solid.

¹H-NMR(DMSO-d₆): δ 2.39 (3H, s), 2.64 (2H, t, J=7.2 Hz), 3.20 (2H, t, J=7. Hz), 5.46 (1H, br s), 6.20 (1H, s), 6.48 (2H, d, J=8.6 Hz), 6.84 (2H, s), 7.21 (2H, d, J=8.9 Hz), 7.30-7.41 (8H, m), 9.83 (1H, s)

ESI-MS(m/z): 485(M+Na)⁺

### EXAMPLE 93

tert-Butyl 2-{2-[(tert-butoxycarbonyl)amino]-1,3-thiazol-4-yl}ethyl[4-({[6-methoxy-4′-(trifluoromethyl)-1,1′-biphenyl-2-yl]carbonyl}amino)phenyl]carbamate was obtained from 6-methoxy-4′-(trifluoromethyl)-1,1′-biphenyl-2-carboxylic acid and tert-butyl 4-aminophenyl(2-{2-[(tert-butoxycarbonyl)amino]-1,3-thiazol-4-yl}ethyl) carbamate in the same manner as in Example 81 as a pale yellow oil.

### EXAMPLE 94

N-(4-{[2-(2-Amino-1,3-thiazol-4-yl)ethyl]amino}phenyl)-6-methoxy-4′-(trifluoromethyl)-1,1′-biphenyl-2-carboxamide was obtained from tert-butyl 2-{2-[(tert-butoxycarbonyl)amino]-1,3-thiazol-4-yl}ethyl[4-({[6-methoxy-4′-(trifluoromethyl)-1,1′-biphenyl-2-yl]carbonyl}amino)phenyl]carbamate in the same manner as in Example 82 as a yellow foam.

¹H-NMR(DMSO-d₆): δ 2.61 (2H, t, J=7.2 Hz), 3.17 (2H, q, J=6.7 Hz), 3.75 (3H, s), 5.43 (1H, t, J=5.6 Hz), 6.18 (1H, s), 6.43 (2H, d, J=8.9 Hz), 6.83 (2H, br s), 7.06 (2H, d, J=8.9 Hz), 7.14 (1H, d, J=6.9 Hz), 7.23 (1H, d, J=7.9 Hz), 7.45-7.50 (3H, m), 7.68 (2H, d, J=8.2 Hz), 9.70 (1H, s)

ESI-MS(m/z): 513(M+H)⁺

Preparation 51

A mixture of 2-(2-methyl-1,3-thiazol-4-yl) ethylamine (6.823 g), 1-fluoro-4-nitrobenzene (8.123 g) and triethylamine (5.829 g) in 1,3-dimethyl-2-imidazolidinone (50 ml) was heated at 50° C. for 16 hours. The reaction mixture was cooled to ambient temperature, poured into water and extracted with ethyl acetate. The organic layer was washed with brine, dried over magnesium sulfate, filtered and concentrated in vacuo. The residue was purified by column chromatography on silica gel eluting with hexane:ethyl acetate (2:1) to give N-[2-(2-methyl-1,3-thiazol-4-yl)ethyl]-4-nitroaniline (7.764 g) as a yellow oil.

¹H-NMR(CDCl₃): δ 2.78 (3H, s), 3.05 (2H, t, J=6.3 Hz), 3.54 (2H, t, J=6.3 Hz), 6.54 (2H, d, J=8.9 Hz), 6.83 (1H, s), 8.09 (2H, d, J=9.2 Hz)

Preparation 52

To a solution of N-[2-(2-methyl-1,3-thiazol-4-yl)ethyl]-4-nitroaniline (7.764 g) and 4-dimethylaminopyridine (1.081 g) in tetrahydrofuran (100 ml) was added di-tert-butyl dicarbonate (8.366 g) and the mixture was heated at 50° C. for 12 hours. The reaction mixture was cooled to ambient temperature and concentrated in vacuo. The residue was dissolved in ethyl acetate and water, and extracted with ethyl acetate. The organic layer was washed with water and brine, dried over magnesium sulfate, filtered and concentrated in vacuo. The residue was purified by column chromatography on silica gel eluting with hexane:ethyl acetate (4:1) to give tert-butyl 2-(2-methyl-1,3-thiazol-4-yl)ethyl(4-nitrophenyl)carbamate (10.62 g) as a dark orange oil.

¹H-NMR(CDCl₃): δ 1.47 (9H, s), 2.60 (3H, s), 3.03 (2H, t, J=7.0 Hz), 4.08 (2H, t, J=7.0 Hz), 6.76 (1H, s), 7.31 (2H, d, J=9.2 Hz), 8.14 (2H, d, J=9.2 Hz)

Preparation 53

A solution of tert-butyl 2-(2-methyl-1,3-thiazol-4-yl)ethyl(4-nitrophenyl)carbamate (10.63 g) in methanol (100 ml) was hydrogenated over 10% palladium on carbon (5.0 g, 50% wet) at ambient temperature under atmospheric pressure of hydrogen for 4.5 hours. The reaction mixture was filtered through a pad of celite, and the filtrate was concentrated in vacuo. The residue was purified by column chromatography on silica gel eluting with chloroform:methanol (19:1) to give tert-butyl 4-aminophenyl[2-(2-methyl-1,3-thiazol-4-yl)ethyl]carbamate (9.295 g) as yellow crystals.

¹H-NMR(CDCl₃): δ 1.39 (9H, s), 2.64 (3H, s), 2.96 (2H, t, J=7.6 Hz), 3.63 (2H, br s), 3.90 (2H, t, J=7.6 Hz), 6.67 (2H, d, J=7.9 Hz), 6.78 (1H, s), 6.90 (2H, d, J=7.9 Hz)

### EXAMPLE 95

To a solution of 6-methyl-4′-(trifluoromethyl)-1,1′-biphenyl-2-carboxylic acid (178 mg) in toluene (2 ml) were added thionyl chloride (151 mg) and N,N-dimethylformamide (1 drop) and the mixture was stirred at 80° C. for 2 hours. The mixture was evaporated in vacuo and the residue was dissolved in tetrahydrofuran (2 ml). The obtained acid chloride solution in tetrahydrofuran was added to a solution of tert-butyl 4-aminophenyl[2-(2-methyl-1,3-thiazol-4-yl)ethyl]carbamate (176.5 mg) and triethylamine (107.1 mg) in tetrahydrofuran (5 ml) at ambient temperature and the mixture was stirred at ambient temperature for 30 minutes. The mixture was poured into water and extracted with ethyl acetate. The organic layer was washed with brine, dried over magnesium sulfate and evaporated in vacuo to give tert-butyl 2-(2-methyl-1,3-thiazol-4-yl)ethyl[4-({[6-methyl-4′-(trifluoromethyl)-1,1′-biphenyl-2-yl]carbonyl}amino)-phenyl]carbamate (350.4 mg) as an orange foam.

### EXAMPLE 96

To a solution of tert-butyl 2-(2-methyl-1,3-thiazol-4-yl)ethyl[4-({[6-methyl-4′-(trifluoromethyl)-1,1′-biphenyl-2-yl]carbonyl}amino)phenyl]carbamate (315.1 mg) in dichloromethane (8 ml) was added trifluoroacetic acid (1.13 ml). The reaction mixture was stirred for 15 hours, quenched with 10% aqueous potassium carbonate solution and extracted with dichloromethane. The organic layer was washed with brine, dried over magnesium sulfate, filtered and concentrated in vacuo. The residue was recrystallized from ethyl acetate-hexane to give 6-methyl-N-(4-{[2-(2-methyl-1,3-thiazol-4-yl)ethyl]amino}phenyl)-4′-(trifluoromethyl)-1,1′-biphenyl-2-carboxamide (215.6 mg) as pale yellow crystals.

¹H-NMR(CDCl₃): δ 2.16 (3H, s), 2.69 (3H, s), 2.98 (2H, t, J=6.6 Hz), 3.40 (2H, t, J=6.6 Hz), 6.47 (2H, d, J=8.6 Hz), 6.67 (1H, s), 6.76 (1H, s), 6.83 (2H, d, J=8.6 Hz), 7.38-7.40 (2H, m), 7.45 (2H, d, J=8.0 Hz), 7.61 (1H, t, J=5.3 Hz), 7.70 (2H, d, J=8.0 Hz)

ESI-MS(m/z): 496(M+H)⁺

### EXAMPLE 97

tert-Butyl 2-(2-methyl-1,3-thiazol-4-yl)ethyl[4-({[5-methyl-4′-(trifluoromethyl)-1,1′-biphenyl-2-yl]carbonyl}amino)phenyl]carbamate was obtained from 5-methyl-4′-(trifluoromethyl)-1,1′-biphenyl-2-carboxylic acid and tert-butyl 4-aminophenyl[2-(2-methyl-1,3-thiazol-4-yl)ethyl]carbamate in the same manner as in Example 95 as a pale yellow foam.

### EXAMPLE 98

5-Methyl-N-(4-{[2-(2-methyl-1,3-thiazol-4-yl)ethyl]amino}phenyl)-4′-(trifluoromethyl)-1,1′-biphenyl-2-carboxamide was obtained from tert-butyl 2-(2-methyl-1,3-thiazol-4-yl)ethyl[4-({[5-methyl-4′-(trifluoromethyl)-1,1′-biphenyl-2-yl]carbonyl}amino)phenyl]carbamate in the same manner as in Example 96 as pale yellow crystals.

¹H-NMR(CDCl₃): δ 2.45 (3H, s), 2.69 (3H, s), 2.99 (2H, t, J=6.6 Hz), 3.42 (2H, t, J=6.6 Hz), 6.50 (2H, d, J=8.6 Hz), 6.68 (1H, s), 6.77 (1H, s), 6.93 (2H, d, J=8.6 Hz), 7.21-7.32 (2H, m), 7.57-7.72 (5H, m)

ESI-MS(m/z): 496(M+H)⁺

Preparation 54

A mixture of tert-butyl 6-(2-aminoethyl)-2-pyridinylcarbamate (0.776 g), 1-fluoro-4-nitrobenzene (0.462 g) and triethylamine (0.69 ml) in 1,3-dimethyl-2-imidazolidinone (10 ml) was heated at 50° C. for 3.5 hours. The reaction mixture was cooled to ambient temperature, poured into water and extracted with ethyl acetate. The organic layer was washed with brine, dried over magnesium sulfate, filtered and concentrated in vacuo. The residue was purified by column chromatography on silica gel eluting with hexane:ethyl acetate (3:2) to give tert-butyl 6-[2-(4-nitroanilino)ethyl]-2-pyridinylcarbamate (0.666 g) as a yellow oil.

¹H-NMR(CDCl₃): δ 1.53 (9H, s), 2.99 (2H, t, J=6.6 Hz), 3.57 (2H, dd, J=12.2, 6.2 Hz), 5.21 (1H, br s), 6.53 (2H, d, J=9.2 Hz), 6.82 (1H, dd, J=7.6, 0.7 Hz), 7.30 (1H, br s), 7.59 (1H, d, J=7.8 Hz), 7.95 (1H, d, J=7.9 Hz), 8.05 (2H, d, J=8.9 Hz)

Preparation 55

A solution of tert-butyl 6-[2-(4-nitroanilino)ethyl]-2-pyridinylcarbamate (553 mg) in methanol (10 ml) was hydrogenated over 10% palladium on carbon (200 mg, 50% wet) at ambient temperature under atmospheric pressure of hydrogen for 2 hours. The reaction mixture was filtered through a short pad of celite, and the filtrate was concentrated in vacuo to give tert-butyl 6-{2-[(4-aminophenyl)amino]ethyl}-2-pyridinylcarbamate (426 mg) as a brown foamy solid.

¹H-NMR(CDCl₃): δ 1.52 (9H, s), 3.07 (2H, br s), 3.55 (2H, br s), 6.64 (2H, brd, J=8.6 Hz), 6.78 (1H, d, J=6.9 Hz), 7.07 (2H, brd, J=8.6 Hz), 7.57 (1H, t, J=7.7 Hz), 7.67 (1H, d, J=7.9 Hz)

ESI-MS(m/z): 329(M+H)⁺

### EXAMPLE 99

To a solution of tert-butyl 6-{2-[(4-aminophenyl)amino]ethyl}-2-pyridinylcarbamate (213 mg), 5-methyl-4′-(trifluoromethyl)-1,1′-biphenyl-2-carboxylic acid (181 mg) and 1-hydroxybenzotriazole (129 mg) in N,N-dimethylformamide (10 ml) was added 1-[3-(dimethylamino)propyl]-3-ethylcarbodiimide hydrochloride (WSC.HCl) (162 mg), followed by addition of triethylamine (92 mg) at ambient temperature. The reaction mixture was stirred for 4 hours and concentrated in vacuo. The residue was dissolved in ethyl acetate and water, and extracted with ethyl acetate. The organic layer was washed with brine, dried over magnesium sulfate, filtered and concentrated in vacuo. The residue was purified by column chromatography on silica gel eluting with chloroform:methanol (39:1) to give tert-butyl 6-(2-{[4-({[5-methyl-4′-(trifluoromethyl)-1,1′-biphenyl-2-yl]carbonyl}amino)phenyl]amino}ethyl)-2-pyridinylcarbamate (339 mg) as a brown foam.

ESI-MS(m/z): 613(M+Na)⁺

### EXAMPLE 100

To a solution of tert-butyl 6-(2-{[4-({[5-methyl-4′-(trifluoromethyl)-1,1′-biphenyl-2-yl]carbonyl}amino)phenyl]-amino}ethyl)-2-pyridinylcarbamate (339 mg) in dichloromethane (10 ml) was added trifluoroacetic acid (0.99 g) by a syringe at 0° C. The reaction mixture was allowed to warm up to ambient temperature and stirred for 12 hours. The reaction mixture was quenched with 10% aqueous potassium carbonate solution. The separated organic layer was washed with brine, dried over magnesium sulfate, filtered and concentrated in vacuo to give N-(4-{[2-(6-amino-2-pyridinyl)ethyl]amino}phenyl)-5-methyl-4′-(trifluoromethyl)-1,1′-biphenyl-2-carboxamide (193 mg) as a

greenish yellow foam.

¹H-NMR(DMSO-d₆): δ 2.41 (3H, s), 2.70 (2H, t, J=7.3 Hz), 3.23 (2H, t, J=7.3 Hz), 5.85 (2H, br s), 6.26 (1H, d, J=7.6 Hz), 6.38 (1H, d, J=6.6 Hz), 6.49 (2H, d, J=8.9 Hz), 7.17-7.34 (5H, m), 7.48 (1H, d, J=7.6 Hz), 7.61 (1H, d, J=7.9 Hz), 7.74 (2H, d, J=8.2 Hz), 9.84 (1H, s)

ESI-MS(m/z): 491(M+H)⁺

### EXAMPLE 101

tert-Butyl 6-{2-[(4-{[(4′,5-dimethyl-1,1′-biphenyl-2-yl)carbonyl]amino}phenyl)amino]ethyl}-2-pyridinylcarbamate was obtained from 4′,5-dimethyl-1,1′-biphenyl-2-carboxylic acid and tert-butyl 6-{2-[(4-aminophenyl)amino]ethyl}-2-pyridinylcarbamate in the same manner as in Example 99 as a dark brown oil.

ESI-MS (m/z): 559(M+Na)⁺

### EXAMPLE 102

N-(4-{[2-(6-Amino-2-pyridinyl)ethyl]amino}phenyl)-4′,5-dimethyl-1,1′-biphenyl-2-carboxamide was obtained tert-butyl 6-{2-[(4-{[(4′, 5-dimethyl-1, 1′-biphenyl-2-yl) carbonyl]amino}-phenyl)amino]ethyl}-2-pyridinylcarbamate in the same manner as in Example 100 as a pale brown foam.

¹H-NMR(CDCl₃): δ 2.39 (3H, s), 2.42 (3H, s), 2.86 (2H, t, J=6.6 Hz), 3.41 (2H, t, J=6.6 Hz), 4.45 (2H, br s), 6.35 (1H, d, J=7.9 Hz), 6.47-6.51 (3H, m), 6.70 (1H, br s), 6.90 (2H, d, J=8.9 Hz), 7.18-7.37 (7H, m), 7.78 (1H, d, J=7.9 Hz)

ESI-MS(m/z): 437(M+H)⁺

Preparation 56

To a solution of tert-butyl 6-(2-hydroxyethyl)-2-pyridinylcarbamate (3.92 g) in tetrahydrofuran (50 ml) was added potassium t-butoxide (1.85 g), and the mixture was stirred at ambient temperature for 1 hour. 1-Fluoro-4-nitrobenzene (2.79 g) in tetrahydrofuran (10 ml) was added and the mixture was heated at 75° C. for 15 hours. The reaction mixture was cooled to ambient temperature, poured into water and extracted with ethyl acetate. The organic layer was washed with brine, dried over magnesium sulfate, filtered and concentrated in vacuo. The residue was purified by column chromatography on silica gel eluting with hexane:ethyl acetate (4:1→1:1) to give tert-butyl 6-[2-(4-nitrophenoxy)ethyl]-2-pyridinylcarbamate (3.86 g) as a yellow oil.

¹H-NMR(CDCl₃): δ 1.51 (9H, s), 3.16 (2H, t, J=6.7 Hz), 4.41 (2H, t, J=6.7 Hz), 6.90 (1H, d, J=7.2 Hz), 6.94 (2H, d, J=9.5 Hz), 7.26 (1H, br s), 7.60 (1H, t, J=7.7 Hz), 8.17 (2H, d, J=9.2 Hz)

Preparation 57

A solution of tert-butyl 6-[2-(4-nitrophenoxy)ethyl]-2-pyridinylcarbamate (3.858 g) in methanol (150 ml) was hydrogenated over 10% palladium on carbon (1.543 g) at ambient temperature under atmospheric pressure of hydrogen for 1 hour. The reaction mixture was filtered through a short pad of celite, and the filtrate was concentrated in vacuo to give tert-butyl 6-[2-(4-aminophenoxy)ethyl]-2-pyridinylcarbamate (3.42 g) as a yellow oil.

¹H-NMR(CDCl₃): δ 1.49 (9H, s), 3.09 (2H, t, J=6.7 Hz), 4.20 (2H, t, J=6.7 Hz), 6.71 (2H, d, J=8.6 Hz), 6.84 (2H, d, J=8.6 Hz), 6.89 (1H, d, J=7.2 Hz), 7.58 (1H, dd, J=8.2, 7.2 Hz), 7.78 (1H, d, J=8.2 Hz)

### EXAMPLE 103

To a solution of tert-butyl 6-[2-(4-aminophenoxy)ethyl]-2-pyridinylcarbamate (0.504 g), 6-methyl-4′-(trifluoromethyl)-1,1′-biphenyl-2-carboxylic acid (0.429 g) and 1-hydroxybenzotriazole (0.248 g) in N,N-dimethylformamide (15 ml) was added 1-[3-(dimethylamino)propyl]-3-ethylcarbodiimide hydrochloride (WSC.HCl) (0.352 g), followed by addition of triethylamine (0.32 ml) at ambient temperature. The reaction mixture was stirred at 50° C. for 12 hours and concentrated in vacuo. The residue was dissolved in ethyl acetate and water, and extracted with ethyl acetate. The organic layer was washed with water and brine, dried over magnesium sulfate, filtered and concentrated in vacuo. The residue was purified by column chromatography on silica gel eluting with hexane:ethyl acetate (2:1) to give tert-butyl 6-{2-[4-({[6-methyl-4′-(trifluoromethyl)-1,1′-biphenyl-2-yl]carbonyl}amino)phenoxy]ethyl}-2-pyridinylcarbamate (0.756 g) as a pale yellow oil.

¹H-NMR(DMSO-d₆): δ 1.45 (9H, s), 2.09 (3H, s), 3.03 (2H, t, J=6.7 Hz), 4.24 (2H, t, J=6.7 Hz), 6.79 (2H, d, J=8.9 Hz), 6.97 (1H, dd, J=5.3, 2.6 Hz), 7.28 (2H, d, J=8.9 Hz), 7.40-7.49 (5H, m), 7.63-7.65 (2H, m), 7.73 (2H, d, J=7.9 Hz), 9.61 (1H, s), 9.99 (1H, s)

### EXAMPLE 104

To a solution of tert-butyl 6-{2-[4-({[6-methyl-4′-(trifluoromethyl)-1,1′-biphenyl-2-yl]carbonyl}amino)phenoxy]-ethyl}-2-pyridinylcarbamate (0.756 g) in dichloromethane (30 ml) was added trifluoroacetic acid (1.5 ml). The reaction mixture was stirred for 15 hours, quenched with 10% aqueous potassium carbonate solution and extracted with dichloromethane. The organic layer was washed with brine, dried over magnesium sulfate, filtered and concentrated in vacuo. The residue was recrystallized from ethyl acetate-diisopropyl ether to give N-{4-[2-(6-amino-2-pyridinyl)ethoxy]phenyl}-6-methyl-4′-(trifluoromethyl)-1,1′-biphenyl-2-carboxamide (0.416 g) as a white solid.

¹H-NMR(DMSO-d₆): δ 2.09 (3H, s), 2.89 (2H, t, J=6.7 Hz), 4.18 (2H, t, J=6.7 Hz), 5.87 (2H, br s), 6.29 (1H, d, J=8.2 Hz), 6.43 (1H, d, J=7.2 Hz), 6.79 (2H, d, J=8.9 Hz), 7.26-7.32 (3H, m), 7.40-7.49 (5H, m), 7.73 (2H, d, J=8.2 Hz), 9.98 (1H, s)

ESI-MS(m/z): 492(M+H)⁺

### EXAMPLE 105

tert-Butyl 6-{2-[4-({[5-methyl-4′-(trifluoromethyl)-1,1′-biphenyl-2-yl]carbonyl}amino)phenoxy]ethyl}-2-pyridinylcarbamate was obtained from 5-methyl-4′-(trifluoromethyl)-1,1′-biphenyl-2-carboxylic acid and tert-butyl 6-[2-(4-aminophenoxy)ethyl]-2-pyridinylcarbamate in the same manner as in Example 103 as a faintly orange foamy solid.

¹H-NMR(CDCl₃): δ 1.51 (9H, s), 2.45 (3H, s), 3.09 (2H, t, J=6.7 Hz), 4.25 (2H, t, J=6.7 Hz), 6.77 (2H, d, J=8.9 Hz), 6.80 (1H, br s), 6.88 (1H, d, J=7.6 Hz), 7.03 (2H, d, J=8.9 Hz), 7.22 (2H, br s), 7.31 (1H, d, J=7.3 Hz), 7.54-7.78 (7H, m)

### EXAMPLE 106

N-{4-[2-(6-Amino-2-pyridinyl)ethoxy]phenyl}-5-methyl-4′-(trifluoromethyl)-1,1′-biphenyl-2-carboxamide was obtained from tert-butyl 6-{2-[4-({[5-methyl-4′-(trifluoromethyl)-1,1′-biphenyl-2-yl]carbonyl}amino)phenoxy]ethyl}-2-pyridinylcarbamate in the same manner as in Example 104 as colorless crystals.

¹H-NMR(DMSO-d₆): δ 2.42 (3H, s), 2.90 (2H, t, J=6.8 Hz), 3.32 (2H, s), 4.20 (2H, t, J=6.8 Hz), 5.83 (1H, br s), 6.29 (1H, d, J=8.2 Hz), 6.43 (1H, d, J=7.3 Hz), 6.83 (2H, d, J=9.2 Hz), 7.26-7.41 (5H, m), 7.52 (1H, d, J=7.6 Hz), 7.61 (2H, d, J=8.2 Hz), 7.74 (2H, d, J=7.9 Hz), 10.09 (1H, s)

ESI-MS(m/z): 492(M+H)⁺

### EXAMPLE 107

To a solution of tert-butyl 6-[2-(4-aminophenoxy)ethyl]-2-pyridinylcarbamate (0.506 g), 4′,6-dimethyl-1,1′-biphenyl-2-carboxylic acid (0.348 g) and 1-hydroxybenzotriazole (0.249 g) in N,N-dimethylformamide (15-ml) was added 1-[3-(dimethylamino)propyl]-3-ethylcarbodiimide hydrochloride (WSC.HCl) (0.353 g), followed by addition of triethylamine (0.32 ml) at ambient temperature. The reaction mixture was stirred at 50° C. for 12 hours and concentrated in vacuo. The residue was dissolved in ethyl acetate and water, and extracted with ethyl acetate. The organic layer was washed with water and brine, dried over magnesium sulfate, filtered and concentrated in vacuo. The residue was purified by column chromatography on silica gel eluting with hexane:ethyl acetate (2:1) to give tert-butyl 6-[2-(4-{[(4′,6-dimethyl-1,1′-biphenyl-2-yl)carbonyl]amino}phenoxy)ethyl]-2-pyridinylcarbamate (0.712 g) as a pale yellow oil.

¹H-NMR(DMSO-d₆): δ 1.45 (9H, s), 2.08 (3H, s), 2.28 (3H, s), 3.02 (2H, t, J=6.7 Hz), 4.24 (2H, t, J=6.7 Hz), 6.79 (2H, d, J=8.9 Hz), 6.95-6.98 (1H, m), 7.14-7.64 (11H, m), 9.61 (1H, s), 9,83 (1H, s)

### EXAMPLE 108

To a solution of tert-butyl 6-[2-(4-{[(4′,6-dimethyl-1,1′-biphenyl-2-yl)carbonyl]amino}phenoxy)ethyl]-2-pyridinylcarbamate (0.712 g) in dichloromethane (30 ml) was added trifluoroacetic acid (1.53 ml). The reaction mixture was stirred for 15 hours, quenched with 10% aqueous potassium carbonate solution and extracted with dichloromethane. The organic layer was washed with brine, dried over magnesium sulfate,, filtered and concentrated in vacuo. The residue was recrystallized from ethyl acetate-diisopropyl ether to give N-{4-[2-(6-amino-2-pyridinyl)ethoxy]phenyl}-4′,6-dimethyl-1,1′-biphenyl-2-carboxamide (0.484 g) as a white solid.

¹H-NMR(DMSO-d₆): δ 2.08 (3H, s), 2.28 (3H, s) 2.88 (2H, t, J=6.7 Hz), 4.18 (2H, t, J=6.7 Hz), 5.82 (2H, br s), 6.27 (1H, d, J=8.2 Hz), 6.42 (1H, d, J=7.2 Hz), 6.78 (2H, d, J=9.2 Hz), 7.14 (4H, br s), 7.25-7.38 (6H, m), 9.83 (1H, s)

ESI-MS(m/z): 438(M+H)⁺

### EXAMPLE 109

To a solution of 4′,5-dimethyl-1,1′-biphenyl-2-carboxylic acid (266 mg), tert-butyl 6-[2-(4-aminophenoxy)ethyl]-2-pyridinylcarbamate (387 mg) and 1-hydroxybenzotriazole (216 mg) in N,N-dimethylformamide (10 ml) was added 1-[3-(dimethylamino)propyl]-3-ethylcarbodiimide hydrochloride (WSC.HCl) (270 mg), followed by addition of triethylamine (155 mg) at ambient temperature. The reaction mixture was stirred at 50° C. for 16 hours and concentrated in vacuo. The residue was dissolved in ethyl acetate and water, and extracted with ethyl acetate. The organic layer was washed with brine, dried over magnesium sulfate, filtered and concentrated in vacuo. The residue was purified by column chromatography on silica gel eluting with hexane:ethyl acetate (2:1) to give tert-butyl 6-[2-(4-{[(4′,5-dimethyl-1,1′-biphenyl-2-yl)carbonyl]amino}phenoxy)ethyl]-2-pyridinylcarbamate (466 mg) as a pale brown foamy solid.

¹H-NMR(CDCl₃): δ 1.52 (9H, s), 2.39 (3H, s), 2.43 (3H, s), 3.08 (2H, t, J=6.7 Hz), 4.25 (2H, t, J=6.7 Hz), 6.75 (2H, d, J=8.9 Hz), 6.79 (1H, br s), 6.88 (1H, d, J=7.6 Hz), 6.99 (2H, d, J=8.9 Hz), 7.16-7.27 (5H, m), 7.35 (2H, d, J=7.9 Hz), 7.57 (1H, t, J=7.8 Hz), 7.78 (2H, t, J=8.4 Hz)

### EXAMPLE 110

To a solution of tert-butyl 6-[2-(4-{[(4′,5-dimethyl-1,1′-biphenyl-2-yl)carbonyl]amino}phenoxy)ethyl]-2-pyridinylcarbamate (527 mg) in dichloromethane (20 ml) was added trifluoroacetic acid (1.59 g) by a syringe at 0° C. The reaction mixture was allowed to warm up to ambient temperature and stirred for 16 hours. The reaction was quenched with 10% aqueous potassium carbonate solution. The separated organic layer was washed with brine, dried over magnesium sulfate, filtered and concentrated in vacuo. The residue was purified by column chromatography on silica gel eluting with chloroform:methanol (19:1) to give N-{4-[2-(6-amino-2-pyridinyl)ethoxy]phenyl}-4′,5-dimethyl-1,1′-biphenyl-2-carboxamide (388 mg) as a pale brown foamy solid.

¹H-NMR(DMSO-d₆): δ 2.29 (3H, s), 2.39 (3H, s), 2.90 (2H, t, J=6.9 Hz), 3.32 (2H, br s), 4.20 (2H, t, J=6.9 Hz), 5.82 (1H, br s), 6.27 (1H, d, J=8.2 Hz), 6.43 (1H, d, J=6.6 Hz), 6.83 (2H, d, J=8.9 Hz), 7.16(2H, d, J=7.9 Hz), 7.23-7.42 (8H, m), 9.95 (1H, br s)

ESI-MS(m/z): 438(M+H)⁺

### EXAMPLE 111

4-Chloro-N-(4-{[2-(2-pyridinyl)ethyl]amino}phenyl)-4′-(trifluoromethyl)-1,1′-biphenyl-2-carboxamide was obtained from 4-chloro-4′-(trifluoromethyl)-1,1′-biphenyl-2-carboxylic acid and tert-butyl 4-aminophenyl[2-(2-pyridinyl)ethyl]carbamate in the same manner as in Example 29.

¹H-NMR(DMSO-d₆): δ 2.96 (2H, t, J=7.32 Hz), 3.30-3.40 (2H, m), 5.57 (1H, t, J=5.68 Hz), 6.52 (2H, d, J=8.74 Hz), 7.19-7.32 (4H, m), 7.50-7.79 (9H, m), 8.51 (1H, d, J=4.30 Hz), 10.04 (1H, s)

APCI-MS(m/z): 496(M+H)⁺

### EXAMPLE 112

4,4′-Dichloro-N-(4-{[2-(2-pyridinyl)ethyl]amino}phenyl)-1,1′-biphenyl-2-carboxamide was obtained from 4,4′-dichloro-1,1′-biphenyl-2-carboxylic acid and tert-butyl 4-aminophenyl[2-(2-pyridinyl)ethyl]carbamate in the same manner as in Example 29.

¹H-NMR(DMSO-d₆): δ 2.96 (2H, t, J=7.36 Hz), 3.30-3.40 (2H, m), 5.56 (1H, t, J=5.76 Hz), 6.52 (2H, d, J=8.78 Hz), 7.19-7.32 (4H, m), 7.44-7.70 (8H, m), 8.51 (1H, d, J=4.56 Hz), 9.98 (1H, s)

APCI-MS(m/z): 462(M+H)⁺

### EXAMPLE 113

4-Chloro-4′-fluoro-N-(4-{[2-(2-pyridinyl)ethyl]amino}-phenyl)-1,1′-biphenyl-2-carboxamide was obtained from 4-chloro-4′-fluoro-1,1′-biphenyl-2-carboxylic acid and tert-butyl 4-aminophenyl[2-(2-pyridinyl)ethyl]carbamate in the same manner as in Example 29.

¹H-NMR(DMSO-d₆): δ 2.96 (2H, t, J=7.34 Hz), 3.39-3.39 (2H, m), 5.55 (1H, t, J=5.74 Hz), 6.52 (2H, d, J=8.80 Hz), 7.18-7.32 (6H, m), 7.41-7.48 (3H, m), 7.58-7.70 (3H, m), 8.51 (1H, d, J=4.44 Hz), 9.94 (1H, s)

APCI-MS(m/z): 446(M+H)⁺

### EXAMPLE 114

4-Chloro-4′-methyl-N-(4-{[2-(2-pyridinyl)ethyl]amino}-phenyl)-1,1′-biphenyl-2-carboxamide was obtained from 4-chloro-4′-methyl-1,1′-biphenyl-2-carboxylic acid and tert-butyl 4-aminophenyl[2-(2-pyridinyl)ethyl]carbamate in the same manner as in Example 29.

¹H-NMR(DMSO-d₆): δ 2.29 (3H, s), 2.96 (2H, t, J=7.32 Hz), 3.30-3.40 (2H, m), 5.57 (1H, t, J=5.68 Hz), 6.52 (2H, d, J=8.74 Hz), 7.19-7.32 (4H, m), 7.50-7.79 (9H, m), 8.51 (1H, d, J=4.30 Hz), 10.04 (1H, s)

APCI-MS(m/z): 442(M+H)⁺

### EXAMPLE 115

A mixture of 2-(4-methylphenyl)-1-cyclohexene-1-carboxylic acid (325 mg), tert-butyl 4-aminophenyl[2-(2-pyridinyl)ethyl]carbamate (494 mg), 1-hydroxybenzotriazole hydrate (242 mg) and 1-[3-(dimethylamino)propyl]-3-ethylcarbodiimide hydrochloride (301 mg) in dichloromethane (8 ml) was stirred at ambient temperature overnight. Trifluoroacetic acid (8 ml) was added to the reaction mixture and the resultant mixture was stirred at ambient temperature for 4 hours. The reaction mixture was concentrated in vacuo. The residue was dissolved in a mixture of ethyl acetate and water, and the solution was adjusted to pH 8.0 with aqueous potassium carbonate solution. The organic layer was washed with brine and dried over magnesium sulfate. The solvent was evaporated in vacuo and the residue was chromatographed on silica gel eluting with ethyl acetate:n-hexane (8:2). The fractions containing the desired product were collected and evaporated in vacuo and the residue was recrystallized from ethyl acetate and diisopropyl ether to give 2-(4-methylphenyl)-N-(4-{[2-(2-pyridinyl)ethyl]amino}phenyl)-1-cyclohexene-1-carboxamide (85 mg).

¹H-NMR(DMSO-d₆): δ 1.70 (4H, br s), 2.22 (3H, s), 2.33 (4H, br s), 2.93 (2H, t, J=7.33 Hz), 3.26-3.34 (2H, m), 5.45 (1H, s), 6.43 (2H, d, J=8.80 Hz), 7.03-7.07 (4H, m), 7.16-7.30 (4H, m), 7.65-7.73 (1H, m), 8.48-8.51 (1H, m), 9.06 (1H, s)

ESI-MS(m/z): 412(M+H)⁺

### EXAMPLE 116

N-(4-{[2-(2-Pyridinyl)ethyl]amino}phenyl)-2-[4-(trifluoromethyl)phenyl]-1-cyclohexene-1-carboxamide was obtained from 2-[4-(trifluoromethyl)phenyl]-1-cyclohexene1-carboxylic acid and tert-butyl 4-aminophenyl[2-(2-pyridinyl)ethyl]carbamate in the same manner as in Example 115.

¹H-NMR(DMSO-d₆): δ 1.72 (4H, br s), 2.38 (4H, br s), 2.93 (2H, t, J=7.38 Hz), 3.26-3.34 (2H, m), 5.52 (1H, s), 6.43 (2H, d, J=8.80 Hz), 7.00 (2H, d, J=8.80 Hz), 7.17-7.30 (2H, m), 7.48 (2H, d, J=8.16 Hz), 7.65-7.73 (3H, m), 8.48-8.51 (1H, m), 9.20 (1H, s)

ESI-MS(m/z): 466(M+H)⁺

### EXAMPLE 117

2-(4-Methoxyphenyl)-N-(4-{[2-(2-pyridinyl)ethyl]amino}-phenyl)-1-cyclohexene-1-carboxamide was obtained from 2-(4-methoxyphenyl)-1-cyclohexene-1-carboxylic acid and tert-butyl 4-aminophenyl[2-(2-pyridinyl)ethyl]carbamate in the same manner as in Example 115.

¹H-NMR(DMSO-d₆): δ 1.69 (4H, br s), 2.33 (4H, br s), 2.93 (2H, t, J=7.39 Hz), 3.29-3.33 (2H, m), 3.63 (3H, s), 5.44 (1H, br s), 6.43 (2H, d, J=8.80 Hz), 6.80 (2H, d, J=8.80 Hz), 7.05 (2H, d, J=8.80 Hz), 7.17-7.30 (4H, m), 7.65-7.73 (1H, m), 8.48-8.51 (1H, m), 9.54 (1H, s)

ESI-MS(m/z): 428(M+H)⁺

### EXAMPLE 118

2-(4-Chlorophenyl)-N-(4-{[2-(2-pyridinyl)ethyl]amino}-phenyl)-1-cyclohexene-1-carboxamide was obtained from 2-(4-chlorophenyl)-1-cyclohexene-1-carboxylic acid and tert-butyl 4-aminophenyl[2-(2-pyridinyl)ethyl]carbamate in the same manner as in Example 115.

¹H-NMR(DMSO-d₆): δ 1.71 (4H, br s), 2.34 (4H, br s), 2.94 (2H, t, J=7.34 Hz), 3.27-3.33 (2H, m), 5.50 (1H, br s), 6.44 (2H, d, J=8.76 Hz), 7.04 (2H, d, J=8.76 Hz), 7.18-7.36 (6H, m), 7.65-7.73 (1H, m), 8.48-8.51 (1H, m), 9.16(1H, s)

ESI-MS(m/z): 432(M+H)⁺

### EXAMPLE 119

A mixture of 2-[4-(dimethylamino) phenyl]-1-cyclohexene-1-carboxylic acid (367 mg), tert-butyl 4-aminophenyl[2-(2-pyridinyl)ethyl]carbamate (494 mg), 1-hydroxybenzotriazole hydrate (242 mg) and 1-[3-(dimethylamino)propyl]-3-ethylcarbodiimide hydrochloride (301 mg) in N,N-dimethylformamide (10 ml) was stirred at ambient temperature overnight. The reaction-mixture was poured into a mixture of ethyl acetate and water, and the mixture was adjusted to pH 8.0 with aqueous potassium carbonate solution. The organic layer was washed with brine and dried over magnesium sulfate. The solvent was evaporated in vacuo and the residue was dissolved in a mixture of dichloromethane (5 ml) and trifluoroacetic acid (8 ml). The resultant mixture was stirred at ambient temperature for 4 hours. The reaction mixture was concentrated in vacuo. The residue was dissolved in a mixture of ethyl acetate and water, and the solution was adjusted to pH 8.0 with aqueous potassium carbonate solution. The organic layer was washed with brine and dried over magnesium sulfate. The solvent was evaporated in vacuo and the residue was chromatographed on silica gel eluting with ethyl acetate:n-hexane (8:2). The fractions containing the desired product were collected and evaporated in vacuo and the residue was recrystallized from ethyl acetate and diisopropyl ether to give 2-[4-(dimethylamino)phenyl]-N-(4-{[2-(2-pyridinyl)ethyl]amino}phenyl)-1-cyclohexene-1-carboxamide (140 mg).

¹H-NMR(DMSO-d₆): δ 1.68 (4H, br s), 2.32 (4H, br s), 2.83 (6H, s), 2.94 (2H, t, J=7.39 Hz), 3.27-3.33 (2H, m), 5.44 (1H, br s), 6.44 (2H, d, J=8.80 Hz), 6.59 (2H, d, J=8.76 Hz), 7.07-7.30 (6H, m), 7.64-7.73 (1H, m), 8.48-8.51 (1H, m), 8.98 (1H, s)

ESI-MS(m/z): 441(M+H)⁺

Preparation 58

To a suspension of 5-nitroindoline (3.28 g), 2-pyridylacetic acid hydrochloride (3.82 g), 1-[3-(dimethylamino)propyl]-3-ethylcarbodiimide hydrochloride (4.22 g) and 1-hydroxybenzotriazole hydrate (3.37 g) in dichloromethane (100 ml) was added dropwise triethylamine (4.45 g) at ambient temperature and the resultant solution was stirred at ambient temperature for 18 hours. The mixture was poured into water and the separated organic layer was washed with water and brine, dried over magnesium sulfate and evaporated in vacuo. The residue was purified by column chromatography on silica gel eluting with ethyl acetate to give 5-nitro-1-(2-pyridinylacetyl)indoline (3.58 g) as a yellow solid.

¹H-NMR(DMSO-d₆): δ 3.26(2H, t, J=8.5 Hz), 4.10(2H, s), 4.33(2H, t, J=8.5 Hz), 7.25-7.35(1H, m), 7.38(1H, d, J=7.8 Hz), 7.75-7.9(1H, m), 8.1-8.2(3H, m), 8.50-8.55(1H, m)

APCI-MS(m/z): 284(M+H)⁺

Preparation 59

To a solution of 5-nitro-1-(2-pyridinylacetyl)indoline (3.54 g) in methanol (50 ml) and tetrahydrofuran (THF) (50 ml) was added 10% palladium on carbon (50% wet, 3.5 g) and the mixture was hydrogenated under hydrogen at atmospheric pressure for 5 hours. After removing the palladium on carbon by filtration, the filtrate was evaporated in vacuo. The residue was purified by column chromatography on silica gel eluting with ethyl acetate:methanol (10:1) to give 1-(2-pyridinylacetyl)-5-indolinamine (2.16 g) as pale brown crystals.

¹H-NMR(DMSO-d₆): δ 3.01(2H, t, J=8.4 Hz), 3.92(2H, s), 4.11(2H, t, J=8.4 Hz), 4.84(2H, br s), 6.32(1H, d, J=8.4 Hz), 6.45(1H, s), 7.1-7.2(1H, m), 7.33(1H, d, J=7.8 Hz), 7.7-7.85(2H, m), 8.48(1H, d, J=4.0 Hz)

APCI-MS(m/z): 254(M+H)⁺

### EXAMPLE 120

To a suspension of 1-(2-pyridinylacetyl)-5-indolinamine (506 mg), 2-(4-fluorophenyl)-1-cyclohexene-1-carboxylic acid (440 mg) and 1-hydroxybenzotriazole hydrate (337 mg) in N,N-dimethylformamide (30 ml) was added dropwise 1-[3-(dimethylamino)propyl]-3-ethylcarbodiimide (341 mg) at ambient temperature and the resultant solution was stirred at the same temperature for 18 hours. The reaction mixture was poured into water and the separated organic layer was washed with water and brine, dried over magnesium sulfate and evaporated in vacuo. The residue was purified by column chromatography on silica gel eluting with ethyl acetate to give 2-(4-fluorophenyl)-N-[1-(2-pyridinylacetyl)-2,3-dihydro-1H-indol-5-yl]-1-cyclohexene-1-carboxamide (620 mg) as white crystals.

¹H-NMR(DMSO-d₆): δ 1.6-1.8(4H, m), 2.25-2.45(4H, m), 3.06(2H, t, J=8.2 Hz), 3.96(2H, s), 4.15(2H, t, J=8.2 Hz), 7.0-7.4(8H, m), 7.7-7.95(2H, m), 8.45-8.55(1H, m), 9.49(1H, s)

negative ESI-MS(m/z): 454(M−H)⁻

Preparation 60

1-{[6-(2,5-Dimethyl-1H-pyrrol-1-yl)-2-pyridinyl]acetyl}-5-nitroindoline was obtained from 5-nitroindoline and [6-(2,5-dimethyl-1H-pyrrol-1-yl)-2-pyridinyl]acetic acid in the same manner as in Preparation 58 as light yellow crystals.

¹H-NMR(DMSO-d₆): δ 2.02(6H, s), 3.25(2H, t, J=8.6 Hz), 4.16(2H, s), 4.30(2H, t, J=8.6 Hz), 5.77(2H, s), 7.31(1H, d, J=8.6 Hz), 7.31(1H, d, J=8.6 Hz), 7.98(1H, dd, J=8.6 Hz, 8.6 Hz), 8.00-8.15(3H, m)

APCI-MS(m/z): 377(M+H)⁺

Preparation 61

1-{[6-(2,5-Dimethyl-1H-pyrrol-1-yl)-2-pyridinyl]acetyl}-5-indolinamine was obtained in the same manner as in Preparation 59 as light yellow crystals.

¹H-NMR(DMSO-d₆): δ 2.22(6H, s), 2.99(2H, t, J=8.4 Hz), 3.98(2H, s), 4.08(2H, t, J=8.4 Hz), 4.84(2H, br s), 5.77(2H, s), 6.32(1H, dd, J=8.5 Hz, 2.2 Hz), 6.45(1H, d, J=2.2 Hz), 7.27(1H, d, J=7.7 Hz), 7.39(1H, d, J=7.3 Hz), 7.73(1H, d, J=8.5 Hz), 7.94(1H, dd, J=7.7 Hz, 7.3 Hz)

ESI-MS(m/z): 369(M+Na)⁺, 347(M+H)⁺

### EXAMPLE 121

To a suspension of 1-{[6-(2,5-dimethyl-1H-pyrrol-1-yl)-2-pyridinyl]acetyl}-5-indolinamine (1.04 g), 2-(4-fluorophenyl)-1-cyclohexene-1-carboxylic acid (661 mg) and 1-hydroxybenzotriazole hydrate (505 mg) in N,N-dimethylformamide (30 ml) was added dropwise 1-[3-(dimethylamino)propyl]-3-ethylcarbodiimide (512 mg) at ambient temperature and the resultant solution was stirred at the same temperature for 18 hours. The reaction mixture was poured into water and the separated organic layer was washed with water and brine, dried over magnesium sulfate and evaporated in vacuo. The residue was purified by column chromatography on silica gel eluting with ethyl acetate to give N-(1-{[6-(2,5-dimethyl-1H-pyrrol-1-yl)-2-pyridinyl]acetyl}-2,3-dihydro-1H-indol-5-yl)-2-(4-fluorophenyl)-1-cyclohexene-1-carboxamide (1.24 g) as a white solid.

¹H-NMR(DMSO-d₆): δ 1.6-1.8(4H, m), 2.01(6H, s), 2.3-2.45(4H, m), 3.05(2H, t, J=8.3 Hz), 4.02(2H, s), 4.12(2H, t, J=8.3 Hz), 5.77(2H, s), 7.0-7.15(3H, m), 7.25-7.4(5H, m), 7.83(1H, d, J=8.7 Hz), 7.94(1H, dd, J=7.7 Hz, 7.7 Hz), 9.48(1H, s)

ESI-MS(m/z): 571(M+Na)⁺, 549(M+H)⁺

### EXAMPLE 122

To a suspension of N-(1-{[6-(2,5-dimethyl-1H-pyrrol-1-yl)-2-pyridinyl]acetyl}-2,3-dihydro-1H-indol-5-yl)-2-(4-fluorophenyl)-1-cyclohexene-1-carboxamide (1.23 g) in a mixture of ethanol (40 ml) and water (10 ml) were added hydroxylamine hydrochloride (1.56 g) and triethylamine (454 mg) at ambient temperature. The mixture was refluxed for 8 hours and evaporated to dryness. The residue was extracted with ethyl acetate and the organic layer was washed with brine, dried over magnesium sulfate, and evaporated in vacuo. The residue was recrystallized from acetonitrile, collected by filtration and washed with acetonitrile to give N-{1-[(6-amino-2-pyridinyl)acetyl]-2,3-dihydro-1H-indol-5-yl}-2-(4-fluorophenyl)-1-cyclohexene-1-carboxamide (630 mg) as white crystals.

¹H-NMR(DMSO-d₆): δ 1.6-1.8(4H, m), 2.25-2.45(4H, m), 3.04(2H, t, J=8.3 Hz), 3.67(2H, s), 4.13(2H, t, J=8.3 Hz), 5.85(2H, br s), 6.29(1H, d, J=8.0 Hz), 6.90(1H, d, J=7.0 Hz), 7.0-7.2(3H, m), 7.25-7.4(4H, m), 7.84(1H, d, J=8.6 Hz), 9.47(1H, s)

negative ESI-MS(m/z): 469(M−H)⁻

### EXAMPLE 123

2-(4-Chlorophenyl)-N-(1-{[6-(2,5-dimethyl-1H-pyrrol-1-yl)-2-pyridinyl]acetyl}-2,3-dihydro-1H-indol-5-yl)-1-cyclohexene-1-carboxamide was obtained in the same manner as in Example 121 as a white solid.

¹H-NMR(DMSO-d₆): δ 1.6-1.8(4H, m), 2.01(6H, s), 2.3-2.5(4H, m), 3.06(2H, t, J=8.3 Hz), 4.03(2H, s), 4.13(2H, t, J=8.3 Hz), 5.77(2H, s), 7.05(1H, dd, J=8.7 Hz, 2.0 Hz), 7.25-7.45(7H, m), 7.84(1H, d, J=8.7 Hz), 7.85-7.95(1H, m), 9.53(1H, s)

negative ESI-MS(m/z): 563(M−H)⁻

### EXAMPLE 124

N-{1-[(6-Amino-2-pyridinyl)acetyl]-2, 3-dihydro-1H-indol-5-yl}-2-(4-chlorophenyl)-1-cyclohexene-1-carboxamide was obtained in the same manner as in Example 122 as white crystals.

¹H-NMR(DMSO-d₆): δ 1.6-1.8(4H, m), 2.25-2.4(4H, m), 3.05(2H, t, J=8.3 Hz), 3.67(2H, s), 4.14(2H, t, J=8.3 Hz), 5.85(2H, br s), 6.29(1H, d, J=8.0 Hz), 6.40(1H, d, J=7.0 Hz), 7.04(1H, dd, J=8.5 Hz, 1.8 Hz), 7.25-7.4(6H, m), 7.85(1H, d, J=8.5 Hz), 9.52(1H, s)

negative ESI-MS(m/z): 485(M−H)⁻

### EXAMPLE 125

N-(1-{[6-(2,5-Dimethyl-1H-pyrrol-1-yl)-2-pyridinyl]acetyl}-2,3-dihydro-1H-indol-5-yl)-2-(4-methylphenyl)-1-cyclohexene-1-carboxamide was obtained in the same manner as in Example 121 as a white solid.

¹H-NMR(DMSO-d₆): δ 1.6-1.8(4H, m), 2.21(3H, s), 2.25-2.4(4H, m), 3.05(2H, d, J=8.4 Hz), 4.02(2H, s), 4.12(2H, d, J=8.4 Hz), 5.77(2H, s), 7.04(2H, d, J=8.1 Hz), 7.17(2H, d, J=8.1 Hz), 7.28(1H, d, J=7.7 Hz), 7.35-7.45(2H, m), 7.82(1H, d, J=8.7 Hz), 7.94(1H, dd, J=7.7 Hz, 7.7 Hz), 9.44(1H, s)

ESI-MS(m/z): 567(M+Na)⁺, 545(M+H)⁺

### EXAMPLE 126

N-{1-[(6-Amino-2-pyridinyl)acetyl]-2,3-dihydro-1H-indol-5-yl}-2-(4-methylphenyl)-1-cyclohexene-1-carboxamide was obtained in the same manner as in Example 122 as white crystals.

¹H-NMR(DMSO-d₆): δ 1.6-1.8(4H, m), 2.21(3H, s), 2.25-2.4(4H, m), 3.04(2H, t, J=8.3 Hz), 3.67(2H, s), 4.13(2H, t, J=8.3 Hz), 5.85(2H, br s), 6.29(1H, d, J=8.1 Hz), 6.40(1H, d, J=7.0 Hz), 7.03(2H, d, J=8.1 Hz), 7.05(1H, s), 7.17(2H, d, J=8.1 Hz), 7.25-7.35(2H, m), 7.84(1H, d, J=8.6 Hz), 9.43(1H, s)

ESI-MS(m/z): 489(M+Na)⁺, 467(M+H)⁺

### EXAMPLE 127

N-(1-{[6-(2,5-Dimethyl-1H-pyrrol-1-yl)-2-pyridinyl]acetyl}-2,3-dihydro-1H-indol-5-yl)-2-(4-methoxyphenyl)-1-cyclohexene-1-carboxamide was obtained in the same manner as in Example 121 as a white solid.

¹H-NMR(DMSO-d₆): δ 1.6-1.8(4H, m), 2.01(6H, s), 2.25-2.4(4H, m), 3.05(2H, t, J=8.3 Hz), 3.67(3H, s), 4.01(2H, s), 4.13(2H, t, J=8.3 Hz), 5.77(2H, s), 6.80(2H, d, J=8.8 Hz), 7.05(1H, dd, J=8.7 Hz, 1.8 Hz), 7.21(2H, d, J=8.7 Hz), 7.28(1H, d, J=7.9 Hz), 7.39(2H, d, J=7.5 Hz), 7.83(1H, d, J=8.7 Hz), 7.94(1H, dd, J=7.8 Hz, 7.8 Hz), 9.43(1H, s)

ESI-MS(m/z): 583(M+Na)⁺, 561(M+H)⁺

### EXAMPLE 128

N-{1-[(6-Amino-2-pyridinyl)acetyl]-2,3-dihydro-1H-indol-5-yl}-2-(4-methoxyphenyl)-1-cyclohexene-1-carboxamide was obtained in the same manner as in Example 122 as white crystals.

¹H-NMR(DMSO-d₆): δ 1.6-1.8(4H, m), 2.25-2.4(4H, m), 3.04(2H, t, J=8.5 Hz), 3.67(3H, s), 4.13(2H, t, J=8.5 Hz), 5.84(2H, br s), 6.29(1H, d, J=8.2 Hz), 6.40(1H, d, J=7.4 Hz), 6.79(2H, d, J=8.8 Hz), 7.00(1H, dd, J=7.4 Hz, 2.1 Hz), 7.20(2H, d, J=8.8 Hz), 7.28(1H, d, J=7.4 Hz), 7.34(1H, d, J=2.1 Hz), 7.84(1H, d, J=8.7 Hz), 9.40(1H, s)

negative ESI-MS(m/z): 481(M−H)⁻

### EXAMPLE 129

N-(1-{[6-(2,5-Dimethyl-1H-pyrrol-1-yl)-2-pyridinyl]acetyl}-2,3-dihydro-1H-indol-5-yl)-2-[4-(trifluoromethyl)phenyl]-1-cyclohexene-1-carboxamide was obtained in the same manner as in Example 121 as a white solid.

¹H-NMR(DMSO-d₆): δ 1.6-1.8(4H, m), 2.01(6H, s), 2.25-2.4(4H, m), 3.04(2H, t, J=8.4 Hz), 4.02(2H, s), 4.12(2H, t, J=8.4 Hz), 5.76(2H, s), 7.00(1H, dd, J=8.6 Hz, 1.8 Hz), 7.28(2H, d, J=7.8 Hz), 7.38(1H, d, J=7.5 Hz), 7.47(2H, d, J=8.2 Hz), 7.62(2H, d, J=8.2 Hz), 7.85(1H, d, J=8.6 Hz), 7.94(1H, dd, J=8.6 Hz, 7.5 Hz), 9.56(1H, s)

ESI-MS(m/z): 621(M+Na)⁺, 599(M+H)⁺

### EXAMPLE 130

N-{1-[(6-Amino-2-pyridinyl)acetyl]-2,3-dihydro-1H-indol-5-yl}-2-[4-(trifluoromethyl)phenyl]-1-cyclohexene-1-carboxamide was obtained in the same manner as in Example 122 as white crystals.

¹H-NMR(DMSO-d₆): δ 1.65-1.85(4H, m), 2.3-2.5(4H, m), 3.03(2H, t, J=8.1 Hz), 3.67(2H, s), 4.13(2H, t, J=8.1 Hz), 5.85(2H, br s), 6.29(1H, d, J=8.0 Hz), 6.40(1H, d, J=7.2 Hz), 6.99(1H, dd, J=8.7 Hz, 1.8 Hz), 7.30(1H, dd, J=8.0 Hz, 7.2 Hz), 7.31(1H, d, J=1.8 Hz), 7.47(2H, d, J=8.3 Hz), 7.62(2H, d, J=8.3 Hz), 7.84(1H, d, J=8.7 Hz), 9.56(1H, s)

ESI-MS(m/z): 543(M+Na)⁺, 521(M+H)⁺

Preparation 62

To a solution of 4-methyl-2-pyrimidinamine (10.0 g) in toluene (200 ml) were added 2,5-hexanedione (11.5 g) and p-toluenesulfonic acid hydrate (1.74 g) at ambient temperature and the mixture was refluxed for 20 hours. The reaction mixture was concentrated to ca. 50 ml and purified by column chromatography on silica gel to give 2-(2,5-dimethyl-1H-pyrrol-1-yl)-4-methylpyrimidine (14.10 g) as a red oil.

¹H-NMR(DMSO-d₆): δ 2.23(6H, s), 2.52(3H, s), 5.81(2H, s), 7.35(1H, d, J=5.1 Hz), 8.73(1H, d, J=5.1 Hz)

ESI-MS(m/z) : 210(M+Na)⁺, 188(M+H)⁺

Preparation 63

To a 1 mol/L solution of sodium bis(trimethylsilyl)amide in tetrahydrofuran (82.2 ml) was added dropwise a solution of 2-(2,5-dimethyl-1H-pyrrol-1-yl)-4-methylpyrimidine (14.0 g) in tetrahydrofuran (100 ml) at 5° C. under a nitrogen atmosphere and the mixture was stirred at 5° C. for 1.5 hours. To the mixture was added carefully crashed Dry Ice (ca. 10 g) and the mixture was stirred at ambient temperature for 30 minutes. The reaction mixture was poured into a mixture of ethyl acetate and water, and adjusted to pH 2 with 6N HCl. The separated organic layer was washed with water and brine, dried over magnesium sulfate, and evaporated in vacuo. The residue was purified by column chromatography on silica gel and triturated with diisopropyl ether to give [2-(2,5-dimethyl-1H-pyrrol-1-yl)-4-pyrimidinyl]acetic acid (8.86 g) as light brown crystals.

¹H-NMR(DMSO-d₆): δ 2.23(6H, s), 3.85(2H, s), 5.82 (2H, s), 7.43(1H, d, J=5.1 Hz), 8.83(1H, d, J=5.1 Hz), 12.72(1H, br)

Preparation 64

1-{[2-(2,5-Dimethyl-1H-pyrrol-1-yl)-4-pyrimidinyl]acetyl}-5-nitroindoline was obtained in the same manner as in Preparation 58 as light yellow crystals.

¹H-NMR(DMSO-d₆): δ 2.21(6H, s), 3.27(2H, t, J=8.5 Hz), 4.21(2H, s), 4.31(2H, t, J=8.5 Hz), 5.80(2H, s), 7.47(1H, d, J=5.1 Hz), 8.1-8.2(3H, m), 8.85(1H, d, J=5.1 Hz)

ESI-MS(m/z): 400(M+Na)⁺, 378(M+H)⁺

Preparation 65

1-{[2-(2,5-Dimethyl-1H-pyrrol-1-yl)-4-pyrimidinyl]acetyl}-5-indolinamine was obtained in the same manner as in Preparation 59 as light yellow crystals.

¹H-NMR(DMSO-d₆): δ 3.02(2H, t, J=8.2 Hz), 4.04(2H, s), 4.10(2H, t, J=8.2 Hz), 4.88(2H, br s), 5.80(2H, s), 6.33(1H, dd, J=8.5 Hz, 1.8 Hz), 6.46(1H, d, J=1.8 Hz), 7.43(1H, d, J=5.1 Hz), 7.73(1H, d, J=8.5 Hz), 8.81(1H, d, J=5.1 Hz)

ESI-MS(m/z): 370(M+Na)⁺, 348(M+H)⁺

### EXAMPLE 131

N-(1-{[2-(2,5-Dimethyl-1H-pyrrol-1-yl)-4-pyrimidinyl]acetyl}-2,3-dihydro-1H-indol-5-yl)-2-(4-fluorophenyl)-1-cyclohexene-1-carboxamide was obtained in the same manner as in Example 121 as a light brown solid.

¹H-NMR(DMSO-d₆): δ 1.6-1.8(4H, m), 2.20(6H, s), 2.25-2.4(4H, m), 3.08(2H, t, J=7.6 Hz), 4.08(2H, s), 4.14(2H, t, J=7.6 Hz), 5.80(2H, s), 6.95-7.15(3H, m), 7.2-7.35(3H, m), 7.43(1H, d, J=5.0 Hz), 7.83(1H, d, J=8.7 Hz), 8.82(1H, d, J=5.0 Hz), 9.50(1H, s)

### EXAMPLE 132

N-{1-[(2-Amino-4-pyrimidinyl)acetyl]-2, 3-dihydro-1H-indol-5-yl}-2-(4-fluorophenyl)-1-cyclohexene-1-carboxamide was obtained in the same manner as in Example 122 as white crystals.

¹H-NMR(DMSO-d₆): δ 1.65-1.9(4H, m), 2.3-2.5(4H, m), 3.06(2H, t, J=8.5 Hz), 3.71(2H, s), 4.12(2H, t, J=8.5 Hz), 6.51(1H, d, J=5.0 Hz), 6.56(2H, br s), 7.0-7.15(3H, m), 7.25-7.4(3H, m), 7.83(1H, d, J=8.7 Hz), 8.14(1H, d, J=5.0 Hz), 9.49(1H, s)

negative ESI-MS(m/z): 470(M−H)⁻

### EXAMPLE 133

2-[4-(Dimethylamino)phenyl]-N-(1-{[6-(2,5-dimethyl-1H-pyrrol-1-yl)-2-pyridinyl]acetyl}-2,3-dihydro-1H-indol-5-yl)-1-cyclohexene-1-carboxamide was obtained in the same manner as in Example 121 as a light brown solid.

¹H-NMR(DMSO-d₆): δ 1.6-1.8(4H, m), 2.01(6H, s), 2.3-2.55(4H, m), 2.82(6H, s), 3.05(2H, t, J=8.3 Hz), 4.02(2H, s), 4.13(2H, t, J=8.3 Hz), 5.76(2H, s), 6.58(2H, d, J=8.9 Hz), 7.07(1H, d, J=7.2 Hz), 7.13(2H, d, J=8.9 Hz), 7.28(1H, d, J=7.8 Hz), 7.37(1H, s), 7.39(1H, d, J=7.2 Hz), 7.83(1H, d, J=8.7 Hz), 7.94(1H, dd, J=8.7 Hz, 7.8 Hz), 9.66(1H, s)

ESI-MS(m/z): 596(M+Na)⁺, 574(M+H)⁺

### EXAMPLE 134

N-{1-[(6-Amino-2-pyridinyl)acetyl]-2,3-dihydro-1H-indol-5-yl}-2-[4-(dimethylamino)phenyl]-1-cyclohexene-1-carboxamide was obtained in the same manner as in Example 122 as white crystals.

¹H-NMR(DMSO-d₆): δ 1.6-1.8(4H, m), 2.2-2.35(4H, m), 2.82(6H, s), 3.05(2H, t, J=8.4 Hz), 3.67(2H, s), 4.13(2H, t, J=8.4 Hz), 5.84(2H, br s), 6.29(1H, d, J=8.1 Hz), 6.40(1H, d, J=7.0 Hz), 6.58(2H, d, J=8.9 Hz), 7.07(1H, d, J=8.7 Hz), 7.13(2H, d, J=8.9 Hz), 7.30(1H, dd, J=8.1 Hz, 7.0 Hz), 7.84(1H, d, J=8.7 Hz), 9.35(1H, s)

ESI-MS(m/z): 518(M+Na)⁺, 496(M+H)⁺

### EXAMPLE 135

2-(4-Ethylphenyl)-N-[1-(2-pyridinylacetyl)-2,3-dihydro-1H-indol-5-yl]-1-cyclohexene-1-carboxamide was obtained in the same manner as in Example 121 as white crystals.

¹H-NMR(DMSO-d₆): δ 1.11(3H, t, J=7.6 Hz), 1.6-1.8(4H, m), 2.25-2.4(4H, m), 2.53(2H, q, J=7.6 Hz), 3.06(2H, t, J=8.3 Hz), 3.96(2H, s), 4.15(2H, t, J=8.3 Hz), 6.9-7.35(7H, m), 7.7-7.9(2H, m), 8.49(1H, d, J=5.0 Hz), 9.37(1H, s)

ESI-MS(m/z): 488(M+Na)⁺, 466(M+H)⁺

### EXAMPLE 136

N-(1-{[6-(2,5-Dimethyl-1H-pyrrol-1-yl)-2-pyridinyl]acetyl}-2,3-dihydro-1H-indol-5-yl)-2-(4-ethylphenyl)-1-cyclohexene-1-carboxamide was obtained in the same manner as in Example 121 as a light brown solid.

¹H-NMR(DMSO-d₆): δ 1.11(3H, t, J=7.6 Hz), 1.6-1.8(4H, m), 2.01(6H, s), 2.3-2.45(4H, m), 2.54(2H, q, J=7.6 Hz), 3.04(2H, t, J=7.8 Hz), 4.02(2H, s), 4.12(2H, t, J=7.8 Hz), 5.76(2H, s), 6.9-7.4(8H, m), 7.8-8.0(2H, m), 9.38(1H, s)

negative ESI-MS(m/z): 557(M−H)⁻

### EXAMPLE 137

N-{1-[(6-Amino-2-pyridinyl)acetyl]-2,3-dihydro-1H-indol-5-yl}-2-(4-ethylphenyl)-1-cyclohexene-1-carboxamide was obtained in the same manner as in Example 122 as white crystals.

¹H-NMR(DMSO-d₆): δ 1.11(3H, t, J=7.6 Hz), 1.6-1.8(4H, m), 2.25-2.4(4H, m), 2.51(2H, q, J=7.6 Hz), 3.12(2H, t, J=8.4 Hz), 4.04(2H, s), 4.15(2H, t, J=8.4 Hz), 6.75(1H, d, J=7.1 Hz), 6.87(1H, d, J=8.7 Hz), 7.04(1H, dd, J=8.7 Hz, 1.6 Hz), 7.07(2H, d, J=8.1 Hz), 7.20(2H, d, J=8.1 Hz), 7.35(1H, d, J=1.6 Hz), 7.78(2H, br s), 7.75-7.9(2H, m), 9.45(1H, s)

ESI-MS(m/z): 503(M+Na)⁺, 481(M+H)⁺

### EXAMPLE 138

N-[1-(2-Pyridinylacetyl)-2,3-dihydro-1H-indol-5-yl]-2-[4-(trifluoromethyl)phenyl]-1-cyclohexene-1-carboxamide was obtained in the same manner as in Example 120.

¹H-NMR(DMSO-d₆): δ 1.64-1.84(4H, m), 2.33-2.48(4H, m), 3.05(2H, t, J=8.4 Hz), 3.96(2H, s), 4.15(2H, t, J=8.4 Hz), 6.96-7.06(1H, m), 7.22-7.38(3H, m), 7.47(2H, d, J=8.2 Hz), 7.62(2H, d, J=8.2 Hz), 7.75(1H, dt, J=1.8 Hz, 7.7 Hz), 7.83(1H, d, J=8.7 Hz), 8.45-8.52(1H, m), 9.56(1H, s)

negative ESI-MS(m/z): 504(M−H)⁻

### EXAMPLE 139

2-(4-Methylphenyl)-N-[1-(2-pyridinylacetyl)-2,3-dihydro-1H-indol-5-yl]-1-cyclohexene-1-carboxamide was obtained in the same manner as in Example 120.

¹H-NMR(DMSO-d₆): δ 1.60-1.81(4H, m), 2.20(3H, s), 2.28-2.42(4H, m), 3.06(2H, t, J=8.3 Hz), 3.96(2H, s), 4.15(2H, t, J=8.3 Hz), 7.00-7.09(3H, m), 7.17(2H, d, J=8.1 Hz), 7.22-7.40(3H, m), 7.75(1H, dt, J=1.8 Hz, 7.7 Hz), 7.83(1H, d, J=8.7 Hz), 8.44-8.52(1H, m), 9.44(1H, s)

negative ESI-MS(m/z): 450(M−H)⁻

### EXAMPLE 140

2-(4-Chlorophenyl)-N-[1-(2-pyridinylacetyl)-2,3-dihydro-1H-indol-5-yl]-1-cyclohexene-1-carboxamide was obtained in the same manner as in Example 120.

¹H-NMR(DMSO-d₆): δ 1.62-1.80(4H, m), 2.39-2.43(4H, m), 3.07(2H, t, J=8.3 Hz), 3.97(2H, s), 4.16(2H, t, J=8.3 Hz), 7.05(1H, dd, J=1.8 Hz, 8.6 Hz), 7.22-7.38(7H, m), 7.75(1H, dt, J=1.8 Hz, 7.6 Hz), 7.84(1H, d, J=8.6 Hz), 8.46-8.52(1H, m), 9.53(1H, s)

negative ESI-MS(m/z): 470(M−H)⁻

### EXAMPLE 141

2-(4-Methoxyphenyl)-N-[1-(2-pyridinylacetyl)-2,3-dihydro-1H-indol-5-yl]-1-cyclohexene-1-carboxamide was obtained in the same manner as in Example 120.

¹H-NMR(DMSO₆): δ 1.60-1.80(4H, m), 2.27-2.40(4H, m), 3.06(2H, t, J=8.3 Hz), 3.67(3H, s), 3.96(2H, s), 4.15(2H, t, J=8.3 Hz), 6.80(2H, d, J=8.7 Hz), 7.01-7.09(1H, m), 7.17-7.40(5H, m), 7.75(1H, dt, J=1.9 Hz, 7.6 Hz), 7.84(1H, d, J=8.7 Hz), 8.45-8.52(1H, m), 9.42(1H, s)

negative ESI-MS(m/z): 466(M−H)⁻

Preparation 66

2-[4-(Dimethylamino)phenyl]-1-cyclohexene-1-carboxylic acid was obtained in the same manner as in Preparation 120.

¹H-NMR(DMSO-d₆): δ 1.53-1.75(4H, m), 2.20-2.37(4H, m), 2.87(6H, s), 6.57-6.68(2H, m), 6.98-7.07(2H, m), 11.84(1H, s)

negative ESI-MS(m/z): 244(M−H)⁻

### EXAMPLE 142

2-[4-(Dimethylamino)phenyl]-N-[1-(2-pyridinylacetyl)-2,3-dihydro-1H-indol-5-yl]-1-cyclohexene-1-carboxamide was obtained in the same manner as in Example 120.

¹H-NMR(DMSO-d₆): δ 1.58-1.80(4H, m), 2.26-2.40(4H, m), 2.81(6H, s), 3.06(2H, t, J=8.3 Hz), 3.96(2H, s), 4.14(2H, t, J=8.3 Hz), 6.58(2H, d, J=8.7 Hz), 7.04-7.19(3H, m), 7.21-7.42(3H, m), 7.74(1H, dt, J=1.8 Hz, 7.6 Hz), 7.84(1H, d, J=8.7 Hz), 8.44-8.52(1H, m), 9.38(1H, s)

negative ESI-MS(m/z): 479(M−H)⁻

Preparation 67

To a suspension of sodium hydride (60% oil dispersion) (5.16 g) in N,N-dimethylformamide (160 ml) was added dropwise a solution of methyl 2-oxocycloheptanecarboxylate (20.0 g) at 10° C. under a nitrogen atmosphere and the mixture was warmed to ambient temperature and stirred for an hour. To this mixture was added dropwise 1,1,2,2,3,3,4,4,4-nonafluoro-1-butanesulfonyl fluoride (39.0 g) at ambient temperature and the mixture was warmed to 35° C. and stirred at 35° C. for 20 hours. The reaction mixture was poured into a mixture of ethyl acetate and ice water and adjusted to pH ca. 2 with 6N hydrochloric acid. The separated organic layer was washed with water and brine dried over magnesium sulfate and evaporated in vacuo. The residue was purified by column chromatography on silica gel eluting with hexane:toluene (1:1) to give methyl 2-{[(nonafluorobutyl)sulfonyl]oxy}-1-cycloheptene-1-carboxylate (29.82 g) as a colorless oil.

¹H-NMR (DMSO-d₆): δ 1.6-1.9(6H, m), 2.6-2.9(4H, m), 3.70(3H, s)

ESI-MS(m/z) : 475(M+Na)⁺

Preparation 68

To a suspension of zinc chloride (17.91 g) in tetrahydrofuran (200 ml) was added dropwise a 1 mol/L solution of tolylmagnesium bromide in tetrahydrofuran (98.6 ml) at 0° C. under a nitrogen atmosphere and the mixture was stirred at 0° C. for 30 minutes. To this suspension were added bis(dibenzylideneacetone) palladium (1.13 g) and 1,1′-bis(diphenylphosphino)ferrocene (1.09 g), followed by dropwise addition of methyl 2-{[(nonafluorobutyl)sulfonyl]oxy}-1-cycloheptene-1-carboxylate (29.72 g) in tetrahydrofuran (90 ml). The mixture was refluxed for 16 hours under a nitrogen atmosphere. The reaction mixture was poured into a mixture of ethyl acetate and ice water and adjusted to pH ca. 2 with 6N hydrochloric acid. The separated organic layer was washed with water and brine, dried over magnesium sulfate and evaporated in vacuo. The residue was purified by column chromatography on silica gel eluting with hexane:toluene (1:3) to give methyl 2-(4-methylphenyl)-1-cycloheptene-1-carboxylate (13.77 g) as a colorless oil.

¹H-NMR(DMSO-d₆): δ 1.6-1.9(6H, m), 2.28(3H, s), 2.5-2.8(4H, m), 3.70(3H, s), 6.95-7.0(2H, m), 7.1-7.15(2H, m)

ESI-MS (m/z): 267(M+Na)⁺

Preparation 69

To a solution of methyl 2-(4-methylphenyl)-1-cycloheptene-1-carboxylate (13.76 g) in ethanol (130 ml) was added 5N aqueous sodium hydroxide solution (22.6 ml) at ambient temperature and the mixture was refluxed for 4 hours. The reaction mixture was cooled to 5° C. and ice-water (60 ml) was added. The mixture was adjusted to pH ca. 7 with 6N hydrochloric acid and concentrated in vacuo. To the residue was added a mixture of ethyl acetate and water and the mixture was adjusted to pH ca. 2 with 6N hydrochloric acid. The separated organic layer was washed with water and brine, dried over magnesium sulfate and evaporated in vacuo. The residue was triturated with hexane to give 2-(4-methylphenyl)-1-cycloheptene-1-carboxylic acid (3.58 g) as white crystals.

¹H-NMR(DMSO-d₆): δ 1.45-1.6(4H, m), 1.7-1.9(2H, m), 2.27(3H, s), 2.4-2.55(4H, m), 7.0-7.15(4H, m), 11.90(1H, br s)

ESI-MS(m/z): 253(M+Na)⁺

### EXAMPLE 143

2-(4-Methylphenyl)-N-[1-(2-pyridinylacetyl)-2,3-dihydro-1H-indol-5-yl]-1-cycloheptene-1-carboxamide was obtained in the same manner as in Example 120 as white crystals.

H-NMR(DMSO-d₆): δ 1.6-1.9(6H, m), 2.21(3H, s), 2.4-2.5(4H, m), 2.85(2H, t, J=7.7 Hz), 3.99(2H, t, J=7.7 Hz), 7.0-7.3(8H, m), 7.37(2H, d, J=8.7 Hz), 7.6-7.7(1H, m), 8.25(1H, s), 8.45(1H, d, J=3.9 Hz), 9.42(1H, s)

ESI-MS(m/z): 488(M+Na)⁺, 466(M+H)⁺

### EXAMPLE 144

To a solution of 2-[4-(trifluoromethyl)phenyl]-1-cyclohexene-1-carboxylic acid (499 mg) in toluene (5 ml) were added thionyl chloride (0.27 ml) and N,N-dimethylformamide (1 drop) and the mixture was stirred at 50° C. for 1 hour. The reaction mixture was evaporated in vacuo and the residue was dissolved in tetrahydrofuran (2 ml). The obtained acid chloride in tetrahydrofuran was added to a solution of tert-butyl 4-aminophenyl(2-{6-[(tert-butoxycarbonyl)amino]-2-pyridinyl}ethyl)carbamate (720 mg) and triethylamine (0.47 ml) in tetrahydrofuran (30 ml) at ambient temperature and the mixture was stirred at the same temperature for 30 minutes. The mixture was poured into water and extracted with ethyl acetate. The organic layer was washed with brine, dried over magnesium sulfate, and evaporated in vacuo. The residue was purified by column chromatography on silica gel by eluting with hexane:ethyl acetate (3:1→2:1) to give tert-butyl 2-{6-[(tert-butoxycarbonyl)amino]-2-pyridinyl}ethyl{4-[({2-[4-(trifluoromethyl)phenyl]-1-cyclohexen-1-yl}carbonyl)amino]phenyl}carbamate (1.123 g) as a yellow foam.

¹H-NMR(DMSO-d₆): δ 1.36(18H, br s), 1.74(4H, br s), 2.40(4H, br s), 2.83(2H, t, J=7.4 Hz), 3.79(2H, t, J=7.4 Hz), 7.02(2H, d, J=8.6 Hz), 7.13(2H, d, J=7.2 Hz), 7.18(2H, d, J=7.9 Hz), 7.31(2H, d, J=8.9 Hz), 7.49(2H, d, J=8.2 Hz), 7.61(2H, d, J=8.6 Hz), 7.73(1H, t, J=7.8 Hz), 9.45(1H, s), 9.69(1H, s)

ESI-MS(m/z): 703(M+Na)⁺

### EXAMPLE 145

To a solution of tert-butyl 2-{6-[(tert-butoxycarbonyl)amino]-2-pyridinyl}ethyl{4-[({2-[4-(trifluoromethyl)phenyl]-1-cyclohexen-1-yl}carbonyl)amino]phenyl}carbamate (1.116 g) in dichloromethane (10 ml) was added trifluoroacetic acid (1.6 ml). The reaction mixture was stirred for 15 hours, quenched with 10% aqueous potassium carbonate solution, and extracted with ethyl acetate-tetrahydrofuran. The organic layer was washed with brine, dried over magnesium sulfate, filtered, and concentrated in vacuo. The residue was recrystallized from ethyl acetate-diisopropyl ether to give N-(4-{[2-(6-amino-2-pyridinyl)ethyl]amino}phenyl)-2-[4-(trifluoromethyl)phenyl]-1-cyclohexene-1-carboxamide (0.646 g) as a white solid.

¹H-NMR(DMSO-d₆): δ 1.72(4H, br s), 2.37(4H, br s), 2.68(2H, t, J=7.2 Hz), 3.21(2H, q, J=7.0 Hz), 5.67(1H, br s), 5.87(2H, br s), 6.27(1H, d, J=8.2 Hz), 6.37(1H, d, J=7.2 Hz), 6.42(2H, d, J=8.6 Hz), 6.99(2H, d, J=8.2 Hz), 7.27(1H, t, J=7.8 Hz), 7.48(2H, d, J=8.2 Hz), 7.62(2H, d, J=8.2 Hz), 9.19(1H, s)

ESI-MS(m/z): 480(M+H)⁺

### EXAMPLE 146

To a solution of tert-butyl 4-aminophenyl(2-{6-[(tert-butoxycarbonyl)amino]-2-pyridinyl}ethyl)carbamate (772 mg), 2-(4-methylphenyl)-1-cyclohexene-1-carboxylic acid (409 mg) and 1-hydroxybenzotriazole hydrate (292 mg) in N,N-dimethylformamide (20 ml) was added 1-[3-(dimethylamino)propyl]-3-ethylcarbodiimide hydrochloride (WSC.HCl) (414 mg), followed by triethylamine (0.38 ml) at ambient temperature. The reaction mixture was stirred at 50° C. for 2 hours and concentrated in vacuo. The residue was dissolved in ethyl acetate and water, and extracted with ethyl acetate. The organic layer was washed with water and brine, dried over magnesium sulfate, filtered, and concentrated in vacuo. The residue was purified by column chromatography on silica gel by eluting with hexane:ethyl acetate (4:1→2:1) to give tert-butyl 2-{6-[(tert-butoxycarbonyl)amino]-2-pyridinyl}ethyl[4-({([2-(4-methylphenyl)-1-cyclohexen-1-yl]carbonyl}amino)phenyl]carbamate (0.557 g) as a pale yellow solid.

### EXAMPLE 147

To a solution of tert-butyl 2-{6-[(tert-butoxycarbonyl)-amino]-2-pyridinyl}ethyl[4-({[2-(4-methylphenyl)-1-cyclohexen-1-yl]carbonyl}amino)phenyl]carbamate (1.116 g) in dichloromethane (10 ml) was added trifluoroacetic acid (1.6 ml). The reaction mixture was stirred for 15 hours, quenched with 10% aqueous potassium carbonate solution, and extracted with ethyl acetate-tetrahydrofuran. The organic layer was washed with brine, dried over magnesium sulfate, filtered, and concentrated in vacuo. The residue was recrystallized from ethyl acetate-diisopropyl ether to give N-(4-{[2-(6-amino-2-pyridinyl)ethyl]amino phenyl)-2-(4-methylphenyl)-1-cyclohexene-1-carboxamide (0.253 g) as a white solid.

¹H-NMR(DMSO-d₆): δ 1.69(4H, br s), 2.21(3H, s), 2.33(4H, br s), 2.68(2H, t, J=7.3 Hz), 3.20(2H, q, J=6.9 Hz), 5.43(1H, t, J=5.6 Hz), 5.82(2H, br s), 6.26(1H, d, J=7.9 Hz), 6.36(1H, d, J=7.2 Hz), 6.42(2H, d, J=8.6 Hz), 7.04(4H, d, J=8.6 Hz), 7.17(2H, d, J=7.9 Hz), 7.25(1H, t, J=7.7 Hz), 9.05(1H, s)

ESI-MS(m/z): 426(M+H)⁺

### EXAMPLE 148

tert-Butyl 2-{6-[(tert-butoxycarbonyl)amino]-2-pyridinyl}ethyl[4-({[2-(4-ethylphenyl)-1-cyclohexen-1-yl]carbonyl}amino)phenyl]carbamate was obtained in the same manner as in Example 146 as a pale yellow foam.

### EXAMPLE 149

N-(4-{[2-(6-Amino-2-pyridinyl)ethyl]amino}phenyl)-2-(4-ethylphenyl)-1-cyclohexene-1-carboxamide was obtained in the same manner as in Example 147 as white crystals.

¹H-NMR(DMSO-d₆): δ 1.12(3H, t, J=7.6 Hz), 1.69(4H, br s), 2.52(2H, q, J=7.6 Hz), 2.68(2H, t, J=7.3 Hz), 3.20(2H, q, J=6.9 Hz), 5.43(1H, t, J=5.6 Hz), 5.82(2H, br s), 6.26(1H, d, J=8.2 Hz), 6.36(1H, d, J=7.2 Hz), 6.41(2H, d, J=8.6 Hz), 7.00(2H, d, J=8.9 Hz), 7.07(2H, d, J=8.2 Hz), 7.20(2H, d, J=8.2 Hz), 7.25(1H, dd, J=7.9, 7.2 Hz), 8.98(1H, s)

ESI-MS(m/z): 440(M+H)⁺

### EXAMPLE 150

To a solution of 2-(4-methylphenyl)-1-cyclohexene-1-carboxylic acid (49.5 mg) in toluene (3 ml) were added thionyl chloride (0.033 ml) and N,N-dimethylformamide (1 drop) and the mixture was stirred at 50° C. for an hour. The mixture was evaporated in vacuo and the residue was dissolved in tetrahydrofuran (1 ml). The acid chloride in tetrahydrofuran was added to a solution of tert-butyl 5-amino-2-pyridinyl(2-{6-[(tert-butoxycarbonyl)amino]-2-pyridinyl}ethyl)carbamate (82 mg) and triethylamine (0.053 ml) in tetrahydrofuran (5 ml) at ambient temperature and the mixture was stirred at the same temperature for 30 minutes. The mixture was poured into water and extracted with ethyl acetate. The organic layer was washed with brine, dried over magnesium sulfate, and evaporated in vacuo. The residue was purified by column chromatography on silica gel by eluting with hexane:ethyl acetate (3:1→2:1) to give tert-butyl 2-{6-[(tert-butoxycarbonyl)amino]-2-pyridinyl}ethyl[5-({[2-(4-methylphenyl)-1-cyclohexen-1-yl]carbonyl}amino)-2-pyridinyl]carbamate (0.117 g) as a yellow foam.

¹H-NMR(DMSO-d₆): δ 1.34-1.45(18H, m), 1.72(4H, br s), 2.21(3H, s), 2.37(4H, br s), 2.81(2H, t, J=7.3 Hz), 4.02(2H, t, J=7.4 Hz), 6.80(1H, t, J=4.1 Hz), 7.06(2H, d, J=8.2 Hz), 7.18(2H, d, J=8.2 Hz), 7.35(1H, d, J=8.9 Hz), 7.59(2H, d, J=4.0 Hz), 7.75(1H, dd, J=8.9, 2.6 Hz), 8.32(1H, d, J=2.3 Hz), 9.55(1H, s), 9.72(1H, s)

ESI-MS(m/z): 628(M+H)⁺

### EXAMPLE 151

To a solution of tert-butyl 2-{6-[(tert-butoxycarbonyl)amino]-2-pyridinyl}ethyl[5-({[2-(4-methylphenyl)-1-cyclohexen-1-yl]carbonyl}amino)-2-pyridinyl]carbamate (107 mg) in dichloromethane (3 ml) was added trifluoroacetic acid (0.52 ml). The reaction mixture was stirred at ambient temperature for 16 hours, quenched with 10% aqueous potassium carbonate solution, and extracted with ethyl acetate-tetrahydrofuran. The organic layer was washed with brine, dried over magnesium sulfate, filtered, and concentrated in vacuo. The residue was recrystallized from ethyl acetate-diisopropyl ether to give N-(6-{[2-(6-amino-2-pyridinyl)ethyl]amino}-3-pyridinyl)-2-(4-methylphenyl)-1-cyclohexene-1-carboxamide (0.043 g) as white crystals.

¹H-NMR(DMSO-d₆): δ 1.70(4H, br s), 2.23(3H, s), 2.34(4H, br s), 2.68(2H, t, J=7.3 Hz), 3.41(2H, q, J=7.3 Hz), 5.85(2H, br s), 6.26(1H, d, J=8.2 Hz), 6.32(1H, d, J=3.6 Hz), 6.35(1H, d, J=1.3 Hz), 7.05(2H, d, J=8.2 Hz), 7.17(2H, d, J=8.2 Hz), 7.25(1H, t, J=8.2 Hz), 7.30(1H, dd, J=8.9, 2.6 Hz), 7.83(1H, d, J=2.6 Hz), 9.12(1H, s)

ESI-MS(m/z): 428(M+H)⁺

### EXAMPLE 152

tert-Butyl 2-{6-[(tert-butoxycarbonyl)amino]-2-pyridinyl}ethyl[5-({[2-(4-ethylphenyl)-1-cyclohexen-1-yl]carbonyl}amino)-2-pyridinyl]carbamate was obtained in the same manner as in Example 150 as a pale yellow foam.

¹H-NMR(DMSO-d₆): δ 1.09(3H, t, J=7.6 Hz), 1.34(9H, s), 1.45(9H, s), 1.72(4H, br s), 2.37(4H, br s), 2.52(2H, q, J=7.6 Hz), 2.80(2H, t, J=7.1 Hz), 4.01(2H, t, J=7.1 Hz), 6.78(2H, t, J=4.1 Hz), 7.08(2H, d, J=8.2 Hz), 7.20(2H, d, J=7.9 Hz), 7.35(1H, d, J=8.9 Hz), 7.58-7.60(2H, m), 7.72(1H, dd, J=8.9, 2.6 Hz), 8.29(1H, d, J=2.3 Hz), 9.55(1H, s), 9.67(1H, s)

ESI-MS(m/z): 664(M+Na)⁺

### EXAMPLE 153

N-(6-{[2-(6-Amino-2-pyridinyl)ethyl]amino}-3-pyridinyl)-2-(4-ethylphenyl)-1-cyclohexene-1-carboxamide was obtained in the same manner as in Example 151 as white crystals.

¹H-NMR(DMSO-d₆): δ 1.12(3H, t, J=7.6 Hz), 1.69(4H, br s), 2.34(4H, br s), 2.53(2H, q, J=7.6 Hz), 2.67(2H, t, J=7.3 Hz), 3.37(2H, t, J=7.3 Hz), 5.82(2H, br s), 6.25(1H, d, J=8.2 Hz), 6.30-6.35(3H, m), 7.09(2H, d, J=8.2 Hz), 7.19(1H, d, J=7.9 Hz), 7.21-7.28(3H, m), 7.79(1H, d, J=2.6 Hz), 9.06(1H, s)

ESI-MS(m/z): 442(M+H)⁺

### EXAMPLE 154

To a solution of tert-butyl 6-[2-(4-aminophenoxy)ethyl]-2-pyridinylcarbamate (609 mg), 2-[4-(trifluoromethyl)phenyl]-1-cyclohexene-1-carboxylic acid (500 mg) and 1-hydroxybenzotriazole hydrate (340 mg) in N,N-dimethylformamide (5 ml) was added 1-[3-(dimethylamino)propyl]-3-ethylcarbodiimide hydrochloride (WSC.HCl) (425.6 mg), followed by triethylamine (0.387 ml) at ambient temperature. The reaction mixture was stirred at 50° C. for 2 hours and concentrated in vacuo. The residue was dissolved in ethyl acetate and water, and extracted with ethyl acetate. The organic layer was washed with water and brine, dried over magnesium sulfate, filtered, and concentrated in vacuo. The residue was purified by column chromatography on silica gel by eluting with hexane:ethyl acetate (6:1→2:1) to give tert-butyl 6-(2-{4-[({2-[4-(trifluoromethyl)phenyl]-1-cyclohexen-1-yl}carbonyl)amino]phenoxy}ethyl)-2-pyridinylcarbamate (0.712 g) as a pale yellow foam.

¹H-NMR(CDCl₃): δ 1.51(9H, s), 1.80(4H, br s), 2.43(2H, br s), 2.54(2H, br s), 3.07(2H, t, J=6.5 Hz), 4.22(2H, t, J=6.8 Hz), 6.42(1H, s), 6.70(2H, d, J=8.9 Hz), 6.85-6.88(3H, m), 7.14(1H, s), 7.41(2H, d, J=7.8 Hz), 7.53-7.60(3H, m), 7.74(1H, d, J=8.4 Hz)

ESI-MS(m/z): 604(M+Na)⁺

### EXAMPLE 155

To a solution of tert-butyl 6-(2-{4-[({2-[4-(trifluoromethyl)phenyl]-1-cyclohexen-1-yl}carbonyl)amino]phenoxy}ethyl)-2-pyridinylcarbamate (700 mg) in dichloromethane (7 ml) was added trifluoroacetic acid (1.39 ml). The reaction mixture was stirred for 7 hours, quenched with 10% aqueous potassium carbonate solution, and extracted with dichloromethane. The organic layer was washed with brine, dried over magnesium sulfate, filtered, and concentrated in vacuo. The residue was recrystallized from ethyl acetate-diisopropyl ether to give N-{4-[2-(6-amino-2-pyridinyl) ethoxy]phenyl}-2-[4-(trifluoromethyl)phenyl]-1-cyclohexene-1-carboxamide (0.473 g) as a white solid.

¹H-NMR(DMSO-d₆): δ 1.74(4H, br s), 2.39(4H, br s), 2.88(2H, t, J=7.0 Hz), 4.16(2H, t, J=7.0 Hz), 5.87(2H, br s), 6.28(1H, d, J=8.4 Hz), 6.42(1H, d, J=7.3 Hz), 6.75(2H, d, J=8.9 Hz), 7.21(2H, d, J=8.9 Hz); 7.28(1H, t, J=7.8 Hz), 7.48(2H, d, J=8.1 Hz), 7.62(2H, .d, J=8.6 Hz), 9.48(1H, s)

ESI-MS(m/z): 482(M+H)⁺

### EXAMPLE 156

tert-Butyl 6-{2-[4-({[2-(4-methylphenyl)-1-cyclohexen-1-yl]carbonyl}amino)phenoxy]ethyl}-2-pyridinylcarbamate was obtained by in the same manner as in Example 154 as a pale yellow foam.

¹H-NMR(DMSO-d₆): δ 1.46(9H, s), 1.70(4H, br s), 2.21(3H, s), 2.34(4H, br s), 3.02(2H, t), 4.22(2H, t), 6.76(2H, d, J=9.2 Hz), 6.96(1H, dd, J=5.9, 2.7 Hz), 7.03(2H, d, J=7.8 Hz), 7.17(2H, d, J=7.8 Hz), 7.24(2H, d, J=8.9 Hz), 7.58-7.69(2H, m), 9.33(1H, s), 9.60(1H, s)

ESI-MS(m/z) : 550(M+Na)⁺

### EXAMPLE 157

N-{4-[2-(6-Amino-2-pyridinyl) ethoxy]phenyl)}-2-(4-methylphenyl)-1-cyclohexene-1-carboxamide was obtained in the same manner as in Example 155 as a pale yellow powder.

¹H-NMR(DMSO-d₆): δ 1.71(4H, br s), 2.34(4H, br s), 2.90(2H, t, J=6.8 Hz), 4.17(2H, t, J=6.8 Hz), 6.07(2H, br s), 6.34(1H, d, J=8.4 Hz), 6.46(1H, d, J=7.8 Hz), 6.76(2H, d, J=8.9 Hz), 7.04(2H, d, J=8.4 Hz), 7.17(2H, d, J=8.4 Hz), 7.24(2H, d, J=8.6 Hz), 7.34(1H, t, J=7.3 Hz), 9.34(1H, s)

ESI-MS(m/z): 428(M+H)⁺

### EXAMPLE 158

tert-Butyl 6-{2-[4-({[2-(4-ethylphenyl)-1-cyclohexen-1-yl]carbonyl}amino)phenoxy]ethyl}-2-pyridinylcarbamate was obtained in the same manner as in Example 154 as a pale yellow foam.

¹H-NMR(CDCl₃): δ 1.21(3H, t, J=7.6 Hz), 1.51(9H, s), 1.77(4H, br s), 2.42(2H, br s), 2.52(2H, br s), 2.62(2H, q, J=7.6 Hz), .3.06(2H, t, J=6.6 Hz), 4.21(2H, t, J=6.6 Hz), 6.74(1H, s), 6.67(2H, d, J=8.9 Hz), 6.78(2H, d, J=8.9 Hz), 6.86(1H, d, J=7.0 Hz), 7.14-7.18(5H, m), 7.56(1H, t, J=7.6 Hz), 7.74(1H, d, J=8.1 Hz)

ESI-MS(m/z): 564(M+Na)⁺

### EXAMPLE 159

N-{4-[2-(6-Amino-2-pyridinyl)ethoxy]phenyl}-2-(4-ethylphenyl)-1-cyclohexene-1-carboxamide was obtained in the same manner as in Example 155 as a pale pink powder.

¹H-NMR(DMSO-d): δ 1.11(3H, t, J=7.6 Hz), 1.71(4H, br s), 2.35(4H, br s), 2.50(2H, q, J=7.6 Hz), 2.87(2H, t, J=7.0 Hz), 4.16(2H, t, J=7.0 Hz), 5.84(2H, br s), 6.28(1H, d, J=7.8 Hz), 6.41(1H, d, J=7.3 Hz), 6.75(2H, d, J=8.9 Hz), 7.07(2H, d, J=8.1 Hz), 7.18-7.22(4H, m), 7.28(1H, t, J=7.8 Hz), 9.29(1H, s)

ESI-MS(m/z): 442(M+H)⁺

### EXAMPLE 160

To a solution of 2-(4-methylphenyl)-1-cyclohexene-1-carboxylic acid (363 mg) in toluene (10 ml) were added thionyl chloride (0.19 ml) and N,N-dimethylformamide (1 drop) and the mixture was stirred at 50° C. for an hour. The mixture was evaporated in vacuo and the residue was dissolved in tetrahydrofuran (5 ml). The acid chloride in tetrahydrofuran was added to a solution of tert-butyl 6-{2-[(5-amino-2-pyridinyl)oxy]ethyl}-2-pyridinylcarbamate (462 mg) and triethylamine (0.39 ml) in tetrahydrofuran (15 ml) at ambient temperature and the mixture was stirred at the same temperature for 30 min. The mixture was poured into water and extracted with ethyl acetate. The organic layer was washed with brine, dried over magnesium sulfate, and evaporated in vacuo. The residue was purified by column chromatography on silica gel by eluting with hexane:ethyl acetate (3:1→2:1) to give tert-butyl 6-(2-{[5-({[2-(4-methylphenyl)-1-cyclohexen-1-yl]carbonyl)}amino)-2-pyridinyl]oxy}ethyl)-2-pyridinylcarbamate (0.731 g) as a yellow foam.

¹H-NMR(DMSO-d₆): δ 1.45(9H, s), 1.70(4H, br s), 2.21(3H, s), 2.35(4H, br s), 3.02(2H, t, J=6.6 Hz), 4.49(2H, t, J=6.6 Hz), 6.63(1H, d, J=8.9 Hz), 6.93(1H, dd, J=5.3, 3.0 Hz), 7.05(2H; d, J=7.9 Hz), 7.17(2H, d, J=8.2 Hz), 7.57-7.63(3H, m), 8.08(1H, d, J=2.6 Hz), 9.49(1H, s), 9.62(1H, s)

ESI-MS(m/z): 551(M+Na)⁺

### EXAMPLE 161

To a solution of tert-butyl 6-(2-{[5-(≡[2-(4-methylphenyl)-1-cyclohexen-1-yl]carbonyl}amino)-2-pyridinyl]oxy}ethyl)-2-pyridinylcarbamate (720 mg) in dichloromethane (30 ml) was added trifluoroacetic acid (2.1 ml). The reaction mixture was stirred for 15 hours, quenched with 10% aqueous potassium carbonate solution, and extracted with ethyl acetate. The organic layer was washed with brine, dried over magnesium sulfate, filtered, and concentrated in vacuo. The residue was recrystallized from ethyl acetate-diisopropyl ether to give N-{6-[2-(6-amino-2-pyridinyl)ethoxy]-3-pyridinyl}-2-(4-methylphenyl)-1-cyclohexene-1-carboxamide (0.505 g) as a white solid.

¹H-NMR(DMSO-d₆): δ 1.71(4H, br s), 2.21(3H, s), 2.35(4H, br s), 2.89(2H, t, J=6.9 Hz), 4.44(2H, t, J=6.9 Hz), 6.03(2H, br s), 6.32(1H, d, J=7.9 Hz), 6.42(1H, d, J=7.2 Hz), 6.64(1H, d, J=8.9 Hz), 7.05(2H, d, J=7.9 Hz), 7.17(2H, d, J=7.9 Hz), 7.32(1H, dd, J=8.2, 7.2 Hz), 7.60(1H, dd, J=8.9, 2.6 Hz), 8.07(1H, d, J=2.6 Hz), 9.49(1H, s)

ESI-MS(m/z): 429(M+H)⁺

### EXAMPLE 162

tert-Butyl 6-[2-({5-[({2-[4-(trifluoromethyl)phenyl]-1-cyclohexen-1-yl}carbonyl)amino]-2-pyridinyl}oxy)ethyl]-2-pyridinylcarbamate was obtained in the same manner as in Example 160 as a white solid.

¹H-NMR(CDCl₃): δ 1.51(9H, s), 1.72-1.90(4H, m), 2.40-2.50(2H, m), 2.50-2.62(2H, m), 3.06(2H, t, J=6.5 Hz), 4.53(2H, t, J=6.5 Hz), 6.44(1H, s), 6.55(1H, d, J=8.6 Hz), 6.85(1H, d, J=7.0 Hz), 7.17(1H, s), 7.34-7.43(3H, m), 7.51-7.63(4H, m), 7.73(1H, d, J=8.4 Hz).

ESI-MS(m/z): 583(M+H)⁺

### EXAMPLE 163

N-{6-[2-(6-Amino-2-pyridinyl)ethoxy]-3-pyridinyl}-2-[4-(trifluoromethyl)phenyl]-1-cyclohexene-1-carboxamide was obtained in the same manner as in Example 161 as a white solid.

¹H-NMR(CDCl₃): δ 1.78-1.81(4H, m), 2.40-2.46(2H, m), 2.49-2.57(2H, m), 3.02(2H, t, J=7.0 Hz), 4.34(2H, s), 4.53(2H, t, J=7.0 Hz), 6.33(1H, d, J=8.4 Hz), 6.43(1H, s), 6.54-6.59(2H, m), 7.26-7.43(4H, m), 7.58-7.63(3H, m).

ESI-MS(m/z): 483(M+H)⁺

### EXAMPLE 164

tert-Butyl 6-[2-{[5-({[2-(4-ethylphenyl)-1-cyclohexen-1-yl]carbonyl}amino)-2-pyridinyl]oxy]ethyl)-2-pyridinylcarbamate was obtained in the same manner as in Example 160 as a white solid.

¹H-NMR(CDCl₃): δ 1.22(3H, t, J=7.6 Hz), 1.51(9H, s), 1.73-1.83(4H, m), 2.40-2.50(2H, m), 2.50-2.58(2H, m), 2.63(2H, q, J=7.6 Hz), 3.06(2H, t, J=7.0 Hz), 4.52(2H, t, J=7.0 Hz), 6.45(1H, s), 6.53(1H, d, J=8.6 Hz), 6.85(1H, d, J=7.6 Hz), 7.15-7.20(5H, m), 7.38(1H, dd, J=8.9, 2.7 Hz), 7.47(1H, d, J=2.7 Hz), 7.55(1H, t, J=7.8 Hz), 7.73(1H, d, J=8.4 Hz).

ESI-MS(m/z): 543(M+H)⁺

### EXAMPLE 165

N-{6-[2-(6-Amino-2-pyridinyl)ethoxy]-3-pyridinyl}-2-(4-ethylphenyl)-1-cyclohexene-1-carboxamide was obtained in the same manner as in Example 161 as a white solid.

¹H-NMR(CDCl₃): δ 1.22(3H, t, J=7.6 Hz), 1.70-1.82(4H, m), 2.40-2.47(2H, m), 2.47-2.56(2H, m), 2.64(2H, q, J=7.6 Hz), 3.02(2H, t, J=6.8 Hz), 4.36(2H, s), 4.51(2H, t, J=6.8 Hz), 6.34(1H, d, J=8.1 Hz), 6.46(1H, s), 6.53-6.58(2H, m), 7.15-7.23(4H, m), 7.34(1H, t, J=8.4 Hz), 7.40(1H, dd, J=8.9, 3.0 Hz), 7.45(1H, d, J=2.4 Hz).

ESI-MS(m/z): 443(M+H)⁺

### EXAMPLE 166

To a solution of tert-butyl 4-aminophenyl(2-{2-[(tert-butoxycarbonyl)amino]-1,3-thiazol-4-yl}ethyl)carbamate (531 mg), 2-[4-(trifluoromethyl)phenyl]-1-cyclohexene-1-carboxylic acid (329 mg) and 1-hydroxybenzotriazole hydrate (223.7 mg) in N,N-dimethylformamide (6.5 ml) was added 1-[3-(dimethylamino)propyl]-3-ethylcarbodiimide hydrochloride (WSC.HCl) (279.5 mg), followed by triethylamine (0.255 ml) at ambient temperature. The reaction mixture was stirred at 50° C. for 12 hours and concentrated in vacuo. The residue was dissolved in ethyl acetate and water, and extracted with ethyl acetate. The organic layer was washed with water and brine, dried over magnesium sulfate, filtered, and concentrated in vacuo. The residue was purified by column chromatography on silica gel by eluting with hexane:ethyl acetate (9:1→4:1→2:1) to give tert-butyl 2-{2-[(tert-butoxycarbonyl)amino]-1,3-thiazol-4-yl}ethyl{4-[({2-[4-(trifluoromethyl)phenyl]-1-cyclohexen-1-yl}carbonyl)amino]phenyl}carbamate (0.609 g) as a pale yellow foam.

¹H-NMR(CDCl₃): δ 1.38(9H, s), 1.48(9H, s), 1.80(4H, br s), 2.44(2H, br s), 2.55(2H, br s), 2.88(2H, t, J=7.0 Hz), 3.84(2H, t, J=7.0 Hz), 6.52(1H, s), 6.74(1H, s), 6.95-7.02(4H, m), 7.42(2H, d, J=8.5 Hz), 7.59(2H, t, J=8.5 Hz)

ESI-MS(m/z): 709(M+Na)⁺

### EXAMPLE 167

To a solution of tert-butyl 2-{2-[(tert-butoxycarbonyl)amino]-1,3-thiazol-4-yl}ethyl{4-[({2-[4-(trifluoromethyl)phenyl]-1-cyclohexen-1-yl}carbonyl)amino]phenyl}carbamate (694.5 mg) in dichloromethane (7 ml) was added trifluoroacetic acid (1.95 ml). The reaction mixture was stirred for 14 hours, quenched with 10% aqueous potassium carbonate solution, and extracted with dichloromethane. The organic layer was washed with brine, dried over magnesium sulfate, filtered, and concentrated in vacuo. The residue was recrystallized from ethyl acetate-diisopropyl ether to give N-(4-{[2-(2-amino-1,3-thiazol-4-yl)ethyl]amino}phenyl)-2-[4-(trifluoromethyl)phenyl]-1-cyclohexene-1-carboxamide (0.359 g) as a pale brown powder.

¹H-NMR(DMSO-d₆): δ 1.73(4H, br s), 2.38(4H, br s), 2.61(2H, t, J=7.2 Hz), 3.16(2H, q, J=7.0 Hz), 5.40(1H, s), 6.18(2H, s), 6.40(1H, d, J=8.8 Hz); 6.84(2H, s), 6.99(2H, d, J=8.8 Hz), 7.48(2H, d, J=8.2 Hz), 7.62(2H, d, J=8.2 Hz), 9.19(1H, s)

ESI-MS(m/z): 509(M+Na)⁺

### EXAMPLE 168

tert-Butyl 2-{2-[(tert-butoxycarbonyl)amino]-1,3-thiazol-4-yl}ethyl [4-({[2-(4-methylphenyl)-1-cyclohexen-1-yl]carbonyl}amino)phenyl]carbamate was obtained in the same manner as in Example 166 as a pale yellow foam.

¹H-NMR(CDCl₃): δ 1.51(9H, s), 1.75(4H, br s), 2.32(3H, s), 2.40(4H, br s), 2.51(4H, br s), 2.91(2H, t, J=6.8 Hz), 3.36(2H, t, J=6.8 Hz), 6.43(2H, d, J=8.6 Hz), 6.45(1H, s), 6.73(1H, s), 6.75(2H, d, J=8.6 Hz), 7.11-7.19(4H, m)

ESI-MS(m/z): 655(M+Na)⁺

### EXAMPLE 169

N-(4-{[2-(2-Amino-1,3-thiazol-4-yl)ethyl]amino}phenyl)-2-(4-methylphenyl)-1-cyclohexene-1-carboxamide was obtained in the same manner as in Example 167 as a pale brown powder.

¹H-NMR(DMSO-d₆): δ 1.70(4H, br s), 2.22(3H, s), 2.34(4H, br s), 2.61(2H, t, J=7.0 Hz), 3.16(2H, t, J=7.0 Hz), 6.19(1H, s), 6.40(2H, d, J=8.9 Hz), 6.87(2H, s), 7.04(4H, d, J=8.4 Hz), 7.18(2H, d, J=8.1 Hz), 9.05(1H, s)

ESI-MS(m/z): 455(M+Na)⁺

### EXAMPLE 170

tert-Butyl 2-{2-[(tert-butoxycarbonyl)amino]-1,3-thiazol-4-yl}ethyl[4-({[2-(4-ethylphenyl)-1-cyclohexen-1-yl]carbonyl}amino)phenyl]carbamate was obtained in the same manner as in Example 166 as a pale yellow foam.

¹H-NMR(CDCl₃): δ 1.21(3H, t, J=7.6 Hz), 1.29(9H, s), 1.48(9H, s), 1.77(4H, br s), 2.43(2H, br s), 2.53(2H, br s), 2.63(2H, q, J=7.6 Hz), 2.88(2H, t, J=7.9 Hz), 3.83(2H, t, J=7.9 Hz), 6.60(1H, s), 6.74(1H, s), 6.86-6.96(4H, m), 7.12-7.24(4H, m)

ESI-MS(m/z): 669(M+Na)⁺

### EXAMPLE 171

N-(4-{[2-(2-Amino-1,3-thiazol-4-yl)ethyl]amino}phenyl)-2-(4-ethylphenyl)-1-cyclohexene-1-carboxamide was obtained in the same manner as in Example 167 as a pale brown powder.

¹H-NMR(DMSO-d₆): δ 1.13(3H, t, J=7.3 Hz), 1.70(4H, br s), 2.33(4H, br s), 2.54(2H, t, J=7.3 Hz), 2.61(2H, t, J=7.3 Hz), 3.16(2H, dd, J=7.3, 5;7 Hz), 5.36(1H, t, J=5.7 Hz), 6.18(1H, s), 6.39(2H, d, J=8.6 Hz), 6.83(2H, s), 6.99(2H, d, J=8.6 Hz), 7.08(2H, d, J=8.2 Hz), 7.20(2H, d, J=8.2 Hz), 8.98(1H, s)

ESI-MS(m/z): 447(M+H)⁺

### EXAMPLE 172

To a solution of tert-butyl 4-[2-(4-aminophenoxy)ethyl]-1,3-thiazol-2-ylcarbamate (578 mg), 2-[4-(trifluoromethyl)phenyl]-1-cyclohexene-1-carboxylic acid(510 mg) and 1-hydroxybenzotriazole hydrate (315 mg) in N,N-dimethylformamide (20 ml) was added 1-[3-(dimethylamino)-propyl]-3-ethylcarbodiimide hydrochloride (WSC.HCl) (395 mg), followed by triethylamine (0.36 ml) at ambient temperature. The reaction mixture was stirred at 50° C. or 2 hours and concentrated in vacuo. The residue was dissolved in ethyl acetate and water, and extracted with ethyl acetate. The organic layer was washed with water and brine, dried over magnesium sulfate, filtered, and concentrated in vacuo. The residue was purified by column chromatography on silica gel by eluting with hexane:ethyl acetate (2:1→3:2) to give tert-butyl 4-(2-{4-[({2-[4-(trifluoromethyl)phenyl]-1-cyclohexen-1-yl}carbonyl)amino]phenoxy}ethyl)-1,3-thiazol-2-ylcarbamate (1.011 g) as a pale yellow foam.

¹H-NMR(DMSO-d₆): δ 1.46(3H, t, J=7.6 Hz), 1.73(94H, sbr s), 2.39(4H, br s), 2.95(2H, t, J=6.6 Hz), 4.13(2H, t, J=6.6 Hz), 6.76(2H, d, J=9.2 Hz), 6.82(1H, s), 7.21(2H, d, J=9.2 Hz), 7.48(2H, d, J=8.2 Hz), 7.62(2H, d, J=8.2 Hz), 9.48(1H, s), 11.36(1H, s)

ESI-MS(m/z) : 610(M+Na)⁺

### EXAMPLE 173

To a solution of tert-butyl 4-(2-{4-[({2-[4-(trifluoromethyl)phenyl]-1-cyclohexen-1-yl}carbonyl)amino]phenoxy}ethyl)-1,3-thiazol-2-ylcarbamate (1.011 g) in dichloromethane (25 ml) was added trifluoroacetic acid (2.6 ml). The reaction mixture was stirred at ambient temperature for 13 hours, quenched with 10% aqueous potassium carbonate solution, and extracted with ethyl acetate-tetrahydrofuran. The organic layer was washed with brine, dried over magnesium sulfate, filtered, and concentrated in vacuo. The residue was recrystallized from ethyl acetate-diisopropyl ether to give N-{4-[2-(2-amino-1,3-thiazol-4-yl)ethoxy]phenyl}-2-[4-(trifluoromethyl)phenyl]-1-cyclohexene-1-carboxamide (0.791 g) as white crystals.

¹H-NMR(DMSO-d₆): δ 1.73(4H, br s), 2.39(4H, br s), 2.91(2H, t, J=6.3 Hz), 4.11(2H, t, J=6.4 Hz), 6.56(1H, s), 6.78(2H, d, J=8.9 Hz), 7.22(2H, d, J=8.9 Hz), 7.48(2H, d, J=8.2 Hz), 7.61(2H, d, J=8.2 Hz), 9.51(1H, s)

ESI-MS(m/z): 487(M+H)⁺

### EXAMPLE 174

tert-Butyl 4-{2-[4-({[2-(4-methylphenyl)-1-cyclohexen-1-yl]carbonyl}amino)phenoxy]ethyl}-1,3-thiazol-2-ylcarbamate (1.213 g) was obtained in the same manner as in Example 172 as a pale yellow foam.

¹H-NMR(DMSO-d₆): δ 1.46(9H, s), 1.69(4H, br s), 2.21(3H, s), 2.34(4H, br s), 2.98(2H, t, J=6.6 Hz), 4.13(2H, t, J=6.6 Hz), 6.76(2H, d, J=8.9 Hz), 6.82(1H, s), 7.04(2H, d, J=7.9 Hz), 7.17(2H, d, J=7.9 Hz), 7.25(2H, d, J=8.9 Hz), 9.33(1H, s)

ESI-MS (m/z): 534(M+H)⁺

### EXAMPLE 175

N-{4-[2-(2-Amino-1,3-thiazol-4-yl)ethoxy]phenyl}-2-(4-methylphenyl)-1-cyclohexene-1-carboxamide was obtained in the same manner as in Example 173 as white crystals.

¹H-NMR(DMSO-d₆): δ 1.70(4H, br s), 2.21(3H, s), 2.34(4H, br s), 2.80(2H, t, J=6.9 Hz), 4.09(2H, t, J=6.9 Hz), 6.23(1H, s), 6.84(2H, br s), 7.04(2H, d, J=8.2 Hz), 7.17(2H, d, J=7.9 Hz), 7.25(2H, d, J=8.9 Hz), 9.33(1H, s)

ESI-MS(m/z): 434(M+H)⁺

### EXAMPLE 176

tert-Butyl 4-{2-[4-({[2-(4-ethylphenyl)-1-cyclohexen-1-yl]carbonyl}amino)phenoxy]ethyl}-1,3-thiazol-2-ylcarbamate was obtained in the same manner as in Example 172 as a pale yellow foam.

¹H-NMR(DMSO-d₆): δ 1.11(3H, t, J=7.6 Hz), 1.46(9H, s), 1.70(4H, br s), 2.35(4H, br s), 2.51(2H, q, J=7.6 Hz), 2.95(2H, t, J=6.7 Hz), 4.13(2H, t, J=6.7 Hz), 6.75(2H, d, J=8.2 Hz), 6.82(1H, s), 7.07(2H, d, J=8.2 Hz), 7.18(2H, d, J=4.6 Hz), 7.21(2H, d, J=5.3 Hz), 9.29(1H, s), 11.36(1H, s)

ESI-MS(m/z): 548(M+H)⁺

### EXAMPLE 177

N-{4-[2-(2-Amino-1,3-thiazol-4-yl)ethoxy]phenyl}-2-(4-ethylphenyl)-1-cyclohexene-1-carboxamide was obtained in the same manner as in Example 173 as white crystals.

¹H-NMR(DMSO-d₆): δ 1.11(3H, t, J=7.6 Hz), 1.70(4H, br s), 2.35(4H, br s), 2.50(2H, q, J=7.6 Hz), 2.80(2H, t, J=6.7 Hz), 4.09(2H, t, J=6.7 Hz), 6.22(1H, s), 6.75(2H, d, J=8.9 Hz), 6.85(2H, s), 7.07(2H, d, J=7.9 Hz), 7.21(2H, d, J=8.9 Hz), 7.19(2H, d, J=7.6 Hz), 9.29(1H, s)

ESI-MS(m/z): 448(M+H)⁺

Preparation 70

A mixture of tert-butyl 4-(2-aminoethyl)-1,3-thiazol-2-ylcarbamate (2.44 g), 2-chloro-5-nitropyridine (2.38 g) and triethylamine (2.8 ml) in N,N-dimethylformamide (20 ml) was heated at 50° C. for 2 hours. The reaction mixture was concentrated in vacuo. To the residue added water and extracted with ethyl acetate and tetrahydrofuran. The organic layer was washed with brine, dried over magnesium sulfate, filtered, and concentrated in vacuo. The residue was recrystallized from ethyl acetate-hexane to give tert-butyl 4-{2-[(5-nitro-2-pyridinyl)amino]ethyl}-1,3-thiazol-2-ylcarbamate (3.596 g) as pale yellow powder.

¹H-NMR(DMSO-d₆): δ 1.47(9H, s), 2.83(2H, t, J=6.8 Hz), 3.66(2H, br s), 6.55(1H, d, J=9.5 Hz), 6.79(1H, s), 8.09(1H, br d, J=8.6 Hz), 8.18(1H, br s, J=3.0 Hz), 8.92(1H, d, J=2.7 Hz), 11.4(1H, s)

ESI-MS(m/z): 388(M+Na)⁺

Preparation 71

To a solution of tert-butyl 4-{2-[(5-nitro-2-pyridinyl)amino]ethyl}-1,3-thiazol-2-ylcarbamate (2.0 g) in tetrahydrofuran (40 ml) was added di-t-butyl dicarbonate (1.43 g) and the mixture was heated at 55° C. for 2 hours. The reaction mixture was cooled to room temperature and concentrated in vacuo. The residue was dissolved in ethyl acetate and water, and extracted with ethyl acetate. The organic layer was washed with water and brine, dried over magnesium sulfate, filtered, and concentrated in vacuo. The residue was purified by column chromatography on silica gel by eluting with hexane:ethyl acetate (4:1) to give tert-butyl 2-{2-[(tert-butoxycarbonyl)amino]-1,3-thiazol-4-yl}ethyl(5-nitro-2-pyridinyl)carbamate (2.059 g) as a brown oil.

¹H-NMR(CDCl₃): δ 1.57(18H, s), 3.04(2H, t, J=7.6 Hz), 4.37(2H, t, J=7.6 Hz), 6.74(1H, s), 8.03(1H, d, J=9.5 Hz), 8.34(1H, dd, J=9.2, 3.0 Hz), 9.17(1H, d, J=3.0 Hz)

ESI-MS(m/z): 488(M+Na)⁺

Preparation 72

A solution of tert-butyl 2-{2-[(tert-butoxycarbonyl)amino]-1,3-thiazol-4-yl)}ethyl(5-nitro-2-pyridinyl)carbamate (1.937 g) in methanol (19 ml) was hydrogenated over 10% palladium on carbon (0.969 g, 50% wet) at ambient temperature under atmospheric pressure of hydrogen for 2 hours. The reaction mixture was filtered through a short pad of celite, and the filtrate was concentrated in vacuo. The residue was purified by column chromatography on silica gel by eluting with hexane:ethyl acetate (1:2) to give tert-butyl 5-amino-2-pyridinyl(2-{2-[(tert-butoxycarbonyl)amino]-1,3-thiazol-4-yl}ethyl)carbamate (1.673 g) as a pale yellow solid.

¹H-NMR(CDCl₃): δ 1.50(18H, s), 2.97(2H, t), 2.63(2H, s), 4.07(2H, t), 6.75(1H, s), 6.95(1H, dd, J=8.6, 3.0 Hz), 7.10(1H, br d, J=8.1 Hz), 7.86(1H, d, J=3.0 Hz)

ESI-MS(m/z): 458(M+Na)⁺

### EXAMPLE 178

To a solution of 2-[4-(trifluoromethyl)phenyl]-1-cyclohexene-1-carboxylic acid (332 mg) in toluene (3.3 ml) were added thionyl chloride (0.179 ml) and N,N-dimethylformamide (1 drop) and the mixture was stirred at 80° C. for an hour. The mixture was evaporated in vacuo and the residue was dissolved in tetrahydrofuran (5.76 ml). The acid chloride in tetrahydrofuran was added to a solution of tert-butyl 5-amino-2-pyridinyl(2-{2-[(tert-butoxycarbonyl)amino]-1,3-thiazol-4-yl}ethyl)carbamate (411.1 mg) and triethylamine (0.197 ml) in tetrahydrofuran at ambient temperature and the mixture was stirred at the same temperature for an hour. The mixture was poured into water and extracted with ethyl acetate. The organic layer was washed with brine, dried over magnesium sulfate, and evaporated in vacuo. The residue was recrystallized from ethyl acetate-hexane to give tert-butyl 2-{2-[(tert-butoxycarbonyl)amino]-1,3-thiazol-4-yl}ethyl{5-[({2-[4-(trifluoromethyl)phenyl]-1-cyclohexen-1-yl}carbonyl)amino]-2-pyridinyl}carbamate (532 mg) as a white solid.

¹H-NMR(DMSO-d₆): δ 1.39(9H, s), 1.47(9H, s), 1.75(4H, br s), 2.41(4H, br s), 2.80(2H, br t, J=7.3 Hz), 3.97(2H, br t, J=6.5 Hz), 7.06(1H, s), 7.33(1H, d, J=8.9 Hz), 7.48(2H, d, J=8.3 Hz), 7.63(2H, d, J=8.6 Hz), 7.70(1H, dd, J=8.9, 2.7 Hz), 8.29(1H, d, J=2.2 Hz)

ESI-MS(m/z): 710(M+Na)⁺

### EXAMPLE 179

To a solution of tert-butyl 2-{2-[(tert-butoxycarbonyl)amino]-1,3-thiazol-4-yl}ethyl{5-[({2-[4-(trifluoromethyl)phenyl]-1-cyclohexen-1-yl}carbonyl)amino]-2-pyridinyl}carbamate (490.5 mg) in dichloromethane (4.9 ml) was added trifluoroacetic acid (0.824 ml). The reaction mixture was stirred for 19 hours, quenched with 10% aqueous potassium carbonate solution, and extracted with dichloromethane. The organic layer was washed with brine, dried over magnesium sulfate, filtered, and concentrated in vacuo. The residue was recrystallized from ethyl acetate-hexane to give N-(6-{[2-(2-amino-1,3-thiazol-4-yl)ethyl]amino}-3-pyridinyl)-2-[4-(trifluoromethyl)phenyl]-1-cyclohexene-1-carboxamide (296 mg) as a pale yellow powder.

¹H-NMR(DMSO-d₆): δ 1.73(4H, br s), 2.38(4H, br s), 2.60(2H, t, J=7.3 Hz), 3.38(2H, br t, J=7.3 Hz), 6.15(1H, s, J=7.3 Hz), 6.32(1H, d, J=8.9 Hz), 6.85(2H, br s), 7.24(1H, dd, J=8.9, 2.4 Hz), 7.48(2H, d, J=8.4 Hz), 7.64(2H, d, J=8.1 Hz), 7.80(1H, dd, J=2.2 Hz), 9.27(1H, s)

ESI-MS(m/z): 488(M+H)⁺

### EXAMPLE 180

tert-Butyl 2-{2-[(tert-butoxycarbonyl)amino]-1,3-thiazol-4-yl}ethyl[5-({[2-(4-methylphenyl)-1-cyclohexen-1-yl]carbonyl}amino)-2-pyridinyl]carbamate was obtained in the same manner as in Example 178 as a pale yellow foam.

¹H-NMR(DMSO-d₆): δ 1.39(9H, s), 1.47(9H, s), 1.73(4H, br s), 1.99(3H, s), 2.37(4H, br s), 2.81(2H, br t, J=7.8 Hz), 3.97(2H, br t, J=8.6 Hz), 7.05(2H, d, J=7.8 Hz), 7.07(1H, s), 7.18(2H, d, J=7.8 Hz), 7.75(1H, dd, J=8.9, 2.4 Hz), 8.31(1H, d, J=2.4 Hz), 9.71(1H, s)

ESI-MS(m/z): 656(M+Na)⁺

### EXAMPLE 181

N-(6-{[2-(2-Amino-1,3-thiazol-4-yl)ethyl]amino}-3-pyridinyl)-2-(4-methylphenyl)-1-cyclohexene-1-carboxamide was obtained in the same manner as in Example 179 as a pale yellow powder.

¹H-NMR(DMSO-d₆): δ 1.71(4H, br s), 2.23(3H, s), 2.34(4H, br s), 2.60(2H, t, J=7.3 Hz), 3.36(2H, t, J=8.1 Hz), 6.15(1H, s), 6.25-6.33(2H, m), 6.81(2H, br s), 7.06(2H, d, J=7.8 Hz), 7.17(2H, d, J=7.8 Hz), 7.30(1H, dd, J=8.6, 2.4 Hz), 7.82(1H, d, J=2.2 Hz), 9.11(1H, s)

ESI-MS(m/z): 456(M+Na)⁺

### EXAMPLE 182

tert-Butyl 2-{2-[(tert-butoxycarbonyl)amino]-1,3-thiazol-4-yl}ethyl[5-({[2-(4-ethylphenyl)-1-cyclohexen-1-yl]carbonyl}amino)-2-pyridinyl]carbamate was obtained in the same manner as in Example 178 as a pale yellow foam.

¹H-NMR(DMSO-d₆): δ 1.09(3H, t, J=7.6 Hz), 1.39(9H, s), 1.47(9H, s), 1.72(4H, br s), 2.37(4H, br s), 2.80(2H, t, J=7.8 Hz), 3.96(2H, t, J=8.1 Hz), 7.06(1H, s), 7.08(2H, d), 7.19(2H, d, J=7.8 Hz), 7.32(1H, d, J=8.6 Hz), 7.72(1H, dd, J=8.9, 2.4 Hz), 8.27(1H, d, J=2.4 Hz), 9.65(1H, s)

ESI-MS(m/z): 670(M+Na)⁺

### EXAMPLE 183

N-(6-{[2-(2-Amino-1,3-thiazol-4-yl)ethyl]amino}-3-pyridinyl)-2-(4-ethylphenyl)-1-cyclohexene-1-carboxamide was obtained in the same manner as in Example 179 as a pale yellow powder.

¹H-NMR(DMSO-d₆): δ 1.13(3H, t, J=7.8 Hz), 1.70(4H, br s), 2.34(4H, br s), 2.50-2.62(4H, m), 3.36(2H, q, J=7.8 Hz), 6.14(1H, s), 6.27-6.32(1H, m), 6.81(2H, s), 7.09(2H, d, J=8.1 Hz), 7.19(2H, d, J=8.4 Hz), 7.24(1H, dd, J=8.9, 2.7 Hz), 7.79(1H, d, J=2.2 Hz), 9.05(1H, s)

ESI-MS(m/z): 470(M+Na)⁺

Preparation 73

To a suspension of sodium hydride in 60% oil (0.245 g) in tetrahydrofuran (9 ml) was added tert-butyl 4-(2-hydroxyethyl)-1,3-thiazol-2-ylcarbamate (1.0 g) at 0° C. After 30 minutes, 2-bromo-5-nitropyridine was added to the reaction mixture at 0° C., and the mixture was stirred at 55° C. for 18 hours. The reaction mixture was poured into water and extracted with ethyl acetate. The organic layer was washed with brine, dried over magnesium sulfate, filtered, and concentrated in vacuo. The residue was purified by column chromatography on silica gel by eluting with hexane:ethyl acetate (8:1→6:1) to give a yellow foam. The foam was recrystallized from ethyl acetate-hexane to give tert-butyl 4-{2-[(5-nitro-2-pyridinyl)oxy]ethyl}-1,3-thiazol-2-ylcarbamate (0.623 g) as a yellow solid.

¹H-NMR(CDCl₃): δ 1.53(9H, s), 3.16(2H, t, J=6.9 Hz), 4.71(2H, t, J=6.9 Hz), 6.60(1H, s), 6.77(1H, d, J=9.5 Hz), 8.31(1H, dd, J=9.5, 3.0 Hz), 9.05(1H, d, J=3.0 Hz)

ESI-MS(m/z): 389(M+Na)⁺

Preparation 74

A solution of tert-butyl 4-{2-[(5-nitro-2-pyridinyl)oxy]ethyl-}-1,3-thiazol-2-ylcarbamate (0.672 g) in ethyl acetate (10 ml) was hydrogenated over 10% palladium on carbon (0.336 g, 50% wet) at ambient temperature under atmospheric pressure of hydrogen for an hour. The reaction mixture was filtered through a short pad of celite, and the filtrate was concentrated in vacuo. The residue was purified by column chromatography on silica gel by eluting with hexane:ethyl acetate (1:1→1:2→1:3) to give tert-butyl 4-{2-[(5-amino-2-pyridinyl)oxy]ethyl}-1,3-thiazol-2-ylcarbamate (0.561 g) as yellow crystals.

¹H-NMR(CDCl₃): δ 1.52(9H, s), 3.13(2H, t, J=6.6 Hz), 4.48(2H, t, J=6.6 Hz), 6.55(1H, d, J=8.9 Hz), 6.59(1H, s), 6.99(1H, dd, J=8.9, 3.2 Hz), 7.63(1H, d, J=3.2 Hz)

ESI-MS (m/z): 359 (M+Na)⁺

### EXAMPLE 184

To a solution of 2-[4-(trifluoromethyl)phenyl]-1-cyclohexene-1-carboxylic acid (275 mg) in toluene (4 ml) were added thionyl chloride (0.15 ml) and N,N-dimethylformamide (1 drop) and the mixture was stirred at 80° C. for an hour. The mixture was evaporated in vacuo and the residue was dissolved in tetrahydrofuran (2 ml). The acid chloride in tetrahydrofuran was added to a solution of tert-butyl 4-{2-[(5-amino-2-pyridinyl)oxy]ethyl}-1,3-thiazol-2-ylcarbamate (263 mg) and triethylamine (0.16 ml) in tetrahydrofuran (3 ml) at ambient temperature and the mixture was stirred at the same temperature for 11 hours. The reaction mixture was poured into water and extracted with ethyl acetate. The organic layer was washed with brine, dried over magnesium sulfate, and evaporated in vacuo. The residue was purified by column chromatography on silica gel by eluting with hexane:ethyl acetate (6:1→4:1→2:1) to give tert-butyl 4-[2-({5-[({2-[4-(trifluoromethyl)phenyl]-1-cyclohexen-1-yl}carbonyl)amino]-2-pyridinyl}oxy)ethyl]-1,3-thiazol-2-ylcarbamate (329 mg) as a yellow solid.

¹H-NMR(CDCl₃): δ 1.52(9H, s), 1.80(4H, br s), 2.43(2H, br s), 2.53(2H, br s), 3.07(2H, t, J=7.0 Hz), 4.47(2H, t, J=7.0 Hz), 6.44(1H, s), 6.55(1H, d, J=8.6 Hz), 6.56(1H, s), 7.36(1H, dd, J=8.6, 2.7 Hz), 7.41(2H, d, J=7.8 Hz), 7.59(2H, d, J=8.1 Hz), 7.61(1H, d, J=2.4 Hz)

ESI-MS(m/z): 611(M+Na)⁺

### EXAMPLE 185

To a solution of tert-butyl 4-[2-({5-[({2-[4-(trifluoromethyl)phenyl]-1-cyclohexen-1-yl}carbonyl)amino]-2-pyridinyl}oxy)ethyl]-1,3-thiazol-2-ylcarbamate (329 mg) in dichloromethane (3.3 ml) was added trifluoroacetic acid (0.647 ml). The reaction mixture was stirred for 14 hours, quenched with 10% aqueous potassium carbonate solution, and extracted with dichloromethane. The organic layer was washed with brine, dried over magnesium sulfate, filtered, and concentrated in vacuo. The residue was recrystallized from ethyl acetate-hexane to give N-{6-[2-(2-amino-1,3-thiazol-4-yl)ethoxy]-3-pyridinyl}-2-[4-(trifluoromethyl)phenyl]-1-cyclohexene-1-carboxamide (257 mg) as pale orange powder.

¹H-NMR(DMSO-₆): δ 1.75(4H, m), 2.40(4H, br s), 2.79(2H, t, J=7.0 Hz), 4.37(2H, t, J=7.0 Hz), 6.20(1H, s), 6.66(1H, d, J=8.6 Hz), 6.84(2H, s), 7.48(2H, d, J=8.1 Hz), 7.58(1H, dd, J=8.6, 2.7 Hz), 7.64(2H, d, J=8.3 Hz), 8.05(1H, d, J=2.7 Hz), 9.63(1H, s)

ESI-MS(m/z): 511(M+Na)⁺

### EXAMPLE 186

tert-Butyl 4-(2-{[5-({[2-(4-methylphenyl)-1-cyclohexen-1-yl]carbonyl}amino)-2-pyridinyl]oxy}ethyl)-1,3-thiazol-2-ylcarbamate was obtained in the same manner as in Example 184 as a pale yellow foam.

¹H-NMR(CDCl₃): δ 1.52(9H, s), 1.77(4H, br s), 2.33(3H, s), 2.42(2H, br s), 2.51(2H, br s), 3.07(2H, t, J=7.0 Hz), 4.47(2H, t, J=6.8 Hz), 6.50(1H, s), 6.55(1H, d, J=8.6 Hz), 6.56(1H, s), 7.16(4H, s), 7.44(1H, d, J=2.2 Hz), 7.48(1H, dd, J=8.9, 2.7 Hz)

ESI-MS(m/z): 557(M+Na)⁺

### EXAMPLE 187

N-{6-[2-(2-Amino-1,3-thiazol-4-yl) ethoxy]-3-pyridinyl}-2-(4-methylphenyl)-1-cyclohexene-1-carboxamide was obtained in the same manner as in Example 185 as a pale yellow powder.

¹H-NMR(DMSO-d₆): δ 1.72(4H, s), 2.22(3H, s), 2.34(4H, s), 2.80(2H, t, J=6.8 Hz), 4.37(2H, t, J=6.8 Hz), 6.20(1H, s), 6.66(1H, d, J=8.9 Hz), 6.85(2H, s), 7.06(2H, d, J=7.8 Hz), 7.18(2H, d, J=8.1 Hz), 7.61(1H, dd, J=8.9, 2.4 Hz), 8.07(1H, d, J=2.2 Hz), 9.48(1H, s)

ESI-MS(m/z): 457(M+Na)⁺

### EXAMPLE 188

tert-Butyl 4-(2-{[5-({[2-(4-ethylphenyl)-1-cyclohexen-1-yl]carbonyl}amino)-2-pyridinyl]oxy}ethyl)-1,3-thiazol-2-ylcarbamate was obtained in the same manner as in Example 184 as a pale yellow foam.

¹H-NMR(CDCl₃): δ 1.21(3H, t, J=7.6 Hz), 1.52(9H, s), 1.77(4H, br s), 2.43(2H, br s), 2.52(2H, br s), 2.61(2H, q, J=7.6 Hz), 3.08(2H, t, J=6.8 Hz), 4.46(2H, t, J=7.0 Hz), 6.47(1H, s), 6.53(1H, d, J=8.9 Hz), 6.56(1H, s), 7.18(4H, s), 7.37(1H, dd, J=8.9, 2.7 Hz), 7.45(1H, d, J=2.2 Hz), 9.26(1H, br s)

ESI-MS(m/z): 571(M+Na)⁺

### EXAMPLE 189

N-{6-[2-(2-Amino-1,3-thiazol-4-yl)ethoxy]-3-pyridinyl}-2-(4-ethylphenyl)-1-cyclohexene-1-carboxamide was obtained in the same manner as in Example 185 as a pale yellow powder.

¹H-NMR(DMSO-d₆): δ 1.10(3H, t, J=7.6 Hz), 1.72(4H, br s), 2.36(4H, br s), 2.37(2H, q, J=7.6 Hz), 2.79(2H, t, J=6.8 Hz), 4.37(2H, t, J=6.8 Hz), 6.20(1H, s), 6.64(1H, d, J=8.9 Hz), 6.86(2H, s), 7.08(2H, d, J=7.8 Hz), 7.19(2H, d, J=7.8 Hz), 7.56(1H, dd, J=8.9, 2.7 Hz), 8.03(1H, d, J=2.4 Hz), 9.42(1H, s)

ESI-MS(m/z): 471(M+Na)⁺

Preparation 75

A solution of tert-butyl 4-(4-nitrophenyl)-1-piperazinecarboxylate (207 mg) in methanol (5 ml) and tetrahydrofuran (2 ml) was hydrogenated over 10% palladium on carbon (40 mg) at ambient temperature under atmospheric pressure of hydrogen for 4 hours. The reaction mixture was filtered through a pad of celite, and the filtrate was concentrated in vacuo to give tert-butyl 4-(4-aminophenyl)-1-piperazinecarboxylate (186 mg) as a dark red tar. The product was used for the next step without further purification..

Preparation 76

To a solution of tert-butyl 4-(4-aminophenyl)-1-piperazinecarboxylate (182 mg), 2-[4-(trifluoromethyl)phenyl]-1-cyclohexene-1-carboxylic acid (187 mg) and 1-hydroxybenzotriazole monohydrate (134 mg) in N,N-dimethylformamide (5 ml) was added 1-[3-(dimethylamino)propyl]-3-ethylcarbodiimide hydrochloride (WSC.HCl) (168 mg), followed by triethylamine (100 mg) at ambient temperature. The reaction mixture was stirred for 3 days and concentrated in vacuo. The residue was dissolved in ethyl acetate and water. The organic layer was washed with brine, dried over magnesium sulfate, filtered, and concentrated in vacuo. The residue was purified by column chromatography on silica gel by eluting with hexane:ethyl acetate (2:1) to give tert-butyl 4-{4-[({2-[4-(trifluoromethyl)phenyl]-1-cyclohexen-1-yl}carbonyl)amino]phenyl}-1-piperazinecarboxylate (321 mg) as white crystals.

¹H-NMR(CDCl₃): δ 7.58(2H, d, J=8.6 Hz), 7.42(2H, d, J=8.2 Hz), 6.85(2H, d, J=8.9 Hz), 6.74(2H, d, J=8.9 Hz), 6.41(1H, br s), 3.54(4H, brt, J=5.1 Hz), 3.02(4H, brt, J=4.9 Hz), 2.54(2H, br s), 2.43(2H, br s), 1.80(4H, br s), 1.47(9H, s)

(+)ESI-MS(m/z): 552(M+Na)⁺

Preparation 77

To a solution of tert-butyl 4-{4-[({2-[4-(trifluoromethyl)phenyl]-1-cyclohexen-1-yl}carbonyl)amino]phenyl}-1-piperazinecarboxylate (313 mg) in dichloromethane (10 ml) was added trifluoroacetic acid (1.033 g). The reaction mixture was stirred for 18 hours, quenched with 10% aqueous potassium carbonate solution, and extracted with dichloromethane. The organic layer was washed with brine, dried over magnesium sulfate, filtered, and concentrated in vacuo to give N-[4-(1-piperazinyl)phenyl]-2-[4-(trifluoromethyl)phenyl]-1-cyclohexene-1-carboxamide (245 mg) as yellow crystals.

¹H-NMR(CDCl₃): δ 7.58(2H, d, J=8.6 Hz), 7.41(2H, d, J=7.9 Hz), 6.84(2H, d, J=8.9 Hz), 6.73(2H, d, J=8.9 Hz), 6.41(1H, br s), 3.04(4H, br s), 3.02(4H, br s), 2.54(2H, br s), 2.43(2H, br s), 1.84(4H, br s)

(+)ESI-MS(m/z): 430(M+H)⁺

### EXAMPLE 190

To a solution of N-[4-(1-piperazinyl)phenyl]-2-[4-(trifluoromethyl)phenyl]-1-cyclohexene-1-carboxamide (360 mg) and 3-formylbenzonitrile (220 mg) in dichloromethane (15 ml) was added sodium triacetoxyborohydride (530 mg) was added at ambient temperature. The reaction mixture was stirred for 3 hours, quenched with 10% aqueous potassium carbonate solution, and extracted with dichloromethane twice. The combined organic layers were washed with brine, dried over magnesium sulfate, and concentrated in vacuo. The residue was recrystallized from diisopropyl ether to give N-{4-[4-(3-cyanobenzyl)-1-piperazinyl]phenyl}-2-[4-(trifluoromethyl)phenyl]-1-cyclohexene-1-carboxamide (355 mg) as colorless crystals.

¹H-NMR(CDCl₃): δ 7.39-7.67(8H, m), 6.83(2H, d, J=8.9 Hz), 6.73(2H, d, J=9.2 Hz), 6.41(1H, br s), 3.57(2H, s), 3.11(4H, br s), 2.57(6H, br s), 2.43(2H, br s), 1.80(4H, br s)

(+)ESI-MS(m/z): 545(M+H)⁺

### EXAMPLE 191

To a solution of N-[4-(1-piperazinyl)phenyl]-2-[4-(trifluoromethyl)phenyl]-1-cyclohexene-1-carboxamide (126 mg), {6-[(tert-butoxycarbonyl)amino]-2-pyridinyl}acetic acid (75 mg) and 1-hydroxybenzotriazole (58 mg) in N,N-dimethylformamide (10 ml) was added 1-[3-(dimethylamino)propyl]-3-ethylcarbodiimide hydrochloride (WSC.HCl) (73 mg), followed by triethylamine (39 mg) at ambient temperature. The reaction mixture was stirred for 12 hours and concentrated in vacuo. The residue was dissolved in ethyl acetate and water, and extracted with ethyl acetate. The organic layer was washed with brine, dried over magnesium sulfate, and concentrated in vacuo. The residue was purified by column chromatography on silica gel by eluting with hexane:ethyl acetate (1:3) to give tert-butyl 6-[2-oxo-2-(4-{4-[({2-[4-(trifluoromethyl)phenyl]-1-cyclohexen-1-yl}carbonyl)amino]phenyl}-1-piperazinyl)ethyl]-2-pyridinylcarbamate (155 mg) as a pale yellow foam.

¹H-NMR(CDCl₃): δ 7.79(1H, d, J=8.2 Hz), 7.55-7.63(3H, m), 7.41(2H, d, J=7.9 Hz), 7.20(1H, br s), 6.94(1H, d, J=7.6 Hz), 6.84(2H, d, J=8.9 Hz), 6.70(2H, d, J=8.9 Hz), 6.46(1H, s), 3.81(2H, s), 3.74(2H, br s), 3.64(2H, br s), 3.02(2H, br s), 2.94(2H, br s), 2.53(2H, br s), 2.43(2H, br s), 1.79(4H, br s), 1.51(9H, s)

(+)ESI-MS(m/z): 664(M+H)⁺, 686(M+Na)⁺

### EXAMPLE 192

To a solution of tert-butyl 6-[2-oxo-2-(4-{4-[({2-[4-(trifluoromethyl)phenyl]-1-cyclohexen-1-yl}carbonyl)amino]phenyl}-1-piperazinyl)ethyl]-2-pyridinylcarbamate (155 mg) in dichloromethane (10 ml) was added trifluoroacetic acid (400 mg). The reaction mixture was stirred for 36 hours, quenched with 10% aqueous potassium carbonate solution, and extracted with dichloromethane. The organic layer was washed with brine, dried over magnesium sulfate, and concentrated in vacuo. The residue was recrystallized from ethyl acetate-diisopropyl ether to give N-(4-{4-[(6-amino-2-pyridinyl)acetyl]-1-piperazinyl}phenyl)-2-[4-(trifluoromethyl)phenyl]-1-cyclohexene-1-carboxamide (83 mg) as pale brown crystals.

¹H-NMR(DMSO-d₆): δ 9.41(1H, s), 7.61(2H, d, J=8.2 Hz), 7.47(2H, d, J=8.2 Hz), 7.28(1H, t, J=7.7 Hz), 7.17(2H, d, J=8.9 Hz), 6.78(2H, d, J=8.9 Hz), 6.35(1H, d, J=7.3 Hz), 6.27(1H, d, J=8.2 Hz), 5.81(2H, br s), 3.62(2H, s), 3.61(4H, br s), 2.96(4H, br s), 2.38(4H, br s), 1.73(4H, br s)

(+)ESI-MS(m/z): 564(M+H)⁺, 586(M+Na)⁺

### EXAMPLE 193

To a solution of N-[4-(1-piperazinyl)phenyl]-2-[4-(trifluoromethyl)phenyl]-1-cyclohexene-1-carboxamide (203 mg), 2-pyridinylacetic acid hydrochloride (90 mg) and 1-hydroxybenzotriazole (73 mg) in N,N-dimethylformamide (10 ml) was added 1-[3-(dimethylamino)propyl]-3-ethylcarbodiimide hydrochloride (WSC.HCl) (109 mg), followed by triethylamine (0.17 ml) at ambient temperature. The reaction mixture was stirred for 12 hours at 40° C. and concentrated in vacuo. The residue was dissolved in ethyl acetate and water, and extracted with ethyl acetate. The organic layer was washed with water and brine, dried over magnesium sulfate, filtered, and concentrated in vacuo. The residue was purified by column chromatography on silica gel by eluting with ethyl acetate:methanol (10:1) to give N-{4-[4-(2-pyridinylacetyl)-1-piperazinyl]phenyl}-2-[4-(trifluoromethyl)phenyl]-1-cyclohexene-1-carboxamide (120 mg) as a brown solid.

¹H-NMR(DMSO-d₆): δ 1.73(4H, br s), 2.38(4H, br s), 2.96(4H, br s), 3.57(2H, br s), 3.64(2H, br s), 3.90(2H, s), 7.15-7.30(4H, m), 7.47(2H, d, J=8.2 Hz), 7.62(2H, d, J=8.2 Hz), 7.72(1H, td, J=7.6, 1,6 Hz), 8.46(1H, d, J=4.0 Hz), 9.42(1H, s),

ESI-MS(m/z): 549(M+H)⁺

### EXAMPLE 194

To a suspension of N-[4-(1-piperazinyl)phenyl]-2-[4-(trifluoromethyl)phenyl]-1-cyclohexene-1-carboxamide (200 mg) and 1,3-thiazole-2-carbaldehyde (105 mg) in dichloromethane (20 ml) was added sodium triacetoxyborohydride (296 mg) at ambient temperature. The reaction mixture was stirred for 20 hours, quenched with 10% aqueous potassium carbonate solution, and extracted with ethyl acetate. The organic layer was washed with brine, dried over magnesium sulfate, and concentrated in vacuo. The residue was recrystallized from ethyl acetate-diisopropyl ether to give N-{4-[4-(1,3-thiazol-2-ylmethyl)-1-piperazinyl]phenyl}-2-[4-(trifluoromethyl)phenyl]-1-cyclohexene-1-carboxamide (178 mg) as a pale brown solid.

¹H-NMR(DMSO-d₆): δ 1.73(4H, br s), 2.38(4H, br s), 2.60(4H, br t, J=4.7 Hz), 3.05(4H, br t, J=4.7 Hz), 3.87(2H, s), 6.77(2H, d, J=8.9 Hz), 7.15(2H, d, J=8.9 Hz), 7.47(2H, d, J=7.9 Hz), 7.61(2H, d, J=8.2 Hz), 7.66(1H, d, J=3.3 Hz), 7.72(1H, d, J=3.3 Hz), 9.39(1H, s)

ESI-MS(m/z): 527(M+H)⁺

### EXAMPLE 195

N-{4-[4-(1H-Pyrrol-2-ylmethyl)-1-piperazinyl]phenyl}-2-[4-(trifluoromethyl)phenyl]-1-cyclohexene-1-carboxamide was obtained in the same manner as in Example 194 as a pale brown solid.

¹H-NMR(DMSO-d₆): δ 1.72(4H, br s), 2.37(4H, br s), 2.44(4H, br s), 3.00 (4H, br s), 3.42(2H, s), 5.88(1H, s), 5.91(1H, q, J=2.6 Hz), 6.62(1H, q, J=2.6 Hz), 6.75(2H, d, J=9.2 Hz), 7.14(2H, d, J=8.9 Hz), 7.47(2H, d, J=7.9 Hz), 7.61(2H, d, J=8.2 Hz), 9.39(1H, s), 10.68(1H, s)

ESI-MS(m/z): 509 (M+H)⁺

### EXAMPLE 196

To a suspension of N-[4-(1-piperazinyl)phenyl]-2-[4-(trifluoromethyl)phenyl]-1-cyclohexene-1-carboxamide (200 mg) and 3-chloro-1-propene (71.3 mg) in acetone (30 ml) was added cesium carbonate (228 mg) at ambient temperature. The reaction mixture was stirred at 70° C. for 14 hours. After cooling, water was added, and extracted with ethyl acetate. The organic layer was washed with brine, dried over magnesium sulfate, and concentrated in vacuo. The residue was recrystallized from ethyl acetate-diisopropyl ether to give N-[4-(4-allyl-1-piperazinyl)phenyl]-2-[4-(trifluoromethyl)phenyl]-1-cyclohexene-1-carboxamide (82 mg) as a pale yellow solid.

¹H-NMR(DMSO-d₆): δ 1.73(4H, br s), 2.38(4H, br s), 2.45(4H, br t, J=4.7 Hz), 2.98(2H, d, J=6.2 Hz), 3.01(4H, br t, J=5.7 Hz), 5.11-5.22(2H, m), 5.74-5.89(1H, m), 6.75(2H, d, J=9.2 Hz), 7.16(2H, d, J=8.9 Hz), 7.48(2H, d, J=7.9 Hz), 7.62(2H, d, J=8.2 Hz), 9.40(1H, s)

ESI-MS(m/z): 470(M+H)⁺

### EXAMPLE 197

N-{4-[4-(1H-Imidazol-5-ylmethyl)-1-piperazinyl]phenyl}-2-[4-(trifluoromethyl)phenyl]-1-cyclohexene-1-carboxamide was obtained in the same manner as in Example 194 as a white solid.

¹H-NMR(DMSO-d₆): δ 1.73(4H, br s), 2.37(4H, br s), 2.50(4H, br s), 3.01(4H, br t, J=4.6 Hz), 3.45(2H, s), 6.75(2H, d, J=9.2 Hz), 6,89(1H, s), 7.15(2H, d, J=9.2 Hz), 7.48(2H, d, J=8.2 Hz), 7.55(1H, d, J=1.0 Hz), 7.61(2H, d, J=8.2 Hz), 9.38(1H, s),

ESI-MS(m/z): 510(M+H)⁺

### EXAMPLE 198

To a solution of N-[4-(1-piperazinyl)phenyl]-2-[4-(trifluoromethyl)phenyl]-1-cyclohexene-1-carboxamide (200 mg) and 2-bromoacetamide (77 mg) in tetrahydrofuran (15 ml) was added triethylamine (78 μl) at ambient temperature. The reaction mixture was stirred at 70° C. for 2 hours. After cooling, the solvent was concentrated in vacuo, and the concentrate was extracted with dichloromethane. The organic layer was washed with brine, dried over magnesium sulfate, and concentrated in vacuo. The residue was recrystallized from ethyl acetate-diisopropyl ether to give N-{4-[4-(2-amino-2-oxoethyl)-1-piperazinyl]phenyl}-2-[4-(trifluoromethyl)phenyl]-1-cyclohexene-1-carboxamide (168 mg) as a pale yellow solid.

¹H-NMR(DMSO-d₆): δ 1.73(4H, br s), 2.38(4H, br s), 2.54(4H, br s), 2.89(2H, s), 3.05(4H, br t, J=4.6 Hz), 6.75(2H, d, J=8.9 Hz), 7.14(2H, br s), 7.16(2H, d, J=8.9 Hz), 7.48(2H, d, J=7.9 Hz), 7.62(2H, d, J=8.2 Hz), 9.39(1H, s),

ESI-MS(m/z) : 487(M+H)⁺

### EXAMPLE 199

To a solution of N-[4-(1-piperazinyl)phenyl]-2-[4-(trifluoromethyl)phenyl]-1-cyclohexene-1-carboxamide (200 mg), (2-(formylamino)-1,3-thiazol-4-yl)acetic acid (91 mg) and 1-hydroxybenzotriazole (76 mg) in dichloromethane (20 ml) was added 1-[3-(dimethylamino)propyl]-3-ethylcarbodiimide hydrochloride (WSC.HCl) (107 mg), followed by triethylamine (0.1 ml) at ambient temperature. The reaction mixture was stirred for 12 hours and extracted with dichloromethane. The organic layer was washed with water and brine, dried over magnesium sulfate, filtered, and concentrated in vacuo. The residue was purified by column chromatography on silica gel by eluting with ethyl acetate:methanol (10:1) to give N-[4-(4-{[2-(formylamino)-1,3-thiazol-4-yl]acetyl}-1-piperazinyl)phenyl]-2-[4-(trifluoromethyl)phenyl]-1-cyclohexene-1-carboxamide (232 mg) as a white solid..

¹H-NMR(DMSO-d₆): δ 1.73(4H, br s), 2.38(4H, br s), 2.98(4H, br s), 3.55-3.64(4H, m), 3.76(2H, s), 6.79(2H, d, J=8.9 Hz), 6.95(1H, s), 7.18(2H, d, J=8.9 Hz), 7.48(2H, d, J=7.9 Hz), 7.62(2H, d, J=8.2 Hz), 8.44(1H, s), 9.42(1H, s), 12.18(1H, s),

ESI-MS(m/z): 598(M+H)⁺

### EXAMPLE 200

To a suspension of N-[4-(4-{[2-(formylamino)-1,3-thiazol-4-yl]acetyl}-1-piperazinyl)phenyl]-2-[4-(trifluoromethyl)phenyl]-1-cyclohexene-1-carboxamide (200 mg) in methanol (5 ml) was added concentrated hydrochloric acid (0.16 ml). The reaction mixture was stirred at 50° C. for 2 hours, quenched with 10% aqueous potassium carbonate solution, and extracted with ethyl acetate-tetrahydrofuran. The organic layer was washed with brine, dried over magnesium sulfate, filtered, and concentrated in vacuo. The residue was recrystallized from ethyl acetate-diisopropyl ether to give N-(4-{4-[(2-amino-1,3-thiazol-4-yl)acetyl]-1-piperazinyl}phenyl)-2-[4-(trifluoromethyl)phenyl]-1-cyclohexene-1-carboxamide (167 mg) as a pale brown solid.

¹H-NMR(DMSO-d₆): δ 1.72(4H, br s), 2.38(4H, br s), 2.97(4H, br s), 3.52(2H, s), 3.56-3.60(4H, m), 6.22(1H, s), 6.79(2H, d, J=8.9 Hz), 6.84(1H, s), 7.17(2H, d, J=9.2 Hz), 7.48(2H, d, J=8.2 Hz), 7.62(2H, d, J=8.2 Hz), 9.42(1H, s)

ESI-MS(m/z): 570(M+H)⁺

### EXAMPLE 201

To a solution of N-[4-(1-piperazinyl)phenyl]-2-[4-(trifluoromethyl)phenyl]-1-cyclohexene-1-carboxamide (200 mg) and 2-{6-[(tert-butoxycarbonyl)amino]-2-pyridinyl}ethyl 4-methylbenzenesulfonate (192 mg) in tetrahydrofuran (15 ml) was added triethylamine (78 ml) at ambient temperature. The reaction mixture was stirred at 70° C. for 30 hours. After cooling, water was added, and extracted with ethyl acetate. The organic layer was washed with brine, dried over magnesium sulfate, and concentrated in vacuo. The residue was purified by column chromatography on silica gel by eluting with ethyl acetate to give tert-butyl 6-[2-(4-{4-[({2-[4-(trifluoromethyl)phenyl]-1-cyclohexen-1-yl}carbonyl)amino]phenyl}-1-piperazinyl)ethyl]-2-pyridinylcarbamate (122 mg) as a pale yellow solid.

¹H-NMR(DMSO-d₆): δ 1.45(9H, s), 1.74(4H, br s), 2.39(4H, br s), 2.51(4H, br s), 2.65(2H, m), 2.79(2H, m), 3.01(4H, br s), 6.75(2H, d, J=8.9 Hz), 6.91(1H, dd, J=5.9, 2.6 Hz), 7.15(2H, d, J=9.2 Hz), 7.48(2H, d, J=7.9 Hz), 7.59-7.63(4H, m), 9.40(1H, s), 9.58(1H, s)

ESI-MS(m/z): 650(M+H)⁺

### EXAMPLE 202

To a solution of tert-butyl 6-[2-(4-{4-[({2-[4-(trifluoromethyl)phenyl]-1-cyclohexen-1-yl}carbonyl)amino]phenyl}-1-piperazinyl)ethyl]-2-pyridinylcarbamate (112 mg) in dichloromethane (5 ml) was added trifluoroacetic acid (0.27 ml). The reaction mixture was stirred at ambient temperature for 14 hours, quenched with 10% aqueous potassium carbonate solution, and extracted with ethyl acetate-tetrahydrofuran. The organic layer was washed with brine, dried over magnesium sulfate, filtered, and concentrated in vacuo. The residue was recrystallized from ethyl acetate-diisopropyl ether to give N-(4-{4-[2-(6-amino-2-pyridinyl)ethyl]-1-piperazinyl}phenyl)-2-[4-(trifluoromethyl)phenyl]-1-cyclohexene-1-carboxamide (68 mg) as a pale brown solid.

¹H-NMR(DMSO-d₆): δ 1.72(4H, br s), 2.38(4H, br s), 2.54(2H, br s), 2.63(4H, br s), 3.01(4H, br s), 3.34(2H, br s), 5.79(2H, br s), 6.24(1H, d, J=8.2 Hz), 6.37(1H, d, J=7.2 Hz), 6.76(2H, d, J=8.9 Hz), 7.15(2H, d, J=8.9 Hz), 7.25(1H, t, J=7.9 Hz), 7.48(2H, d, J=7.9 Hz), 7.62(2H, d, J=8.6 Hz), 9.40(1H, s)

ESI-MS(m/z): 550(M+H)⁺

### EXAMPLE 203

N-{4-[4-(1H-Imidazol-2-ylmethyl)-1-piperazinyl]phenyl}-2-[4-(trifluoromethyl)phenyl]-1-cyclohexene-1-carboxamide was obtained in the same manner as in Example 194 as a pale yellow solid.

¹H-NMR(DMSO-d₆): δ 1.73(4H, br s), 2.38(4H, br s), 2.50(4H, br s), 3.01(4H, br t, J=4.6 Hz), 3.51(2H, s), 6.75(2H, d, J=8.9 Hz), 6.91(1H, s), 7.14(2H, d, J=9.2 Hz), 7.47(2H, d, J=8.2 Hz), 7.61(2H, d, J=8.2 Hz), 9.40(1H, s)

ESI-MS(m/z): 510(M+H)⁺

### EXAMPLE 204

N-{4-[4-(3-Chlorobenzyl)-1-piperazinyl]phenyl}-2-[4-(trifluoromethyl)phenyl]-1-cyclohexene-1-carboxamide was obtained in the same manner as in Example 194 as a white solid.

¹H-NMR(DMSO-d₆): δ 1.73(4H, br s), 2.38(4H, br s), 2.47(4H, br s), 3.02(4H, br t, J=4.6 Hz), 3.51(2H, s), 6.75(2H, d, J=8.9 Hz), 7.15(2H, d, J=8.6 Hz), 7.26-7.39(4H, m), 7.48(2H, d, J=7.9 Hz), 7.60(2H, d, J=8.2 Hz), 9.39(1H, s)

ESI-MS(m/z): 555(M+H)⁺

### EXAMPLE 205

N-{4-[4-(3-Methylbenzyl)-1-piperazinyl]phenyl}-2-[4-(trifluoromethyl)phenyl]-1-cyclohexene-1-carboxamide was obtained in the same manner as in Example 194 as a white solid .

¹H-NMR(DMSO-d₆): δ 1.73(4H, br s), 2.29(3H, s), 2.37(4H, br s), 2.45(4H, br s), 3.01(4H, br s), 3.44(2H, s), 6.75(2H, d, J=8.9 Hz), 7.04-7.23(6H, m), 7.47(2H, d, J=8.2 Hz), 7.61(2H, d, J=8.2 Hz), 9.38(1H, s)

ESI-MS(m/z): 534(M+H)⁺

### EXAMPLE 206

N-{4-[4-(3-Methoxybenzyl)-1-piperazinyl]phenyl}-2-[4-(trifluoromethyl)phenyl]-1-cyclohexene-1-carboxamide was obtained in the same manner as in Example 194 as a white solid.

¹H-NMR(DMSO-d₆): δ 1.73(4H, br s), 2.37(4H, br s), 2.46(4H, br s), 3.02(4H, br s), 3.46(2H, s), 3.73(3H, s), 6.75(2H, d, J=8.9 Hz), 6.79-6.83(1H, m), 6.87(2H, d, J=7.9 Hz), 7.14(2H, d, J=8.9 Hz), 7.23(1H, dd, J=8.2, 7.9 Hz), 7.48(2H, d, J=7.9 Hz), 7.61(2H, d, J=8.2 Hz), 9.38(1H, s)

ESI-MS(m/z): 550(M+H)⁺

### EXAMPLE 207

To a solution of N-[4-(1-piperazinyl)phenyl]-2-[4-(trifluoromethyl)phenyl]-1-cyclohexene-1-carboxamide (200 mg) and ethyl bromoacetate (117 mg) in acetone (10 ml) was added a potassium carbonate (193 mg) at ambient temperature. The reaction mixture was stirred at 70° C. for 2 hours. After cooling, acetone was evaporated and the concentrate was extracted with ethyl acetate. The organic layer was washed with brine, dried over magnesium sulfate, and concentrated in vacuo. The residue was purified by column chromatography on silica gel by eluting with ethyl acetate to give ethyl (4-{4-[({2-[4-(trifluoromethyl)phenyl]-1-cyclohexen-1-yl}carbonyl)amino]phenyl}-1-piperazinyl)acetate (141 mg) as a white solid.

¹H-NMR(DMSO-d₆): δ 1.19(3H, t, J=7.1 Hz), 1.73(4H, br s), 2.38(4H, br s), 2.60(4H, br t, J=4.7 Hz), 3.01(4H, br t, J=4.9 Hz), 3.24(2H, s), 4.08(2H, q, J=7.1 Hz), 6.76(2H, d, J=8.9 Hz), 7.16(2H, d, J=9.2 Hz), 7.48(2H, d, J=7.9 Hz), 7.62(2H, d, J=8.2 Hz), 9.40(1H, s)

ESI-MS(m/z): 516(M+H)⁺

### EXAMPLE 208

To a suspension of N-[4-(1-piperazinyl)phenyl]-2-[4-(trifluoromethyl)phenyl]-1-cyclohexene-1-carboxamide (200 mg) and triethylamine (0.1 ml) in dichloromethane (10 ml) was added 3-cyanobenzoyl chloride (93 mg) at 0° C. The reaction mixture was stirred for 2 hours at ambient temperature, poured into water, and extracted with dichloromethane. The organic layer was washed with brine, dried over magnesium sulfate, and concentrated in vacuo. The residue was recrystallized from ethyl acetate-diisopropyl ether to give N-{4-[4-(3-cyanobenzoyl)-1-piperazinyl]phenyl}-2-[4-(trifluoromethyl)phenyl]-1-cyclohexene-1-carboxamide (203 mg) as a white solid.

¹H-NMR(DMSO-d₆): δ 1.72(4H, br s), 2.38(4H, br s), 3.06(4H, br s), 3.42(2H, br s), 3.73(2H, br s), 6.80(2H, d, J=9.2 Hz), 7.18(2H, d, J=9.2 Hz), 7.48(2H, d, J=7.9 Hz), 7.61(2H, d, J=8.9 Hz), 7.68(1H, d, J=8.2 Hz), 7.76(1H, dt, J=7.9, 1,3 Hz), 7.91-7.95(2H, m), 9.43(1H, s)

ESI-MS(m/z): 581(M+Na)⁺

### EXAMPLE 209

N-{4-[4-(3,4-Dimethoxybenzyl)-1-piperazinyl]phenyl}-2-[4-(trifluoromethyl)phenyl]-1-cyclohexene-1-carboxamide was obtained in the same manner as in Example 194 as a white solid

¹H-NMR(DMSO-d): δ 1.73(4H, br s), 2.38(4H, br s), 2.45(4H, br s), 3.01(4H, br s), 3.42(2H, s), 3.72(3H, s), 3.73(3H, s), 6.77-6.90(5H, m), 7.15(2H, d, J=8.9 Hz), 7.48(2H, d, J=8.2 Hz), 7.62(2H, d, J=8.6 Hz), 9.40(1H, s)

ESI-MS(m/z): 580(M+H)⁺

### EXAMPLE 210

N-[4-(4-Allyl-1-piperazinyl)phenyl]-2-(4-methylphenyl)-1-cyclohexene-1-carboxamide was obtained in the same manner as in Example 196 as a pale brown solid.

¹H-NMR(DMSO-d₆): δ 1.70(4H, br s), 2.21(3H, s), 2.33(4H, br s), 2.45(4H, br t, J=4.6 Hz), 2.96(2H, d, J=6.2 Hz), 2.99(4H, br s), 5.11-5.23(2H, m), 5.74-5.89(1H, m), 6.75(2H, d, J=8.9 Hz), 7.04(2H, d, J=8.2 Hz), 7.17(2H, d, J=7.9 Hz), 7.19(2H, d, J=8.9 Hz), 9.25(1H, s)

ESI-MS(m/z): 416(M+H)⁺

### EXAMPLE 211

2-(4-Methylphenyl)-N-{4-[4-(1H-pyrrol-2-ylmethyl)-1-piperazinyl]phenyl}-1-cyclohexene-1-carboxamide was obtained in the same manner as in Example 194 as a brown foam.

¹H-NMR(DMSO-d₆): δ 1.70(4H, br s), 2.20(3H, s), 2.33(4H, br s), 2.43(4H, br s), 3.00(4H, br s), 3.42(2H, s), 5.88(1H, br s), 5.91-5.93(1H, m), 6.63(1H, d, J=1.6 Hz), 6.74(2H, d, J=9.2 Hz), 7.03(2H, d, J=7.9 Hz), 7.16-7.20(4H, m), 9.25(1H, s), 10.68(1H, s)

ESI-MS(m/z): 477(M+Na)⁺

Preparation 78

tert-Butyl 4-(5-amino-2-pyridinyl)-1-piperazinecarboxylate was obtained in the same manner as in Preparation 75 as a dark purple oil.

Preparation 79

To a solution of 2-[4-(trifluoromethyl)phenyl]-1-cyclohexene-1-carboxylic acid (555 mg) in toluene (10 ml) were added thionyl chloride (366 mg) and N,N-dimethylformamide (2 drops) and the mixture was stirred at 50° C. for an hour. The mixture was evaporated in vacuo and the residue was dissolved in tetrahydrofuran (5 ml). The acid chloride in tetrahydrofuran was added to a solution of tert-butyl 4-(5-amino-2-pyridinyl)-1-piperazinecarboxylate (571 mg) and triethylamine (250 mg) in tetrahydrofuran (20 ml) at ambient temperature and the mixture was stirred at the same temperature for an hour. The mixture was poured into water and extracted with ethyl acetate. The organic layer was washed with brine, dried over magnesium sulfate, and evaporated in vacuo to give tert-butyl 4-{5-[({2-[4-(trifluoromethyl)phenyl]-1-cyclohexen-1-yl}carbonyl)amino]-2-pyridinyl}-1-piparazinecarboxylate (1.088 g) as pale purple crystals.

¹H-NMR(CDCl₃): δ 7.63(1H, d, J=2.6 Hz), 7.59(2H, d, J=8.2 Hz), 7.41(2H, d, J=8.2 Hz), 7.36(1H, dd, J=8.9 and 2.6 Hz), 6.51(1H, d, J=8.9 Hz), 6.38(1H, br s), 3.47-3.51(4H, m), 3.39-3.44(4H, m), 2.53(2H, br s), 2.42(2H, br s), 1.80(4H, br s), 1.47(9H, s)

(+)ESI-MS(m/z): 531(M+H)⁺, 553(M+Na)⁺

Preparation 80

N-[6-(1-Piperazinyl)-3-pyridinyl]-2-[4-(trifluoromethyl)phenyl]-1-cyclohexene-1-carboxamide was obtained in the same manner as in Preparation 77 as a dark purple oil.

¹H-NMR(DMSO-d₆): δ 9.43(1H, s), 7.98(1H, d, J=2.6 Hz), 7.64(2H, d, J=8.6 Hz), 7.48(2H, d, J=8.3 Hz), 7.42(1H, d, J=2.3 Hz), 6.67(1H, d, J=9.2 Hz), 3.29(4H, t, J=4.9 Hz), 2.76(4H, t, J=4.9 Hz), 2.39(4H, br s), 1.73(4H, br s)

(+)ESI-MS(m/z): 431(M+H)⁺

### EXAMPLE 212

N-{6-[4-(3-Cyanobenzyl)-1-piperazinyl]-3-pyridinyl}-2-[4-(trifluoromethyl)phenyl]-1-cyclohexene-1-carboxamide was obtained in the same manner as in Example 212 as a dark purple oil.

¹H-NMR(DMSO-d₆): δ 9.44(1H, s), 7.98(1H, d, J=2.3 Hz), 7.43-7.75(9H, m), 6.68(1H, d, J=9.2 Hz), 3.56(2H, s), 3.37(4H, br s), 2.39-2.43(8H, m), 1.74(4H, br s)

(+)ESI-MS(m/z): 546(M+H)⁺, 568(M+Na)⁺

### EXAMPLE 213

To a solution of N-[6-(1-piperazinyl)-3-pyridinyl]-2-[4-(trifluoromethyl)phenyl]-1-cyclohexene-1-carboxamide (250 mg), 2-pyridinylacetic acid hydrochloride (101 mg) and 1-hydroxybenzotriazole (116 mg) in N,N-dimethylformamide (10 ml) was added 1-[3-(dimethylamino)propyl]-3-ethylcarbodiimide hydrochloride (WSC.HCl) (145 mg), followed by triethylamine (153 mg) at ambient temperature. The reaction mixture was stirred for 12 hours and concentrated in vacuo. The residue was dissolved in ethyl acetate and water, and extracted with ethyl acetate. The organic layer was washed with brine, dried over magnesium sulfate, and concentrated in vacuo. The residue was purified by column chromatography on silica gel by eluting with ethyl acetate to give N-{6-[4-(2-pyridinylacetyl)-1-piperazinyl]-3-pyridinyl}-2-[4-(trifluoromethyl)phenyl]-1-cyclohexene-1-carboxamide (232 mg) as a colorless sticky oil.

¹H-NMR(DMSO-d₆): δ 9.47(1H, s), 8.47(1H, d, J=3.0 Hz), 8.01(1H, d, J=2.6 Hz), 7.72(1H, dt, J=7.6 and 1.7 Hz), 7.63(2H, d, J=8.2 Hz), 7.46-7.50(3H, m), 7.29(1H, d, J=7.9 Hz), 7.23(1H, dd, J=7.6 and 4.9 Hz), 6.73(1H, d, J=9.2 Hz), 3.91(2H, s), 3.61(2H, br s), 3.54(2H, br s), 3.33(4H, br s), 2.39(4H, br s), 1.74(4H, br s)

(+)ESI-MS(m/z): 572(M+Na)⁺

### EXAMPLE 214

tert-Butyl 6-[2-oxo-2-(4-{5-[({2-[4-(trifluoromethyl)phenyl]-1-cyclohexen-1-yl}carbonyl)amino]-2-pyridinyl}-1-piperazinyl)ethyl]-2-pyridinylcarbamate (478 mg) was obtained in the same manner as in Example 191 as a pale yellow foam.

¹H-NMR (CDCl₃): δ 7.78(1H, d, J=8.2 Hz), 7.62(2H, d, J=2.3 Hz), 7.58(2H, d, J=8.2 Hz), 7.41(2H, d, J=8.2 Hz), 7.36(1H, dd, J=8.9 and 2.6 Hz), 7.13(1H, br s), 6.95(1H, d, J=7.6 Hz), 6.48(1H, d, J=8.9 Hz), 6.40(1H, br s), 3.81(2H, s), 3.71(2H, brt, J=5.3 Hz), 3.61(2H, brt, J=5.3 Hz), 3.39(4H, brt, J=5.3 Hz), 2.53(2H, br s), 2.42(2H, br s), 1.79(4H, br s), 1.51(9H, s)

(+)ESI-MS(m/z): 687(M+Na)⁺

### EXAMPLE 215

N-(6-{4-[(6-Amino-2-pyridinyl)acetyl]-1-piperazinyl}-3-pyridinyl)-2-[4-(trifluoromethyl)phenyl]-1-cyclohexene-1-carboxamide was obtained in the same manner as in Example 192 as colorless crystals.

¹H-NMR(DMSO-d₆): δ 9.46(1H, s), 8.00(1H, d, J=2.3 Hz), 7.63(2H, d, J=8.6 Hz), 7.47(3H, d, J=8.6 Hz), 7.28(1H, t, J=7.7 Hz), 6.72(1H, d, J=9.2 Hz), 6.36(1H, d, J=7.3 Hz), 6.27(1H, d, J=8.2 Hz), 5.83(2H, br s), 3.62(2H, s), 3.52-3.60(4H, m), 3.32(4H, br s), 2.39(4H, br s), 1.73(4H, br s)

(+)ESI-MS(m/z): 565(M+H)⁺

Preparation 81

A solution of 2-chloro-4-nitrobenzoic acid (7.5 g) in dimethyl malonate (90 ml) was degassed with argon for 15 min. Copper(I) bromide (0.54 g) was added in one portion. Sodium methoxide (4.83 g) was added in one portion with stirring. After being stirred for 15 minutes, the reaction mixture was heated to 70° C. for 24 hours. Water (90 ml) was added to the cooled reaction mixture, followed by hexane (90 ml). The aqueous layer was separated. Toluene (90 ml) was added to the aqueous layer, and the biphasic mixture was filtered through Celite to remove insoluble substances. The aqueous layer was separated, acidified with 6N hydrochloric acid. A pale brown precipitate formed, and the mixture was stirred for 18 hours. The obtained product was collected by filtration and dried to give 2-[2-methoxy-1-(methoxycarbonyl)-2-oxoethyl]-4-nitrobenzoic acid (8.3 g) as a pale brown solid.

¹H-NMR(DMSO-d₆): δ 3.70(6H, s), 5.82(1H, s), 8.17(1H, d, J=8.6 Hz), 8.22(1H, d, J=2.2 Hz), 8.31(1H, dd, J=8.6, 2.2 Hz)

Preparation 82

To a solution of 2-[2-methoxy-1-(methoxycarbonyl)-2-oxoethyl]-4-nitrobenzoic acid (3.0 g) in methanol (24 ml) was added sodium hydroxide (2.02 g) in water (24 ml) over 85 minutes at ambient temperature. After 3 hours, methanol was removed under vacuum, and the concentrate was acidified with concentrated hydrochloric acid (4.48 ml) at ambient temperature. The resulting white aqueous suspension was extracted twice with ethyl acetate (30 ml and 15 ml), the combined organic layers were dried over magnesium sulfate and concentrated to 11 ml. The resulting ethyl acetate slurry was heated to 65° C. for 6 hours, filtered off at room temperature and dried to give 2-(carboxymethyl)-4-nitrobenzoic acid (1.93 g) as a white solid.

¹H-NMR(DMSO-d₆): δ 4.10(2H, s), 8.09(1H, d, J=8.6 Hz), 8.21(1H, dd, J=8.6, 2.2 Hz), 8.27(1H, d, J=2.2 Hz)

Preparation 83

To a solution of 2-(carboxymethyl)-4-nitrobenzoic acid (1.92 g) in tetrahydrofuran (42 ml) was added sodium borohydride (0.968 g) in portions. The contents were cooled to 0° C., and boron trifluoride diethyl etherate (3.63 g) was added dropwise over an hour and stirred at ambient temperature for 16 hours. The reaction mixture was cooled to 0° C. and quenched with 1N aqueous sodium hydroxide (34 ml). The reaction mixture was stirred for 3 hours, tetrahydrofuran was removed under vacuum. The resulting aqueous suspension was cooled to 0° C., and the product was filtered off and dried to give 2-[2-(hydroxymethyl)-5-nitrophenyl]ethanol (1.44 g) as a white solid.

¹H-NMR(DMSO-d₆): δ 2.82(2H, t, J=6.0 Hz), 3.62-3.69(2H, m), 4.66(2H, d, J=3.8 Hz), 4.76(1H, t, J=5.0 Hz), 5.45(1H, s), 7.68(1H, d, J=9.2 Hz), 8.05-8.09(2H, m)

Preparation 84

To a solution of 2-[2-(hydroxymethyl)-5-nitrophenyl]ethanol (1.941 g) and triethylamine (3.43 ml) in methylene chloride (55.5 ml) was added methanesulfonyl chloride (1.75 ml) at 0° C. for 30 minutes. The reaction mixture was washed with 10% aqueous hydrohloric acid, saturated aqueous sodium bicarbonate, and brine. The organic layer was dried with magnesium sulfate, and methylene chloride was removed under vacuum. The residue was purified by column chromatography on silica gel by eluting with chloroform to give 2-{2-[(methylsulfonyl)oxy]ethyl}-4-nitrobenzyl methanesulfonate (2.922 g) as a white solid.

¹H-NMR(CDCl₃): δ 3.00(3H, s), 3.15(3H, s), 3.30(2H, t, J=6.5 Hz), 4.56(2H, t, J=6.5 Hz), 4.69(2H, s), 7.58(1H, d, J=8.1 Hz), 8.10-8.17(2H, m)

Preparation 85

To a solution of 2-{2-[(methylsulfonyl)oxy]ethyl}-4-nitrobenzyl methanesulfonate (2.12 g) in tetrahydrofuran (10.6 ml) was added triethylamine (4.18 ml) and N-acetylethylenediamine (3.06 g). After stirring for an hour, the reaction mixture was heated to 60° C. for 16 hour. The mixture was poured into water and extracted with ethyl acetate. The organic layer was washed with brine, dried over magnesium sulfate, and evaporated in vacuo. The residue was recrystallized from ethyl acetate and hexane to give N-[2-(6-nitro-3,4-dihydro-2(1H)-isoquinolinyl)ethyl]acetamide (1.24 g) as a yellow powder.

¹H-NMR(CDCl₃): δ 1.98(3H, s), 2.69(2H, t, J=5.9 Hz), 2.80(2H, t, J=5.9 Hz), 3.00(2H, t, J=5.1 Hz), 3.46(2H, q, J=5.1 Hz), 3.72(2H, s), 6.01(1H, br s), 7.18(1H, d, J=8.9 Hz), 7.95-8.00(2H, m)

ESI-MS(m/z): 264(M+H)⁺

Preparation 86

A solution of N-[2-(6-nitro-3,4-dihydro-2(1H)-isoquinolinyl)ethyl]acetamide (1.23 g) in ethyl acetate (12 ml) was hydrogenated over 10% palladium on carbon (0.61 g, 50% wet) at ambient temperature under atmospheric pressure of hydrogen for 3 hours. The reaction mixture was filtered through a short pad of celite, and the filtrate was concentrated in vacuo to give N-[2-(6-amino-3,4-dihydro-2(1H)-isoquinolinyl)ethyl]acetamide (1.09 g) as a pale yellow foam.

¹H-NMR(CDCl₃): δ 1.93(3H, s), 2.60(2H, t, J=6.5 Hz), 2.69(2H, t, J=5.7 Hz), 2.75-2.82(2H, m), 3.39(2H, q, J=5.7 Hz), 3.45-3.70(4H, m), 6.43(1H, d, J=2.3 Hz), 6.48(1H, dd, J=7.8, 2.3 Hz), 6.81(1H, d, J=7.8 Hz)

ESI-MS(m/z): 234(M+H)⁺

### EXAMPLE 216

To a solution of 2-[4-(trifluoromethyl)phenyl]-1-cyclohexene-1-carboxylic acid (229 mg) in toluene (4.6 ml) were added thionyl chloride (0.08 ml) and N,N-dimethylformamide (1 drop) and the mixture was stirred at 80° C. for an hour. The mixture was evaporated in vacuo and the residue was dissolved in tetrahydrofuran (1.2 ml). The acid chloride in tetrahydrofuran was added to a solution of N-[2-(6-amino-3,4-dihydro-2(1H)-isoquinolinyl)ethyl]acetamide (1.09 g) and triethylamine (0.136 ml) in tetrahydrofuran (3.6 ml) at ambient temperature and the mixture was stirred at the same temperature for 2 hours. The reaction mixture was poured into water and extracted with ethyl acetate. The organic layer was washed with brine, dried over magnesium sulfate, and evaporated in vacuo. The residue was recrystallized from ethyl acetate and hexane to give N-{2-[2-(acetylamino)ethyl]-1,2,3,4-tetrahydro-6-isoquinolinyl}-2-[4-(trifluoromethyl)phenyl]-1-cyclohexene-1-carboxamide (0.251 g) as a white powder.

¹H-NMR(DMSO-d₆): δ 1.74(4H, br s), 1.78(3H, s), 2.39(4H, br s), 2.46(2H, t, J=7.0 Hz), 2.64(4H, dd, J=14.6, 4.1 Hz), 3.21(2H, q, J=12.4, 5.9 Hz), 3.45(2H, s), 6.85(1H, d, J=8.4 Hz), 7.01(1H, dd, J=8.4, 2.2 Hz), 7.10(1H, s), 7.47(2H, d, J=8.1 Hz), 7.62(2H, d, J=8.1 Hz), 7.78(1H, t, J=5.4 Hz), 9.50(1H, s)

ESI-MS(m/z): 486(M+H)⁺

### EXAMPLE 217

N-{2-[2-(Acetylamino)ethyl]-1,2,3,4-tetrahydro-6-isoquinolinyl}-2-(4-methylphenyl)-1-cyclohexene-1-carboxamide was obtained in the same manner as in Example 216 as a pale yellow powder.

¹H-NMR(DMSO-d₆): δ 1.71(4H, br s), 1.78(3H, s), 2.21 (3H, s), 2.34(6H, br s), 2.51-2.69(4H, m), 3.22(2H, dd, J=12.2, 6.2 Hz), 3.47(2H, br s), 6.58(2H, d, J=8.1 Hz), 7.02-7.05(3H, m), 7.15-7.18 (3H, m), 7.79(1H, br s), 9.37(1H, s)

ESI-MS(m/z): 432(M+H)⁺

### EXAMPLE 218

N-(2-[2-(Acetylamino)ethyl]-1,2,3,4-tetrahydro-6-isoquinolinyl}-2-(4-ethylphenyl)-1-cyclohexene-1-carboxamide was obtained in the same manner as in Example 216 as a pale yellow powder.

¹H-NMR(DMSO-d₆): δ 1.11(3H, t, J=7.6 Hz), 1.70(4H, br s), 1.78(3H, s), 2.35(4H, br s), 2.43-2.55(4H, m), 2.64(4H, dd, J=15.0, 4.3 Hz), 3.21(2H, dd, J=13.0, 6.8 Hz), 3.45(2H, s), 6.84(2H, d, J=8.4 Hz), 6.99-7.20(6H, m), 7.78(1H, br t, J=5.1 Hz), 9.32(1H, s)

ESI-MS(m/z): 445(M+H)⁺

Preparation 87

To a solution of 2-{2-[(methylsulfonyl)oxy]ethyl}-4-nitrobenzyl methanesulfonate (500 mg) in tetrahydrofuran (2.5 ml) were added triethylamine (0.493 ml) and 2-phenylethanamine (206 mg) and the mixture was stirred at 60° C. for 13 hours. To the reaction mixture was added water and extracted with ethyl acetate. The organic layer was washed with brine, dried over magnesium sulfate, filtered, and concentrated in vacuo. The residue was purified by column chromatography on silica gel by eluting with hexane:ethyl acetate (4:1) to give 6-nitro-2-(2-phenylethyl)-1,2,3,4-tetrahydroisoquinoline (228 mg) as a brown foam.

¹H-NMR(CDCl₃): δ 2.80-3.10(8H, m), 3.79(2H, s), 7.15-7.39(6H, m), 2.95-8.05(2H, m)

ESI-MS(m/z): 283(M+H)⁺

Preparation 88

A solution of 6-nitro-2-(2-phenylethyl)-1,2,3,4-tetrahydroisoquinoline (220 mg) in methanol (3.3 ml) was hydrogenated over 10% palladium on carbon (110 mg, 50% wet) at ambient temperature under atmospheric pressure of hydrogen for an hour. The reaction mixture was filtered through a short pad of celite, and the filtrate was concentrated in vacuo. The residue was recrystallized from ethyl acetate-hexane to give 2-(2-phenylethyl)-1,2,3,4-tetrahydro-6-isoquinolinamine (196 mg) as a yellow powder.

¹H-NMR(DMSO-d₆): δ 2.87-3.98(1H, m), 3.09-3.35(3H, m), 3.68(1H, br s), 3.20-3.55(2H, m), 3.68(1H, m), 4.16(1H, m), 4.42(1H, m), 6.44(1H, s), 6.53(1H, dd, J=6.5, 2.4 Hz), 6.87(1H, d, J=8.6 Hz), 7.24-7.39(5H, m)

ESI-MS(m/z): 253(M+H)⁺

### EXAMPLE 219

To a solution of 2-[4-(trifluoromethyl)phenyl)-1-cyclohexene-1-carboxylic acid (274 mg) in toluene (1.37 ml) were added thionyl chloride (0.147 ml) and N,N-dimethylformamide (1 drop) and the mixture was stirred at 80° C. for an hour. The mixture was evaporated in vacuo and the residue was dissolved in tetrahydrofuran (1.5 ml). The acid chloride in tetrahydrofuran was added to a solution of 2-(2-phenylethyl)-1,2,3,4-tetrahydro-6-isoquinolinamine (197 mg) and triethylamine (0.163 ml) in tetrahydrofuran (1.5 ml) at ambient temperature and the mixture was stirred at the same temperature for 2 hours. The mixture was poured into water and extracted with ethyl acetate. The organic layer was washed with brine, dried over magnesium sulfate, and evaporated in vacuo. The residue was recrystallized from ethyl acetate and hexane to give N-[2-(2-phenylethyl)-1,2,3,4-tetrahydro-6-isoquinolinyl]-2-[4-(trifluoromethyl)phenyl]-1-cyclohexene-1-carboxamide (107 mg) as a pale brown powder.

¹H-NMR(DMSO-d₆): δ 1.73(4H, br s), 2.39(4H, br s), 2.60-2.85(8H, m), 3.52(2H, s), 6.87(1H, d, J=8.1 Hz), 7.02(1H, d, J=8.3 Hz), 7.10(1H, s), 7.14-7.30(5H, m), 7.47(2H, d, J=8.1 Hz), 7.62(2H, d, J=7.8 Hz), 9.51(1H, s)

ESI-MS(m/z): 505(M+H)⁺

Preparation 89

6-Nitro-2-[2-(2-pyridinyl)ethyl]-1,2,3,4-tetrahydroisoquinoline was obtained in the same manner as in Preparation 87 as a pale yellow foam.

¹NMR(CDCl₃): δ 2.86(2H, t, J=5.7 Hz), 2.95-3.05(4H, m), 3.07-3.13(2H, m), 3.82(2H, s), 7.10-7.24(3H, m), 7.61(1H, td, J=7.6, 1.9 Hz), 7.93-7.99(2H, m), 8.52-8.56(1H, m)

ESI-MS(m/z): 284(M+H)⁺

Preparation 90

2-[2-(2-Pyridinyl)ethyl]-1,2,3,4-tetrahydro-6-isoquinolinamine was obtained in the same manner as in Preparation 88 as a pale yellow foam.

¹H-NMR(CDCl₃): δ 2.75-2.85(4H, m), 2.88-2.95(2H, m), 3.06-3.13(2H, m), 3.52(2H, br s), 3.64(2H, s), 6.44-6.51(2H, m), 6.82(1H, d, J=7.8 Hz), 7.08-7.14(1H, m), 7.22(1H, d, J=8.1 Hz), 7.59(1H, td, J=7.6, 1.9 Hz), 8.51-8.55(1H, m)

ESI-MS(m/z): 254(M+H)⁺

### EXAMPLE 220

N-{2-[2-(2-Pyridinyl)ethyl]-1,2,3,4-tetrahydro-6-isoquinolinyl}-2-[4-(trifluoromethyl)phenyl]-1-cyclohexene-1-carboxamide was obtained in the same manner as in Example 219 as a pale yellow powder.

¹H-NMR(DMSO-d₆): δ 1.74(4H, br s), 1.99(4H, br s), 4.66(4H, br s), 2.75-2.79(2H, m), 2.96(2H, t, J=14.6 Hz), 3.52(2H, s), 6.86(1H, d, J=8.1 Hz), 7.01(1H, dd, J=8.4, 1.9 Hz), 7.10(1H, br s), 7.15-7.21(1H, m), 7.30(1H, d, J=8.1 Hz), 7.47(2H, d, J=8.4 Hz), 7.62(2H, d, J=8.4 Hz), 7.67(1H, td, J=7.6, 1.9 Hz), 8.46(1H, br d, J=4.1 Hz), 9.50(1H, s)

ESI-MS(m/z): 506(M+H)⁺

Preparation 91

tert-Butyl 6-[2-(6-nitro-3,4-dihydro-2(1H)-isoquinolinyl)ethyl]-2-pyridinylcarbamate (329 mg) was obtained in the same manner as in Preparation 87 as a pale brown foam.

¹H-NMR(CDCl₃): δ 1.52(9H, s), 2.83(2H, t, J=5.9 Hz), 2.93(4H, br s), 2.30(2H, t, J=5.9 Hz), 3.78(2H, s), 6.86(1H, dd, J=7.6, 1.1 Hz), 7.15-7.20(2H, m), 7.57(1H, t, J=5.4 Hz), 7.75(1H, d, J=8.1 Hz), 7.94-7.99(2H, m)

ESI-MS(m/z): 399(M+H)⁺

Preparation 92

tert-Butyl 6-[2-(6-amino-3,4-dihydro-2(1H)-isoquinolinyl)ethyl]-2-pyridinylcarbamate was obtained in the same manner as in Preparation 88 as a pale yellow powder.

¹H-NMR(DMSO-d₆): δ 2.75-2.85(4H, m), 2.88-2.95(2H, m), 3.06-3.13(2H, m), 3.52(2H, br s), 3.64(2H, s), 6.44-6.51(2H, m), 6.82(1H, d, J=7.8 Hz), 7.08-7.14(1H, m), 7.22(1H, d, J=8.1 Hz), 7.59(1H, td, J=7.6, 1.9 Hz), 8.51-8.55(1H, m)

ESI-MS(m/z) : 369(M+H)⁺

### EXAMPLE 221

tert-Butyl 6-{2-[6-[({2-[4-(trifluoromethyl)phenyl]-1-cyclohexen-1-yl}carbonyl)amino]-3,4-dihydro-2(1H)-isoquinolinyl]ethyl}-2-pyridinylcarbamate was obtained in the same manner as in Example 219 as a pale yellow powder.

¹H-NMR(CDCl₃): δ 1.50(9H, s), 1.70-1.81(4H, m), 2.32-2.54(5H, m), 2.83(2H, br s), 3.01(5H, br s), 3.86(1H, br s), 6.55(1H, br s), 6.81(1H, d, J=8.1 Hz), 6.82(2H, d, J=7.8 Hz), 7.24(2H, d, J=8.0 Hz), 7.43(2H, t, J=8.3 Hz), 7.50-7.65(3H, m), 7.75(1H, d, J=7.8 Hz)

ESI-MS(m/z): 621(M+H)⁺

### EXAMPLE 222

To a solution of tert-butyl 6-{2-[6-[({2-[4-(trifluoromethyl)phenyl]-1-cyclohexen-1-yl}carbonyl)amino]-3,4-dihydro-2(1H)-isoquinolinyl]ethyl}-2-pyridinylcarbamate (105 mg) in dichloromethane (1 ml) was added trifluoroacetic acid (0.13 ml). The reaction mixture was stirred for 12 hours, quenched with 10% aqueous potassium carbonate solution, and extracted with dichloromethane. The organic layer was washed with brine, dried over magnesium sulfate, filtered, and concentrated in vacuo. The residue was recrystallized from ethyl acetate-hexane to give N-{2-[2-(6-amino-2-pyridinyl)ethyl]-1,2,3,4-tetrahydro-6-isoquinolinyl}-2-[4-(trifluoromethyl)phenyl]-1-cyclohexene-1-carboxamide (54 mg) as a pale yellow powder.

¹H-NMR(DMSO-d₆): δ 1.73(4H, br s), 2.39(4H, br s), 2.67(4H, br s), 2.72(4H, br s), 3.52(2H, s), 5.79(2H, s), 6.24(1H, d, J=8.1 Hz), 6.38(1H, d, J=7.3 Hz), 6.87(1H, d, J=8.4 Hz), 7.02(1H, dd, J=8.1, 1.9 Hz), 7.10(1H, d, J=1.6 Hz), 7.25(1H, t, J=7.0 Hz), 7.47(2H, d, J=7.8 Hz), 7.62(2H, d, J=7.8 Hz), 9.51(1H, s)

ESI-MS(m/z): 521(M+H)⁺

Preparation 93

To a suspension of sodium hydride (60% oil dispersion) (3.66 g) in N,N-dimethylformamide (150 ml) was added dropwise a solution of methyl 2-oxocyclopentanecarboxylate (11.84 g) at 10° C. under nitrogen and the mixture was stirred at ambient temperature for 1.5 hours. To this solution was added dropwise 1,1,2,2,3,3,4,4,4-nonafluoro-1-butanesulfonyl fluoride (27.7 g) over 2 hours and the mixture was stirred at ambient temperature for 18 hours. The mixture was poured into a mixture of ethyl acetate, water and 6N hydrochloric acid. The separated organic layer was washed with water and brine, dried over magnesium sulfate, and evaporated in vacuo. The residue was purified by column chromatography on silica gel eluting with hexane:toluene (1:1) to give methyl 2-{[(nonafluorobutyl)sulfonyl]oxy}-1-cyclopentene-1-carboxylate (14.20 g) as a colorless oil.

¹H-NMR(DMSO-d₆): δ 1.9-2.1(2H, m), 2.65-2.85(4H, m), 3.71(3H, s)

ESI-MS(m/z): 462(M+Na)⁺, 440(M+H)⁺

Preparation 94

To a suspension of zinc chloride (9.05 g) in tetrahydrofuran (120 ml) was added dropwise a 1.0 mol/L solution of p-tolylmagnesium bromide in tetrahydrofuran (49.9 ml) at 0° C. and the mixture was stirred vigorously at the same temperature for 30 minutes. To the suspension were added bis(dibenzylideneacetone)palladium (573 mg) and 1,1′-diphenylphosphino)ferrocene (553 mg), followed by dropwise addition of a solution of methyl 2-{[(nonafluorobutyl)-sulfonyl]oxy}-1-cyclopentene-1-carboxylate (14.10 g) in tetrahydrofuran (50 ml) at 0° C. The mixture was refluxed for 4 hours under nitrogen and poured into a mixture of ethyl acetate, water and 6N hydrochloric acid. The separated organic layer was washed with water and brine, dried over magnesium sulfate, and evaporated in vacuo. The residue was purified by column chromatography on silica gel eluting with hexane:toluene (1:1) to give methyl 2-(4-methylphenyl)-1-cyclopentene-1-carboxylate (6.74 g) as a colorless oil.

¹H-NMR(DMSO-d₆): δ 1.8-2.0(2H, m), 2.30(3H, s), 2.6-2.9(4H, m), 3.54(3H, s), 7.14(2H, d, J=8.2 Hz), 7.24(2H, d, J=8.2 Hz)

ESI-MS(m/z): 239(M+Na)⁺

Preparation 95

To a solution of methyl 2-(4-methylphenyl)-1-cyclopentene-1-carboxylate (6.73 g) in ethanol (67 ml) was added 5N aqueous sodium hydroxide solution (13.4 ml) and the mixture was refluxed for 4 hours. The mixture was cooled to ambient temperature and neutralized by addition of 6N hydrochloric acid. The mixture was concentrated in vacuo to remove ethanol and the residue was adjusted to pH ca. 2 by addition of 6N hydrochloric acid. The residue was extracted with ethyl acetate and the separated organic layer was washed with water and brine, dried over magnesium sulfate, and evaporated in vacuo. The residue was triturated with hexane and the solids were collected by filtration and washed with hexane to give 2-(4-methylphenyl)-1-cyclopentene-1-carboxylic acid (4.48 g) as pale purple crystals.

¹H-NMR(DMSO-d₆): δ 1.8-2.0(2H, m), 2.29(3H, s), 2.7-2.9(4H, m), 7.12(2H, d, J=8.2 Hz), 7.25(2H, d, J=8.2 Hz)

ESI-MS(m/z): 225(M+Na)⁺

### EXAMPLE 223

To a solution of 4-aminophenyl(2-(2-pyridinyl)ethyl)formamide (1.95 g), 2-(4-methylphenyl)-1-cyclopentene-1-carboxylic acid (1.80 g) and benzotriazol-1-yl-oxytripyrrolidinophosphonium hexafluorophosphate (PyBOP) (5.05 g) in N,N-dimethylformamide (40 ml) was added-diisopropylethylamine (2.09 g) at ambient temperature and the mixture was stirred at the same temperature for 16 hours. The mixture was poured into a mixture of ethyl acetate, water and 6N hydrochloric acid, and the separated organic layer was washed with water and brine, dried over magnesium sulfate, and evaporated in vacuo. The residue was purified by column chromatography on silica gel eluting with ethyl acetate to give N-(4-{formyl[2-(2-pyridinyl)ethyl]amino}phenyl)-2-(4-methylphenyl)-1-cyclopentene-1-carboxamide (2.78 g) as a pale brown powder.

¹H-NMR(DMSO-d₆): δ 1.9-2.1(2H, m), 2.26(3H, s), 2.7-2.9(6H, m), 4.0-4.1(2H, m), 7.1-7.4(8H, m), 7.55-7.75(3H, m), 8.30(1H, s), 8.46(1H, d, J=5.0 Hz), 10.01(1H, s)

ESI-MS(m/z): 448(M+Na)⁺, 426(M+H)⁺

### EXAMPLE 224

To a solution of N-(4-{formyl[2-(2-pyridinyl)ethyl]amino}phenyl)-2-(4-methylphenyl)-1-cyclopentene-1-carboxamide (2.75 g) in methanol (15 ml) was added concentrated hydrochloric acid (2.7 ml) and the mixture was stirred at 30° C. for 16 hours. To the mixture was added a mixture of ethyl acetate and water and adjusted to pH 8 by addition of 50% aqueous potassium carbonate solution. The separated organic layer was washed with water and brine, dried over magnesium sulfate, and evaporated in vacuo. The residue was purified by column chromatography on silica gel eluting with ethyl acetate:methanol (10:1) and crystallized from ethyl acetate to give 2-(4-methylphenyl)-N-(4-{[2-(2-pyridinyl)ethyl]amino }phenyl)-1-cyclopentene-1-carboxamide (1.69 g) as white crystals.

¹H-NMR(DMSO-d₆): δ 1.9-2.1(2H, m), 2.26(3H, s), 2.7-2.85(4H, m), 3.00(2H, t, J=7.4 Hz), 3.34(2H, td, J=7.4 and 5.8 Hz), 5.52(1H, t, J=5.8 Hz), 6.52(2H, d, J=8.8 Hz), 7.11(2H, d, J=8.8 Hz), 7.15-7.35(6H, m), 7.65-7.75(1H, m), 8.45-8.5(1H, m), 9.49(1H, s)

ESI-MS(m/z): 420(M+Na)⁺, 398(M+H)⁺

Preparation 96

To a solution of methyl 2-{[(nonafluorobutyl)sulfonyl]-oxy}-1-cyclopentene-1-carboxylate (3.08 g) in toluene (80 ml) were added tetrakis(triphenylphosphine)palladium (419 mg) and lithium chloride (923 mg) and the mixture was stirred at ambient temperature for 10 minutes. To the mixture were added 4-(trifluoromethyl)phenylboronic acid (1.65 g) and a solution of sodium carbonate (2.0 g) in water (20 ml) and the mixture was stirred vigorously at 100° C. for 16 hours. The mixture was poured into a mixture of ethyl acetate, water and active-charcoal (10 g) and adjusted to pH 2 by addition of 6N hydrochloric acid. The active-charcoal was removed by filtration and the separated organic layer was washed with water and brine, dried over magnesium sulfate, and evaporated in vacuo. The residue was purified by column chromatography on silica gel eluting with hexane:tolune (1:2) to give methyl 2-[4-(trifluoromethyl)phenyl]-1-cyclopentene-1-carboxylate (1.68 g) as a pale green oil.

¹H-NMR(DMSO-d₆): δ 1.9-2.05(2H, m), 2.75-3.0(4H, m), 3.56(3H, s), 7.54(2H, d, J=8.2 Hz), 7.70(2H, d, J=8.2 Hz)

ESI-MS(m/z): 293(M+Na)⁺

Preparation 97

2-(4-(Trifluoromethyl)phenyl)-1-cyclopentene-1-carboxylic acid was obtained in the same manner as in Preparation 95 as white crystals.

¹H-NMR(DMSO-d₆): δ 1.85-2.05(2H, m), 2.7-2.95(4H, m), 7.54(2H, d, J=8.2 Hz), 7.74(2H, d, J=8.2 Hz), 12.55(1H, br)

negative ESI-MS(m/z): 255(M−H)⁻

### EXAMPLE 225

N-(4-{Formyl[2-(2-pyridinyl)ethyl]amino}phenyl)-2-[4-(trifluoromethyl)phenyl]-1-cyclopentene-1-carboxamide was obtained in the same manner as in Example 223 as a brown amorphous solid.

¹H-NMR(DMSO-d₆): δ 1.9-2.15(2H, m), 2.85-3.1(6H, m), 4.08(2H, t, J=7.6 Hz), 7.1-7.3(6H, m), 7.55-7.8(5H, m), 8.31(1H, s), 8.46(1H, d, J=4.7 Hz), 10.09(1H, s)

ESI-MS(m/z): 502(M+Na)⁺, 480(M+H)⁺

### EXAMPLE 226

N-(4-{[2-(2-Pyridinyl)ethyl]amino}phenyl)-2-[4-(trifluoromethyl)phenyl]-1-cyclopentene-1-carboxamide was obtained in the same manner as in Example 224 as yellow crystals.

¹H-NMR(DMSO-d₆): δ 1.95-2.1(2H, m), 2.75-2.95(4H, m), 2.96(2H, t, J=7.4 Hz), 3.34(2H, td, J=7.4 and 5.8 Hz), 5.54(1H, t, J=5.8 Hz), 6.52(2H, d, J=8.8 Hz), 7.26(2H, d, J=8.8 Hz), 7.15-7.3(2H, m), 7.58(2H, d, J=8.4 Hz), 7.68(2H, d, J=8.4 Hz), 7.7-7.8(1H, m), 8.5-8.55(1H, m), 9.58(1H, s)

ESI-MS(m/z): 474(M+Na)⁺, 452(M+H)⁺

Preparation 98

Ethyl 2-(4-ethylphenyl)-1-cyclohexene-1-carboxylate was obtained in the same manner as in Preparation 96 as a pale green oil.

¹H-NMR(DMSO-d₆): δ 0.76(3H, t, J=7.1 Hz), 1.14(3H, t, J=7.6 Hz), 1.6-1.8(4H, m), 2.25-2.4(4H, m), 2.58(2H, q, J=7.1 Hz), 3.78(2H, q, J=7.6 Hz), 7.02(2H, d, J=8.1 Hz), 7.13(2H, d, J=8.1 Hz)

ESI-MS(m/z): 281(M+Na)⁺

Preparation 99

2-(4-Ethylphenyl)-1-cyclohexene-1-carboxylic acid was obtained in the same manner as in Preparation 95 as white crystals.

¹H-NMR(DMSO-d₆): δ 1.17(3H, t, J=7.6 Hz), 1.55-1.75(4H, m), 2.25-2.4(4H, m), 2.57(2H, q, J=7.6 Hz), 7.0-7.2(4H, m), 119.4(1H, br)

negative ESI-MS(m/z): 229(M−H)⁻

### EXAMPLE 227

2-(4-Ethylphenyl)-N-(4-{formyl[2-(2-pyridinyl)ethyl]amino}phenyl)-1-cyclohexene-1-carboxamide was obtained in the same manner as in Example 223 as a brown powder.

¹H-NMR(DMSO-d₆): δ 1.10(3H, t, J=7.5 Hz), 1.6-1.8(4H, m), 2.3-2.45(4H, m), 2.53(2H, q, J=7.5 Hz), 2.85(2H, t, J=7.3 Hz), 4.03(2H, t, J=7.3 Hz), 7.1-7.25(9H, m), 7.39(2H, d, J=8.8 Hz), 8.07(1H, s), 8.4-8.5(1H, m), 9.60(1H, s)

ESI-MS(m/z): 476(M+Na)⁺, 454(M+H)⁺

### EXAMPLE 228

2-(4-Ethylphenyl)-N-(4-{[2-(2-pyridinyl)ethyl]amino}phenyl)-1-cyclohexene-1-carboxamide was obtained in the same manner as in Example 224 as white crystals.

¹H-NMR(DMSO-d₆): δ 1.12(3H, t, J=7.6 Hz), 1.6-1.8(4H, m), 2.3-2.45(4H, m), 2.52(2H, q, J=7.6 Hz), 2.93(2H, t, J=7.4 Hz), 3.29(2H, td, J=7.4 and 5.7 Hz), 5.44(1H, t, J=5.7 Hz), 6.42(2H, d, J=8.8 Hz), 7.00(2H, d, J=8.8 Hz), 7.08(2H, d, J=8.1 Hz), 7.20(2H, d, J=8.1 Hz), 7.2-7.3(2H, m), 7.75-7.85(1H, m), 9.00(1H, s)

ESI-MS(m/z): 448(M+Na)⁺, 426(M+H)⁺

Preparation 100

To a suspension of sodium hydride (60% oil dispersion) (5.16 g) in N,N-dimethylformamide (160 ml) was added dropwise a solution of methyl 2-oxocycloheptanecarboxylate (20.0 g) at 10° C. under nitrogen and the mixture was warmed to ambient temperature and stirred for an hour. To this mixture was added dropwise 1,1,2,2,3,3,4,4,4-nonafluoro-1-butanesulfonyl fluoride (39.0 g) at ambient temperature and the mixture was warmed to 35° C. and stirred at the same temperature for 20 hours. The mixture was poured into a mixture of ethyl acetate and ice water and adjusted to pH ca. 2 by addition of 6N hydrochloric acid. The separated organic layer was washed with water and brine, dried over magnesium sulfate and evaporated in vacuo. The residue was purified by column chromatography on silica gel eluting with hexane:toluene (1:1) to give methyl 2-{[(nonafluorobutyl)sulfonyl]oxy}-1-cycloheptene-1-carboxylate (29.82 g) as a colorless oil.

¹H-NMR(DMSO-d₆): δ 1.6-1.9(6H, m), 2.6-2.9(4H, m), 3.70(3H, s)

ESI-MS(m/z): 475(M+Na)⁺

Preparation 101

To a suspension of zinc chloride (17.91 g) in tetrahydrofuran (200 ml) was added dropwise a 1 mol/L solution of p-tolylmagnesium bromide in tetrahydrofuran (98.6 ml) at 0° C. under nitrogen and the mixture was stirred at the same temperature for 30 minutes. To this suspension were added bis(dibenzylideneacetone) palladium (1.13 g) and 1,1′-bis(diphenylphosphino)ferrocene (1.09 g), followed by dropwise addition of methyl 2-{[(nonafluorobutyl)sulfonyl]oxy}-1-cycloheptene-1-carboxylate (29.72 g) in tetrahydrofuran (90 ml) and the mixture was refluxed for 16 hours under nitrogen. The mixture was poured into a mixture of ethyl acetate and ice water and adjusted to pH ca. 2 by addition of 6N hydrochloric acid. The separated organic layer was washed with water and brine, dried over magnesium sulfate and evaporated in vacuo. The residue was purified by column chromatography on silica gel eluting with hexane:toluene (1:3) to give methyl 2-(4-methylphenyl)-1-cycloheptene-1-carboxylate (13.77 g) as a colorless oil.

¹H-NMR(DMSO-d₆): δ 1.6-1.9(6H, m), 2.28(3H, s), 2.5-2.5(4H, m), 3.70(3H, s), 6.95-7.0(2H, m), 7.1-7.15(2H, m)

ESI-MS(m/z): 267(M+Na)⁺

Preparation 102

To a solution of methyl 2-(4-methylphenyl)-1-cycloheptene-1-carboxylate (13.76 g) in ethanol (130 ml) was added 5N aqueous sodium hydroxide solution (22.6 ml) at ambient temperature and the mixture was refluxed for 4 hours. The mixture was cooled to 5° C. and ice-water (60 ml) was added. The mixture was adjusted to pH ca. 7 by addition of 6N hydrochloric acid and concentrated in vacuo. The residue was poured into a mixture of ethyl acetate and water and adjusted to pH ca. 2 by addition of 6N hydrochloric acid. The separated organic layer was washed with water and brine, dried over magnesium sulfate and evaporated in vacuo. The residue was triturated with hexane and the solids were collected by filtration and washed with hexane to 2-(4-methylphenyl)-1-cycloheptene-1-carboxylic acid (3.58 g) as white crystals.

¹H-NMR(DMSO-d₆): δ 1.45-1.6(4H, m), 1.7-1.9(2H, m), 2.27(3H, s), 2.4-2.55(4H, m), 7.0-7.15(4H, m), 11.90(1H, br s)

ESI-MS(m/z):253(M+Na)⁺

### EXAMPLE 229

N-(4-{Formyl[2-(2-pyridinyl)ethyl]amino}phenyl)-2-(4-methylphenyl)-1-cycloheptene-1-carboxamide was obtained in the same manner as in Example 223 as a brown powder.

¹H-NMR(DMSO-d₆): δ 1.6-1.9(6H, m), 2.21(3H, s), 2.4-2.5(4H, m), 2.85(2H, t, J=7.7 Hz), 3.99(2H, t, J=7.7 Hz), 7.0-7.3(8H, m), 7.37(2H, d, J=8.7 Hz), 7.6-7.7(1H, m), 8.25(1H, s), 8.45(1H, d, J=3.9 Hz), 9.42(1H, s)

ESI-MS(m/z): 448(M+Na)⁺, 426(M+H)⁺

### EXAMPLE 230

2-(4-Methylphenyl)-N-(4-{[2-(2-pyridinyl)ethyl]amino}-phenyl)-1-cycloheptene-1-carboxamide was obtained in the same manner as in Example 224 as white crystals.

¹H-NMR(DMSO-d₆): δ 1.55-1.9(6H, m), 2.22(3H, s), 2.4-2.5(4H, m), 2.93(2H, t, J=7.1 Hz), 3.29(2H, t, J=7.1 and 5.8 Hz), 5.44(1H, t, J=5.8 Hz), 6.42(2H, d, J=8.8 Hz), 6.99(2H, d, J=8.8 Hz), 7.03(2H, d, J=8.2 Hz), 7.15(2H, d, J=8.2 Hz), 7.2-7.35(2H, m), 7.6.5-7.75(1H, m), 8.50(1H, d, J=5.1 Hz), 8.86(1H, s)

ESI-MS(m/z): 448(M+Na)⁺, 426(M+H)⁺

Preparation 103

Methyl 2-[4-(trifluoromethyl)phenyl]-1-cycloheptene-1-carboxylate was obtained in the same manner as in Preparation 96 as a pale green oil.

¹H-NMR(DMSO-d₆): δ 1.5-1.7(4H, m), 1.75-1.9(2H, s), 2.5-2.65(4H, m), 3.34(3H, s), 7.30(2H, d, J=8.2 Hz), 7.67(2H, d, J=8.2 Hz)

ESI-MS(m/z): 321(M+Na)⁺, 299(M+H)⁺

Preparation 104

2-[4-(Trifluoromethyl)phenyl]-1-cycloheptene-1-carboxylic acid was obtained in the same manner as in Preparation 95 as white crystals.

¹H-NMR(DMSO-d₆): δ 1.5-1.9(6H, m), 2.4-2.5(4H, m), 7.34(2H, d, J=8.2 Hz), 7.65(2H, d, J=8.2 Hz), 12.12(1H, br s)

negative ESI-MS(m/z): 283(M−H)⁻

### EXAMPLE 231

N-(4-{Formyl[2-(2-pyridinyl)ethyl]amino}phenyl)-2-[4-(trifluoromethyl)phenyl]-1-cycloheptene-1-carboxamide was obtained in the same manner as in Example 223 as a brown powder.

¹H-NMR(DMSO-d₆): δ 1.6-1.9(6H, m), 2.4-2.6(4H, m), 2.85(2H, t, J=7.0 Hz), 4.06(2H, t, J=7.0 Hz), 7.1-7.7(11H, m), 8.25(1H, s), 8.4-8.5(1H, m), 9.56(1H, s)

ESI-MS(m/z): 530(M+Na)⁺, 508(M+H)⁺

### EXAMPLE 232

N-(4-{[2-(2-Pyridinyl)ethyl]amino}phenyl)-2-[4-(trifluoromethyl)phenyl]-1-cycloheptene-1-carboxamide was obtained in the same manner as in Example 224 as white crystals.

¹H-NMR(DMSO-d₆): δ 1.6-1.9(6H, m), 2.4-2.6(4H, m), 2.93(2H, t, J=7.0 Hz), 3.27(2H, td, J=7.0 and 5.7 Hz), 5.47(1H, t, J=5.7 Hz), 6.41(2H, d, J=8.7 Hz), 6.91(2H, d, J=8.7 Hz), 7.15-7.3(3H, m), 7.44(2H, d, J=8.1 Hz), 7.62(2H, d, J=8.1 Hz), 8.45-8.5(1H, m), 8.99(1H, s)

ESI-MS(m/z): 502(M+Na)⁺, 480(M+H)⁺

Preparation 105

Ethyl 2-{[(nonafluorobutyl)sulfonyl]oxy}-1-cyclooctene-1-carboxylate was obtained in the same manner as in Preparation 93 as a colorless oil.

¹H-NMR(DMSO-d₆): δ 1.23(3H, t, J=7.1 Hz), 1.4-1.8(8H, m), 2.45-2.65(4H, m), 4.18(2H, q, J=7.1 Hz)

ESI-MS(m/z): 503(M+Na)⁺, 481(M+H)⁺

Preparation 106

Ethyl 2-(4-methylphenyl)-1-cyclooctene-1-carboxylate was obtained in the same manner as in Preparation 96 as a colorless oil.

¹H-NMR(DMSO-d₆): δ 1.6-1.9(6H, m), 2.28(3H, s), 2.5-2.5(4H, m), 3.70(3H, s), 6.95-7.0(2H, m), 7.1-7.15(2H, m)

ESI-MS(m/z) : 267(M+Na)⁺

Preparation 107

2-(4-Methylphenyl)-1-cyclooctene-1-carboxylic acid was obtained in the same manner as in Preparation 95 as white crystals.

¹H-NMR(DMSO-d₆): δ 6 1.4-1.8(8H, m), 2.28(3H, s), 2.4-2.6(4H, m), 7.0-7.15(4H, m), 11.82(1H, br s)

negative ESI-MS(m/z): 243(M−H)⁻

### EXAMPLE 233

N-(4-{Formyl[2-(2-pyridinyl)ethyl]amino}phenyl)-2-(4-methylphenyl)-1-cyclooctene-1-carboxamide was obtained in the same manner as in Example 223 as a brown powder.

¹H-NMR(DMSO-d₆): δ 1.4-1.9(8H, m), 2.21(3H, s), 2.3-2.5(4H, m), 2.84(2H, t, J=8.3 Hz), 3.35(2H, t, J=8.3 Hz), 6.9-7.4(9H, m), 7.6-7.8(2H, m), 8.24(1H, s), 8.4-8.5(1H, m), 9.34(1H, s)

ESI-MS(m/z): 490(M+Na)⁺

### EXAMPLE 234

2-(4-Methylphenyl)-N-(4-{[2-(2-pyridinyl)ethyl]amino}-phenyl)-1-cyclooctene-1-carboxamide was obtained in the same manner as in Example 224 as a brown powder.

¹H-NMR(DMSO-d₆): δ 1.4-1.8(8H, m), 2.32(3H, s), 2.35-2.6(4H, m), 2.93(2H, t, J=7.2 Hz), 3.27(2H, td, J=7.2 and 5.7 Hz), 5.41(1H, t, J=5.7 Hz), 6.40(2H, d, J=8.8 Hz), 6.95(2H, d, J=8.8 Hz), 7.05(2H, d, J=8.1 Hz), 7.19(2H, d, J=8.1 Hz), 7.15-7.3(2H, m), 7.6-7.7(1H, m), 8.49(1H, d, J=4.8 Hz), 8.73(1H, s)

ESI-MS(m/z): 462(M+Na)⁺, 440(M+H)⁺

Preparation 108

Ethyl 2-[4-(trifluoromethyl)phenyl]-1-cyclooctene-1-carboxylate was obtained in the same manner as in Preparation 96 as a colorless oil.

¹H-NMR(DMSO-d₆): δ 1.6-1.9(6H, m), 2.28(3H, s), 2.5-2.5(4H, m), 3.70(3H, s), 6.95-7.0(2H, m), 7.1-7.15(2H, m)

ESI-MS(m/z): 267(M+Na)⁺

Preparation 109

2-[4-(Trifluoromethyl)phenyl]-1-cyclooctene-1-carboxylic acid was obtained in the same manner as in Preparation 95 as white crystals.

¹H-NMR(DMSO-d₆): δ 1.4-1.8(8H, m), 2.4-2.6(4H, m), 7.36(2H, d, J=8.0 Hz), 7.67(2H, d, J=8.0 Hz), 12.05(1H, br s)

ESI-MS(m/z): 321(M+Na)⁺

### EXAMPLE 235

N-(4-{Formyl[2-(2-pyridinyl)ethyl]amino}phenyl)-2-[4-(trifluoromethyl)phenyl]-1-cyclooctene-1-carboxamide was obtained in the same manner as in Example 223 as a brown powder.

¹H-NMR(DMSO-d₆): δ 1.4-1.9(8H, m), 2.4-2.6(4H, m), 2.8-2.9(2H, m), 4.0-4.1(2H, m), 7.0-7.7(15H, m), 8.22(1H, s), 8.4-8.45(1H, m), 9.56(1H, s), 9.49(1H, s)

negative ESI-MS(m/z): 520(M−H)⁻

### EXAMPLE 236

N-(4-{[2-(2-Pyridinyl)ethyl]amino}phenyl)-2-[4-(trifluoromethyl)phenyl]-1-cyclooctene-1-carboxamide was obtained in the same manner as in Example 224 as white crystals.

¹H-NMR(DMSO-d₆): δ 1.5-1.9(8H, m), 2.35-2.6(4H, m), 2.93(2H, t, J=7.1 Hz), 3.29(2H, t, J=7.1 Hz), 5.62(1H, br), 6.39(2H, d, J=8.7 Hz), 6.88(2H, d, J=8.7 Hz), 7.15-7.3(2H, m), 7.48(2H, d, J=8.1 Hz), 7.64(2H, d, J=8.l Hz), 7.65-7.75(1H, m), 8.45-8.55(1H, m), 8.90(1H, s)

ESI-MS(m/z): 516(M+Na)⁺, 494(M+H)⁺

### EXAMPLE 237

To a suspension of 2-(4-methylphenyl)-1-cyclohexene-1-carboxylic acid (648 mg) in toluene (30 ml) were added thionyl chloride (535 mg) and N,N-dimethylformamide (5 drops) and the mixture was stirred at 70° C. for 4 hours. The resulting solution was evaporated in vacuo to give the acid chloride as an orange oil. To a solution of 4-[2-(2-pyridinyl)ethoxy]aniline (642 mg) and triethylamine (455 mg) in dichloromethane (30 ml) was added dropwise a solution of the acid chloride in dichloromethane (20 ml) at ambient temperature and the mixture was stirred at the same temperature for 16 hours under nitrogen. Water (20 ml) was added and the separated organic layer was washed with water and brine, dried over magnesium sulfate and evaporated in vacuo. The residue was purified by column chromatography on silica gel eluting with hexane:ethyl acetate (1:1) to give 2-(4-methylphenyl)-N-{4-(2-(2-pyridinyl)ethoxy]phenyl}-1-cyclohexene-1-carboxamide (944 mg) as white crystals.

¹H-NMR(DMSO-d₆): δ 1.6-1.8(4H, m), 2.21(3H, s), 2.25-2.4(4H, m), 3.13(2H, t, J=6.6 Hz), 4.26(2H, t, J=6.6 Hz), 6.75(2H, d, J=9.0 Hz), 7.04(2H, d, J=8.0 Hz), 7.18(2H, d, J=8.0 Hz), 7.24(2H, d, J=9.0 Hz), 7.25-7.35(2H, m), 7.65-7.75(1H, m), 8.49(1H, d, J=4.0 Hz), 9.34(1H, s)

APCI-MS(m/z): 413(M+H)⁺

### EXAMPLE 238

2-(4-Methylphenyl)-N-{6-[2-(2-pyridinyl)ethoxy]-3-pyridinyl}-1-cyclohexene-1-carboxamide was obtained in the same manner as in Example 237 as white crystals.

¹H-NMR(DMSO-d₆): δ 1.6-1.8(4H, m), 2.21(3H, s), 2.3-2.45(4H, m), 3.13(2H, t, J=6.8 Hz), 4.52(2H, t, J=6.8 Hz), 6.63(1H, d, J=8.8 Hz), 7.05(2H, d, J=8.1 Hz), 7.20(2H, d, J=8.1 Hz), 7.2-7.3(1H, m), 7.3(1H, d, J=7.8 Hz), 7.6-7.8(2H, m), 8.07(1H, d, J=2.5 Hz), 8.49(1H, d, J=4.1 Hz), 9.49(1H, s)

ESI-MS(m/z): 436(M+Na)⁺, 414(M+H)⁺

Preparation 110

To a solution of 4-nitroaniline (6.91 g), [6-(2,5-dimethyl-1H-pyrrol-1-yl)-2-pyridinyl]acetic acid (11.50 g) and benzotriazol-1-yl-oxytripyrrolidinophosphonium hexafluorophosphate (PyBOP) (31.2 g) in N,N-dimethylformamide (150 ml) was added dropwise diisopropylamine (12.90 g) at ambient temperature and the mixture was stirred at the same temperature for 24 hours. The mixture was poured into a mixture of ethyl acetate, water and 6N hydrochloric acid and the separated organic layer was washed with water and brine, dried over magnesium sulfate, and evaporated in vacuo. The residue was purified by column chromatography on silica gel eluting with ethyl acetate to give 2-[6-(2,5-dimethyl-1H-pyrrol-1-yl)-2-pyridinyl]-N-(4-nitrophenyl)acetamide (5.29 g) as a brown powder.

¹H-NMR(DMSO-d₆): δ 2.02(6H, s), 4.01(2H, s), 5.77(2H, s), 7.31(1H, d, J=7.6 Hz), 7.45(1H, d, J=7.4 Hz), 7.8-7.9(2H, m), 7.97(1H, dd, J=7.6 and 7.4 Hz), 8.15-8.25(2H, m), 10.87(1H, s)

ESI-MS(m/z): 373(M+Na)⁺

Preparation 111

To a solution of 2-[6-(2,5-dimethyl-1H-pyrrol-1-yl)-2-pyridinyl]-N-(4-nitrophenyl)acetamide (5.90 g) in tetrahydrofuran (100 ml) and methanol (100 ml) was added 5% palladium on carbon (3 g, 50% wet) and the mixture was hydrogenated at ambient temperature under atmospheric pressure of hydrogen for 6 hours. Palladium on carbon was removed by filtration and the filtrate was evaporated in vacuo to give N-(4-aminophenyl)-2-[6-(2,5-dimethyl-1H-pyrrol-1-yl)-2-pyridinyl]acetamide (4.25 g) as a brown powder.

¹H-NMR(DMSO-d₆): δ 2.04(6H, s), 3.79(2H, s), 4.84(2H, br s), 5.78(2H, s), 6.45-6.55(2H, m), 7.15-7.25(2H, m), 7.27(1H, d, J=7.7 Hz), 7.41(1H, d, J=7.6 Hz), 7.93(1H, d, J=7.7 and 7.6 Hz), 9.80(1H, s)

negative ESI-MS(m/z): 319(M−H)⁻

### EXAMPLE 239

N-[4-({[6-(2,5-Dimethyl-1H-pyrrol-1-yl)-2-pyridinyl]acetyl}amino)phenyl]-2-(4-methylphenyl)-1-cyclohexene-1-carboxamide was obtained in the same manner as in Preparation 110 as a brown powder.

¹H-NMR(DMSO-d₆): δ 1.65-1.8(4H, m) 2.02(6H, s), 2.21(3H, s), 2.3-2.4(4H, m), 3.83(2H, s), 5.77(2H, s), 7.03(2H, d, J=8.0 Hz), 7.17(2H, d, J=8.0 Hz), 7.15-7.25(1H, m), 7.28(2H, d, J=8.8 Hz), 7.39(2H, d, J=8.8 Hz), 7.93(1H, dd, J=7.7 and 7.6 Hz), 9.44(1H, s), 10.11 (1H, s)

ESI-MS(m/z): 541(M+Na)⁺

### EXAMPLE 240

To a suspension of N-[4-({[6-(2,5-dimethyl-1H-pyrrol-1-yl)-2-pyridinyl]acetyl}amino)phenyl]-2-(4-methylphenyl)-1-cyclohexene-1-carboxamide (1.62 g) in ethanol (32 ml) and water (8 ml) were added hydroxylamine hydrochloride (2.17 g) and triethylamine (632 mg) and the mixture was refluxed for 6 hours. The mixture was poured into a mixture of ethyl acetate and water and adjusted to pH 8 by addition of 50% aqueous potassium carbonate solution. The separated organic layer was washed with water and brine, dried over magnesium sulfate, and evaporated in vacuo. The residue was purified by column chromatography on silica gel eluting with ethyl acetate and crystallized from acetonitrile to give N-(4-{[(6-amino-2-pyridinyl)acetyl]amino}phenyl)-2-(4-methylphenyl)-1-cyclohexene-1-carboxamide (827 mg) as white crystals. ¹H-NMR(DMSO-d₆): δ 1.6-1.8(4H, m), 2.20(3H, s), 2.3-2.4(4H, m), 3.51(2H, s), 5.89(2H, br s), 6.29(1H, d, J=8.2 Hz), 6.44(1H, d, J=7.1 Hz), 7.03(2H, d, J=8.1 Hz), 7.2-7.3(1H, m), 7.28(2H, d, J=8.9 Hz), 7.42(2H, d, J=8.9 Hz), 9.42(1H, s), 10.08(1H, s)

ESI-MS(m/z): 463(M+Na)⁺, 441(M+H)⁺

### EXAMPLE 241

N-[4-({[6-(2,5-Dimethyl-1H-pyrrol-1-yl)-2-pyridinyl]acetyl}amino)phenyl]-2-[4-(trifluoromethyl)phenyl]-1-cyclohexene-1-carboxamide was obtained in the same manner as in Preparation 110 as a brown powder.

¹H-NMR(DMSO-d₆): δ 1.7-1.85(4H, m), 2.02(6H, s), 2.35-2.5(4H, m), 3.83(2H, s), 5.76(2H, s), 7.2-7.35(4H, m), 7.40(2H, d, J=9.0 Hz), 7.47(2H, d, J=8.3 Hz), 7.62(2H, d, J=8.3 Hz), 7.93(1H, dd, J=7.8 and 7.8 Hz), 9.56(1H, s), 10.13(1H, s)

ESI-MS(m/z): 595(M+Na)⁺

### EXAMPLE 242

N-(4-{[(6-Amino-2-pyridinyl)acetyl]amino}phenyl)-2-[4-(trifluoromethyl)phenyl]-1-cyclohexene-1-carboxamide was obtained in the same manner as in Example 240 as white crystals.

¹H-NMR(DMSO-d₆): δ 1.65-1.9(4H, m), 2.35-2.5(4H, m), 3.51(2H, s), 5.88(2H, br s), 6.29(1H, d, J=8.0 Hz), 6.43(1H, d, J=7.0 Hz), 7.23(2H, d, J=9.1 Hz), 7.30(1H, dd, J=8.0 and 7.0 Hz), 7.39(2H, d, J=8.6 Hz), 7.47(2H, d, J=9.1 Hz), 7.62(2H, d, J=8.0 Hz), 9.55(1H, s), 10.08(1H, s)

ESI-MS(m/z): 495(M+H)⁺

Preparation 112

To a mixture of methyl 5-ethoxy-2-{[(trifluoromethyl)sulfonyl]oxy}benzoate (5.0 g), lithium chloride (1.9 g) and tetrakis(triphenylphosphine)palladium(0) (0.9 g) in toluene (60 ml) was added a solution of sodium carbonate (4.2 g) in water (21 ml) under stirring and followed by 4-(trifluoromethyl)phenylboronic acid (3.2 g). The mixture was stirred at 100° C. for 8 hours. To the reaction mixture were added activated charcoal and toluene (50 ml) and the mixture was stirred for 30 minutes. The insoluble materials were removed by filtration on celite pad and the separated organic layer was washed with water and evaporated in vacuo. The residue was dissolved in ethanol (50 ml) and treated with a solution of sodium hydroxide (1.5 g) in water (15 ml). The mixture was stirred at 90° C. for 10 hours and concentrated in vacuo. To the residue was added a mixture of ethyl acetate and water and the mixture was adjusted to pH 2 with 6N hydrochloric acid. The separated organic layer was washed with water, dried over magnesium sulfate and evaporated in vacuo. The residue was triturated with hexane:toluene (5:1) and collected by filtration to give 4-ethoxy-4′-(trifluoromethyl)-1,1′-biphenyl-2-carboxylic acid (2.88 g) as white crystals.

¹H-NMR(DMSO-d₆): δ 1.36(3H, t, J=7.0 Hz), 4.12(2H, q, J=7.0 Hz), 7.17(1H, dd, J=2.7 Hz, 8.5 Hz), 7.30(1H, d, J=2.7 Hz), 7.33(1H, d, J=8.5 Hz), 7.50(2H, d, J=8.1 Hz), 7.74(2H, d, J=8.1 Hz), 12.93(1H, s)

### EXAMPLE 243

1-[3-(Dimethylamino)propyl]-3-ethylcarbodiimide (0 .17 g) was added to a solution of tert-butyl 4-aminophenyl[2-(2-pyridinyl)ethyl]carbamate (0.31 g), 4-ethoxy-4′-(trifluoromethyl)-1,1′-biphenyl-2-carboxylic acid (0.25 g), 1-hydroxybenzotriazole hydrate (0.17 g) and 4-dimethylaminopyridine (2.4 mg) in dichloromethane (3 ml) under ice-cooling and the mixture was stirred at ambient temperature for 18 hours. To the reaction mixture was added a solution of 10% hydrogen chloride in methanol (9 ml) and the mixture was stirred at the same temperature for 24 hours. The reaction mixture was poured into a mixture of ethyl acetate, tetrahydrofuran and water, and the mixture was adjusted to pH 9 with 20% aqueous potassium carbonate solution. The separated organic layer was washed with water, dried over magnesium sulfate and evaporated in vacuo. The residue was triturated with a mixture of ethyl acetate and diethyl ether to give 4-ethoxy-N-(4-{[2-(2-pyridinyl)ethyl]amino}phenyl)-4′-(trifluoromethyl)-1,1′-biphenyl-2-carboxamide (0.26 g).

¹H-NMR(DMSO-d₆): δ 1.37(3H, t, J=6.9 Hz), 2.96(2H, t, J=7.2 Hz), 3.28-3.41(2H, m), 4.14(2H, q, J=6.9 Hz), 5.54(1H, t, J=5.6 Hz), 6.52(2H, d, J=8.7 Hz), 7.09-7.33(6H, m), 7.41(1H, d, J=8.8 Hz), 7.59(2H, d, J=8.1 Hz), 7.64-7.75(3H, m), 8.48-8.53(1H, m), 9.92(1H, s)

(+)ESI-MS: 506(M+H)⁺, 528(M+Na)⁺

Preparation 113

4-Ethoxy-4′-methyl-1,1′-biphenyl-2-carboxylic acid was obtained in the same manner as in Preparation 112.

¹H-NMR(DMSO-d₆): δ 1.35(3H, t, J=6.9 Hz), 2.32(3H, s), 4,09(2H, q, J=6.9 Hz), 7.09(1H, dd, J=2.8 Hz, 8.4 Hz), 7.16-7.21(5H, m), 7.26(1H, d, J=8.4 Hz), 12.73(1H, br-s)

### EXAMPLE 244

4-Ethoxy-4′-methyl-N-(4-{[2-(2-pyridinyl)ethyl]amino}phenyl)-1,1′-biphenyl-2-carboxamide was obtained in the same manner as in Example 243.

¹H-NMR(DMSO-d₆): δ 1.35(3H, t, J=6.9 Hz), 2.28(3H, s), 2.96(2H, t, J=7.1 Hz), 3.28-3.44(2H, m), 4.11(2H, q, J=6.9 Hz), 5.51(1H, t, J=5.6 Hz), 6.51(2H, d, J=8.7 Hz), 6.92-7.35(11H, m), 7.69(1H, dt, J=1.5 Hz, 7.6 Hz), 8.48-8.53(1H, m), 9.79(1H, s)

(+)ESI-MS: 452(M+H)⁺, 474(M+Na)⁺

Preparation 114

4′-Chloro-4-ethoxy-1,1′-biphenyl-2-carboxylic acid was obtained in the same manner as in Preparation 112.

¹H-NMR(DMSO-d₆): δ 1.35(3H, t, J=7.0 Hz), 4.10(2H, q, J=7.0 Hz), 7.13(1H, dd, J=2.8 Hz, 8.5 Hz), 7.21-7.33(4H, m), 7.43(2H, d, J=8.5 Hz), 12.86(1H, s)

### EXAMPLE 245

4′-Chloro-4-ethoxy-N-(4-{[2-(2-pyridinyl)ethyl]amino}phenyl)-1,1′-biphenyl-2-carboxamide was obtained in the same manner as in Example 243.

¹H-NMR(DMSO-d₆): δ 1.36(3H, t, J=6.9 Hz), 2.97(2H, t, J=7.2 Hz), 3.29-3.40(2H, m), 4.12(2H, q, J=6.9 Hz), 5.53(1H, t, J=5.7 Hz), 6.52(2H, d, J=8.8 Hz), 7.05-7.12(2H, m), 7.17-7.44(5H, m), 7.40(4H, s), 7.70(1H, dt, J=1.7 Hz, 7.6 Hz), 8.48-8.53(1H, m), 9.85(1H, s)

(+)ESI-MS: 472(M+H)⁺, 494(M+Na)⁺

Preparation 115

4-Ethoxy-4′-fluoro-1,1′-biphenyl-2-carboxylic acid was obtained in the same manner as in Preparation 112.

¹H-NMR(DMSO-d₆): δ 1.35(3H, t, J=6.9 Hz), 4.10(2H, q, J=6.9 Hz), 7.08-7.35(7H, m), 12.83(1H, s)

### EXAMPLE 246

4-Ethoxy-4′-fluoro-N-(4-{[2-(2-pyridinyl)ethyl]amino}-phenyl)-1,1′-biphenyl-2-carboxamide was obtained in the same manner as in Example 243.

¹H-NMR(DMSO-d₆): δ 1.36(3H, t, J=6.9 Hz), 2.96(2H, t, J=7.2 Hz), 3.28-3.40(2H, m), 4.12(2H, q, J=6.9 Hz), 5.52(1H, t, J=5.7 Hz), 6.51(2H, d, J=8.7 Hz), 7.03-7.45(11H, m), 7.70(1H, dt, J=1.6 Hz, 7.6 Hz), 8.48-8.53(1H, m), 9.80(1H, s)

(+)ESI-MS: 456(M+H)⁺, 478(M+Na)⁺

Preparation 116

4-Isopropoxy-4′-(trifluoromethyl)-1,1′-biphenyl-2-carboxylic acid was obtained in the same manner as in Preparation 112.

¹H-NMR(DMSO-d₆): δ 1.21(6H, d, J=6.0 Hz), 4.65-4.78(1H, m), 7.16(1H, dd, J=2.6 Hz, 8.5 Hz), 7.28(1H, d, J=2.6 Hz), 7.32(1H, d, J=8.5 Hz), 7.50(2H, d, J=8.0 Hz), 7.73(2H, d, J=8.0 Hz), 12.88(1H, s)

### EXAMPLE 247

4-Isopropoxy-N-(4-{[2-(2-pyridinyl)ethyl]amino}phenyl)-4′-(trifluoromethyl)-1,1′-biphenyl-2-carboxamide was obtained in the same manner as in Example 243.

¹H-NMR(DMSO-d₆): δ 1.32(6H, d, J=6.0 Hz), 2.96(2H, t, J=7.3 Hz), 3.28-3.40(2H, m), 4.68-4.81(1H, m), 5.54(1H, t, J=5.7 Hz), 6.51(2H, d, J=8.8 Hz), 7.06-7.32(6H, m), 7.40(1H, d, J=8.3 Hz), 7.59(2H, d, J=8.1 Hz), 7.64-7.74(3H, m), 8.48-8.52(1H, m), 9.92(1H, s)

(−)ESI-MS: 518(M−H)⁻

Preparation 117

4-Isopropoxy-4′-methyl-1,1′-biphenyl-2-carboxylic acid was obtained in the same manner as in Preparation 112.

¹H-NMR(DMSO-d₆): δ 1.29(6H, d, J=6.0 Hz), 2.32(3H, s), 4.61-4.74(1H, m), 7.09(1H, dd, J=2.8 Hz, 8.4 Hz), 7.14-7.23(5H, m), 7.25(1H, d, J=8.4 Hz), 12.73(1H, s)

### EXAMPLE 248

4-Isopropoxy-4′-methyl-N-(4-{[2-(2-pyridinyl)ethyl]amino}phenyl)-1,1′-biphenyl-2-carboxamide was obtained in the same manner as in Example 243.

¹H-NMR(DMSO-d₆): δ 1.30(6H, d, J=6.0 Hz), 2.28(3H, s), 2.96(2H, t, J=7.2 Hz), 3.28-3.40(2H, m), 4.63-4.76(1H, m), 5.51(1H, t, J=5.7 Hz), 6.51(2H, d, J=8.7 Hz), 6.96-7.33(11H, m), 7.70(1H, dt, J=1.6 Hz, 7.6 Hz), 8.48-8.53(1H, m), 9.79(1H, s)

(−)ESI-MS: 464(M−H)⁻

Preparation 118

4′-Chloro-4-isopropoxy-1,1′-biphenyl-2-carboxylic acid was obtained in the same manner as in Preparation 112.

¹H-NMR(DMSO-d₆): δ 1.30(6H, d, J=6.0 Hz), 4.62-4.76(1H, m), 7.12(1H, dd, J=2.7 Hz, 8.5 Hz), 7.21-7.33(4H, m), 7.43(2H, d, J=8.5 Hz), 12.85(1H, s)

### EXAMPLE 249

4′-Chloro-4-isopropoxy-N-(4-{[2-(2-pyridinyl)ethyl]-amino}phenyl)-1,1′-biphenyl-2-carboxamide was obtained in the same manner as in Example 243.

¹H-NMR(DMSO-d₆): δ 1.31(6H, d, J=6.0 Hz), 2.97(2H, d, J=7.2 Hz), 3.28-3.41(2H, m), 4.64-4.79(1H, m), 5.53(1H, t, J=5.7 Hz), 6.52(2H, d, J=8.8 Hz), 7.01-7.11(2H, m), 7.17-7.44(5H, m), 7.40(4H, s), 7.70(1H, dt, J=1.8 Hz, 7.6 Hz), 8.48-8.53(1H, m), 9.86(1H, s)

Preparation 119

4′-Fluoro-4-isopropoxy-1,1′-biphenyl-2-carboxylic acid was obtained in the same manner as in Preparation 112.

¹H-NMR(DMSO-d₆): δ 1.30(6H, d, J=6.0 Hz), 4.62-4.75(1H, m), 7.07-7.16(1H, m), 7.18-7.36(6H, m), 12.82(1H, s)

### EXAMPLE 250

4′-Fluoro-4-isopropoxy-N-(4-{[2-(2-pyridinyl)ethyl]-amino}phenyl)-1,1′-biphenyl-2-carboxamide was obtained in the same manner as in Example 243.

¹H-NMR(DMSO-d₆): δ 1.31(6H, d, J=6.0 Hz), 2.97(2H, t, J=7.2 Hz), 3.28-3.40(2H, m), 4.63-4.78(1H, m), 5.52(1H, t, J=5.6 Hz), 6.52(2H, d, J=8.7 Hz), 7.00-7.46(11H, m), 7.64-7.75(1H, m), 8.48-8.53(1H, m), 9.82(1H, s)

(+)ESI-MS: 470(M+H)⁺, 492(M+Na)⁺

Preparation 120

To a mixture of methyl 5-acetyl-2-{[(trifluoromethyl)-sulfonyl]oxy}benzoate (9.0 g), lithium chloride (3.5 g) and tetrakis(triphenylphosphine)palladium(0) (1.6 g) in toluene (108 ml) was added a solution of sodium carbonate (7.6 g) in water (38 ml) under stirring and followed by 4-(trifluoromethyl)phenylboronic acid (5.8 g). The mixture was stirred at 100° C. for 6 hours. The reaction mixture was poured into a mixture of ethyl acetate and water. The separated organic layer was washed with water and evaporated in vacuo. The residue was purified by column chromatography on silica gel using a mixture of hexane and ethyl acetate (4:1) as an eluent. The eluted fractions containing the desired product were collected and evaporated in vacuo to give methyl 4-acetyl-4′-(trifluoromethyl)-1,1′-biphenyl-2-carboxylate (8.38 g).

¹H-NMR(DMSO-d₆): δ 2.67(3H, s), 3.67(3H, s), 7.56(2H, d, J=8.2 Hz), 7.64(1H, d, J=8.0 Hz), 7.82(2H, d, J=8.2 Hz), 8.22(1H, dd, J=1.8 Hz, 8.0 Hz), 8.35(1H, d, J=1.8 Hz)

Preparation 121

A mixture of methyl 4-acetyl-4′-(trifluoromethyl)-1,1′-biphenyl-2-carboxylate (1.5 g) and sodium hydroxide (0.47 g) in a mixture of water (5.0 ml) and ethanol (10.0 ml) was stirred under reflux for 8 hours. The solvent was removed by evaporation. The residue was dissolved in water and the solution was adjusted to pH 2 with 6N hydrochloric acid. The mixture was extracted with ethyl acetate. The extract was washed with brine, dried over magnesium sulfate and evaporated in vacuo. The residue was triturated with hexane to give 4-acetyl-4′-(trifluoromethyl)-1,1′-biphenyl-2-carboxylic acid (1.33 g).

¹H-NMR(DMSO-d₆): δ 2.67(3H, s), 7.50-7.67(3H, m), 7.84(2H, d, J=8.2 Hz), 8.17(1H, dd, J=1.8 Hz, 8.0 Hz), 8.34(1H, d, J=1.8 Hz), 13.14(1H, s)

### EXAMPLE 251

4-Acetyl-N-(4-{[2-(2-pyridinyl) ethyl]amino}phenyl)-4′-(trifluoromethyl)-1,1′-biphenyl-2-carboxamide was obtained in the same manner as in Example 243.

¹H-NMR(DMSO-d₆): δ 2.68(3H, s), 2.97(2H, t, J=7.3 Hz), 3.31-3.38(2H, m), 5.58(1H, t, J=5.7 Hz), 6.53(2H, d, J=8.9 Hz), 7.19-7.24(3H, m), 7.30(1H, d, J=7.8 Hz), 7.64-7.73(4H, m), 7.80(2H, d, J=8.3 Hz), 8.11-8.15(2H, m), 8.49-8.52(1H, m), 10.07(1H, s)

(+)ESI-MS: 504(M+H)⁺, 526(M+Na)⁺

Preparation 122

Methyl 4-acetyl-4′-methyl-1,1′-biphenyl-2-carboxylate was obtained in the same manner as in Preparation 120.

¹H-NMR(DMSO-d₆): δ 2.36(3H, s), 2.64(3H, s), 3.65(3H, s), 7.19-7.30(4H, m), 7.58(1H, d, J=8.1 Hz), 8.15(1H, dd, J=1.8 Hz, 8.1 Hz), 8.23(1H, d, J=1.8 Hz)

Preparation 123

4-Acetyl-4′-methyl-1,1′-biphenyl-2-carboxylic acid was obtained in the same manner as in Preparation 121.

¹H-NMR(DMSO-d₆): δ 2.36(3H, s), 2.64(3H, s), 7.20-7.31(4H, m), 7.52(1H, d, J=8.0 Hz), 8.05-8.13(1H, m), 8.22(1H, d, J=1.8 Hz), 12.99(1H, s)

### EXAMPLE 252

4-Acetyl-4′-methyl-N-(4-{[2-(2-pyridinyl)ethyl]amino}-phenyl)-1,1′-biphenyl-2-carboxamide was obtained in the same manner as in Example 243.

¹H-NMR(DMSO-d₆): δ 2.31(3H, s), 2.65(3H, s), 2.97(2H, t, J=7.3 Hz), 3.31-3.38(2H, m), 5.55(1H, t, J=5.8 Hz), 6.53(2H, d, J=8.9 Hz), 7.20-7.26(1H, m), 7.22(2H, d, J=8.1 Hz), 7.25(2H, d, J=8.9 Hz), 7.31(1H, d, J=7.8 Hz), 7.39(2H, d, J=8.1 Hz), 7.58(1H, d, J=8.0 Hz), 7.67-7.73(1H, m), 8.04(1H, d, J=1.8 Hz), 8.07(1H, dd, J=1.8 Hz, 8.0 Hz), 8.49-8.52(1H, m), 9.96(1H, s)

(+)ESI-MS: 450(M+H)⁺, 472(M+Na)⁺

Preparation 124

A solution of methyl 4-acetyl-4′-(trifluoromethyl)-1,1′-biphenyl-2-carboxylate (6.3 g) in tetrahydrofuran (63 ml) was added a mixture of methyltriphenylphosphonium bromide (21.6 g) and potassium tert-butoxide (6.6 g) in tetrahydrofuran (216 ml) and the mixture was stirred under reflux for 5 hours. The reaction mixture was poured into water and the mixture was adjusted to pH 2 with 6N hydrochloric acid. The mixture was extracted with ethyl acetate. The extract was washed with water, dried over magnesium sulfate and evaporated in vacuo. The residue was purified by column chromatography on silica gel using a mixture of hexane and ethyl acetate (9:1) as an eluent. The eluted fractions containing the desired product were collected and evaporated in vacuo to give methyl 4-isopropenyl-4′-(trifluoromethyl)-1,1′-biphenyl-2-carboxylate (3.25 g).

¹H-NMR(DMSO-d₆): δ 2.17(3H, s), 3.63(3H, s), 5.24(1H, s), 5.58(1H, s), 7.46(1H, d, J=8.1 Hz), 7.52(1H, d, J=8.1 Hz), 7.76-7.83(3H, m), 7.91(1H, d, J=1.9 Hz)

Preparation 125

4-Isopropenyl-4′-(trifluoromethyl)-1,1′-biphenyl-2-carboxylic acid was obtained in the same manner as in Preparation 121.

¹H-NMR(DMSO-d₆): δ 2.17(3H, s), 5.22(1H, s), 5.56(1H, s), 7.41(1H, d, J=8.1 Hz), 7.55(2H, d, J=8.0 Hz), 7.72-7.80(3H, m), 7.90(1H, d, J=1.9 Hz), 12.96(1H, s)

### EXAMPLE 253

4-(1-Methoxy-1-methylethyl)-N-(4-{[2-(2-pyridinyl)ethyl]amino}phenyl)-4′-(trifluoromethyl)-1,1′-biphenyl-2-carboxamide was obtained in the same manner as in Example 243.

¹H-NMR(DMSO-d₆): δ 1.53(6H, s), 2.97(2H, t, J=7.3 Hz), 3.09(3H, s), 3.31-3.37(2H, m), 5.55(1H, t, J=5.7 Hz), 6.52(2H, d, J=8.9 Hz), 7.19-7.23(3H, m), 7.30(1H, d, J=7.8 Hz), 7.48(1H, d, J=8.1 Hz), 7.55(1H, d, J=1.8 Hz), 7.59(1H, dd, J=1.8 Hz, 8.1 Hz), 7.65(2H, d, J=8.2 Hz), 7.66-7.72(1H, m), 7.75(2H, d, J=8.2 Hz), 8.49-8.52(1H, m), 9.89(1H, s)

(+)ESI-MS: 534(M+H)⁺, 556(M+Na)⁺

Preparation 126

Methyl 4-isopropenyl-4′-methyl-1,1′-biphenyl-2-carboxylate was obtained in the same manner as in Preparation 124.

¹H-NMR(DMSO-d₆): δ 2.15(3H, s), 2.35(3H, s), 3.61(3H, s), 5.20(1H, s), 5.53(1H, s), 7.15-7.22(4H, m),7.40(1H, d, J=8.0 Hz), 7.73(1H, dd, J=9 Hz, 8.0 Hz), 7.78(1H, d, J=1.9 Hz)

Preparation 127

4-Isopropenyl-4′-methyl-1,1′-biphenyl-2-carboxylic acid was obtained in the same manner as in Preparation 121.

¹H-NMR(DMSO-d₆): δ 2.15(3H, s), 2.34(3H, s), 5.18(1H, s), 5.52(1H, s), 7.17-7.27(4H, m), 7.35(1H, d, J=8.0 Hz), 7.68(1H, dd, J=2.0 Hz, 8.0 Hz), 7.76(1H, d, J=2.0 Hz), 12.77(1H, s)

### EXAMPLE 254

4-(1-Methoxy-1-methylethyl)-4′-methyl-N-(4-{[2-(2-pyridinyl)ethyl]amino}phenyl)-1,1′-biphenyl-2-carboxamide was obtained in the same manner as in Example 243.

¹H-NMR(DMSO-d₆): δ 1.51(6H, s), 2.29(3H, s), 2.96(2H, t, J=7.2 Hz), 3.07(3H, s), 3.27-3.40(2H, m), 5.51(1H, t, J=5.7 Hz), 6.51(2H, d, J=8.8 Hz), 7.13-7.29(5H, m), 7.30-7.47(5H, m), 7.52(1H, dd, J=1.9 Hz, 8.0 Hz), 7.70(1H, dt, J=1.9 Hz, 7.6 Hz), 8.48-8.53(1H, m), 9.76(1H, s)

(−)ESI-MS: 478(M−H)⁻

Preparation 128

To a solution of 4-isopropenyl-4′-(trifluoromethyl)-1,1′-biphenyl-2-carboxylic acid (2.0 g) in methanol (20 ml) was added 10% palladium on carbon (0.5 g, 50% wet) . The mixture was stirred at ambient temperature for 6 hours under hydrogen atmosphere. The catalyst was filtered off and the solvent was removed by concentration to give 4-isopropyl-4′-(trifluoromethyl)-1,1′-biphenyl-2-carboxylic acid (2.00 g).

¹H-NMR(DMSO-d₆): δ 1.26(6H, d, J=6.9 Hz), 2.89-3.11(1H, m), 7.33(1H, d, J=7.9 Hz), 7.47-7.56(3H, m), 7.68(1H, d, J=1.7 Hz), 7.75(2H, d, J=8.2 Hz), 12.84(1H, s)

### EXAMPLE 255

4-Isopropyl-N-(4-{[2-(2-pyridinyl)ethyl]amino}phenyl)-4′-(trifluoromethyl)-1,1′-biphenyl-2-carboxamide was obtained in the same manner as in Example 243.

¹H-NMR(DMSO-d₆): δ 1.28(6H, d, J=6.9 Hz), 2.94-3.04(3H, m), 3.30-3.37(2H, m), 5.55(1H, t, J=5.8 Hz), 6.51(2H, d, J=8.9 Hz), 7.19-7.23(3H, m), 7.30(1H, d, J=7.8 Hz), 7.39-7.48(3H, m), 7.62(2H, d, J=8.2 Hz), 7.70(1H, dt, J=1.8 Hz, 7.6 Hz), 7.74(2H, d, J=8.2 Hz), 8.49-8.52(1H, m), 9.89(1H, s)

(+)ESI-MS: 504(M+H)⁺, 526(M+Na)⁺

Preparation 129

4-Isopropyl-4′-methyl-1,1′-biphenyl-2-carboxylic acid was obtained in the same manner as in Preparation 128.

¹H-NMR(DMSO-d₆): δ 1.24(6H, d, J=6.8 Hz), 2.33(3H, s), 2.87-3.07(1H, m), 7.20(4H, s), 7.27(1H, d, J=7.9 Hz), 7.43(1H, dd, J=1.8 Hz, 7.9 Hz), 7.54(1H, d, J=1.8 Hz), 12.67(1H, s)

### EXAMPLE 256

4-Isopropyl-4′-methyl-N-(4-{[2-(2-pyridinyl)ethyl]-amino}phenyl)-1,1′-biphenyl-2-carboxamide was obtained in the same manner as in Example 243.

¹H-NMR(DMSO-d₆): δ 1.26(6H, d, J=6.9 Hz), 2.28(3H, s), 2.92-3.02(3H, m), 3.30-3.39(2H, m), 5.52(1H, t, J=5.8 Hz), 6.51(2H, d, J=8.9 Hz), 7.16(2H, d, J=8.0 Hz), 7.19-7.22(1H, m), 7.24(2H, d, J=8.9 Hz), 7.28-7.35(5H, m), 7.37-7.41(1H, m), 7.70(1H, dt, J=1.8 Hz, 7.6 Hz), 8.49-8.52(1H, m), 9.77(1H, s)

(+)ESI-MS: 450(M+H)⁺, 472(M+Na)⁺

Preparation 130

Methyl 4-acetyl-4′-fluoro-1,1′-biphenyl-2-carboxylate was obtained in the same manner as in Preparation 120.

¹H-NMR(DMSO-d₆): δ 2.65(3H, s), 3.65(3H, s), 7.24-7.43(4H, m), 7.60(1H, d, J=8.0 Hz), 8.17(1H, dd, J=1.8 Hz, 8.0 Hz), 8.28(1H, d, J=1.8 Hz)

Preparation 131

Methyl 4′-fluoro-4-isopropenyl-1,1′-biphenyl-2-carboxylate was obtained in the same manner as in Preparation 124.

¹H-NMR(DMSO-d₆): δ 2.16(3H, s), 3.62(3H, s), 5.21(1H, s), 5.55(1H, s), 7.20-7.38(4H, m), 7.41(1H, d, J=8.1 Hz), 7.75(1H, dd, J=2.0 Hz, 8.1 Hz), 7.83(1H, d, J=2.0 Hz)

Preparation 132

4′-Fluoro-4-isopropenyl-1,1′-biphenyl-2-carboxylic acid was obtained in the same manner as in Preparation 121.

¹H-NMR(DMSO-d₆): δ 2.16(3H, s), 5.20(1H, s), 5.53(1H, s), 7.18-7.41(5H, m), 7.70(1H, dd, J=2.0 Hz, 8.1 Hz), 7.82(1H, d, J=2.0 Hz), 12.88(1H, s)

Preparation 133

4′-Fluoro-4-isopropyl-1,1′-biphenyl-2-carboxylic acid was obtained in the same manner as in Preparation 128.

¹H-NMR(DMSO-d₆): δ 1.24(6H, d, J=6.9 Hz), 2.88-3.10(1H, m), 7.16-7.38(5H, m), 7.45(1H, dd, J=1.8 Hz, 7.9 Hz), 7.60(1H, d, J=1.8 Hz), 12.76(1H, s)

### EXAMPLE 257

4′-Fluoro-4-isopropyl-N-(4-{[2-(2-pyridinyl)ethyl]-amino}phenyl)-1,1′-biphenyl-2-carboxamide was obtained in the same manner as in Example 243.

¹H-NMR(DMSO-d₆): δ 1.27(6H, d, J=6.9 Hz), 2.94-3.03(3H, m), 3.30-3.37(2H, m), 5.53(1H, t, J=5.8 Hz), 6.51(2H, d, J=8.9 Hz), 7.16-7.24(5H, m), 7.30(1H, d, J=7.8 Hz), 7.33-7.38(2H, m), 7.39-7.47(3H, m), 7.70(1H, dt, J=1.8 Hz, 7.6 Hz), 8.49-8.52(1H, m), 9.78(1H, s)

(+)ESI-MS: 454(M+H)⁺, 476(M+Na)⁺

Preparation 134

Methyl 4-acetyl-1,1′-biphenyl-2-carboxylate was obtained in the same manner as in Preparation 120.

¹H-NMR(DMSO-d₆): δ 2.65(3H, s), 3.63(3H, s), 7.31-7.37(2H, m), 7.40-7.50(3H, m), 7.60(1H, d, J=8.0 Hz), 8.17(1H, dd, J=1.8 Hz, 8.0 Hz), 8.27(1H, d, J=1.8 Hz)

Preparation 135

Methyl 4-isopropenyl-1,1′-biphenyl-2-carboxylate carboxylate was obtained in the same manner as in Preparation 124.

¹H-NMR(DMSO-d₆): δ 2.16(3H, s), 3.59(3H, s), 5.21(1H, s), 5.55(1H, s), 7.26-7.32(2H, m), 7.36-7.45(4H, m), 7.75(1H, dd, J=2.0 Hz, 8.1 Hz), 7.81(1H, d, J=2.0 Hz)

Preparation 136

4-Isopropenyl-1,1′-biphenyl-2-carboxylic acid was obtained in the same manner as in Preparation 121.

¹H-NMR(DMSO-d₆): δ 2.16(3H, s), 5.20(1H, s), 5.54(1H, s), 7.29-7.47(6H, m), 7.70(1H, dd, J=2.0 Hz, 8.0 Hz), 7.80(1H, d, J=2.0 Hz), 12.84(1H, s)

Preparation 137

4-Isopropyl-1,1′-biphenyl-2-carboxylic acid was obtained in the same manner as in Preparation 128.

¹H-NMR(DMSO-d₆): δ 1.25(6H, d, J=6.9 Hz), 2.88-3.08(1H, m), 7.27-7.47(7H, m), 7.58(1H, d, J=1.8 Hz), 12.71(1H, br-s)

### EXAMPLE 258

4-Isopropyl-N-(4-{[2-(2-pyridinyl)ethyl]amino}phenyl)-1,1′-biphenyl-2-carboxamide was obtained in the same manner as in Example 243.

¹H-NMR(DMSO-d₆): δ 1.27(6H, d, J=6.9 Hz), 2.93-3.04(3H, m), 3.29-3.37(2H, m), 5.51(1H, t, J=5.8 Hz), 6.50(2H, d, J=8.9 Hz), 7.18-7.23(3H, m), 7.24-7.32(2H, m), 7.33-7.38(4H, m), 7.39-7.45(3H, m), 7.70(1H, dt, J=1.9 Hz, 7.6 Hz), 8.49-8.52(1H, m), 9.75(1H, s)

(+)ESI-MS: 436(M+H)⁺, 458(M+Na)⁺

### EXAMPLE 259

N-(4-{[2-(6-Amino-2-pyridinyl)ethyl]amino}phenyl)-4-ethyl-4′-(trifluoromethyl)-1,1′-biphenyl-2-carboxamide was obtained in the same manner as in Example 243.

¹H-NMR(DMSO-d₆): δ 1.26(3H, t, J=7.5 Hz), 2.65-2.79(4H, m), 3.08-3.34(2H, m), 5.52(1H, t, J=5.5 Hz), 5.82(2H, s), 6.27(1H, d, J=8.2 Hz), 6.39(1H, d, J=7.1 Hz), 6.50(2H, d, J=8.7 Hz), 7.18-7.31(3H, m), 7.37-7.48(3H, m), 7.62(2H, d, J=8.2 Hz), 7.74(2H, d, J=8.2 Hz), 9.89(1H, s)

(+)ESI-MS: 505(M+H)⁺, 527(M+Na)⁺

### EXAMPLE 260

N-(4-{[2-(6-Amino-2-pyridinyl)ethyl]amino}phenyl)-4-isopropyl-4′-(trifluoromethyl)-1,1′-biphenyl-2-carboxamide was obtained in the same manner as in Example 243.

¹H-NMR(DMSO-d₆): δ 1.28(6H, d, J=6.9 Hz), 2.71(2H, t, J=7.3 Hz), 2.97-3.07(1H, m), 3.21-3.27(2H, m), 5.53(1H, t, J=5.6 Hz), 5.83(2H, s), 6.27(1H, d, J=8.2 Hz), 6.39(1H, d, J=7.2 Hz), 6.50(2H, d, J=8.8 Hz), 7.20(2H, d, J=8.8 Hz), 7.26-7.29(1H, m), 7.39-7.48(3H, m), 7.62(2H, d, J=8.2 Hz), 7.74(2H, d, J=8.2 Hz), 9.88(1H, s)

(+)ESI-MS: 519(M+H)⁺, 541(M+Na)⁺

### EXAMPLE 261

N-(4-{[2-(6-Amino-2-pyridinyl)ethyl]amino}phenyl)-5-methyl-1,1′-biphenyl-2-carboxamide was obtained in the same manner as in Example 243.

¹H-NMR(DMSO-d₆): δ 2.40(3H, s), 2.70(2H, t, J=7.2 Hz), 3.18-3.32(2H, m), 5.48(1H, t, J=5.4 Hz), 5.82(2H, s), 6.27(1H, d, J=8.1 Hz), 6.38(1H, d, J=7.1 Hz), 6.48(2H, d, J=8.8 Hz), 7.18(2H, d, J=8.8 Hz), 7.21-7.46(9H, m), 9.66(1H, s)

(+)ESI-MS: 423(M+H)⁺, 445(M+Na)⁺

### EXAMPLE 262

N-(4-{[2-(6-Amino-2-pyridinyl)ethyl]amino}phenyl)-5-chloro-4′-(trifluoromethyl)-1,1′-biphenyl-2-carboxamide was obtained in the same manner as in Example 243.

¹H-NMR(DMSO-d₆): δ 2.71(2H, t, J=7.2 Hz), 3.19-3.30(2H, m), 5.55(1H, t, J=5.6 Hz), 5.82(2H, s), 6.27(1H, d, J=8.1 Hz), 6.38(1H, d, J=7.0 Hz), 6.50(2H, d, J=8.8 Hz), 7.18(2H, d, J=8.8 Hz), 7.22-7.29(1H, m), 7.56-7.68(5H, m)!. 7.78(2H, d, J=8.3 Hz), 9.95(1H, s)

(+)ESI-MS: 511(M+H)⁺, 533(M+Na)⁺

### EXAMPLE 263

4,5-Dimethyl-N-(4-{[2-(2-pyridinyl)ethyl]amino}phenyl)-4′-(trifluoromethyl)-1,1′-biphenyl-2-carboxamide was obtained in the same manner as in Example 243.

¹H-NMR (DMSO-d₆): δ 2.32(6H, s), 2.96(2H, t, J=7.3 Hz), 3.30-3.36(2H, m), 5.53(1H, t, J=5.7 Hz), 6.51(2H, d, J=8.7 Hz), 7.19-7.24(3H, m), 7.26(1H, s), 7.30(1H, d, J=7.5 Hz), 7.37(1H, s), 7.59(2H, d, J=8.1 Hz), 7.67-7.74(3H, m), 8.49-8.52(1H, m), 9.84(1H, s)

(+)ESI-MS: 490(M+H)⁺, 512(M+Na)⁺

### EXAMPLE 264

4,4′,5-Trimethyl-N-(4-{[2-(2-pyridinyl)ethyl]amino}-phenyl)-1,1′-biphenyl-2-carboxamide was obtained in the same manner as in Example 243.

¹H-NMR(DMSO-d₆): δ 2.29(9H, s), 2.96(2H, t, J=7.2 Hz), 3.28-3.39(2H, m), 5.49(1H, t, J=5.7 Hz), 6.50(2H, d, J=8.8 Hz.), 7.11-7.33(10H, m), 7.70(1H, dt, J=1.9 Hz, 7.7 Hz), 8.48-8.53(1H, m), 9.68(1H, s)

(+)ESI-MS: 436(M+H)⁺, 458(M+Na)⁺

Preparation 138

4′-Chloro-4,5-dimethyl-1,1′-biphenyl-2-carboxylic acid was obtained in the same manner as in Preparation 112.

¹H-NMR(DMSO-d₆): δ 2.29(6H, s), 7.13(1H, s), 7.26-7.31(2H, m), 7.40-7.45(2H, m), 7.58(1H, s), 12.59(1H, s)

### EXAMPLE 265

4′-Chloro-4,5-dimethyl-N-(4-{[2-(2-pyridinyl)ethyl]amino}phenyl)-1,1′-biphenyl-2-carboxamide was obtained in the same manner as in Example 243.

¹H-NMR(DMSO-d₆): δ 2.30(6H, s), 2.96(2H, t,. J=7.2 Hz), 3.31-3.39(2H, m), 5.51(1H, t, J=5.7 Hz), 6.51(2H, d, J=8.8 Hz), 7.17-7.34(6H, m), 7.41(4H, s), 7.70(1H, dt, J=1.9 Hz, 7.6 Hz), 8.48-8.52(1H, m), 9.75(1H, s)

(+)ESI-MS: 456(M+H)⁺, 478(M+Na)⁺

Preparation 139

4′-Fluoro-4,5-dimethyl-1,1′-biphenyl-2-carboxylic acid was obtained in the same manner as in Preparation 112.

¹H-NMR(DMSO-d₆): δ 2.28(6H, s), 7.12(1H, s), 7.17-7.23(2H, m), 7.28-7.32(2H, m), 7.56(1H, s), 12.54(1H, s)

### EXAMPLE 266

4′-Fluoro-4,5-dimethyl-N-(4-{[2-(2-pyridinyl)ethyl]-amino}phenyl)-1,1′-biphenyl-2-carboxamide was obtained in the same manner as in Example 243.

¹H-NMR(DMSO-d₆): δ 2.22(6H, s), 2.96(2H, t, J=7.2 Hz), 3.29-3.37(2H, m), 5:51(1H, t, J=5.7 Hz), 6.50(2H, d, J=8.8 Hz), 7.16-7.23(6H, m), 7.28-7.32(2H, m), 7.39-7.44(2H, m), 7.67-7.73(1H, m), 8.51(1H, dd, J=0.7 Hz, 4.7 Hz), 9.72(1H, s)

(+)ESI-MS: 440(M+H)⁺, 462(M+Na)⁺

Preparation 140

4,5-Dimethyl-1,1′-biphenyl-2-carboxylic acid was obtained in the same manner as in Preparation 112.

¹H-NMR(DMSO-d₆): δ 2.29(6H, s), 7.14(1H, s), 7.25-7.41(5H, m), 7.53(1H, s), 12.48(1H, s)

### EXAMPLE 267

4,5-Dimethyl-N-(4-{[2-(2-pyridinyl)ethyl]amino}phenyl)-1,1′-biphenyl-2-carboxamide was obtained in the same manner as in Example 243.

¹H-NMR(DMSO-d₆): δ 2.30(6H, s), 2.96(2H, t, J=7.2 Hz), 3.27-3.38(2H, m), 5.49(1H, t, J=5.7 Hz), 6.50(2H, d, J=8.8 Hz), 7.15-7.45(11H, m), 7.70(1H, dt, J=1.8 Hz, 7.6 Hz), 8.48-8.52(1H, m), 9.67(1H, s)

(+)ESI-MS: 422(M+H)⁺, 444(M+Na)⁺

Preparation 141

3-[4-(Trifluoromethyl)phenyl]-2-naphthoic acid was obtained in the same manner as in Preparation 112.

¹H-NMR(DMSO-d₆): δ 7.62-7.69(4H, m), 7.80(2H, d, J=8.1 Hz), 7.98(1H, s), 8.04(1H, d, J=8.0 Hz), 8.13(1H, d, J=7.8 Hz), 8.51(1H, s), 12.99(1H, s)

### EXAMPLE 268

N-(4-{[2-(2-Pyridinyl)ethyl]amino}phenyl)-3-[4-(trifluoromethyl)phenyl]-2-naphthamide was obtained in the same manner as in Example 243.

¹H-NMR(DMSO-d₆): δ 2.98(2H, t, J=7.2 Hz), 3.33-3.39(2H, m)), 5.57(1H, t, J=5.8 Hz), 6.55(2H, d, J=8.8 Hz), 7.19-7.23(1H, m), 7.28-7.33(3H, m), 7.62-7.66(2H, m), 7.69-7.75(3H, m), 7.77-7.81(2H, m), 8.02-8.12(3H, m), 8.22(1H, s), 8.50-8.53(1H, m), 10.20(1H, s)

(+)ESI-MS: 512(M+H)⁺, 534(M+Na)⁺

### EXAMPLE 269

4,5-Dimethoxy-N-(4-{[2-(2-pyridinyl)ethyl]amino}phenyl)-4′-(trifluoromethyl)-1,1′-biphenyl-2-carboxamide was obtained in the same manner as in Example 243.

¹H-NMR(DMSO-d₆): δ 2.96(2H, t, J=7.1 Hz), 3.27-3.44(2H, m), 3.86(3H, s), 3.87(3H, s), 5.52(1H, t, J=5.6 Hz), 6.51(2H, d, J=8.7 Hz), 7.03(1H, s), 7.16-7.25(4H, m), 7.30(1H, d, J=7.7 Hz), 7.58-7.75(5H, m), 8.48-8.53(1H, m), 9.74(1H, s)

(+)ESI-MS: 522(M+H)⁺, 544(M+Na)⁺

### EXAMPLE 270

4,5-Dimethoxy-4′-methyl-N-(4-{[2-(2-pyridinyl)ethyl]-amino}phenyl)-1,1′-biphenyl-2-carboxamide was obtained in the same manner as in Example 243.

¹H-NMR(DMSO-d₆): δ 2.29(3H, s), 2.96(2H, t, J=7.2 Hz), 3.27-3.35(2H, m), 3.83(6H, s), 5.49(1H, t, J=5.7 Hz), 6.50(2H, d, J=8.8 Hz), 6.93(1H, s), 7.05-7.36(9H, m), 7.69(1H, dt, J=1.6 Hz, 7.6 Hz), 8.48-8.53(1H, m), 9.58(1H, s)

(+)ESI-MS: 468(M+H)⁺, 490(M+Na)⁺

Preparation 142

3-Methyl-4′-(trifluoromethyl)-1,1′-biphenyl-2-carboxylic acid was obtained in the same manner as in Preparation 112.

¹H-NMR(DMSO-d₆): δ 2.37(3H, s), 7.26(1H, d, J=7.4 Hz), 7.31-7.48(2H, m), 7.60(2H, d, J=8.2 Hz), 7.80(2H, d, J=8.2 Hz), 13.14(1H, s)

(−)ESI-MS: 279(M−H)⁻

### EXAMPLE 271

1-[3-(Dimethylamino)propyl]-3-ethylcarbodiimide (0.31 g) was added to a solution of 4-aminophenyl(2-(2-pyridinyl)ethyl)formamide (0.4 g), 3-methyl-4′-(trifluoromethyl)-1,1′-biphenyl-2-carboxylic acid (0.56 g), 1-hydroxybenzotriazole (0.27 g) and 4-dimethylaminopyridine (20 mg) in 1,3-dimethyl-2-imidazolidinone (4 ml) at ambient temperature and the mixture was stirred at 120° C. for 20 hours. The reaction mixture was poured into a mixture of ethyl acetate and water. The separated organic layer was washed with water, dried over magnesium sulfate and evaporated in vacuo. The residue was purified by column chromatography on silica gel using ethyl acetate as an eluent. The eluted fractions containing the desired product were collected and evaporated in vacuo to give N-(4-{formyl[2-(2-pyridinyl)ethyl]amino}phenyl)-3-methyl-4′-(trifluoromethyl)-1,1′-biphenyl-2-carboxamide (70.0 mg).

¹H-NMR(DMSO-d₆): δ 2.38(3H, s), 2.87(2H, t, J=7.5 Hz), 4.06(2H, t, J=7.5 Hz), 7.13-7.26(4H, m), 7.31(1H, d, J=7.0 Hz), 7.37-7.56(4H, m), 7.60-7.80(5H, m), 8.29(1H, s), 8.42-8.48(1H, m), 10.45(1H, s)

### EXAMPLE 272

A mixture of N-(4-{formyl[2-(2-pyridinyl) ethyl]amino}-phenyl)-3-methyl-4′-(trifluoromethyl)-1,1′-biphenyl-2-carboxamide (0.3 g) and conc. hydrochloric acid (0.3 ml) in methanol (1.5 ml) was stirred at ambient temperature for 20 hours. The reaction mixture was poured into a water and the mixture was adjusted to pH 9 with 20% aqueous potassium carbonate solution. The mixture was extracted with ethyl acetate. The extract was washed with brine, dried over magnesium sulfate and evaporated in vacuo. The residue was purified by column chromatography on silica gel using a mixture of ethyl acetate and diisopropyl ether (2:1) as an eluent. The eluted fractions containing the desired product were collected and evaporated in vacuo to give 3-methyl-N-(4-{[2-(2-pyridinyl)ethyl]amino}phenyl)-4′-(trifluoromethyl)-1,1′-biphenyl-2-carboxamide (45.0 mg).

¹H-NMR(DMSO-d₆): δ 2.37(3H, s), 2.95(2H, t, J=7.2 Hz), 3.26-3.39(2H, m), 5.53(1H, t, J=5.6 Hz), 6.49(2H, d, J=8.7 Hz), 7.13(2H, d, J=8.7 Hz), 7.16-7.50(5H, m), 7.63-7.79(5H, m), 8.48-8.54(1H, m), 9.89(1H, s)

(−)ESI-MS: 474(M−H)⁻

### EXAMPLE 273

4′-(Dimethylamino)-4-methyl-N-(4-{[2-(2-pyridinyl)ethyl]amino}phenyl)-1,1′-biphenyl-2-carboxamide was obtained in the same manner as in Example 243.

¹H-NMR(DMSO-d₆): δ 2.37(3H, s), 2.88(6H, s), 2.97(2H, t, J=7.3 Hz), 3.30-3.40(2H, m), 5.49(1H, t, J=5.8 Hz), 6.52(2H, d, J=8.8 Hz), 6.70(2H, d, J=8.8 Hz), 7.18-7.34(9H, m), 7.67-7.73(1H, m), 8.49-8.54(1H, m), 9.73(1H, s)

(+)ESI-MS: 451(M+H)⁺, 473(M+Na)⁺

### EXAMPLE 274

4′-(Dimethylamino)-5-methyl-N-(4-{[2-(2-pyridinyl)ethyl]amino}phenyl)-1,1′-biphenyl-2-carboxamide was obtained in the same manner as in Example 243.

¹H-NMR(DMSO-d₆): δ 2.37(3H, s), 2.88(6H, s), 2.96(2H, t, J=7.3 Hz), 3.29-3.40(2H, m), 5.48(1H, t, J=5.8 Hz), 6.51(2H, d, J=8.8 Hz), 6.70(2H, d, J=8.8 Hz), 7.14-7.37(9H, m), 7.67-7.73(1H, m), 8.49-8.53(1H, m), 9.63(1H, s)

(+)ESI-MS: 451(M+H)⁺, 473(M+Na)⁺

### EXAMPLE 275

5-Chloro-4′-(dimethylamino)-N-(4-{[2-(2-pyridinyl)ethyl]amino}phenyl)-1,1′-biphenyl-2-carboxamide was obtained in the same manner as in Example 243.

¹H-NMR(DMSO-d₆): δ 2.90(6H, s), 2.96(2H, t, J=7.3 Hz), 3.27-3.42(2H, m), 5.54(1H, t, J=5.6 Hz), 6.52(2H, d, J=8.8 Hz), 6.71(2H, d, J=8.8 Hz), 7.17-7.52(9H, m), 7.70(1H, dt, J=1.8 Hz, 7.6 Hz), 8.47-8.54(1H, m), 9.83(1H, s)

(+)ESI-MS: 471(M+H)⁺, 493(M+Na)⁺

### EXAMPLE 276

To a solution of 4-methyl-4′-(trifluoromethyl)-1,1′-biphenyl-2-carboxylic acid (221 mg) in toluene (5 ml) were added thionyl chloride (188 mg) and N,N-dimethylformamide (1 drop) and the mixture was stirred at 80° C. for 30 minutes. The mixture was evaporated in vacuo and the residue was dissolved in tetrahydrofuran (5 ml). The acid chloride in tetrahydrofuran was added to a solution of tert-butyl 6-[2-(4-aminophenoxy)ethyl]-2-pyridinylcarbamate (236 mg) and triethylamine (160 mg) in tetrahydrofuran (5 ml) at ambient temperature and the mixture was stirred at the same temperature for an hour. The mixture was poured into water and extracted with ethyl acetate. The organic layer was washed with brine, dried over magnesium sulfate, and evaporated in vacuo. The residue was purified by column chromatography on silica gel by eluting with ethyl acetate:hexane (1:3) to give tert-butyl 6-{2-[4-({[4-methyl-4′-(trifluoromethyl)-1,1′-biphenyl-2-yl]carbonyl}amino)phenoxy]-ethyl}-2-pyridinylcarbamate (386 mg) as a colorless foam.

¹H-NMR(CDCl₃): δ 1.51(9H, s), 2.45(3H, s), 3.08(2H, t, J=6.6 Hz), 4.25(2H, t, J=6.6 Hz), 6.77(2H, d, J=8.9 Hz), 6.78(1H, s), 6.88(1H, d, J=7.6 Hz), 7.03(2H, d, J=8.9 Hz), 7.33(2H, t, J=6.9 Hz), 7.55-7.68(6H, m), 7.76(1H, d, J=8.3 Hz)

### EXAMPLE 277

To a solution of tert-butyl 6-{2-[4-({[4-methyl-4′-(trifluoromethyl)-1,1′-biphenyl-2-yl]carbonyl}amino)phenoxy]-ethyl}-2-pyridinylcarbamate (378 mg) in dichloromethane (10 ml) was added trifluoroacetic acid (1.00 ml). The reaction mixture was stirred for 19 hours, quenched with 10% aqueous potassium carbonate solution, and extracted with dichloromethane. The organic layer was washed with brine, dried over magnesium sulfate, filtered, and concentrated in vacuo. The residue was recrystallized from ethyl acetate-diisopropyl ether to give N-{4-[2-(6-amino-2-pyridinyl) ethoxy]phenyl}-4-methyl-4′-(trifluoromethyl)-1,1′-biphenyl-2-carboxamide (246 mg) as colorless crystals.

¹H-NMR(CDCl₃): δ 2.45(3H, s), 3.04(2H, t, J=6.9 Hz), 4.25(2H, t, J=6.9 Hz), 4.38(2H, br s), 6.35(1H, d, J=8.2 Hz), 6.59(1H, d, J=7.3 Hz), 6.79(2H, d, J=8.9 Hz), 6.80(1H, s), 7.03(2H, d, J=8.2 Hz), 7.29-7.38(3H, m), 7.55-7.68(5H, m)

ESI-MS(m/z): 492(M+H)⁺

### EXAMPLE 278

tert-Butyl 6-[2-(4-{[(4,4′-dimethyl-1,1′-biphenyl-2-yl)carbonyl]amino}phenoxy)ethyl]-2-pyridinylcarbamate (375 mg) was obtained in the same manner as in Example 276.

¹H-NMR(CDCl₃): δ 1.52(9H, s), 2.38(3H, s), 2.43(3H, s), 3.08′(2H, t, J=6.7 Hz), 4.24(2H, t, J=6.7 Hz), 6.74-6.77(3H, m), 6.88(1H, d, J=7.3 Hz), 6.99(2H, d, J=9.2 Hz), 7.21-7.35(6H, m), 7.57(1H, t, J=7.8 Hz), 7.68(1H, s), 7.76(1H, d, J=7.9 Hz)

### EXAMPLE 279

To a solution of tert-butyl 6-[2-(4-{[(4,4′-dimethyl-1,1′-biphenyl-2-yl)carbonyl]amino}phenoxy)ethyl]-2-pyridinylcarbamate (379 mg) in dichloromethane (10 ml) was added trifluoroacetic acid (1.00 ml). The reaction mixture was stirred for 40 hours, quenched with 10% aqueous potassium carbonate solution, and extracted with dichloromethane. The organic layer was washed with brine, dried over magnesium sulfate, filtered, and concentrated in vacuo. The residue was purified by column chromatography on silica gel by eluting with chloroform:methanol (19:1) to give N-{4-[2-(6-amino-2-pyridinyl)ethoxy]phenyl}-4,4′-dimethyl-1,1′-biphenyl-2-carboxamide (246 mg) as a colorless foam.

¹H-NMR(CDCl₃): δ 2.38(3H, s), 2.43(3H, s), 3.05(2H, t, J=6.7 Hz), 4.24(2H, t, J=6.7 Hz), 4.58(2H, br s), 6.37(1H, d, J=8.2 Hz), 6.59(1H, d, J=7.3 Hz), 6.77(2H, d, J=8.9 Hz), 6.78(1H, s), 6.99(2H, d, J=8.9 Hz), 7.21-7.40(7H, m), 7.68(1H, s)

ESI-MS(m/z): 438(M+H)⁺

### EXAMPLE 280

tert-Butyl 6-[2-(4-{[(4′-chloro-4-methyl-1,1′-biphenyl-2-yl)carbonyl]amino}phenoxy)ethyl]-2-pyridinylcarbamate was obtained in the same manner as in Example 276 as a yellow foam;

¹H-NMR(CDCl₃): δ 1.52(9H, s), 2.43(3H, s), 3.09(2H, t, J=6.7 Hz), 4.26(2H, t, J=6.7 Hz), 6.78(1H, s), 6.79(2H, d, J=8.9 Hz), 6.89(1H, d, J=7.3 Hz), 7.07(2H, d, J=8.9 Hz), 7.24-7.38(8H, m), 7.54-7.60(2H, m), 7.76(1H, d, J=7.9 Hz)

### EXAMPLE 281

N-{4-[2-(6-Amino-2-pyridinyl)ethoxy]phenyl}-4′-chloro-4-methyl-1,1′-biphenyl-2-carboxamide was obtained in the same manner as in Example 277 as colorless crystals.

¹H-NMR(CDCl₃):δ 2.43(3H, s), 3.05(2H, t, J=6.9 Hz), 4.26(2H, t, J=6.9 Hz), 4.38(2H, br s), 6.35(1H, d, J=7.9 Hz), 6.59(1H, d, J=7.3 Hz), 6.79(1H, s), 6.80(2H, d, J=8.9 Hz), 7.06(2H, d, J=8.9 Hz), 7.25-7.38(6H, m), 7.60(1H, s)

ESI-MS(m/z): 458, 460(M+H)⁺

Preparation 143

A mixture of 2-(2-methyl-1,3-thiazol-4-yl)ethanamine (6.823 g), 1-fluoro-4-nitrobenzene (8.123 g) and triethylamine (5.829 g) in 1,3-dimethyl-2-imidazolidinone (50 ml) was heated to 50° C. for 16 hours. The reaction mixture was cooled to ambient temperature, poured into water and extracted with ethyl acetate. The organic layer was washed with brine, dried over magnesium sulfate, filtered, and concentrated in vacuo. The residue was purified by column chromatography on silica gel by eluting with hexane:ethyl acetate (2:1) to give N-[2-(2-methyl-1,3-thiazol-4-yl)ethyl]-4-nitroaniline (7.764 g) as a yellow oil.

¹H-NMR(CDCl₃): δ 2.71 (3H; s), 3.05 (2H, t, J=6.3 Hz), 3.50-3.59 (2H, m), 5.20-5.31 (1H, m), 6.54 (2H, d, J=8.9 Hz), 6.83 (1H, s), 8.09 (2H, d, J=9.2 Hz)

Preparation 144

To a solution of N-[2-(2-methyl-1,3-thiazol-4-yl)ethyl]-4-nitroaniline (7.764 g) and 4-(N,N-dimethylamino)pyridine (1.081 mg) in tetrahydrofuran (100 ml) was added di-t-butyl dicarbonate (8.366 g) and heated to 50° C. for 12hours. The reaction mixture was cooled to ambient temperature and concentrated in vacuo. The residue was dissolved in ethyl acetate and water, and extracted with ethyl acetate. The organic layer was washed with water and brine, dried over magnesium sulfate, filtered, and concentrated in vacuo. The residue was purified by column chromatography on silica gel by eluting with hexane:ethyl acetate (4:1) to give tert-butyl 2-(2-methyl-1,3-thiazol-4-yl)ethyl(4-nitrophenyl)carbamate (10.62 g) as a dark orange oil.

¹H-NMR(CDCl₃): δ 1.47 (9H, s), 2.60 (3H, s), 3.03 (2H, t, J=7.0 Hz), 4.08 (2H, t, J=7.0 Hz), 6.76 (1H, s), 7.31 (2H, d, J=9.2 Hz), 8.14 (2H, d, J=9.2 Hz)

Preparation 145

A solution of tert-butyl 2-(2-methyl-1,3-thiazol-4-yl)ethyl(4-nitrophenyl)carbamate (10.63 g) in methanol (100 ml) was hydrogenated over 10% palladium on carbon (5.00 g, 50% wet) at ambient temperature under atmospheric pressure of hydrogen for 4.5 hours. The reaction mixture was filtered through a short pad of celite, and the filtrate was concentrated in vacuo. The residue was purified by column chromatography on silica gel by eluting with chloroform:methanol (19:1) to give tert-butyl 4-aminophenyl[2-(2-methyl-1,3-thiazol-4-yl)ethyl]carbamate (9.295 g) as yellow crystals.

¹H-NMR(CDCl₃): δ 1.39 (9H, s), 2.64 (3H, s), 2.96 (2H, t, J=7.6 Hz), 3.51-3.76 (2H, m), 3.90 (2H, t, J=7.6 Hz), 6.67 (2H, d, J=7.9 Hz), 6.78 (1H, s), 6.90 (2H, brd, J=7.9 Hz)

### EXAMPLE 282

To a solution of 4′,6-dimethyl[1, ′-biphenyl]-2-carboxylic acid (226.27 mg) in toluene (2 ml) were added thionyl chloride (145.6 mg) and N,N-dimethylformamide (1 drop) and the mixture was stirred at 80° C. for 2 hours. The mixture was evaporated in vacuo and the residue was dissolved in tetrahydrofuran (2 ml). The acid chloride in tetrahydrofuran was added to a solution of tert-butyl 4-aminophenyl[2-(2-methyl-1,3-thiazol-4-yl)ethyl]carbamate (170 mg) and triethylamine (103.2 mg) in tetrahydrofuran (5 ml) at ambient temperature and the mixture was stirred at the same temperature for 30 minutes. The mixture was poured into 10% aqueous potassium carbonate solution and extracted with ethyl acetate. The organic layer was washed with brine, dried over magnesium sulfate, and evaporated in vacuo to give tert-butyl 4-{[(4′, 6-dimethyl-1,1′-biphenyl-2-yl) carbonyl]amino}phenyl[2-(2-methyl-1,3-thiazol-4-yl)ethyl]carbamate (276.2 mg) as a yellow foam.

### EXAMPLE 283

To a solution of tert-butyl 4-{[(4′,6-dimethyl-1,1′-biphenyl-2-yl)carbonyl]amino}phenyl[2-(2-methyl-1,3-thiazol-4-yl)ethyl]carbamate (276.2 mg) in dichloromethane (8 ml) was added trifluoroacetic acid (0.982 ml). The reaction mixture was stirred for 24 hours, quenched with 10% aqueous potassium carbonate solution, and extracted with dichloromethane. The organic layer was washed with brine, dried over magnesium sulfate, filtered, and concentrated in vacuo. The residue was recrystallized from ethyl acetate-hexane to give 4′,6-dimethyl-N-(4-{[2-(2-methyl-1,3-thiazol-4-yl)ethyl]amino}phenyl)-1,1′-biphenyl-2-carboxamide (186.8 mg) as a pale brown foam.

¹H-NMR(DMSO-d₆): δ 2.16 (3H, s), 2.41 (3H, s), 2.69 (3H, s), 2.98 (2H, t, J=6.6 Hz), 3.40 (2H, t, J=6.6 Hz), 6.46 (2H, t, J=8.9 Hz), 6.71 (1H, s), 6.76 (1H, s), 6.81 (2H, d, J=8.9 Hz), 7.18-7.38 (6H, m), 7.74 (1H, dd, J=6.6, 2.3 Hz)

ESI-MS(m/z): 442(M+H)⁺

Preparation 146

4-[2-(4-Nitroanilino)ethyl]-1,3-thiazole was obtained in the same manner as in Preparation 143 as a brown oil.

¹H-NMR(CDCl₃): δ 3.17 (2H, t, J=6.4 Hz), 3.60 (2H, q, J=6.1 Hz), 6.53-8.09 (4H, AaBb), 7.08 (1H, d, J=2.0 Hz), 8.80 (1H, s)

Preparation 147

tert-Butyl 4-nitrophenyl[2-(1,3-thiazol-4-yl) ethyl]carbamate was obtained in the same manner as in Preparation 144 as a yellow oil.

¹H-NMR(CDCl₃): δ 1.46 (9H, s), 3.14 (2H, t, J=6.8 Hz), 4.11 (2H, t, J=7.1 Hz), 7.01(1H, d, J=2.0 Hz), 7.26-8.16 (4H, AaBb), 8.69 (1H, d, J=2.0 Hz)

Preparation 148

tert-Butyl 4-aminophenyl[2-(1,3-thiazol-4-yl)ethyl]carbamate was obtained in the same manner as in Preparation 145 as an orange oil.

¹H-NMR(CDCl₃): δ 1.39 (9H, s), 3.07 (2H, t, J=7.4 Hz), 3.93 (2H, t, J=7.4 Hz), 6.11 (2H, d, J=8.6 Hz), 6.9 (2H, br s), 7.00 (1H, br s), 8.7 (1H, d, J=2.0 Hz)

### EXAMPLE 284

To a solution of 5-methyl-4′-(trifluoromethyl)-1,1′-biphenyl-2-carboxylic acid (212 mg) in toluene (5 ml) were added thionyl chloride (0.11 ml) and N,N-dimethylformamide (1 drop) and the mixture was stirred at 100° C. for 2 hours. The mixture was evaporated in vacuo and the residue was dissolved in tetrahydrofuran (2 ml). The acid chloride in tetrahydrofuran was added to a solution of tert-butyl 4-aminophenyl[2-(1,3-thiazol-4-yl)ethyl]carbamate (201 mg) and triethylamine (0.18 ml) in tetrahydrofuran (5 ml) at ambient temperature and the mixture was stirred at the same temperature for 2 hours. The reaction mixture was poured into water and extracted with ethyl acetate. The organic layer was washed with brine, dried over magnesium sulfate, and evaporated in vacuo. The residue was purified by column chromatography on silica gel by eluting with hexane:ethyl acetate (3:1) to give tert-butyl 4-({[5-methyl-4′-(trifluoromethyl)-1,1′-biphenyl-2-yl]carbonyl}amino)phenyl[2-(1,3-thiazol-4-yl)ethyl]carbamate (291 mg) as a yellow foam.

### EXAMPLE 285

To a solution of tert-butyl 4-({[5-methyl-4′-(trifluoromethyl)-1,1′-biphenyl-2-yl]carbonyl}amino)phenyl[2-(1,3-thiazol-4-yl)ethyl]carbamate (291 mg) in dichloromethane (15 ml) was added trifluoroacetic acid (0.77 ml). The reaction mixture was stirred for 15 hours, quenched with 10% aqueous potassium carbonate solution, and extracted with dichloromethane. The organic layer was washed with brine, dried over magnesium sulfate, filtered, and concentrated in vacuo. The residue was recrystallized from ethyl acetate-diisopropyl ether to give 5-methyl-N-(4-{[2-(1,3-thiazol-4-yl)ethyl]amino}phenyl)-4′-(trifluoromethyl)-1,1′-biphenyl-2-carboxamide (161 mg) as a pale yellow solid.

¹H-NMR(DMSO-d₆): δ 2.41 (3H, s), 2.99 (2H, t, J=7.2 Hz), 3.31 (2H, q, J=6.9 Hz), 5.51 (1H, t, J=5.9 Hz), 6.50 (2H, d, J=8.9 Hz), 7.19 (2H, d, J=8.9 Hz), 7.29 (1H, s), 7.32 (1H, d, J=8.2 Hz), 7.41 (1H, d, J=1.6 Hz), 7.48 (1H, d, J=7.6 Hz), 7.61 (2H, d, J=7.9 Hz), 7.74 (2H, d, J=8.2 Hz), 9.03 (1H, d, J=2.0 Hz), 9.82 (1H, s)

ESI-MS(m/z): 482(M+H)⁺

### EXAMPLE 286

tert-Butyl 4-({[4-methyl-4′-(trifluoromethyl)-1,1′-biphenyl-2-yl]carbonyl}amino)phenyl[2-(1,3-thiazol-4-yl)ethyl]carbamate was obtained in the same manner as in Example 284 as a yellow foam.

### EXAMPLE 287

4-Methyl-N-(4-{[2-(1,3-thiazol-4-yl)ethyl]amino}phenyl)-4′-(trifluoromethyl)-1,1′-biphenyl-2-carboxamide was obtained in the same manner as in Example 285 as a pale yellow solid.

¹H-NMR(DMSO-d₆): δ 2.41 (3H, s), 2.99 (2H, t, J=7.2 Hz),. 3.31 (2H, q, J=6.9 Hz), 5.55 (1H, t, J=5.6 Hz), 6.50 (2H, d, J=8.9 Hz), 7.21 (2H, d, J=8.6 Hz), 7.38-7.41 (4H, m), 7.60 (2H, d, J=7.9 Hz), 7.73 (2H, d, J=8.2 Hz), 9.03 (1H, d, J=2.0 Hz), 9.54 (1H, s)

ESI-MS(m/z): 482(M+H)⁺

### EXAMPLE 288

tert-Butyl 4-{[(4′,6-dimethyl-1,1′-biphenyl-2-yl)carbonyl]amino}phenyl[2-(1,3-thiazol-4-yl)ethyl]carbamate was obtained in the same manner as in Example 284 as a yellow foam.

### EXAMPLE 289

4′,6-Dimethyl-N-(4-{[2-(1,3-thiazol-4-yl)ethyl]amino}phenyl)-1,1′-biphenyl-2-carboxamide was obtained in the same manner as in Example 285 as a yellow solid.

¹H-NMR(DMSO-d₆): δ 2.07 (3H, s), 2.29 (3H, s), 2.97 (2H, t, J=7.2 Hz), 3.28 (2H, q, J=6.9 Hz), 5.45 (1H, t, J=5.6 Hz), 6.45 (2H, d, J-8.9 Hz), 7.08-7.14 (6H, m), 7.28-7.40 (4H, m), 9.02 (1H, d, J=1.6 Hz), 9.54 (1H, s)

ESI-MS(m/z): 428(M+H)⁺

### EXAMPLE 290

tert-Butyl 4-{[(4′,5-dimethyl-1,1′-biphenyl-2-yl)carbonyl]amino}phenyl[2-(1,3-thiazol-4-yl)ethyl]carbamate was obtained in the same manner as in Example 284 as a yellow foam.

### EXAMPLE 291

4′,5-Dimethyl-N-(4-{[2-(1,3-thiazol-4-yl)ethyl]amino}phenyl)-1,1′-biphenyl-2-carboxamide was obtained in the same manner as in Example 285 as a yellow solid.

¹H-NMR(DMSO-d₆): δ 2.29 (3H, s), 2.38 (3H, s), 2.99 (2H, t, J=7.2 Hz), 3.31 (2H, q, J=6.9 Hz), 5.47 (1H, t, J=5.6 Hz), 6.49 (2H, d, J=8.9 Hz), 7.16 (2H, d, J=7.9 Hz), 7.21-7.23 (4H, m), 7.30-7.41 (4H, m), 9.03 (1H, d, J=2.0 Hz), 9.68 (1H, s)

ESI-MS(m/z): 428(M+H)⁺

### EXAMPLE 292

To a solution of 4′-chloro-4-methyl-1,1′-biphenyl-2-carboxylic acid (167 mg) in toluene (5 ml) were added thionyl chloride (161 mg) and N,N-dimethylformamide (1 drop) and the mixture was stirred at 100° C. for an hour. The mixture was evaporated in vacuo and the residue was dissolved in tetrahydrofuran (5 ml). The acid chloride in tetrahydrofuran was added to a solution of tert-butyl 4-aminophenyl[2-(1,3-thiazol-4-yl)ethyl]carbamate (196 mg) and triethylamine (137 mg) in tetrahydrofuran (10 ml) at ambient temperature and the mixture was stirred at the same temperature for an hour. The mixture was poured into water and extracted with ethyl acetate. The organic layer was washed with brine, dried over magnesium sulfate, and evaporated in vacuo. The residue was purified by column chromatography on silica gel by eluting with ethyl acetate:hexane (2:3) to give tert-butyl 4-{[(4′-chloro-4-methyl-1,1′-biphenyl-2-yl)carbonyl]amino}phenyl[2-(1,3-thiazol-4-yl)ethyl]carbamate (332 mg) as a yellow tar.

¹H-NMR(CDCl₃): δ 1.40(9H, s), 2.44(3H, s), 3.06(2H, t, J=6.6 Hz), 3.97(2H, t, J=6.6 Hz), 6.94-7.02(4H, m), 7.14(2H, d, J=8.9 Hz), 7.27-7.38(6H, m), 7.62(1H, s), 8.69(1H, s)

### EXAMPLE 293

To a solution of tert-butyl 4-{[(4′-chloro-4-methyl-1,1′-biphenyl-2-yl)carbonyl]amino}phenyl[2-(1,3-thiazol-4-yl)ethyl]carbamate (294 mg) in dichloromethane (10 ml) was added trifluoroacetic acid (0.65 ml). The reaction mixture was stirred for 17 hours, quenched with 10% aqueous potassium carbonate solution, and extracted with dichloromethane. The organic layer was washed with brine, dried over magnesium sulfate, filtered, and concentrated in vacuo. The residue was recrystallized from ethyl acetate-diisopropyl ether to give 4′-chloro-4-methyl-N-(4-{[2-(1,3-thiazol-4-yl)ethyl]amino}phenyl)-1,1′-biphenyl-2-carboxamide (160 mg) as colorless crystals.

¹H-NMR(CDCl₃): δ 2.43(3H, s), 3.11(2H, t, J=6.6 Hz), 3.47(2H, t, J=6.6 Hz), 4.04(1H, br s), 6.52(2H, d, J=8.9 Hz), 6.71(1H, br s), 6.97(2H, d, J=8.9 Hz), 7.02(1H, s), 7.28-7.42(6H, m), 7.60(1H, s), 8.77(1H, s)

ESI-MS(m/z): 448, 450(M+H)⁺

### EXAMPLE 294

tert-Butyl 4-{[(4,4′-dimethyl-1,1′-biphenyl-2-yl)carbonyl]amino}phenyl[2-(1,3-thiazol-4-yl)ethyl]carbamate was obtained in the same manner as in Example 292 as a yellow tar.

¹H-NMR(CDCl₃): δ 1.39(9H, s), 2.39(3H, s), 2.44(3H, s), 3.05(2H, t, J=7.3 Hz), 3.95(2H, t, J=7.3 Hz), 6.91-6.99(4H, m), 7.06(2H, d, J=8.6 Hz), 7.22-7.35(6H, m), 7.70(1H, s), 8.68(1H, s)

### EXAMPLE 295

4,4′-Dimethyl-N-(4-{[2-(1,3-thiazol-4-yl)ethyl]amino}phenyl)-1,1′-biphenyl-2-carboxamide was obtained in the same manner as in Example 293 as pale yellow crystals.

¹H-NMR(CDCl₃): δ 2.38(3H, s), 2.42(3H, s), 3.10(2H, t, J=6.6 Hz), 3.46(2H, t, J=6.6 Hz), 3.99(1H, br s), 6.50(2H, d, J=8.6 Hz), 6.71(1H, br s), 6.91(2H, d, J=8.9 Hz), 7.01(1H, s), 7.20-7.36(6H, m), 7.67(1H, s), 8.77(1H, s)

ESI-MS(m/z): 428(M+H)⁺

### EXAMPLE 296

tert-Butyl 4-{[(4′,5-dimethyl-1,1′-biphenyl-2-yl) carbonyl]amino}phenyl [2-(2-methyl-1,3-thiazol-4-yl) ethyl]carbamate was obtained in the same manner as in Example 282 as a pale yellow oil.

### EXAMPLE 297

4′,5-Dimethyl-N-(4-{[2-(2-methyl-1,3-thiazol-4-yl)ethyl]amino}phenyl)-1,1′-biphenyl-2-carboxamide was obtained in the same manner as in Example 283 as a pale brown foam.

¹H-NMR(DMSO-d₆): δ 2.39 (3H, s), 2.42 (3H, s), 2.69(3H, s), 2.99 (2H, t, J=6.6 Hz), 3.41 (2H, t, J=6.6 Hz), 6.49 (2H, t, J=8.9 Hz), 6.71 (1H, s), 6.77 (1H, s), 6.91 (2H, d, J=8.9 Hz), 7.19-7.26 (4H, m), 7.35 (2H, d, J=7.9), 7.78 (1H, d, J=7.9 Hz)

ESI-MS(m/z): 442(M+H)⁺

### EXAMPLE 298

tert-Butyl 4-{[(4,4′-dimethyl-1,1′-biphenyl-2-yl) carbonyl]amino}phenyl [2-(2-methyl-1,3-thiazol-4-yl)ethyl]carbamate was obtained in the same manner as in Example 282 as a pale yellow oil.

### EXAMPLE 299

4,4′-Dimethyl-N-(4-{[2-(2-methyl-1,3-thiazol-4-yl)ethyl]amino}phenyl)-1,1′-biphenyl-2-carboxamide was obtained in the same manner as in Example 283 as a pale brown foam.

¹H-NMR(DMSO-d₆): δ 2.39 (3H, s), 2.43 (3H, s), 2.70 (3H, s), 3.00 (2H, t, J=6.6 Hz), 3.42 (2H, t, J=6.6 Hz), 6.50 (2H, t, J=8.9 Hz), 6.71 (1H, s), 6.78 (1H, s), 7.21-7.36 (6H, m), 7.67 (1H, s)

ESI-MS(m/z): 442(M+H)⁺

### EXAMPLE 300

tert-Butyl 4-({[6-methoxy-4′-(trifluoromethyl)-1,1′-biphenyl-2-yl]carbonyl}amino) phenyl [2-(2-methyl-1,3-thiazol-4-yl) ethyl]carbamate was obtained in the same manner as in Example 282 as a pale yellow oil.

### EXAMPLE 301

6-Methoxy-N-(4-{[2-(2-methyl-1,3-thiazol-4-yl)ethyl]amino}phenyl)-4′-(trifluoromethyl)-1,1′-biphenyl-2-carboxamide was obtained in the same manner as in Example 283 as a pale brown foam.

¹H-NMR(DMSO-d₆): δ 2.69(3H, s), 2.98(2H, t, J=6.6 Hz), 3.40(2H, t, J=6.6 Hz), 3.79(3H, s), 6.47(2H, d, J=8.7 Hz), 6.57(1H, s), 6.76(1H, s), 6.80(2H, d, J=8.7 Hz), 7.09(1H, dd, J=7.9, 1.3 Hz), 7.38-7.49(2H, m), 7.53(2H, d, J=8.2 Hz), 7.67(2H, d, J=8.2 Hz)

ESI-MS(m/z) : 512(M+H)⁺

### EXAMPLE 302

tert-Butyl 2-(2-methyl-1,3-thiazol-4-yl)ethyl[4-({[4-methyl-4′-(trifluoromethyl)-1,1′-biphenyl-2-yl]carbonyl}amino)phenyl]carbamate was obtained in the same manner as in Example 282 as a pale yellow oil.

### EXAMPLE 303

4-Methyl-N-(4-{[2-(2-methyl-1,3-thiazol-4-yl)ethyl]amino}phenyl)-4′-(trifluoromethyl)-1,1′-biphenyl-2-carboxamide was obtained in the same manner as in Example 283 as a pale brown foam.

¹H-NMR(DMSO-d₆): δ 2.45(3H, s), 2.69(3H, s), 2.99(2H, t, J=6.6 Hz), 3.42(2H, t, J=6.6 Hz), 6.51(2H, d, J=8.9 Hz), 6.71(1H, s), 6.77(1H, s), 6.93(2H, d, J=8.9 Hz), 7.29-7.37(2H, m), 7.56-7.68(5H, m)

ESI-MS(m/z): 496(M+H)⁺

### EXAMPLE 304

tert-Butyl 4-{[(4′-chloro-5-methyl-1,1′-biphenyl-2-yl)carbonyl]amino}phenyl [2-(2-methyl-1,3-thiazol-4-yl) ethyl]carbamate was obtained in the same manner as in Example 282 as a pale yellow oil.

### EXAMPLE 305

4′-Chloro-5-methyl-N-(4-{[2-(2-methyl-1,3-thiazol-4-yl)ethyl]amino}phenyl)-1,1′-biphenyl-2-carboxamide was obtained in the same manner as in Example 283 as a pale brown foam.

¹H-NMR(DMSO-d₆): δ 2.43(3H, s), 2.70(3H, s), 3.00(2H, t, J=6.6 Hz), 3.42(2H, t, J=6.6 Hz), 6.52(2H, d, J=8.9 Hz), 6.70(1H, s), 6.77(1H, s), 6.97(2H, d, J=8.9 Hz), 7.18-7.40(6H, m), 7.70(1H, d, J=7.6 Hz)

ESI-MS(m/z): 462(M+H)⁺

### EXAMPLE 306

tert-Butyl 4-{[(4′-chloro-4-methyl-1,1′-biphenyl-2-yl)carbonyl]amino}phenyl[2-(2-methyl-1,3-thiazol-4-yl)ethyl]carbamate was obtained in the same manner as in Example 282 as a pale yellow oil.

### EXAMPLE 307

4′-Chloro-4-methyl-N-(4-{[2-(2-methyl-1,3-thiazol-4-yl)ethyl]amino}phenyl)-1,1′-biphenyl-2-carboxamide was obtained in the same manner as in Example 283 as a pale brown foam.

¹H-NMR(DMSO-d₆): δ 2.43(3H, s), 2.69(3H, s), 2.70(2H, t, J=6.6 Hz), 3.42(2H, t, J=6.6 Hz), 6.52(2H, d, J=8.9 Hz), 6.71(1H, s), 6.77(1H, s), 6.96(2H, d, J=8.9 Hz), 7.25-7.48(6H, m), 7.60(1H, d, J=0.7 Hz)

ESI-MS(m/z): 462(M+H)⁺

Preparation 149

To a solution of tert-butyl 4-(2-hydroxyethyl)-1,3-thiazol-2-ylcarbamate (4.36 g) in tetrahydrofuran (90 ml) was added potassium tert-butoxide (2.00 g), and the mixture was stirred at ambient temperature for an hour. 1-Fluoro-4-nitrobenzene (3.02 g) in tetrahydrofuran (10 ml) was added and heated to 75° C. for 24 hours. The reaction mixture was cooled to ambient temperature, poured into water and extracted with ethyl acetate. The organic layer was washed with brine, dried over magnesium sulfate, filtered, and concentrated in vacuo. The residue was purified by column chromatography on silica gel by eluting with hexane:ethyl acetate (4:1→2:1) to give tert-butyl 4-[2-(4-nitrophenoxy)ethyl]-1,3-thiazol-2-ylcarbamate (4.75 g) as a yellow solid.

¹H-NMR(CDCl₃): δ 1.54(9H, s), 3.21(2H, t, J=6.6 Hz), 4.33(2H, t, J=6.6 Hz), 6.63(1H, s), 6.92(2H, d, J=9.2 Hz), 8.16(2H, d, J=9.2 Hz), 9.57(1H, br s)

Preparation 150

A solution of tert-butyl 4-[2-(4-nitrophenoxy)ethyl]-1,3-thiazol-2-ylcarbamate (2.00 g) in methanol (80 ml) and tetrahydrofuran (30 ml) was hydrogenated over 10% palladium on carbon (0.8 g) at ambient temperature under atmospheric pressure of hydrogen for an hour. The reaction mixture was filtered with a pad of celite, and the filtrate was concentrated in vacuo. The residue was purified by column chromatography on silica gel by eluting with hexane:ethyl acetate (1:1) to give tert-butyl 4-[2-(4-aminophenoxy)ethyl]-1,3-thiazol-2-ylcarbamate (1.43 g) as a yellow oil.

¹H-NMR(CDCl₃): δ 1.53(9H, s), 3.12(2H, t, J=6.9 Hz), 4.16(2H, t, J=6.9 Hz), 6.60(2H, d, J=8.9 Hz), 6.61(1H, s), 6.73(2H, d, J=8.9 Hz)

### EXAMPLE 308

To a solution of tert-butyl 4-[2-(4-aminophenoxy)ethyl]-1,3-thiazol-2-ylcarbamate (329 m g), 5-methyl-4′-(trifluoromethyl)-1,1′-biphenyl-2-carboxylic acid (2.212 g) and 1-hydroxybenzotriazole (1.123 g) in N,N-dimethylformamide (15 ml) was added 1-[3-(dimethylamino)propyl]-3-ethylcarbodiimide hydrochloride (WSC.HCl) (174 mg), followed by triethylamine (0.16 ml) at ambient temperature. The reaction mixture was stirred for 12 hours and concentrated in vacuo. The residue was dissolved in ethyl acetate and water, and extracted with ethyl acetate. The organic layer was washed with water and brine, dried over magnesium sulfate, filtered, and concentrated in vacuo. The residue was purified by column chromatography on silica gel by eluting with hexane:ethyl acetate (1:1) to give tert-butyl 4-{2-[4-({[5-methyl-4′-(trifluoromethyl)-1,1′-biphenyl-2-yl]carbonyl}amino)phenoxy]ethyl}-1,3-thiazol-2-ylcarbamate (387 mg) as a pale yellow foam.

### EXAMPLE 309

To a solution of tert-butyl 4-{2-[4-({[5-methyl-4′-(trifluoromethyl)-1,1′-biphenyl-2-yl]carbonyl}amino)phenoxy]-ethyl}-1,3-thiazol-2-ylcarbamate (476 mg) in dichloromethane (30 ml) was added trifluoroacetic acid (1.2 ml). The reaction mixture was stirred for 15 hours, quenched with 10% aqueous potassium carbonate solution, and extracted with dichloromethane. The organic layer was washed with brine, dried over magnesium sulfate, filtered, and concentrated in vacuo. The residue was recrystallized from ethyl acetate-diisopropyl ether to give N-{4-[2-(2-amino-1,3-thiazol-4-yl) ethoxy]phenyl}-5-methyl-4′-(trifluoromethyl)-1,1′-biphenyl-2-carboxamide (320 mg) as white crystals.

¹H-NMR(DMSO-d₆): δ 2.42(3H, s), 2.94(2H, t, J=6.3 Hz), 4.15(2H, t, J=6.3 Hz), 6.56(1H, s), 6.86(2H, d, J=8.9 Hz), 7.31-7.42(4H, m), 7.50(1H, d, J=7.6 Hz), 7.60(2H, d, J=7.6 Hz), 7.72(2H, d, J=8.2 Hz), 10.10(1H, s)

ESI-MS(m/z): 498(M+H)⁺

### EXAMPLE 310

tert-Butyl 4-{2-[4-({[4-methyl-4′-(trifluoromethyl)-1,1′-biphenyl-2-yl]carbonyl}amino)phenoxy]ethyl}-1,3-thiazol-2-ylcarbamate was obtained in the same manner as in Example 308 as a pale yellow foam.

### EXAMPLE 311

N-{4-[2-(2-Amino-1,3-thiazol-4-yl)ethoxy]phenyl}-4-methyl-4′-(trifluoromethyl)-1,1′-biphenyl-2-carboxamide was obtained in the same manner as in Example 309 as white crystals.

¹H-NMR(DMSO-d₆): δ 2.42(3H, s), 2.83(2H, t, J=6.9 Hz), 4.14(2H, t, J=6.9 Hz), 6.24(1H, s), 6.84(2H, d, J=9.2 Hz), 6.85(2H, s), 7.39-7.43(5H, m), 7.59(2H, d, J=7.9 Hz), 7.73(2H, d, J=8.2 Hz), 10.17(1H, s)

ESI-MS (m/z): 498 (M+H)⁺

### EXAMPLE 312

tert-Butyl 4-[2-(4-{[(4′,5-dimethyl-1,1′-biphenyl-2-yl)carbonyl]amino}phenoxy)ethyl]-1,3-thiazol-2-ylcarbamate was obtained in the same manner as in Example 308 as a pale yellow foam.

### EXAMPLE 313

N-{4-[2-(2-Amino-1,3-thiazol-4-yl)ethoxy]phenyl}-4′,5-dimethyl-1,1′-biphenyl-2-carboxamide was obtained in the same manner as in Example 309 as white crystals.

¹H-NMR(DMSO-d₆): δ 2.28(3H, s), 2.39(3H, s), 2.82(2H, t, J=6.7 Hz), 4.13(2H, t, J=6.7 Hz), 6.24(1H, s), 6.82(2H, s), 6.83(2H, d, J=8.9 Hz), 7.30(2H, d, J=7.9 Hz), 7.38-7.42(3H, m), 9.95(1H, s)

ESI-MS(m/z): 444(M+H)⁺

### EXAMPLE 314

tert-Butyl 4-[2-(4-{[(4,4′-dimethyl-1,1′-biphenyl-2-yl)carbonyl]amino}phenoxy)ethyl]-1,3-thiazol-2-ylcarbamate was obtained in the same manner as in Example 308 as a pale yellow foam.

### EXAMPLE 315

N-{4-[2-(2-Amino-1,3-thiazol-4-yl)ethoxy]phenyl}-4,4′-dimethyl-1,1′-biphenyl-2-carboxamide was obtained in the same manner as in Example 309 as white crystals.

¹H-NMR(DMSO-d₆): δ 2.28(3H, s), 2.39(3H, s), 2.83(2H, t, J=6.9 Hz), 4.14(2H, t, J=6.9 Hz), 6.24(1H, s), 6.83(2H, d, J=8.9 Hz), 6.85(2H, s), 7.14(2H, d, J=7.9 Hz), 7.28-7.32(5H, m), 7.42(2H, d, J=8.9 Hz), 10.04(1H, s)

ESI-MS(m/z): 444(M+H)⁺

### EXAMPLE 316

tert-Butyl 4-[2-(4-{[(4′-chloro-5-methyl-1,1′-biphenyl-2-yl) carbonyl]amino}phenoxy)ethyl]-1,3-thiazol-2-ylcarbamate was obtained in the same manner as in Example 308 as a pale yellow foam.

### EXAMPLE 317

N-{4-[2-(2-Amino-1,3-thiazol-4-yl)ethoxy]phenyl)-4′-chloro-5-methyl-1,1′-biphenyl-2-carboxamide was obtained in the same manner as in Example 309 as pale brown crystals.

¹H-NMR(DMSO-d₆): δ 2.40(3H, s), 2.83(2H, t, J=6.9 Hz), 4.13(2H, t, J=6.9 Hz),6.24(1H, s), 6.83(2H, d, J=8.9 Hz), 6.85(2H, s), 7.26-7.30(2H, m), 7.38-7.47(7H, m), 10.01(1H, s)

ESI-MS(m/z): 464(M+H)⁺

### EXAMPLE 318

tert-Butyl 4-[2-(4-{[(4′-chloro-4-methyl-1,1′-biphenyl-2-yl)carbonyl]amino}phenoxy)ethyl]-1,3-thiazol-2-ylcarbamate was obtained in the same manner as in Example 308 as a pale yellow foam.

### EXAMPLE 319

N-{4-[2-(2-Amino-1,3-thiazol-4-yl) ethoxy]phenyl}-4′-chloro-4-methyl-1,1′-biphenyl-2-carboxamide was obtained in the same manner as in Example 309 as pale yellow crystals.

¹H-NMR(DMSO-d₆): δ 2.40(3H, s), 2.83(2H, t, J=6.9 Hz), 4.14(2H, t, J=6.9 Hz), 6.25(1H, s), 6.83(2H, d, J=8.9 Hz), 6.84(2H, s), 7.31-7.43(9H, m), 10.09(1H, s)

ESI-MS(m/z): 464(M+H)⁺

### EXAMPLE 320

To a solution of 4,4′-dimethyl-1,1′-biphenyl-2-carboxylic acid (123 mg) in toluene (4 ml) was added thionyl chloride (0.08 ml) and N,N-dimethylformamide (1 drop) and the mixture was stirred at 80° C. for an hour. The mixture was evaporated in vacuo and the residue was dissolved in tetrahydrofuran (2 ml). The acid chloride in tetrahydrofuran was added to a solution of tert-butyl 6-{2-[(5-amino-2-pyridinyl)oxy]ethyl}-2-pyridinylcarbamate (150 mg) and triethylamine (0;127 ml) in tetrahydrofuran (3 ml) at ambient temperature and the mixture was stirred at the same temperature for an hour. The mixture was poured into water and extracted with ethyl acetate. The organic layer was washed with brine, dried over magnesium sulfate, and evaporated in vacuo to give tert-butyl 6-(2-[(5-{[(4,4′-dimethyl-1,1′-biphenyl-2-yl)carbonyl]amino}-2-pyridinyl)oxy]ethyl}-2-pyridinylcarbamate (244 mg) as a pale yellow foam.

¹H-NMR(CDCl₃): δ 1.51(9H, s), 2.40(3H, s), 2.44(3H, s), 3.09(2H, t, J=6.8 Hz), 4.56(1H, t, J=7.0 Hz), 6.61(1H, d, J=10.0 Hz), 6.77(1H, br s), 6.87(1H, d, J=7.0 Hz), 7.16-7.35(7H, m), 7.55(1H, d, J=8.1 Hz), 7.59-7.64(2H, m), 7.69(1H, br s), 7.74(1H, d, J=8.1 Hz)

ESI-MS(m/z): 539(M+H)⁺

### EXAMPLE 321

To a solution of tert-butyl 6-{2-[(5-{[(4,4′-dimethyl-1,1′-biphenyl-2-yl)carbonyl]amino}-2-pyridinyl)oxy]ethyl}-2-pyridinylcarbamate (244 mg) in dichloromethane (10 ml) was added trifluoroacetic acid (0.873 ml). The reaction mixture was stirred for 14 hours, quenched with 10% aqueous potassium carbonate solution, and extracted with dichloromethane. The organic layer was washed with brine, dried over magnesium sulfate, filtered, and concentrated in vacuo. The residue was recrystallized from tetrahydrofuran-diisopropyl ether to give N-{6-[2-(6-amino-2-pyridinyl)ethoxy]-3-pyridinyl}-4,4′-dimethyl-1,1′-biphenyl-2-carboxamide (110 mg) as a white powder.

¹H-NMR(DMSO-d₆): δ 2.40(3H, s), 2.44(3H, s), 3.04(2H, t, J=7.0 Hz), 4.40(2H, br s), 4.55(2H, t, J=6.8 Hz), 6.34(1H, d, J=7.8 Hz), 6.58(1H, d, J=7.3 Hz), 6.63(1H, d, J=8.6 Hz), 6.78(1H, s), 7.22-7.37(7H, m), 7.59-7.69(3H, m)

ESI-MS(m/z): 439(M+H)⁺

### EXAMPLE 322

tert-Butyl 6-(2-{[5-({[4-methyl-4′-(trifluoromethyl)-1,1′-biphenyl-2-yl]carbonyl}amino)-2-pyridinyl]oxy}ethyl)-2-pyridinylcarbamate was obtained in the same manner as in Example 320 as a pale yellow foam.

¹H-NMR(CDCl₃): δ 1.51(9H, s), 2.46(3H, s), 3.08(2H, t, J=6.8 Hz), 4.56(2H, t, J=7.0 Hz); 6.62(1H, d, J=8.9 Hz), 6.81(1H, s); 6.87(1H, d, J=7.3 Hz), 7.30-7.37(3H, m), 7.45-7.63(5H,′m), 7.67(2H, d, J=8.4 Hz), 7.75(1H, d, J=8.4 Hz), 7.81(1H, 7.81, J=2.7 Hz)

ESI-MS(m/z): 593(M+H)⁺

### EXAMPLE 323

N-{6-[2-(6-Amino-2-pyridinyl)ethoxy]-3-pyridinyl}-4-methyl-4′-(trifluoromethyl)-1,1′-biphenyl-2-carboxamide was obtained in the same manner as in Example 321 as a pale yellow powder.

¹H-NMR(CDCl₃): δ 2.46(3H, s), 3.05(2H, t, J=6.8 Hz), 4.45(2H, br s), 4.57(2H, t, J=7.0 Hz), 6.35(1H, d, J=8.1 Hz), 6.58(1H, d, J=7.6 Hz), 6.65(1H, d, J=8.6 Hz), 6.78(1H, br s), 7.30-7.40(3H, m), 7.55-7.61(4H, m), 7.68(2H, d, J=7.8 Hz), 7.81(1H, d, J=2.4 Hz)

ESI-MS(m/z): 493(M+H)⁺

### EXAMPLE 324

tert-Butyl 6-{2-[(5-{[(4′-chloro-4-methyl-1,1 ′-biphenyl-2-yl)carbonyl]amino}-2-pyridinyl)oxy]ethyl}-2-pyridinylcarbamate was obtained in the same manner as in Example 320 as a pale yellow foam.

¹H-NMR(CDCl₃): δ 1.51(9H, s), 2.45(3H, s), 3.09(2H, t, J=7.0 Hz), 4.58(2H, t, J=6.8 Hz),6.64(1H, d, J=8.9 Hz), 6.76(1H, s), 6.88(1H, d, J=7.3 Hz), 7.15-7.37(4H, m), 7.39(4H, s), 7.54-7.68(2H, m), 7.72-7.77(2H, m)

ESI-MS(m/z): 560(M+H)⁺

### EXAMPLE 325

N-{6-[2-(6-Amino-2-pyridinyl)ethoxy]-3-pyridinyl}-4′-chloro-4-methyl-1,1′-biphenyl-2-carboxamide was obtained in the same manner as in Example 321 as a pale yellow powder.

¹H-NMR(CDCl₃): δ 2.45(3H, s), 3.05(2H, t, J=6.8 Hz), 4.42(2H, br s), 4.57(2H, t, J=6.8 Hz), 6.35(1H, d, J=8.4 Hz), 6.59(1H, d, J=7.0 Hz), 6.66(1H, d, J=8.9 Hz), 6.77(1H, br s), 7.30-7.40(7H, m), 7.63(1H, d, J=2.2 Hz), 7.66(1H, d, J=3.0 Hz), 7.77(1H, d, J=3.0 Hz)

ESI-MS(m/z): 459(M+H)⁺

### EXAMPLE 326

To a solution of 4-methyl-4′-(trifluoromethyl)-1,1′-biphenyl-2-carboxylic acid (117 mg) in toluene(4 ml) was added thionyl chloride (0.06 ml) and N,N-dimethylformamide (1 drop) and the mixture was stirred at 80° C. for an hour. The mixture was evaporated in vacuo and the residue was dissolved in tetrahydrofuran (2 ml). The acid chloride in tetrahydrofuran was added to a solution of tert-butyl 5-amino-2-pyridinyl(2-{6-[(tert-butoxycarbonyl)amino]-2-pyridinyl}ethyl)carbamate (150 mg) and triethylamine (0.097 ml) in tetrahydrofuran (3 ml) at ambient temperature and the mixture was stirred at the same temperature for an hour. The mixture was poured into water and extracted with ethyl acetate. The organic layer was washed with brine, dried over magnesium sulfate, and evaporated in vacuo to give tert-butyl (2-{6-[(tert-butoxycarbonyl)amino]-2-pyridinyl}ethyl)[5-({[4-methyl-4′-(trifluoromethyl)-1,1′-biphenyl-2-yl]carbonyl}amino)-2-pyridinyl]carbamate (241 mg) as a pale yellow foam.

¹H-NMR(CDCl₃):δ 1.44(9H, s), 1.51(9H, s), 2.47(3H, s), 2.93(2H, t, J=7.6 Hz), 4.19(2H, t, J=7.6 Hz), 6.78(1H, d, J=6.5 Hz), 7.02(1H, s), 7.16(1H, s), 7.23-7.71(11H, m), 8.11(1H, d, J=2.7 Hz)

ESI-MS(m/z): 692(M+H)⁺

### EXAMPLE 327

To a solution of tert-butyl 2-{6-[(tert-butoxycarbonyl)amino]-2-pyridinyl}ethyl)[5-({[4-methyl-4′-(trifluoromethyl)-1,1′-biphenyl-2-yl]carbonyl}amino)-2-pyridinyl]carbamate (241 mg) in dichloromethane (15 ml) was added trifluoroacetic acid (0.671 ml). The reaction mixture was stirred for 14 hours, quenched with 10% aqueous potassium carbonate solution, and extracted with dichloromethane. The organic layer was washed with brine, dried over magnesium sulfate, filtered, and concentrated in vacuo. The residue was recrystallized from ethyl acetate-diisopropyl ether to give N-(6-{[2-(6-amino-2-pyridinyl)ethyl]amino}-3-pyridinyl)-4-methyl-4′-(trifluoromethyl)-1,1′-biphenyl-2-carboxamide (127 mg) as pale gray powder.

¹H-NMR(CDCl₃): δ 2.46(3H, s), 2.89(2H, t, J=6.5 Hz), 3.60(2H, t, J=6.5 Hz), 4.85(2H, br s), 6.33-6.38(2H, m), 6.52(1H, d, J=7.0 Hz), 6.69(1H, br s), 7.29-7.43(4H, m), 7.56-7.60(3H, m), 7.66-7.71(3H, m)

ESI-MS(m/z): 492(M+H)⁺

### EXAMPLE 328

tert-Butyl (2-{6-[(tert-butoxycarbonyl)amino]-2-pyridinyl}ethyl)[5-({[4′-chloro-4-methyl-1,1′-biphenyl-2-yl]carbonyl}amino)-2-pyridinyl]carbamate was obtained in the same manner as in Example 326 as a pale yellow foam.

¹H-NMR(CDCl₃): δ 1.45(9H, s), 1.51(9H, s), 2.46(3H, s), 2.94(2H, t, J=7.6 Hz), 4.20(2H, t, J=7.3 Hz), 6.79(1H, d, J=6.5 Hz), 6.94(1H, br s), 7.13-7.55(9H, m), 7.64(1H, br s), 7.70(2H, d, J=8.6 Hz), 8.06(1H, d, J=2.4 Hz)

ESI-MS(m/z): 659(M+H)⁺

### EXAMPLE 329

N-(6-{[2-(6-Amino-2-pyridinyl)ethyl]amino}-3-pyridinyl)-4′-chloro-4-methyl-1,1′-biphenyl-2-carboxamide was obtained in the same manner as in Example 327 as a pale yellow powder.

¹H-NMR(CDCl₃): δ 2.43(3H, s), 2.87(2H, t, J=6.5 Hz), 3.59(2H, t, J=6.6 Hz), 4.48(2H, br s), 6.34(2H, d, J=8.4 Hz), 6.51(1H, d, J=7.3 Hz), 6.72(1H, br s), 7.26-7.39(7H, m), 7.50(1H, dd, J=8.9, 2.7 Hz), 7.60(1H, br s), 7.64(1H, d, J=2.7 Hz)

ESI-MS(m/z)): 458(M+H)⁺

Preparation 151

A mixture of 2-(2-methyl-1,3-thiazol-4-yl)ethanol (5.00 g), 1-fluoro-4-nitrobenzene (5.91 g) and potassium tert-butoxide (4.7 g) in tetrahydrofuran (70 ml) was heated to 70° C. for 1.5 hours. The mixture was poured into water and extracted with ethyl acetate. The organic layer was washed with brine, dried over magnesium sulfate, and evaporated in vacuo. The residue was purified by column chromatography on silica gel by eluting with hexane:ethyl acetate (2:1) to give 2-methyl-4-[2-(4-nitrophenoxy)ethyl]-1,3-thiazole (3.57 g) as-a pale yellow oil.

¹H-NMR(CDCl₃): δ 2.70(3H, s), 3.24(2H, t, J=6.5 Hz), 4.39(2H, t, J=6.5 Hz), 6.89(1H, s), 6.95(2H, d, J=9.2 Hz), 8.18(2H, d, J=9.2 Hz)

ESI-MS(m/z): 265(M+H)⁺

Preparation 152

A solution of 2-methyl-4-[2-(4-nitrophenoxy) ethyl]-1,3-thiazole (3.57 g) in methanol (15 ml) was hydrogenated over 10% palladium on carbon (1.79 g, 50% wet) at ambient temperature under atmospheric pressure of hydrogen for 2.0 hours. The reaction mixture was filtered through a short pad of celite, and the filtrate was concentrated in vacuo. The residue was purified by column chromatography on silica gel by eluting with hexane:ethyl acetate (2:1-1:1) to give 4-[2-(2-methyl-1,3-thiazol-4-yl)ethoxy]aniline (3.06 g) as a pale yellow oil.

¹H-NMR(CDCl₃): δ 2.69(3H, s), 3.17(2H, t, J=6.5 Hz), 3.42(2H, br s), 4.21(2H, t, J=6.5 Hz), 6.62(2H, d, J=8.9 Hz), 6.75(2H, d, J=8.9 Hz), 6.87(1H, s)

ESI-MS(m/z): 235(M+H)⁺

### EXAMPLE 330

To a solution of 4,4′-dimethyl-1,1′-biphenyl-2-carboxylic acid (209 mg) in toluene (4 ml) was added thionyl chloride (0.134 ml) and N,N-dimethylformamide (1 drop) and the mixture was stirred at 80° C. for an hour. The mixture was evaporated in vacuo and the residue was dissolved in tetrahydrofuran (2 ml). The acid chloride in tetrahydrofuran was added to a solution of 4-[2-(2-methyl-1,3-thiazol-4-yl)ethoxy]aniline (180 mg) and triethylamine (0.214 ml) in tetrahydrofuran (3 ml) at ambient temperature and the mixture was stirred at the same temperature for an hour. The mixture was poured into water and extracted with ethyl acetate. The organic layer was washed with brine, dried over magnesium sulfate, and evaporated in vacuo. The residue was recrystallized from ethyl acetate and hexane to give 4,4′-dimethyl-N-{4-[2-(2-methyl-1,3-thiazol-4-yl)ethoxy]phenyl}-1,1′-biphenyl-2-carboxamide (172 mg) as a white powder.

¹H-NMR(CDCl₃): δ 2.38(3H, s), 2.43(3H, s), 2.68(3H, s), 3.17(2H, t, J=6.6 Hz), 4.23(2H, t, J=6.6 Hz), 6.75-6.79(3H, m), 6.85(1H, s), 6.99(2H, d, J=8.9 Hz), 7.20-7.36(6H, m), 7.67(1H, s)

ESI-MS(m/z): 443(M+H)⁺

### EXAMPLE 331

4-Methyl-N-{4-[2-(2-methyl-1,3-thiazol-4-yl)ethoxy]phenyl}-4′-(trifluoromethyl)-1,1′-biphenyl-2-carboxamide was obtained in the same manner as in Example 330 as a pale brown powder.

¹H-NMR(CDCl₃): δ 2.46(3H, s), 2.69(3H, s), 3.18(2H, t, J=6.6 Hz), 4.23(2H, t, J=6.6 Hz), 6.75-6.81(3H, m), 6.85(1H, s), 7.03(2H, d, J=9.2 Hz), 7.22(2H, m), 7.56-7.73(5H, m)

ESI-MS(m/z): 497(M+H)⁺

### EXAMPLE 332

4′-Chloro-4-methyl-N-{4-[2-(2-methyl-1,3-thiazol-4-yl)ethoxy]phenyl}-1,1′-biphenyl-2-carboxamide was obtained in the same manner as in Example 330 as a pale brown powder.

¹H-NMR(CDCl₃): δ 2.44(3H, s), 2.69(3H, s), 3.18(2H, t, J=6.6 Hz), 4.24(2H, t, J=6.6 Hz), 6.78-6.86(4H, m), 7.07(2H, d, J=9.2 Hz), 7.29-7.39(6H, m), 7.60(1H, s)

ESI-MS(m/z): 463(M+H)⁺

### EXAMPLE 333

4′, 5-Dimethyl-N-{4-[2-(2-methyl-1,3-thiazol-4-yl)ethoxy]phenyl}-1,1′-biphenyl-2-carboxamide was obtained in the same manner as in Example 330 as a pale brown powder.

¹H-NMR(CDCl₃): δ 2.39(3H, s), 2.43(3H, s), 2.69(3H, s), 3.17(2H, t, J=6.6 Hz), 4.23(2H, t, J=6.6 Hz), 6.74-6.79(3H, m), 6.85(1H, s), 6.99(2H, d, J=9.2 Hz), 7.19-7.36(5H, m), 7.79(1H, d, J=7.9 Hz)

ESI-MS(m/z): 443(M+H)⁺

### EXAMPLE 334

5-Methyl-N-{4-[2-(2-methyl-1,3-thiazol-4-yl)ethoxy]phenyl}-4 ′-(trifluoromethyl)-1,1′-biphenyl-2-carboxamide was obtained in the same manner as in Example 330 as a pale brown powder.

¹H-NMR(CDCl₃): δ 2.45(3H, s), 2.69(3H, s), 3.18(2H, t, J=7.0 Hz), 4.23(2H, t, J=6.5 Hz), 6.75-6.80(3H, m), 6.85(1H, s), 7.03(2H, d, J=9.2 Hz), 7.22(1H, s), 7.31(1H, d, J=7.8 Hz), 7.58(2H, d, J=8.1 Hz), 7.65-7.23(3H, m)

ESI-MS(m/z): 497(M+H)⁺

### EXAMPLE 335

4′-Chloro-5-methyl-N-{4-[2-(2-methyl-1,3-thiazol-4-yl)ethoxy]phenyl}-1,1′-biphenyl-2-carboxamide was obtained in the same manner as in Example 330 as a pale brown powder.

¹H-NMR(CDCl₃): δ 2.44(3H, s), 2.69(3H, s), 3.18(2H, t, J=7.0 Hz), 4.24(2H, t, J=6.8 Hz), 6.78-6.82(4H, m), 6.86(1H, s), 7.07(2H, d, J=8.9 Hz), 7.19(1H, s), 7.28(1H, d, J=8.9 Hz), 7.39(4H, s), 7.47(2H, d, J=7.8 Hz)

ESI-MS(m/z): 463(M+H)⁺

Preparation 153

To a mixture of 3-bromo-2-thiophenecarbaldehyde (2.0 g), 4-(trifluoromethyl)phenylboronic acid (2.58 g) and triethylamine (3.33 g) in N,N-dimethylformamide (40 ml) were added tetrakis(triphenylphosphine) palladium(0) (363 mg) and 2M sodium carbonate aqueous solution (14.7 ml), under nitrogen at ambient temperature. The mixture was heated to 100° C. and stirred for 3 hours. The mixture was poured into water and extracted with ethyl acetate. The separated organic layer was washed with water and brine, dried over magnesium sulfate, and evaporated in vacuo. The residue was purified by column chromatography on silica gel and crystallized from hexane to give 3-[4-(trifluoromethyl)phenyl]-2-thiophenecarbaldehyde (1.69 g) as white crystals. The second crop (0.67 g) was obtained from the filtrate.

¹H-NMR(DMSO-d₆): δ 7.50(1H, d, J=5.0 Hz), 7.88(4H, s), 8.23(1H, dd, J=5.0 and 1.2 Hz), 9.83(1H, d, J=1.2 Hz)

APCI-MS (m/z): 257 (M+H)⁺

Preparation 154

To a solution of 3-[4-(trifluoromethyl)phenyl]-2-thiophenecarbaldehyde (690 mg) in acetone (7 ml) and tert-butanol (7 ml) were added 2-pentene (947 mg) and sodium dihydrogenphosphate dihydrate (420 mg) at ambient temperature. To this mixture was added portionwise sodium chlorite (730 mg) at ambient temperature and the mixture was stirred at the same temperature for 4 hours. The mixture was poured into a mixture of ethyl acetate and water and adjusted to pH 2 by addition of 6N hydrochloric acid. The separated organic layer was washed with water and brine, dried over magnesium sulfate, and evaporated in vacuo. The residue was triturated with hexane:diisopropyl ether (1:1), collected by filtration, washed with hexane, and dried in vacuo to give 3-[4-(trifluoromethyl)phenyl]-2-thiophenecarboxylic acid (670 mg) as white crystals.

¹H-NMR(DMSO-d₆): δ 7.24(1H, d, J=5.1 Hz), 7.68(2H, d, J=8.4 Hz), 7.76(2H, d, J=8.4 Hz), 8.23(1H, d, J=8.1 Hz)

APCI-MS(m/z): 273(M+H)⁺

### EXAMPLE 336

To a suspension of N-(4-aminobenzyl)-2-pyridinecarboxamide (227 mg), 3-[4-(trifluoromethyl)phenyl]-2-thiophenecarboxylic acid (212 mg) and 1-hydroxybenzotriazole hydrate (158 mg) in dichloromethane (40 ml) was added 1-[3-(dimethylamino)propyl]-3-ethylcarbodiimide (155 mg) at ambient temperature. The resulting solution was stirred at same temperature for 20 hours and poured into water. The separated organic layer was washed with brine, dried over magnesium sulfate and evaporated in vacuo. The residue was purified by column chromatography on silica gel by eluting with hexane:ethyl acetate (1:2) to give N-{4-[({3-[4-(trifluoromethyl}phenyl]2-thienyl}carbonyl]amino) benzyl]-2-pyridinecarboxamide (198 mg) as a white solid.

¹H-NMR(DMSO-d₆): δ 4.44(2H, d, J=6.4 Hz), 7.26(2H, d, J=8.4 Hz), 7.38(1H, d, J=5.0 Hz), 7.46(2H, d, J=8.4 Hz), 7.6-8.1(8H, m), 8.65(1H, d, J=4.8 Hz), 9.30(1H, d, J=6.3 Hz), 10.23(1H, s)

APCI-MS(m/z): 482(M+H)⁺

Preparation 155

To a solution of diisopropylamine (6.07 g) in tetrahydrofuran was added dropwise n-butyllithium (1.57M hexane solution) (38.2 ml) at −60° C. under nitrogen and the mixture was stirred at the same temperature for 30 minutes. To this lithium diisopropylamide solution was added dropwise a solution of 2-(2,5-dimethyl-1H-pyrrol-1-yl)-6-methylpyridine (9.31 g) in tetrahydrofuran (40 ml) at −60° C. under nitrogen and the mixture was stirred at the same temperature for 1.5 hours. To the mixture was added a solution of oxirane (ca. 5M toluene solution) (20 ml) at −60° C. and the resultant yellow solution was warmed gradually to ambient temperature. The mixture was poured into ethyl acetate and water and the separated organic layer was washed with water, brine, dried over magnesium sulfate, and evaporated in vacuo. The residue was purified by column chromatography on silica gel eluting with hexane:ethyl acetate (1:3) to give 3-[6-(2,5-dimethyl-1H-pyrrol-1-yl)-2-pyridinyl]-1-propanol (8.66 g) as a yellow oil.

¹H-NMR(DMSO-d₆): δ 1.8-1.95(2H, m), 2.06(6H, s), 2.80(2H, t, J=7.9 Hz), 3.45(2H, td, J=6.5 and 5.2 Hz), 4.49(1H, t, J=5.2 Hz), 5.79(2H, s), 7.18(1H, d, J=7.7 Hz), 7.29(1H, d, J=7.7 Hz), 7.87(1H, dd, J=7.7 and 7.7 Hz)

ESI-MS(m/z): 253(M+Na)⁺, 231(M+H)⁺

Preparation 156

To a suspension of potassium tert-butoxide (4.15 g) in tetrahydrofuran (100 ml) were added a solution of 3-[6-(2,5-dimethyl-1H-pyrrol-1-yl)-2-pyridinyl]-1-propanol (8.51 g) in tetrahydrofuran (20 ml), followed by addition of 1-fluoro-4-nitrobenzene (5.21 g) at ambient temperature and the mixture was refluxed for 5 hours under nitrogen. The mixture was poured into ethyl acetate and water and the separated organic layer was washed with water, brine, dried over magnesium sulfate, and evaporated in vacuo. The residue was purified by column chromatography on silica gel eluting with hexane:ethyl acetate (2:1) to give 2-(2,5-dimethyl-1H-pyrrol-1-yl)-6-[3-(4-nitrophenoxy)propyl]pyridine (7.70 g) as a yellow oil.

¹H-NMR(DMSO-d₆): δ 2.04(6H, s), 2.15-2.3(2H, m), 2.96(2H, t, J=7.2 Hz), 4.17(2H, t, J=7.0 Hz), 5.79(2H, s), 7.05-7.15(2H, m), 7.22(1H, d, J=7.9 Hz), 7.34(1H, d, J=7.4 Hz), 7.89(1H, dd, J=7.9 and 7.4 Hz), 8.15-8.25(2H, m)

ESI-MS(m/z): 374(M+Na)⁺, 352(M+H)⁺

Preparation 157

To a solution of 2-(2,5-dimethyl-1H-pyrrol-1-yl)-6-[3-(4-nitrophenoxy)propyl]pyridine (7.52 g) in tetrahydrofuran (60 ml) and methanol (60 ml) was added hydrogenated over 10% palladium on carbon (3 g, 50% wet) at ambient temperature under atmospheric pressure of hydrogen for 4 hours. The reaction mixture was filtered through a short pad of celite, and the filtrate was concentrated in vacuo. The residue was purified by column chromatography on silica gel by eluting with hexane:ethyl acetate (1:1) to give 4-{3-[6-(2,5-dimethyl-1H-pyrrol-1-yl)-2-pyridinyl]propoxy}aniline (5.44 g) as a yellow oil.

¹H-NMR(DMSO-d₆): δ 2.04(6H, s), 2.0-2.15(2H, m), 2.91(2H, t, J=7.9 Hz), 3.84(2H, t, J=6.9 Hz), 4.56(2H, br s), 5.79(2H, s), 6.45-6.65(4H, m), 7.20(1H, d, J=7.8 Hz), 7.33(1H, d, J=7.1 Hz), 7.88(1H, dd, J-7.8 and 7.1 Hz)

ESI-MS(m/z): 344(M+Na)⁺, 322(M+H)⁺

### EXAMPLE 337

To a solution of 4-{3-[6-(2,5-dimethyl-1H-pyrrol-1-yl)-2-pyridinyl]propoxy}aniline (642 mg), 5-methyl-4′-(trifluoromethyl)-1,1′-biphenyl-2-carboxylic acid (560 mg) and 1-hydroxybenzotriazole hydrate (336 mg) in N,N-dimethylformamide (20 ml) was added 1-[3-(dimethylamino)propyl]-3-ethylcarbodiimide (341 mg) at ambient temperature and the mixture was stirred for 16 hours at the same temperature. The mixture was poured into ethyl acetate and water and the separated organic layer was washed with water, brine, dried over magnesium sulfate, and evaporated in vacuo. The residue was purified by column chromatography on silica gel eluting with hexane:ethyl acetate (1:3) to give N-(4-{3-[6-(2,5-dimethyl-1H-pyrrol-1-yl)-2-pyridinyl]propoxy}phenyl)-5-methyl-4′-(trifluoromethyl)-1,1′-biphenyl-2-carboxamide (950 mg) as a brown powder.

¹H-NMR(DMSO-d₆): δ 2.04(6H, s), 2.1-2.25(2H, m), 2.42(3H, s), 2.92(2H, t, J=7.8 Hz), 3.96(2H, t, J=6.3 Hz), 5.78(2H, s), 6.81(2H, d, J=9.0 Hz), 7.21(1H, d, J=7.4 Hz), 7.35-7.5(6H, m), 7.52(1H, d, J=7.6 Hz), 7.61(2H, d, J=8.4 Hz), 7.74(2H, d, J=8.4 Hz), 7.88(1H, dd, J=7.6 and 7.4 Hz), 10.07(1H, s)

ESI-MS(m/z): 606(M+Na)⁺, 584(M+H)⁺

### EXAMPLE 338

To a suspension of N-(4-{3-[6-(2,5-dimethyl-1H-pyrrol-1-yl)-2-pyridinyl]propoxy}phenyl)-5-methyl-4′-(trifluoromethyl)-1,1′-biphenyl-2-carboxamide (920 mg) in a mixture of ethanol (20 ml) and water (5 ml) were added hydroxylamine hydrochloride (1.1 g) and triethylamine (319 mg) at ambient temperature. The mixture was refluxed for 6 hours and evaporated to dryness. The residue was extracted from ethyl acetate and the organic layer was washed with brine, dried over magnesium sulfate, and evaporated in vacuo. The residue was purified by column chromatography on silica gel eluting with ethyl acetate to give N-{4-[3-(6-amino-2-pyridinyl)propoxy]phenyl}-5-methyl-4′-(trifluoromethyl)-1,1′-biphenyl-2-carboxamide (497 mg) as white crystals.

¹H-NMR(DMSO-d₆): δ 1.9-2.1(2H, m), 2.42(3H, s), 2.61(2H, t, J=7.1 Hz), 3.92(2H, t, J=6.3 Hz), 5.77(1H, br s), 6.24(1H, d, J=7.7 Hz), 6.34(1H, d, J=7.7 Hz), 6.55(1H, s), 6.83(2H, d, J=9.0 Hz), 7.25(1H, dd, J=7.7 and 7.1 Hz), 7.3-7.45(6H, m), 7.52(1H, d, J=7.6 Hz), 7.61(2H, d, J=8.3 Hz), 7.74(2H, d, J=8.3 Hz), 10.08(1H, s)

ESI-MS(m/z): 528(M+Na)⁺, 506(M+H)⁺

### EXAMPLE 339

5-Methyl-N-[4-(2-pyridinylmethyl)phenyl]-4′-(trifluoromethyl)-1,1′-biphenyl-2-carboxamide was obtained in the same manner as in Example 24.

¹H-NMR(DMSO-d₆): δ 2.42 (3H, s), 4.01 (2H, s), 7.15-7.68 (13H, m), 7.73 (2H, d, J=8.4 Hz), 8.46 (1H, d, J=5.2 Hz), 10.23 (1H, s)

### EXAMPLE 340

N-(4-{2-[6-(Acetylamino)-2-pyridinyl]ethyl}phenyl)-5-methyl-4′-(trifluoromethyl)-1,1′-biphenyl-2-carboxamide was obtained in the same manner as in Example 24.

¹H-NMR(DMSO-d₆): δ 2.09 (3H, s), 2.42(3H, s), 2.90 (4H, s), 6.93 (1H, d, J=7.4 Hz), 7.10 (2H, d , J=8.4 Hz), 7.33-7.67 (8H, m), 7.74 (2H, d, J=8.3 Hz), 7.90 (1H, d, J=8.2 Hz), 10.19 (1H, s), 10.41 (1H, s)

### EXAMPLE 341

A solution of N-(4-{2-[6-(acetylamino)-2-pyridinyl]ethyl}phenyl)-5-methyl-4′-(trifluoromethyl)-1,1′-biphenyl-2-carboxamide (485 mg) and 6N hydrochloric acid (5 ml) in methanol (10 ml) was refluxed under stirring for 4 hours. The resultant mixture was evaporated in vacuo and the residue was dissolved in a mixture of ethyl acetate and water. The mixture was adjusted to pH 9.0 with 20% aqueous potassium carbonate solution and the organic layer was washed with brine and dried over magnesium sulfate. The solvent was evaporated in vacuo and the residue was recrystallized from ethyl acetate and diisopropyl ether to give N-{4-[2-(6-amino-2-pyridinyl)ethyl]phenyl}-5-methyl-4′-(trifluoromethyl)-1,1′-biphenyl-2-carboxamide (320 mg).

¹H-NMR(DMSO-d₆): δ 2.48 (3H, s), 2.68-2.88 (4H, m), 5.79 (2H, s), 6.24 (1H, d, J=8.2 Hz), 6.32 (1H, d, J=7.1 Hz), 7.10 (2H, d, J=8.4 Hz), 7.20-7.43 (5H, m), 7.52 (1H, d, J=7.6 Hz), 7.61 (2H, d, J=8.1 Hz), 7.74 (2H, d, J=8.3 Hz), 10.18 (1H, s)

APCI-MS(m/z): 490(M+H)⁺

### EXAMPLE 342

5-Methyl-N-{4-[2-(4-pyrimidinyl)ethyl]phenyl}-4′-(trifluoromethyl)-1,1′-biphenyl-2-carboxamide was obtained in the same manner as in Example 24.

¹H-NMR(DMSO-d₆): δ 2.42 (3H, s), 2.98-3.06 (4H, m), 7.11 (2H, d, J=8.4 Hz), 7.32-7.54 (6H, m), 7.61 (2H, d, J=8.2 Hz), 7.74 (2H, d, J=8.3 Hz), 8.64 (1H, d, J=5.1 Hz), 9.08 (1H, d, J=1.0 Hz), 10.19 (1H, s)

### EXAMPLE 343

N-(4-{2-[2-(Acetylamino)-4-pyrimidinyl]ethyl}phenyl)-4′-chloro-5-methyl-1,1′-biphenyl-2-carboxamide was obtained in the same manner as in Example 24.

¹H-NMR(DMSO-d₆): δ 2.19 (3H, s),2.40 (3H, s), 2.73-2.77 (2H, m), 2.83-2.87 (2H, m), 6.44 (1H, d, J=5.1 Hz), 6.49 (1H, s), 7.02(1H, d, J=5.1 Hz), 7.12 (2H, d, J=8.3 Hz), 7.27-7.49 (6H, m), 8.47 (1H, d, J=5.0 Hz), 10.12 (1H, s), 10.42 (1H, s)

### EXAMPLE 344

N-{4-[2-(2-Amino-4-pyrimidinyl)ethyl]phenyl}-4′-chloro-5-methyl-1,1′-biphenyl-2-carboxamide was obtained in the same manner as in Example 341.

¹H-NMR(DMSO-d₆): δ 2.40 (3H, s), 2.70-2.77 (2H, m), 2.83-2.90 (2H, m), 6.43-6.49 (3H, m), 7.12 (2H, d, J=8.4 Hz), 7.29 (2H, d, J=9.2 Hz), 7.40-7.49 (5H, m), 8.10 (1H, d, J=5.0 Hz), 10.12 (1H, s)

Preparation 158

A mixture of 2-pyridinylacetic acid dihydrochloride (3.47 g), 1,4-phenylenediamine (4.32 g), 1-hydroxybenzotriazole hydrate (2.84 g) and 1-[3-(dimethylamino)propyl]-3-ethylcarbodiimide hydrochloride (3.21 g) in N,N-dimethylformamide (20 ml) was stirred at ambient temperature for 14 hours. The reaction mixture was poured into a mixture of ethyl acetate and water. The organic layer was washed with 5% aqueous potassium carbonate solution and brine, and dried over magnesium sulfate. The solvent was evaporated in vacuo and the residue was chromatographed on silica gel eluting with ethyl acetate and n-hexane(8:2) and then ethyl acetate. The fractions containing the desired product were collected and concentrated in vacuo and the resulting precipitate was collected by filtration to give N-(4-aminophenyl)-2-(2-pyridinyl)acetamide (506 mg).

¹H-NMR(DMSO-d₆)): δ 3.75 (2H, s), 4.84 (2H, s), 6.47-6.51 (2H, m), 7.22-7.27 (3H, m), 7.38 (1H, d, J=7.8 Hz), 7.72-7.74 (1H, m), 8.48-8.50 (1H, m), 9.80 (1H, s)

### EXAMPLE 345

4′-Chloro-5-methyl-N-{4-[(2-pyridinylacetyl)amino]phenyl}-1,1′-biphenyl-2-carboxamide was obtained in the same manner as in Example 24.

¹H-NMR(DMSO-d₆): δ 2.40 (3H, s), 3.82 (2H, s), 7.25-7.33 (3H, m), 7.40-7.75 (10H, m), 7.73-7.75 (1H, m), 8.49-8.50 (1H, m), 10.13 (1H, s), 10.19 (1H, s)

Preparation 159

4′,5-Dimethyl-1,1′-biphenyl-2-carbonyl chloride was obtained in the same manner as in Preparation 10.

### EXAMPLE 346

4′,5-Dimethyl-N-{4-[3-(2-pyridinyl)propanoyl]phenyl}-1,1′-biphenyl-2-carboxamide was obtained in the same manner as in Example 20.

¹H-NMR(DMSO-d₆): δ 2.27 (3H, s), 2.41 (3H, s), 3.09 (2H, t, J=6.8 Hz), 3.43 (2H, t, J=6.8 Hz), 6.56 (2H, d, J=8.7 Hz), 6.50-7.14 (1H, m), 7.14-7.34 (5H, m), 7.63-7.72 (3H, m), 7.92 (2H, d, J=8.7 Hz), 8.43-8.45 (1H, m), 10.48 (1H, s)

### EXAMPLE 347

N-{4-[1-Hydroxy-3-(2-pyridinyl)propyl]phenyl}-4′,5-dimethyl-1,1′-biphenyl-2-carboxamide was obtained in the same manner as in Example 21.

¹H-NMR(DMSO-d₆): δ 1.90-1.99 (2H, m), 2.28 (3H, s), 2.40 (3H, s), 2.49-2.51 (2H, m), 4.46-4.54 (1H, m), 5.22 (1H, d, J=4.4 Hz), 7.14-7.48 (11H, m), 7.47 (2H, d ,J=8.6 Hz), 7.62-7.70 (1H, m), 8.45 (1H, d, J=4.6 Hz), 10.08 (1H, s)

### EXAMPLE 348

4′,5-Dimethyl-N-{4-[3-(2-pyridinyl)propyl]phenyl}-1,1′-biphenyl-2-carboxamide was obtained in the same manner as in Example 22.

¹H-NMR(DMSO-d₆): δ 1.85-1.97 (2H, m), 2.28 (3H, s), 2.39 (3H, s), 2.50-2.55 (2H, m), 2.68-2.75 (2H, m), 7.07-7.46 (13H, m), 7.63-7.72 (1H, m), 8.47 (1H, d ,J=4.3 Hz), 10.05 (1H, s)

(+)ESI-MS(m/z): 421(M+H), 443(M+Na)⁺

Preparation 160

N-(4-Acetylphenyl)-4′,5-dimethyl-1,1′-biphenyl-2-carboxamide was obtained in the same manner as in Preparation 17.

¹H-NMR(DMSO-d₆): δ 2.27 (3H, s), 2.40 (3H, s), 2.48 (3H, s), 7.15 (2H, d, J=8.0 Hz), 7.27-7.33 (4H, m), 7.48 (1H, d, J=8.2 Hz), 7.67 (2H, d, J=8.7 Hz), 7.88 (2H, d, J=8.7 Hz), 10.49 (1H, s)

### EXAMPLE 349

N-(4-{(2E)-3-[6-(Acetylamino)-2-pyridinyl]-2-propenoyl}phenyl)-4′,5-dimethyl-1,1′-biphenyl-2-carboxamide was obtained in the same manner as in Example 25.

¹H-NMR(DMSO-d₆): δ 2.07 (3H, s), 2.27 (3H,. s), 2.41 (3H, s), 2.99 (2H, t, J=7.2 Hz), 3.41 (2H, t, J=7.2 Hz), 7.01 (1H, d, J=7.3 Hz), 7.15 (2H, d, J=8.1 Hz), 7.22-7.32 (4H, m), 7.47(1H, d, J=8.2 Hz), 7.61-7.63 (3H, m), 7.86-7.94 (3H, m), 10.32 (1H, s), 10.48 (1H, s)

### EXAMPLE 350

A solution of N-(4-{(2E)-3-[6-(acetylamino)-2-pyridinyl]-2-propenoyl }phenyl)-4′,5-dimethyl-1,1′-biphenyl-2-carboxamide (980 mg) in methanol (30 ml) was hydrogenated over 10% palladium on carbon (400 mg) under an atmospheric pressure of hydrogen at ambient temperature under stirring for 7 hours. After removal of the catalyst, the solvent was evaporated in vacuo and the residue was chromatographed on silica gel eluting with ethyl acetate and n-hexane (6:4-7:3). The fractions containing the desired product were collected and evaporated in vacuo to give N-(4-{3-[6-(acetylamino)-2-pyridinyl]propanoyl}phenyl)-4′,5-dimethyl-1,1′-biphenyl-2-carboxamide (600 mg).

¹H-NMR(DMSO-d₆):δ 2.07 (3H, s), 2.27 (3H, s), 2.41 (3H, s), 2.99 (2H, t, J=7.2 Hz), 3.41 (2H, t, J=7.2 Hz), 7.01 (1H, d, J=7.3 Hz), 7.15 (2H, d, J=8.1 Hz), 7.22-7.32 (4H, m), 7.47 (1H, d, J=8.2 Hz), 7.61-7.67 (3H, m), 7.86-7.94 (3H, in), 10.32 (1H, s), 10.48 (1H, s)

### EXAMPLE 351

N-(4-{3-[6-(Acetylamino)-2-pyridinyl]-1-hydroxypropyl}phenyl)-4′,5-dimethyl-1,1′-biphenyl-2-carboxamide was obtained in the same manner as in Example 21.

¹H-NMR(DMSO-d₆): δ 1.87-2.08 (2H, m), 2.10 (3H, s), 2.28 (3H, s), 2.40 (3H, s), 2.58-2.70 (2H, m), 4.50-4.55 (1H, m), 5.19 (1H, d, J=4.3 Hz), 6.92 (1H, d, J=7.3 Hz), 7.08-7.30 (8H, m), 7.41-7.49 (3H, m), 7.59-7.67 (1H, m), 7.87 (1H, d, J=8.2 Hz), 10.09 (1H, s), 10.34 (1H, s)

### EXAMPLE 352

N-(4-{3-[6-(Acetylamino)-2-pyridinyl]propyl}phenyl)-4′,5-dimethyl-1,1′-biphenyl-2-carboxamide was obtained in the same manner as in Example 22.

¹H-NMR(DMSO-d₆): δ 1.83-1.94 (2H, m), 1.99 (3H, s), 2.28 (3H, s),2.40 (3H, s), 2.49-2.67 (4H, m ), 6.94 (1H, d, J=7.3 Hz), 7.08-7.60 (11H, m), 7.64-7.68 (1H, m), 7.90 (1H, d, J=8.2 Hz), 10.07 (1H, s), 10.36 (1H, s)

### EXAMPLE 353

N-{4-[3-(6-Amino-2-pyridinyl)propyl]phenyl}-4′,5-dimethyl-1,1′-biphenyl-2-carboxamide was obtained in the same manner as in Example 28.

¹H-NMR(DMSO-d₆): δ 1.77-1.92 (2H, m), 2.28 (3H, s),2.42 (3H, s), 2.46-2.56 (4H, m),5.75 (2H, s), 6.23 (1H, d, J=8.0 Hz), 6.32 (1H, d, J=7.1 Hz), 7.06-7.34 (10H, m), 7.42 (2H, dd, J=1.6 and 8.3 Hz), 10.05 (1H, s)

### EXAMPLE 354

A mixture of 2-pyridylethanol (560 mg) and potassium tert-butoxide (406 mg) in tetrahydrofuran (30 ml) was stirred at ambient temperature for 30 minutes. N-(4-Fluoro-3-nitrophenyl)-5-methyl-4′-(trifluoromethyl)-1,1′-biphenyl-2-carboxamide (1.26 g) was added to the above mixture and the mixture was refluxed under stirring for 6 hours. The reaction mixture was poured into a mixture of ethyl acetate and water and extracted with ethyl acetate. The extract was washed with brine and dried over magnesium sulfate. The solvent was evaporated in vacuo and the residue was chromatographed on silica gel eluting with ethyl acetate and n-hexane(5:5). The fractions containing the desired product were collected and evaporated in vacuo and the residue was recrystallized from ethyl acetate and diisopropyl ether to give 5-methyl-N-{3-nitro-4-[2-(2-pyridinyl)ethoxy]phenyl}-4′-(trifluoromethyl)-1,1′-biphenyl-2-carboxamide (522 mg).

¹H-NMR(DMSO-d₆): δ 2.43 (3H, s), 3.18 (2H, t, J=6.6 Hz), 4.49 (2H, t, J=6.6 Hz), 7.10-7.39 (5H, m), 7.56-7.77 (7H, m), 8.11 (1H, d, J=2.5 Hz), 8.48-8.51 (1H, m), 10.51 (1H, s)

### EXAMPLE 355

A mixture of 5-methyl-N-{3-nitro-4-[2-(2-pyridinyl)ethoxy]phenyl}-4′-(trifluoromethyl)-1,1′-biphenyl-2-carboxamide (590 mg) in methanol (30 ml) and tetrahydrofuran (30 ml) was hydrogenated over 10% palladium on carbon (250 mg) under an atmospheric pressure of hydrogen at ambient temperature under stirring for 8 hours. After removal of the catalyst, the solvent was evaporated in vacuo and the residue was chromatographed on silica gel eluting with ethyl acetate and n-hexane (8:2). The fractions containing the desired product were collected and evaporated in vacuo to give N-{3-amino-4-[2-(2-pyridinyl)ethoxy]phenyl}-5-methyl-4′-(trifluoromethyl)-1,1′-biphenyl-2-carboxamide (232 mg).

¹H-NMR(DMSO-d₆): δ 2.41 (3H, s), 3.16 (2H, t, J=6.5 Hz), 4.23 (2H, t, J=6.5 Hz), 4.63 (2H, s), 6.60-6.71 (2H, m), 6.95 (1H, d, J=2.2 Hz), 7.21-7.41 (4H, m), 7.47 (1H, d, J=7.6 Hz), 7.60 (2H, d, J=8.1 Hz), 7.68-7.76 (3H, m), 8.49-8.51 (1H, m), 9.91 (1H, s)

### EXAMPLE 356

A solution of N-{3-amino-4-[2-(2-pyridinyl)ethoxy]phenyl}-5-methyl-4′-(trifluoromethyl)-1,1′-biphenyl-2-carboxamide (230 mg) and acetic anhydride (191 mg) in ethyl acetate (20 ml) was refluxed under stirring for 9 hours. The reaction mixture was poured into a mixture of ethyl acetate and water and adjusted to pH 8.0 with 10% aqueous potassium carbonate solution. The organic layer was washed with brine and dried over magnesium sulfate. The solvent was evaporated in vacuo and the residue was chromatographed on silica gel eluting with ethyl acetate and n-hexane(8:2). The fractions containing the desired product were collected and evaporated in vacuo and the residue was recrystallized from ethyl acetate and diisopropyl ether to give N-{3-(acetylamino)-4-[2-(2-pyridinyl)ethoxy]phenyl}-5-methyl-4′-(trifluoromethyl)-1,1′-biphenyl-2-carboxamide (140 mg).

¹H-NMR(DMSO-d₆): δ 2.03 (3H, s), 2.41 (3H, s), 3.20 (2H, t, J=6.6 Hz), 4.33 (2H, t, J=6.6 Hz), 6.97 (1H, d, J=8.9 Hz), 7.23-7.42 (5H, m), 7.50 (1H, d, J=7.6 Hz), 7.60 (2H, d, J=8.2 Hz), 7.70-7.78 (3H, m), 8.06 (1H, s), 8.52 (1H, d, J=4.1 Hz),8.83 (1H, s), 10.16 (1H, s)

Preparation 161

5-Methyl-N-(4-nitrophenyl)-4′-(trifluoromethyl)-1,1′-biphenyl-2-carboxamide was obtained in the same manner as in Preparation 17.

¹H-NMR(DMSO-d₆): δ 2.44 (3H, s), 7.39 (2H, d, J=10.0 Hz), 7.58-7.64 (3H, m), 7.72-7.82 (4H, m), 8.16-8.23 (2H, m), 10.92 (1H, s)

Preparation 162

A solution of 5-methyl-N-(4-nitrophenyl)-4′-(trifluoromethyl)-1,1′-biphenyl-2-carboxamide (800 mg) in methanol (30 ml) was hydrogenated over 10% palladium on carbon (400 mg) under an atmospheric pressure of hydrogen at ambient temperature under stirring for 5 hours. After removal of the catalyst, the solvent was evaporated in vacuo to give N-(4-aminophenyl)-5-methyl-4′-(trifluoromethyl)-1,1′-biphenyl-2-carboxamide (740 mg).

¹H-NMR(DMSO-d₆): δ 2.41 (3H, s), 4.87 (2H, s), 6.45 (2H, d, J=8.5 Hz), 7.15 (2H, d, J=8.5 Hz), 7.21-7.34 (2H, m), 7.48 (1H, d, J=7.6 Hz), 7.61 (2H, d, J=8.2 Hz), 7.74 (2H, d, J=8.2 Hz), 9.78 (1H, s)

### EXAMPLE 357

A mixture of N-(4-aminophenyl)-5-methyl-4′-(trifluoromethyl)-1,1′-biphenyl-2-carboxamide (371 mg), 2-pyridinylacetic acid dihydrochloride (183 mg), 1-hydroxybenzotriazole hydrate (142 mg) and 1-[3-(dimethylamino)propyl]-3-ethylcarbodiimide hydrochloride (163 mg) in N,N-dimethylformamide (15 ml) was stirred at ambient temperature for 14 hours. The reaction mixture was poured into a mixture of ethyl acetate and water. The organic layer was washed with 5% aqueous potassium carbonate solution and brine, and dried over magnesium sulfate. The solvent was evaporated in vacuo and the residue was recrystallized from ethyl acetate and diisopropyl ether to give 5-methyl-N-{4-[(2-pyridinylacetyl)amino]phenyl}-4′-(trifluoromethyl)-1,1′-biphenyl-2-carboxamide (285 mg).

¹H-NMR(DMSO-d₆): δ 2.42 (3H, s), 3.82 (2H, s), 7.24-7.59 (11H, m), 7.63-7.95 (3H, m), 8.50 (1H, d, J=4.5 Hz), 10.20 (2H, s)

(+)ESI-MS(m/z): 490(M+H)⁺, 512(M+Na)⁺

### EXAMPLE 358

N-[4-({[6-(2,5-Dimethyl-1H-pyrrol-1-yl)-2-pyridinyl]acetyl}amino)phenyl]-5-methyl-4′-(trifluoromethyl)-1,1′-biphenyl-2-carboxamide was obtained in the same manner as in Example 357.

¹H-NMR(DMSO-d₆): δ 2.03 (6H, s), 2.42 (3H, s), 3.85 (2H, s), 5.77 (2H, s), 7.27-7.63 (9H, m),7.61 (2H, d, J=8.2 Hz), 7.94 (2H, d, J=8.3 Hz), 7.90-7.98 (1H, m), 10.19 (2H, s)

### EXAMPLE 359

A mixture of N-[4-({[6-(2,5-dimethyl-1H-pyrrol-1-yl)-2-pyridinyl]acetyl}amino)phenyl]-5-methyl-4′-(trifluoromethyl)-1,1′-biphenyl-2-carboxamide (420 mg), hydroxylamine hydrochloride (501 mg) and triethylamine (146 mg) in water (10 ml) and ethanol (20 ml) was refluxed under stirring for 9.5 hours. The reaction mixture was evaporated in vacuo and the residue was dissolved in a mixture of water and ethyl acetate and adjusted to pH 8.0 with 10% aqueous potassium carbonate solution. The organic layer was washed with brine and dried over magnesium sulfate. The solvent was evaporated in vacuo and the residue was chromatographed on silica gel eluting with ethyl acetate and n-hexane (7:3). The fractions containing the desired product were collected and concentrated in vacuo and the resulting precipitate was collected by filtration to give N-(4-{[(6-amino-2-pyridinyl)acetyl]amino}phenyl)-5-methyl-4′-(trifluoromethyl)-1,1′-biphenyl-2-carboxamide (156 mg).

¹H-NMR(DMSO-d₆): δ 2.42 (3H, s), 3.54 (2H, s); 5.89 (2H, s), 6.31 (1H, d, J=8.0 Hz), 6.46 (1H, d , J=7.1 Hz), 7.29-7.36 (3H, m), 7.44 (2H, d, J=9.0 Hz), 7.61 (2H, d, J=8.1 Hz), 7.74 (2H, d, J=8.2 Hz), 10.14 (1H, s), 10.17 (1H, s)

(−)ESI-MS(m/z): 503(M−H)⁻

Preparation 163

N-(4-Aminobenzyl)-2-pyridine carboxamide was obtained in the same manner as in Preparation 14.

¹H-NMR(DMSO-d₆): δ 4.31-4.35 (2H, m), 4.95 (2H, s), 6.50 (2H, d, J=8.3 Hz), 7.00 (2H, d, J=8.3 Hz), 7.57-7.60 (1H, m), 7.97-8.06 (2H, m), 8.61-8.63 (1H, m), 8.98-9.00 (1H, m)

### EXAMPLE 360

N-[4-({[5-Methyl-4′-(trifluoromethyl)-1,1′-biphenyl-2-yl]carbonyl}amino)benzyl]-2-pyridinecarboxamide was obtained in the same manner as in Example 24.

¹H-NMR(DMSO-d₆): δ 2.42 (3H, s), 4.43 (2H, d, J=6.4 Hz), 7.24 (2H, d, J=8.4 Hz), 7.35 (2H, d, J=8.3 Hz), 7.46 (1H, d, J=8.4 Hz), 7.51-7.63 (5H, m), 7.74 (2H, d, J=8.3 Hz), 7.99-8.07 (2H, m), 8.64-8.66 (1H, m), 9.27 (1H, t, J=6.4 Hz), 10.25 (1H, s)

### EXAMPLE 361

A mixture of 2-(4-(trifluoromethyl)phenyl)-1-cyclohexene-1-carboxylic acid (405 mg) and N²-[2-(2-pyridinyl)ethyl]-2,5-pyridinediamine (355 mg), 1-hydroxybenzotriazole hydrate (241 mg) 1-[3-(dimethylamino)propyl]-3-ethylcarbodiimide (244 mg) and 4-(dimethyamino)pyridine (4 mg) in N,N-dimethylformamide (15 ml) was stirred at ambient temperature overnight. The reaction mixture was poured into a mixture of ethyl acetate and water, and the organic layer was washed with brine and dried over magnesium sulfate. The solvent was evaporated in vacuo and the residue was chromatographed on silica gel eluting with ethyl acetate and methanol (96:4). The fractions containing the desired product were collected and evaporated in vacuo and the residue was recrystallized from ethyl acetate and diisopropyl ether to give N-(6-{[2-(2-pyridinyl)ethyl]amino}-3-pyridinyl)-2-[4-(trifluoromethyl)phenyl]-1-cyclohexene-1-carboxamide (497 mg).

¹H-NMR(DMSO-d₆): δ 1.73 (4H, br.s), 2.40 (4H, br.s), 2.95 (2H, t, J=7.3 Hz), 3.41-3.52 (2H, m), 6.23-6.37 (2H, m), 7.08-7.30 (3H, m), 7.50 (2H, d, J=8.1 Hz), 7.63-7.72 (3H, m), 7.86 (1H, d J=2.2 Hz), 8.49 (1H, d, J=4.3 Hz), 8.49 (1H, d, J=4.3 Hz), 9.30 (1H, s)

(+)ESI-MS: 467(M+H)⁺, 489(M+Na)⁺

### EXAMPLE 362

N-{6-[2-(2-Pyridinyl)ethoxy]-3-pyridinyl}-2-[4-(trifluoromethyl)phenyl]-1-cyclohexene-1-carboxamide was obtained in the same manner as in Example 361.

¹H-NMR(DMSO-d₆): δ 1.74 (4H, br.s), 2.41(4H, br.s), 3.14(2H, t, J=6.7 Hz), 6.70(2H, t, J=6.7 Hz), 6.64 (1H, d, J=8.9 Hz), *7.19-7.33 (2H, m), 7.47-7.80 (6H, m), 8.08 (1H, d, J=2.4 Hz), 8.50 (1H, d, J=4.3 Hz), 9.65 (1H, s)

(+)ESI-MS: 468(M+H)⁺, 490(M+Na)⁺

### EXAMPLE 363

N-{4-[2-(2-Pyridinyl)ethoxy]phenyl}-2-[4-(trifluoromethyl)phenyl]-1-cyclohexene-1-carboxamide was obtained in the same manner as in Example 361.

¹H-NMR (DMSO-d₆): δ 1.73 (4H, br. s ) , 2.39 (4H, br. s) , 3.14 (2H, t, J=6.6 Hz), 4.27 (2H, t, J=6.6 Hz), 6.77 (2H, d, J=9.0 Hz), 7.20-7.25 (3H, m), 7.33 (1H, d J=7.7 Hz), 7.49 (2H, d, J=8.2 Hz), 7.61-7.75 (3H, m), 8.49-8.51 (1H, m), 9.49 (1H, s)

(+)ESI-MS: 467(M+H)⁺, 489(M+Na)⁺

### EXAMPLE 364

2-(4-Methylphenyl)-N-(6-{[2-(2-pyridinyl)ethyl]amino}-3-pyridinyl)-1-cyclohexene-1-carboxamide was obtained in the same manner as in Example 361.

¹H-NMR(DMSO-d₆): δ 1.70 (4H, br.s), 2.23 (3H, s), 2.34 (4H, br.s), 2.93 (2H, t, J=7.4 Hz), 3.49-3.56 (2H, m), 6.23-6.38 (2H, m), 7.07 (2H, d, J=8.1 Hz), 7.16-7.34 (5H, m), 7.64-7.72 (1H, m), 7.85 (1H, d, J=2.5 Hz), 8.47-8.51 (1H, m), 9.13 (1H, s)

(+)ESI-MS: 413(M+H)⁺, 435(M+Na)⁺

### EXAMPLE 365

2-(4-Ethylphenyl)-N-(6-{[2-(2-pyridinyl)ethyl]amino}-3-pyridinyl)-1-cyclohexene-1-carboxamide was obtained in the same manner as in Example 361.

¹H-NMR(DMSO-d₆): δ 1.27 (3H, t, J=7.5 Hz), 1.70 (4H, br.s), 2.35 (4H, br.s), 2.53 (2H, q, J=7.5 Hz), 2.94 (2H, t, J=7.4 Hz), 3.46-3.56 (2H, m), 6.13-6.38 (2H, m), 7.10 (2H, d, J=8.1 Hz), 7.17-7.29 (5H, m), 7.63-7.72 (1H, m), 7.82 (1H, d, J=2.4 Hz), 8.47-8.50 (1H, m), 9.08 (1H, s)

### EXAMPLE 366

A mixture of 2-(4-methylphenyl)-1-cyclohexene-1-carboxylic acid (1.08 g) and tert-butyl 4-(4-aminophenyl)-1-piperazinecarboxylate (1.39 g), 1-hydroxybenzotriazole hydrate (803 mg), 1-[3-(dimethylamino)propyl]-3-ethylcarbodiimide (814 mg) and 4-(dimethyamino)pyridine (12 mg) in N,N-dimethylformamide (20 ml) was stirred at ambient temperature overnight. The reaction mixture was poured into a mixture of ethyl acetate and water, and the organic layer was washed with brine and dried over magnesium sulfate. The solvent was evaporated in vacuo and the residue was chromatographed on silica gel eluting with ethyl acetate and n-hexane (5:5). The fractions containing the desired product were collected and evaporated in vacuo to give tert-butyl 4-[4-({[2-(4-methylphenyl)-1-cyclohexen-1-yl]carbonyl}amino)phenyl]-1-piperazinecarboxylate (2.24 g).

¹H-NMR(DMSO-d₆): δ 1.41 (9H, s), 1.71 (4H, br,s), 2.21 (3H, s), 2.34 (4H, br,s), 2.93-2.98 (4H, m), 3.39-3.44 (4H, m), 6.79 (2H, d, J=9.0 Hz), 7.04 (2H. d, J=8.0 Hz), 7.16-7.43 (4H, m), 9.29 (1H, s)

Preparation 164

A mixture of tert-butyl 4-[4-({[2-(4-methylphenyl)-1-cyclohexen-1-yl]carbonyl}amino) phenyl]-1-piperazinecarboxylate (2.2 g) and trifluoroacetic acid (10.7 ml) in dichloromethane (5 ml ) was stirred at ambient temperature for 4 hours. The reaction mixture was evaporated in vacuo and the residue was dissolved in a mixture of ethyl acetate and water and adjusted to pH 8.0 with aqueous potassium carbonate solution. The organic layer was washed with brine and dried over magnesium sulfate. The solvent was evaporated in vacuo and the residue was crystallized from ethyl acetate and diisopropyl ether to give 2-(4-methylphenyl)-N-[4-(1-piperazinyl)phenyl]-1-cyclohexene-1-carboxamide (1.19 g).

¹H-NMR(DMSO-d₆): δ 1.70 (4H, br.s), 2.21 (3H, s), 2.34 (4H, br.s), 2.78-2.81 (4H, m), 2.89-2.94 (4H, m), 6.74 (2H, d, J=9.0 Hz), 7.03.(2H, d, J=8.0 Hz), 7.16-7.22 (4H, m), 9.25.(1H, s)

### EXAMPLE 367

A mixture of 2-(4-methylphenyl)-N-[4-(1-piperazinyl)phenyl]-1-cyclohexene-1-carboxamide (298 mg), 3-cyanobenzaldehyde (210 mg) and sodium triacetoxyborohydride (510 mg) in dichloromethane (20 ml) was stirred at ambient temperature for 14 hours. Water (20 ml) was added to the reaction mixture and adjusted to pH 8.5 with 10% aqueous potassium carbonate solution and the mixture was stirred for 30 minutes. The organic layer was washed with brine and dried over magnesium sulfate. The solvent was evaporated in vacuo and the residue was chromatographed on silica gel eluting with ethyl acetate and n-hexane (7:3-9:1). The fractions containing the desired product were collected and evaporated in vacuo and the residue was recrystallized from ethyl acetate and diisopropyl ether to give N-{4-[4-(3-cyanobenzyl)-1-piperazinyl]phenyl}-2-(4-methylphenyl)-1-cyclohexene-1-carboxamide (297 mg).

¹H-NMR(DMSO-d₆): δ 1.70 (4H, br.s), 2.21 (3H, s), 2.34 (4H, br.s), 2.49-2.50 (4H, m), 3.03 (4H, m), 3.57 (2H, s), 6.76 (2H, d, J=9.0 Hz), 7.04 (2H, d, J=8.0 Hz), 7.16-7.23 (4H, m), 7.51-7.59 (1H, m), 7.66-7.76 (3H, m), 9.26 (1H, s)

(+)ESI-MS: 491(M+H)⁺, 513(M+Na)⁺

### EXAMPLE 368

tert-Butyl 4-[5-({[2-(4-methylphenyl)-1-cyclohexen-1-yl]carbonyl}amino)-2-pyridinyl]-1-piperazinecarboxylate was obtained in the same manner as in Example 366.

¹H-NMR(DMSO-d₆): δ 1.41 (9H, s), 1.70 (4H, br.s), 2.22 (3H, s), 2.35 (4H, br.s), 3.36 (8H, m),6.72 (1H, d, J=9.1 Hz), 7.05 (2H, d, J=8.1 Hz), 7.17 (2H, d, J=8.1 Hz), 7.20-7.28 (1H, m), 7.50-7.57 (1H, m), 8.03 (1H, m), 9.32 (1H, s)

Preparation 165

2-(4-Methylphenyl)-N-[6-(1-piperazinyl)-3-pyridinyl]-1-cyclohexene-1-carboxamide was obtained in the same manner as in Preparation 164.

¹H-NMR(DMSO-d₆): δ 1.71 (4H, br.s), 2.22 (3H, s), 2.35 (4H, br.s), 2.71-2.75 (4H, m), 3.25-3.28 (4H, m), 6.66 (1H, d, J=9.1 Hz), 7.05 (2H, d, J=8.1 Hz), 7.18 (2H, d, J=8.1 Hz), 7.48 (1H, dd, J=2.5 and 9.1 Hz), 8.00 (1H, d, J=2.5 Hz), 9.28 (1H, s)

### EXAMPLE 369

N-{6-[4-(3-Cyanobenzyl)-1-piperazinyl]-3-pyridinyl}-2-(4-methylphenyl)-1-cyclohexene-1-carboxamide was obtained in the same manner as in Example 367.

¹H-NMR(DMSO-d₆): δ 1.70 (4H, br.s), 2.22 (3H, s), 2.35 (4H, br.s), 2.43-2.50 (4H, m), 3.34-3.39 (4H, m), 3.56 (2H, s ), 6.69 (1H, d, J=9.1 Hz), 7.05 (2H, d, J=8.1 Hz), 7.18 (2H, d, J=8.1 Hz), 7.47-7.59 (2H, m), 7.67-7.76 (3H, m), 8.00 (1H, d, J=2.5 Hz), 9.30-(1H, s)

### EXAMPLE 370

N-[4-(4-{[6-(Acetylamino)-2-pyridinyl]methyl}-1-piperazinyl)phenyl]-2-(4-methylphenyl)-1-cyclohexene-1-carboxamide was obtained in the same manner as in Example 367.

¹H-NMR(DMSO-d₆):δ 1.70 (4H, br.s), 2.08 (3H, s), 2.21 (3H, s), 2.34 (4H, br.s), 2.50-2.52 (4H, m), 3.03 (4H, s), 3.53 (2H, s), 6.76 (2H, d, ′J=9.0 Hz), 7.04 (2H, d, J=8.1 Hz), 7.12-7.22 (5H, m), 7.75 (1H, d, J=8.0 Hz), 7.96 (1H, d , J=8.0 Hz), 9.25 (1H, s), 10.51 (1H, s)

(+)ESI-MS: 524(M+H)⁺, 546(M+Na)⁺

### EXAMPLE 371

A mixture of N-[4-(4-{[6-(acetylamino)-2-pyridinyl]methyl}-1-piperazinyl)phenyl]-2-(4-methylphenyl)-1-cyclohexene-1-carboxamide (295 mg) and 6N hydrochloric acid (10 ml) in methanol (10 ml) was refluxed under stirring for 5 hours. The reaction mixture was evaporated in vacuo and the residue was dissolved in a mixture of ethyl acetate and water and adjusted to pH 8.0 with aqueous potassium carbonate solution. The organic layer was washed with brine and dried over magnesium sulfate. The solvent was evaporated in vacuo and the residue was recrystallized from ethyl acetate and diisopropyl ether to give N-(4-{4-[(6-amino-2-pyridinyl)methyl]-1-piperazinyl}phenyl)-2-(4-methylphenyl)-1-cyclohexene-1-carboxamide (180 mg).

¹H-NMR(DMSO-d₆): δ 1.70 (4H, br.s), 2.21 (3H, s), 2.34 (4H, br.s), 2.50-2.51 (4H, m), 3.35-3.36 (4H, m), 3.36 (2H, s ), 5.85 (2H, s), 6.30 (1H, d, J=8.1 Hz), 6.55 (1H, d, J=7.2 Hz), 6.76 (2H, d, J=9.0 Hz), 7.04 (2H, d, J=8.0 Hz), 7.16-7.36 (5H, m), 9.25 (1H, s)

(+)ESI-MS: 482(M+H)⁺, 504(M+Na)⁺

### EXAMPLE 372

2-(4-Methylphenyl)-N-{6-[4-(2-pyridinylmethyl)-1-piperazinyl]-3-pyridinyl}-1-cyclohexene-1-carboxamide was obtained in the same manner as in Example 367.

¹H-NMR(DMSO-d₆): δ 1.70 (4H, br.s), 2.22 (3H, s), 2.35(4H, br.s), 2.47-2.51 (4H, m), 3.36-3.39 (4H, m), 3.62 (2H, s), 6.69 (1H, d, J=9.1 Hz), 7.06 (2H, d, J=8.0 Hz), 7.17 (2H, d, J=8.0 Hz), 7.25-7.28 (1H, m ), 7.45-7.50 (2H, m), 7.74-7.77 (1H, m), 8.01 (1H, d J=2.6 Hz), 8.50 (1H, m ), 9.27 (1H, s)

(+)ESI-MS: 468(M+H)⁺, 490(M+Na)⁺

### EXAMPLE 373

A mixture of 4-cyanobenzyl chloride (150 mg) and sodium iodide (180 mg) in acetone (30 ml) was stirred at ambient temperature for 2 hours. 2-(4-Methylphenyl)-N-[4-(1-piperazinyl)phenyl]-1-cyclohexene-1-carboxamide (334 mg) and powder potassium carbonate (273 mg) were added to the above mixture and the obtained mixture was refluxed under stirring for 3 hours. The reaction mixture was evaporated in vacuo and the residue was dissolved in a mixture of ethyl acetate and water, and the organic layer was washed with brine and dried over magnesium sulfate. The solvent was evaporated in vacuo and the residue was recrystallized from ethyl acetate and diisopropyl ether to give N-{4-[4-(4-cyanobenzyl)-1-piperazinyl]phenyl}-2-(4-methylphenyl)-1-cyclohexene-1-carboxamide (324 mg).

¹H-NMR(DMSO-d₆): δ 1.70 (4H, br.s), 2.21 (3H, s), 2.34 (4H, br.s), 2.49-2.50 (4H, m), 3.03 (4H, m), 3.60 (2H, s), 6.86 (2H, d, J=9.0 Hz), 7.04 (2H, d, J=8.1 Hz), 7.16-7.23 (4H, m ), 7.53 (2H, d, J=8.1 Hz), 7.80 (2H, d, J=8.1 Hz), 9.26 (1H, s)

### EXAMPLE 374

A mixture of 2-cyanobenzyl chloride (149 mg) and sodium iodide (130 mg) in acetone (30 ml) was stirred at ambient temperature for 2 hours. 2-(4-Methylphenyl)-N-[4-(1-piperazinyl)phenyl]-1-cyclohexene-1-carboxamide (333 mg) and powder potassium carbonate (273 mg) was added to the above mixture and the obtained mixture was refluxed under stirring for 3 hours. The reaction mixture was evaporated in vacuo and the residue was dissolved in a mixture of ethyl acetate and water, and the organic layer was washed with brine and dried over magnesium sulfate. The solvent was evaporated in vacuo and the residue was chromatographed on silica gel eluting with ethyl acetate and n-hexane (6:4). The fractions containing the desired product were collected and evaporated in vacuo and the residue was recrystallized from ethyl acetate and diisopropyl ether to give N-{4-[4-(2-cyanobenzyl)-1-piperazinyl]phenyl}-2-(4-methylphenyl)-1-cyclohexene-1-carboxamide (85 mg).

¹H-NMR(DMSO-d₆): δ 1.70 (4H, br.s), 2.21 (3H, s), 2.34 (4H, br.s), 2.50-2.56 (4H, m), 3.00-3.02 (4H, m), 3.69 (2H, s), 6.76 (2H, d, J=9.0 Hz), 7.04 (2H, d, J=8.0 Hz), 7.16-7.22 (4H, m), 7.47-7.51 (1H, m), 7.58-7.80 (2H, m), 7.82 (1H, d, J=6.9 Hz), 9.26 (1H, s)

(+)ESI-MS: 491(M+H)⁺, 513(M+Na)⁺

### EXAMPLE 375

A mixture of 2-(4-methylphenyl)-1-cyclohexene-1-carboxylic acid (270 mg) and 4-(4-benzyl-1-piperazinyl)phenylamine (280 mg), 1-hydroxybenzotriazole hydrate (161 mg), 1-[3-(dimethylamino)propyl]-3-ethylcarbodiimide (163 mg) and 4-(dimethyamino)pyridine (2.4 mg) in N,N-dimethylformamide (20 ml) was stirred at ambient temperature overnight. The reaction mixture was poured into a mixture of ethyl acetate and water and the organic layer was washed with brine and dried over magnesium sulfate. The solvent was evaporated in vacuo and the residue was recrystallized from ethyl acetate and diisopropyl ether to give N-[4-(4-benzyl-1-piperazinyl)phenyl]-2-(4-methylphenyl)-1-cyclohexene-1-carboxamide (304 mg).

¹H-NMR(DMSO-d₆): δ 1.70 (4H, s), 2.21 (3H, s), 2.45 (4H, br.s), 2.47-2.51 (4H, m), 2.99-3.04 (4H, m), 3.50 (2H, s), 6.75 (2H, d, J=9.0 Hz), 7.04 (2H, d, J=8.0 Hz), 7.16-7.38 (9H, m), 9.25 (1H, s)

(+)ESI-MS: 466(M+H)⁺, 488(M+Na)⁺

### EXAMPLE 376

N-[4-(4-Benzyl-1-piperazinyl)phenyl]-2-[4-(trifluoromethyl)phenyl]-1-cyclohexene-1-carboxamide was obtained in the same manner as in Example 375.

¹H-NMR(DMSO-d₄): δ 1.73 (4H, s), 2.38 (4H, br. s), 2.45-2.51 (4H, m), 3.00-3.02 (2H, m), 3.50 (2H, s), 6.75 (2H, d, J=8.0 Hz), 7.16 (2H, d, J=8.9 Hz), 7.22-7.38 (5H, m), 7.48 (2H, d, J=8.2 Hz), 7.62 (2H, d, J=8.2 Hz), 9.40 (1H, s)

(+)ESI-MS: 520(M+H)⁺, 542(M+Na)⁺

Preparation 166

A mixture of 2,3-dihydro-1H-inden-2-ylamine hydrochloride (1.7 g) and picolinic acid (1.3 g), 1-hydroxybenzotriazole hydrate (1.69 g), 1-[3-(dimethylamino)propyl]-3-ethylcarbodiimide (1.71 g) and 4-(dimethyamino)pyridine (24.5 mg) in N,N-dimethylformamide (20 ml) was stirred at ambient temperature for 15 hours. The reaction mixture was poured into a mixture of ethyl acetate and water and the organic layer was washed with brine and dried over magnesium sulfate. The solvent was evaporated in vacuo to give N-(2,3-dihydro-1H-inden-2-yl)-2-pyridinecarboxamide (2.99 g).

¹H-NMR(DMSO-d₆): δ 3.00-3.11 (2H, m), 3.17-3.29 (2H, m), 4.69-4.87 (1H, m), 7.14-7.25 (2H, m), 7.60-7.61 (2H, m), 8.01-8.08 (2H, m), 8.63 (1H, d, J=4.7 Hz), 8.91 (1H, d, J=7.8 Hz), 13.67 (1H, br.s)

Preparation 167

N-(2,3-Dihydro-1H-inden-2-yl)-2-pyridinecarboxamide (2.95 g) was portionwise added to fuming nitric acid (d=1.52) (50 ml) at −30° C. to −100° C. and the resultant mixture was stirred at −10° C. to −5° C. for 15 minutes. The reaction mixture was poured into ice-water and adjusted to pH 8.0 with aqueous potassium carbonate solution and extracted with ethyl acetate and tetrahydrofuran. The organic layer was washed with brine and dried over magnesium sulfate. The solvent was evaporated in vacuo and the residue was chromatographed on silica gel eluting with ethyl acetate and n-hexane (6:4). The fractions containing the desired product were collected and evaporated in vacuo and the residue was recrystallized from ethyl acetate and diisopropyl ether to give N-(5-nitro-2,3-dihydro-1H-inden-2-yl)-2-pyridinecarboxamide (1.16 g).

¹H-NMR(DMSO-d₆): δ 3.09-3.40 (4H, m), 4.74-4.93 (1H, m), 7.50 (1H, d J=8.1 Hz), 7.57-7.64 (1H, m), 7.96-8.09 (3H, m), 8.61-8.69 (1H, m), 9.05 (1H, d, J=7.7 Hz)

Preparation 168

A mixture of N-(5-nitro-2,3-dihydro-1H-inden-2-yl)-2-pyridinecarboxamide (623 mg) in methanol (20 ml) and tetrahydrofuran (20 ml) was hydrogenated over 10% palladium on carbon (300 mg) under an atmospheric pressure of hydrogen at ambient temperature under stirring for 7 hours. After removal of the catalyst, the solvent was evaporated in vacuo to give N-(5-amino-2,3-dihydro-1H-inden-2-yl)-2-pyridinecarboxamide (557 mg).

¹H-NMR(DMSO-d₆): δ 2.79-3.11 (4H, m), 4.58-4.77 (1H, m), 4.85 (2H, s), 5.73-5.76 (1H, m), 6.36-6.45 (2H, m), 6.81 (1H, d, J=7.9 Hz), 7.56-7.63 (1H, m), 7.95-8.08 (2H, m), 8.62 (1H, d, J=5.8 Hz), 8.77 (1H, d, J=8.0 Hz)

### EXAMPLE 377

A mixture of 2-[4-(trifluoromethyl)phenyl]-1-cyclohexene-1-carboxylic acid (270 mg) and N-(5-amino-2,3-dihydro-1H-inden-2-yl)-2-pyridinecarboxamide (266 mg), 1-hydroxybenzotriazole hydrate (242 mg), 1-[3-(dimethylamino)propyl]-3-ethylcarbodiimide (163 mg) and 4-(dimethyamino)pyridine (2.5 mg) in N,N-dimethylformamide (8 ml) was stirred at ambient temperature for 14 hours. The reaction mixture was poured into a mixture of ethyl acetate and water and the organic layer was washed with brine and dried over magnesium sulfate. The solvent was evaporated in vacuo and the residue was chromatographed on silica gel eluting with ethyl acetate and n-hexane (9:4). The fractions containing the desired product were collected and evaporated in vacuo and the residue was recrystallized from ethyl acetate and diisopropyl ether to give N-{5-[({2-[4-(trifluoromethyl)phenyl]-1-cyclohexen-1-yl}carbonyl)amino]-2,3-dihydro-1H-inden-2-yl}-2-pyridinecarboxamide (202 mg).

¹H-NMR(DMSO-d₆): δ 1.74 (4H, br.s), 2.40 (4H, br.s), 2.86-3.16 (4H, m), 4.62-4.80 (1H, m), 7.01-7.10 (2H, m), 7.28 (1H, s), 8.50 (2H, d, J=8.1 Hz), 7.56-7.66 (3H, m), 7.95-8.07 (2H, m), 8.62 (1H, d, J=4.6 Hz), 8.86 (1H, d J=8.0 Hz), 9.57 (1H, s)

### EXAMPLE 378

Methyl 5-[({2-[4-(trifluoromethyl)phenyl]-1-cyclohexen-1-yl}carbonyl)amino]-2,3-dihydro-1H-inden-2-ylcarbamate was obtained in the same manner as in Example 377.

¹H-NMR(DMSO-d₆): δ 1.73 (4H, br.s), 2.49 (4H, br.s) , 2.61-2.82 (2H, m), 2.96-3.07 (2H, m), 3.52 (3H, s), 4.11-4.25 (1H, m), 6.96-7.06 (2H, m), 7.06 (1H, s), 7.40-7.50 (1H, m), 7.48 (2H, d, J=8.3 Hz), 7.63 (2H, d J=8.3 Hz), 9.53 (1H, s)

(+)ESI-MS: 459(M+H)⁺, 481(M+Na)⁺

### EXAMPLE 379.

Methyl 5-({[2-(4-methylphenyl)-1-cyclohexen-1-yl]carbonyl}amino)-2,3-dihydro-1H-inden-2-ylcarbamate was obtained in the same manner as in Example 377.

¹H-NMR(DMSO-d₆): δ 1.71 (4H, br.s), 2.21 (3H, s), 2.49 (4H, br.s), 2.60-2.91 (2H, m ), 2.95-3.08 (2H, m), 3.52 (2H, s), 4.14-4.25 (1H, m), 6.96-7.09 (4H, m), 7.18 (2H, d J=8.0 Hz), 7.29 (1H, s), 7.40-7.43 (1H, m), 9.39 (1H, s)

(+)ESI-MS: 405(M+H)⁺, 427(M+Na)⁺

### EXAMPLE 380

N-[5-({[2-(4-Methylphenyl)-1-cyclohexen-1-yl]carbonyl}amino)-2,3-dihydro-1H-inden-2-yl]-2-pyridinecarboxamide was obtained in the same manner as in

### EXAMPLE 377.

¹H-NMR(DMSO-d₆): δ 1.71 (4H, br.s), 2.22 (3H, s), 2.49 (4H, br.s), 2.85-3.16 (4H, m), 7.01-7.12 (3H, m), 7.19 (2H, d, J=8.0 Hz), 7.34 (1H, s), 7.56-7.62 (1H, m), 7.95-8.07 (2H, m), 8.62 (1H, d, J=4.6 Hz), 8.85 (1H, d, J=8.0 Hz), 9.43 (1H, s)

(+)ESI-MS: 452(M+H)⁺, 474(M+Na)⁺

Preparation 169

N-(2,3-Dihydro-1H-inden-2-yl)-2-(2-pyridinyl)acetamide was obtained in the same manner as in Preparation 166.

¹H-NMR(DMSO-d₆): δ 2.73-2.89 (2H, m), 3.12-3.36 (2H, m), 3.61 (2H, s), 4.38-4.55 (1H, m), 7.12-7.26 (4H, m), 7.33 (1H, d, J=7.8 Hz), 7.68-7.72 (1H, m), 8.45-8.47 (2H, m)

Preparation 170

N-(5-Nitro-2,3-dihydro-1H-inden-2-yl)-2-(2-pyridinyl)acetamide was obtained in the same manner as in Preparation 167.

¹H-NMR(DMSO-d₆): δ 2.84-3.00 (2H, m), 3.24-3.36 (2H, m), 3.59 (2H, s), 4.45-4.61(1H, m), 7.20-7.27 (1H, m), 7.32 (1H, d, J=7.9 Hz), 7.51 (1H, d, J=8.2 Hz), 7.68-7.73.(1H, m), 8.03-8.11 (2H, m), 8.41-8.68 (2H, m)

Preparation 171

N-(5-Amino-2,3-dihydro-1H-inden-2-yl)-2-(2-pyridinyl)acetamide was obtained in the same manner as in

Preparation 168.

¹H-NMR(DMSO-d₆): δ 2.79-3.11 (4H, m), 3.61 (2H, s), 4.58-4.77 (1H, m), 4.85 (2H, s), 5.73-5.76 (1H, m), 6.36-6.45 (2H, m), 6.87 (1H, d, J=7.9 Hz), 7.56-7.63 (1H, m), 7.95-8.08 (2H, m), 8.62 (1H, d, J=5.8 Hz), 8.77 (1H, d, J=8.0 Hz)

### EXAMPLE 381

2-(4-Methylphenyl)-N-{2-[(2-pyridinylacetyl)amino]-2,3-dihydro-1H-inden-5-yl}-1-cyclohexene-1-carboxamide was obtained in the same manner as in Example 377.

¹H-NMR(DMSO-d₆): δ 1.71 (4H, br.s), 2.24 (3H, s), 2.49 (4H, br.s), 2.49-2.72 (2H, m), 2.96-3.13 (2H, m), 3.58 (2H, s), 4.36-4.46 (1H, m), 6.99-7.33 (9H, m), 7.67-7.75 (1H, m), 8.40-8.46 (2H, m), 9.41 (1H, s)

(+)ESI-Ms: 466(M+H)⁺, 488(M+Na)⁺

Preparation 172

Methyl 2,3-dihydro-1H-inden-2-ylcarbamate (1.7 g) was portionwise added to fuming nitric acid (d=1.52) (20 ml) at −30° C. to −10° C. and the resultant mixture was stirred at −10° C. to −5° C. for 15 minutes. The reaction mixture was poured into ice-water and adjusted to pH 8.0 with aqueous potassium carbonate solution and extracted with ethyl acetate and tetrahydrofuran. The organic layer was washed with brine and dried over magnesium sulfate. The solvent was evaporated in vacuo and the residue was chromatographed on silica gel eluting with ethyl acetate and n-hexane (6:4). The fractions containing the desired product were collected and evaporated in vacuo to give methyl 4,6-dinitro-2,3-dihydro-1H-inden-2-ylcarbamate (1.21 g).

¹H-NMR(DMSO-d₆): δ 3.30-3.48 (2H, m), 3.50-3.75 (2H, m), 3.79 (3H, s), 5.50-5.70 (1H, m), 8.09 (2H, s)

Preparation 173

Methyl 4,6-diamino-2,3-dihydro-1H-inden-2-ylcarbamate was obtained in the same manner as in Preparation 168.

¹H-NMR(DMSO-d₆): δ 2.50-2.59 (2H, m), 2.82-2.93 (2H, m), 3.33 (3H, s), 4.04-4.58 (3H, m), 6.34-6.39 (2H, m), 7.31-7.42 (1H, m)

### EXAMPLE 382

A mixture of 2-[4-(trifluoromethyl)phenyl]-1-cyclohexene-1-carboxylic acid (270 mg) and methyl 4,6-diamino-2,3-dihydro-1H-inden-2-ylcarbamate (232 mg), 1-hydroxybenzotriazole hydrate (161 mg), 1-[3-(dimethylamino) propyl]-3-ethylcarbodiimide (163 mg) and 4-(dimethylamino)pyridine (2.5 mg) in N,N-dimethylformamide (8 ml) was stirred at ambient temperature for 14 hours. The reaction mixture was poured into a mixture of ethyl acetate and water and the organic layer was washed with brine and dried over magnesium sulfate. The solvent was evaporated in vacuo and the residue was chromatographed on silica gel eluting with ethyl acetate and n-hexane (9:4). The fractions containing the desired product were collected and evaporated in vacuo and the residue was recrystallized from ethyl acetate and diisopropyl ether to give methyl 6-amino-4-[({2-[4-(trifluoromethyl)phenyl]-1-cyclohexen-1-yl}carbonyl)amino]-2,3-dihydro-1H-inden-2-ylcarbamate (156 mg).

¹H-NMR(DMSO-d₆): δ 1.73 (4H, br.s), 2.49 (4H, br.s), 2.50-2.73 (2H, m), 2.84-2.98 (2H, m), 3.33 (3H, s), 4.04-4.18 (1H, m), 4.29 (2H, s), 6.37 (1H, s), 6.46 (1H, s), 7.37 (1H, d, J=6.8 Hz), 7.51 (2H, d, J=8.2 Hz), 7.67 (2H, d, J=8.2 Hz), 8.78 (1H, s)

### EXAMPLE 383

Methyl 6-amino-4-({[2-(4-methylphenyl)-1-cyclohexen-1-yl]carbonyl}amino)-2,3-dihydro-1H-inden-2-ylcarbamate was obtained in the same manner as in Example 382.

¹H-NMR(DMSO-d₆): δ 1.70 (4H, br.s), 2.22 (3H, s), 2.49 (4H, br.s), 2.56-2.63 (2H, m), 2.83-2.98 (2H, m), 3.3.4 (3H, s), 4.05-4.17 (3H, m), 6.43 (1H, s), 6.56 (1H, s), 7.09 (2H, d, J=8.1 Hz), 7.16 (2H, d, J=8.1 Hz), 7.39 (1H, d, J=6.9 Hz), 8.52 (1H, s)

Preparation 174

A mixture of 2-formylbenzoic acid (3.0 g), 2-(2-aminoethyl)pyridine (3.66 g) and sodium triacetoxyborohydride (12.7 g) in dichloromethane (50 ml) was stirred at ambient temperature for 14 hours. Water (30 ml) was added to a reaction mixture and adjusted to pH 8.5 with 10% aqueous potassium carbonate solution and stirred for 30 minutes. The organic layer was washed with brine and dried over magnesium sulfate. The solvent was evaporated in vacuo and the residue was recrystallized from ethyl acetate and diisopropyl ether to give 2-[2-(2-pyridinyl)ethyl]-1-isoindolinone (4.16 g).

¹H-NMR(DMSO-d₆): δ 3.13 (2H, t, J=7.1 Hz), 3.92 (2H, t, J=7.1 Hz), 4.43 (2H, s), 7.22-7.29 (1H, m), 7.31 (1H, d, J=7.8 Hz), 7.50-7.70 (5H, m), 8.48-8.51 (1H, m)

Preparation 175

2-[2-(2-Pyridinyl)ethyl]-1-isoindolinone (1.71 g) was portionwise added to fuming nitric acid (d=1.52) (15 ml) at −30° C. to −10° C. and the resultant mixture was stirred at −10° C. to −5° C. for 20 minutes. The reaction mixture was poured into ice-water and adjusted to pH 8.0 with aqueous potassium carbonate solution and extracted with ethyl acetate and tetrahydrofuran. The organic layer was washed with brine and dried over magnesium sulfate. The solvent was concentrated in vacuo and the precipitate was collected by filtration to give 6-nitro-2-[2-(2-pyridinyl)ethyl]-1-isoindolinone (1.76 g)

¹H-NMR(DMSO-d₆): δ 3.11 (2H, t, J=7.0 Hz), 3.94 (2H, t, J=7.0 Hz), 4.61 (2H, s), 7.19-7.25 (1H, m), 7.32 (1H, d, J=7.8 Hz), 7.66-7.70 (1H, m), 7.87 (1H, d, J=8.0 Hz), 8.30 (1H, d, J=2.1 Hz), 8.41-8.49 (2H, m)

Preparation 176

A mixture of 6-nitro-2-[2-(2-pyridinyl)ethyl]-1-isoindolinone (760 mg) in methanol (20 ml) and tetrahydrofuran (10 ml) was hydrogenated over 10% palladium on carbon (300 mg) under an atmospheric pressure of hydrogen at ambient temperature under stirring for 6 hours. After removal of the catalyst, the solvent was evaporated in vacuo and the residue was washed with diisopropyl ether to give 6-amino-2-[2-(2-pyridinyl)ethyl]-1-isoindolinone (545 mg).

¹H-NMR(DMSO-d₆): δ 3.04 (2H, t, J=7.5 Hz), 3.84 (2H, t, J=7.5 Hz), 4.20 (2H, s), 5.27 (2H, s), 6.78-6.80 (2H, m), 7.13-7.30 (3H, m), 7.64-7.69 (1H, m), 8.48-8.50 (1H, m)

### EXAMPLE 384

A mixture of 2-[4-(trifluoromethyl)phenyl]-1-cyclohexene-1-carboxylic acid (270 mg) and 6-amino-2-[2-(2-pyridinyl) ethyl]-1-isoindolinone (270 mg), 1-[3-(dimethylamino)propyl]-3-ethylcarbodiimide (163 mg) and 4-(dimethyamino)pyridine (2.5 mg) in N,N-dimethylformamide (30 ml) was stirred at ambient temperature overnight. The reaction mixture was poured into a mixture of ethyl acetate and water and the organic layer was washed with brine and dried over magnesium sulfate. The'solvent was evaporated in vacuo and the residue was chromatographed on silica gel eluting with ethyl acetate and methanol (96:4). The fractions containing the desired product were collected and evaporated in vacuo and the residue was recrystallized from ethyl acetate and diisopropyl ether to give N-{3-oxo-2-[2-(2-pyridinyl)ethyl]-2,3-dihydro-1H-isoindol-5-yl}-2-[4-(trifluoromethyl)phenyl]-1-cyclohexene-1-carboxamide (57 mg).

¹H-NMR(DMSO-d₆): δ 1.75 (4H, br.s), 2.50 (4H, br.s), 3.04 (2H, t, J=7.2 Hz), 3.85 (2H, t, J=7.2 Hz), 4.30 (2H, s), 7.18-7.30 (2H, m), 7.38-7.72 (7H, m), 7.78 (1H, s), 8.47 (1H, d, J=4.2 Hz), 9.87 (1H, s)

(+)ESI-MS: 506(M+H)⁺, 528(M+Na)⁺

### EXAMPLE 385

1-[3-(Dimethylamino)propyl]-3-ethylcarbodiimide (0.17 g) was added to a solution of 4-(2-pyridinylmethyl)aniline (0.18 g), 2-[4-(trifluoromethyl)phenyl]-1-cyclohexene-1-carboxylic acid (0.3 g), 1-hydroxybenzotriazole (0.15 g) and 4-dimethylaminopyridine (6 mg) in tetrahydrofuran (3 ml) under ice-cooling and the mixture was stirred at ambient temperature for 18 hours. The reaction mixture was poured into water and the mixture was extracted with ethyl acetate. The organic layer was washed with water, dried over magnesium sulfate and evaporated in vacuo. The residue was crystallized from a mixture of ethyl acetate and diisopropyl ether to give N-[4-(2-pyridinylmethyl)phenyl]-2-[4-(trifluoromethyl)phenyl]-1-cyclohexene-1-carboxamide (0.15 g).

¹H-NMR(DMSO-d₆): δ 1.63-1.81(4H, m), 2.34-2.47(4H, m), 3.97(2H, s), 7.10(2H, d; J=8.4 Hz), 7.14-7.23(2H, m), 7.25(2H, d, J=8.4 Hz), 7.48(2H, d, J=8.1 Hz), 7.61(2H, d, J=8.1 Hz), 7.63-7.70(1H, m), 8.45(1H, dd, J=0.7 Hz, 4.7 Hz), 9.59(1H, s)

(+)ESI-MS: 437(M+H)⁺, 459(M+Na)⁺

### EXAMPLE 386

N-{4-[4-(2-Pyridinyl)-1-piperazinyl]phenyl}-2-[4-(trifluoromethyl)phenyl]-1-cyclohexene-1-carboxamide was obtained in the same manner as in Example 385.

¹H-NMR(DMSO-d₆): δ 1.68-1.80(4H, m), 2.36-2.44(4H, m), 3.08-3.16(4H, m), 3.56-3.64(4H, m), 6.63-6.69(1H, m), 6.81-6.90(3H, m), 7.20(2H, d, J=9.0 Hz), 7.49(2H, d, J=8.1 Hz), 7.52-7.57(1H, m), 7.62(2H,-d, J=8.1 Hz), 8.11-8.16(1H, m), 9.41(1H, s)

(+)ESI-MS: 507(M+H)⁺, 529(M+Na)⁺

### EXAMPLE 387

To a solution of 2-[4-(trifluoromethyl)phenyl]-1-cyclohexene-1-carboxylic acid (698 mg) in toluene (8 ml) were added thionyl chloride (0.38 ml) and N,N-dimethylformamide (2 drops) and the mixture was stirred at 50° C. for an hour. The mixture was evaporated in vacuo and the residue was dissolved in tetrahydrofuran (1 ml). The acid chloride in tetrahydrofuran was added to a solution of tert-butyl 5-amino-2-pyridinyl(2-{6-[(tert-butoxycarbonyl)amino]-2-pyridinyl}ethyl)carbamate (1.11 g) and triethylamine (0.54 ml) in tetrahydrofuran (8 ml) at ambient temperature and the mixture was stirred at the same temperature for an hour. The mixture was poured into water and extracted with ethyl acetate. The organic layer was washed with brine, dried over magnesium sulfate, and evaporated in vacuo. The residue was purified by column chromatography on silica gel by eluting with hexane:ethyl acetate (2:1→1:1) to give tert-butyl 2-{6-[(tert-butoxycarbonyl)amino]-2-pyridinyl}ethyl{5-[({2-[4-(trifluoromethyl)phenyl]-1-cyclohexen-1-yl}carbonyl)amino]-2-pyridinyl}carbamate (1.458 g) as a white solid.

¹H-NMR(CDCl₃): δ 1.43(9H, s), 1.48(9H, s), 1.75-1.85(4H, m), 2.40-2.50(2H, m), 2.50-2.60(2H, m), 3.05(2H, dd, J=8.9,6.5 Hz), 6.52(1H, s), 7.04(2H, d, J=8.1 Hz), 7.35-7.45(4H, m), 7.55-7.62(3H, m), 7.89(1H, d, J=2.4 Hz)

ESI-MS(m/z): 704(M+Na)⁺

### EXAMPLE 388

To a solution of tert-butyl 2-{6-[(tert-butoxycarbonyl)amino]-2-pyridinyl}ethyl{5-[({2-[4-(trifluoromethyl)phenyl]-1-cyclohexen-1-yl}carbonyl)amino]-2-pyridinyl}carbamate (1.465 g) in dichloromethane (3 ml) was added trifluoroacetic acid (2.48 ml). The reaction mixture was stirred at ambient temperature for 22 hours, quenched with 10% aqueous potassium carbonate solution, and extracted with dichloromethane. The organic layer was washed with brine, dried over magnesium sulfate, filtered, and concentrated in vacuo. The residue was recrystallized from ethyl acetate-diisopropyl ether to give N-(6-{[2-(6-amino-2-pyridinyl)ethyl]amino}-3-pyridinyl)-2-[4-(trifluoromethyl)phenyl]-1-cyclohexene-1-carboxamide (680 mg) as white crystals.

¹H-NMR(CDCl₃): δ 1.70-1.90(4H, m), 2.40-2.48(2H, m), 2.48-2.5(2H, m), 2.85(2H, t, J=6.5 Hz), 3.54(2H, t, J=6.5 Hz), 4.43(2H, s), 4.98(1H, s), 6.26(2H, d, J=8.9 Hz), 6.31-6.36(2H, m), 6.49(1H, d, J=7.3 Hz), 7.22(1H, dd, J=8.9,2.7 Hz), 7.41(2H, d, J=8.1 Hz), 7.52(1H, d, J=2.7 Hz), 7.59(2H, d, J=8.4 Hz)

ESI-MS(m/z): 482(M+H)⁺

### EXAMPLE 389

To a solution of N-[4-(1-piperazinyl)phenyl]-2-[4-(trifluoromethyl)phenyl]-1-cyclohexene-1-carboxamide (300 mg) and (1-trityl-1H-1,2,4-triazol-3-yl)methyl methanesulfonate (381 mg) in tetrahydrofuran (10 ml) was added triethylamine (92 mg) at ambient temperature. The mixture was stirred at the same temperature for 9 hours and poured into water followed by the extraction with ethyl acetate. The organic layer was washed with brine, dried over magnesium sulfate and concentrated in vacuo. The residue was purified by column chromatography on silica gel by eluting with chloroform:methanol (9:1) to give 2-[4-(trifluoromethyl)phenyl]-N-(4-{4-[(1-trityl-1H-1,2,4-triazol-3-yl)methyl]-1-piperazinyl}phenyl)-1-cyclohexene-1-carboxamide (337 mg) as white crystals.

¹H-NMR(CDCl₃): δ 1.79(4H, br s), 2.42(2H, br s), 2.54(2H, br s), 2.64(4H, br s), 3.10(4H, br s), 3.76(2H, s), 6.40(1H, s), 6.72(2H, d, J=8.9 Hz), 6.82(2H, d, J=9.2 Hz), 7.11-7.17(5H, m), 7.25-7.35(10H, m), 7.41(2H, d, J=7.9 Hz), 7.58(2H, d, J=7.9 Hz), 7.92(1H, s)

ESI-MS(m/z): 775(M+Na)⁺

### EXAMPLE 390

To a solution of 2-[4-(trifluoromethyl)phenyl]-N-(4-{4-[(1-trityl-1H-1,2,4-triazol-3-yl)methyl]-1-piperazinyl}phenyl)-1-cyclohexene-1-carboxamide (312 mg) in methanol (3 ml) was added 35% hydrochloric acid (230 mg). The mixture was stirred at ambient temperature for 3 hours. Ethyl acetate and 10% aqueous potassium carbonate solution were. added, then the separated organic layer was washed with brine, dried over magnesium sulfate, and concentrated in vacuo. The residue was purified by column chromatography on silica gel by eluting with chloroform:methanol (19:1 to 9:1) to give N-{4-[4-(1H-1,2,4-triazol-3-ylmethyl)-1-piperazinyl]phenyl}-2-[4-(trifluoromethyl)phenyl]-1-cyclohexene-1-carboxamide (200 mg) as pale yellow crystals.

¹H-NMR(DMSO-d₆): δ 1.73(4H, br s), 2.50(8H, br s), 3.02(4H, br s), 3.67(2H, br s), 6.75(2H, d, J=8.9 Hz), 7.15(2H, d, J=8.9 Hz), 7.47(2H, d, J=7.9 Hz), 7.61(2H, d, J=8.2 Hz), 7.86(0.55H, br s), 8.48(0.45H, br s), 9.39(1H, s), 13.86(1H, br s)

ESI-MS(m/z): 533(M+Na)⁺

### EXAMPLE 391

To a solution of N-[4-(1-piperazinyl)phenyl]-2-[4-(trifluoromethyl)phenyl]-1-cyclohexene-1-carboxamide (300 mg) and 2-vinylpyridine (88 mg) in 2-propanol was added acetic acid (0.04 ml). The reaction mixture was refluxed for 20 hours, cooled, and concentrated in vacuo. The residue was crystallized from ethyl acetate-diisopropyl ether to give N-(4-{4-[2-(2-pyridinyl)ethyl]-1-piperazinyl}phenyl)-2-[4-(trifluoromethyl)phenyl]-1-cyclohexene-1-carboxamide (230 mg) as a white solid.

¹H-NMR(DMSO-d₆): δ 1.73(4H, br-s), 2.38(4H, br s), 3.21(4H, br s), 3.24(6H, m), 3.53(2H, br s), 6.85(2H, d, J=8.9 Hz), 7.22(2H, d, J=8.6 Hz), 7.30(1H, dd, J=7.6,4.9 Hz), 7.36(2H, d, J=7.9 Hz), 7.48(2H, d, J=8.2 Hz), 7.62(2H, d, J=8.2 Hz), 7.78(1H, td, J=7.6,1.6 Hz), 8.53(1H, d, J=4.6 Hz), 9.49(1H, s)

ESI-MS (m/z): 535 (M+H)⁺

Preparation 177

To a solution of 2-methylpiperazine (5.02 g) in N,N-dimethylimidazolidinone (20 ml) was added 1-fluoro-4-nitrobenzene (2.36 g) at ambient temperature. The reaction was heated to 50° C. and stirred for an hour. The reaction mixture was poured into water, then extracted with ethyl acetate. The separated organic layer was washed with water (three times) and brine, dried over magnesium sulfate and concentrated in vacuo to yield 3-methyl-1-(4-nitrophenyl)piperazine (3.35 g) as yellow crystals.

¹H-NMR(CDCl₃): δ 1.16(3H, d, J=6.3 Hz), 2.58(1H, dd, J=10.2 and 12.2 Hz), 2.86-3.04(3H, m), 3.10-3.19(1H, m), 3.70-3.83(2H, m), 6.82(2H, d, J=9.6 Hz), 8.12(2H, d, J=9.2 Hz)

(+)ESI-MS(m/z): 222(M+H)⁺

Preparation 178

To a solution of 3-methyl-1-(4-nitrophenyl)piperazine (1.09 g) and di-tert-butyl dicarbonate (1.20 g) in tetrahydrofuran (20 ml) was added 4-(N,N-dimethyl)aminopyridine (30 mg) as a solid at ambient temperature. The reaction mixture was stirred at ambient temperature for 19 hours and concentrated in vacuo. The residue was purified by column chromatography on silica gel by eluting with hexane:ethyl acetate (3:1) to give tert-butyl 2-methyl-4-(4-nitrophenyl)-1-piperazinecarboxylate (1.55 g) as orange colored crystals.

¹H-NMR(CDCl₃): δ 1.23(3H, d, J=6.6 Hz), 1.49(9H, s), 3.13(1H, dt, J=4.0 and 10.6 Hz), 3.36(1H, dt, J=3.3 and 10.2 Hz), 3.61(1H, d, J=12.9 Hz), 3.74(1H, brd, J=12.2 Hz), 3.94(1H, dt, J=3.9 and 7.6 Hz), 4.34(1H, br s), 6.76(2H, d, J=9.6 Hz), 8.13(2H, d, J=9.3 Hz)

(+)ESI-MS(m/z): 344(M+Na)⁺

Preparation 179

A solution of tert-butyl 2-methyl-4-(4-nitrophenyl)-1-piperazinecarboxylate (166 mg) in methanol (5 ml) was hydrogenated over 10% palladium on carbon (33 mg) at ambient temperature under atmospheric pressure of hydrogen for 40 minutes. The reaction mixture was filtered through a pad of celite, and the filtrate was concentrated in vacuo to give tert-butyl 4-(4-aminophenyl)-2-methyl-1-piperazinecarboxylate (151 mg) as a dark red tar. The product was used for the next step without further purification.

Preparation 180

To a solution of 2-[4-(trifluoromethyl)phenyl]-1-cyclohexene-1-carboxylic acid (140 mg) in toluene (10 ml) were added thionyl chloride (92 mg) and N,N-dimethylformamide (2 drops) and the mixture was stirred at 50° C. for 40 minutes. The mixture was evaporated in vacuo and the residue was dissolved in tetrahydrofuran (10 ml). The acid chloride in tetrahydrofuran was added to a solution of tert-butyl 4-(4-aminophenyl)-2-methyl-1-piperazinecarboxylate (151 mg) and triethylamine (58 mg) in tetrahydrofuran (10 ml) at ambient temperature and the mixture was stirred at the same temperature for 30 minutes. The mixture was poured into water and extracted with ethyl acetate. The organic layer was washed with brine, dried over magnesium sulfate, and evaporated in vacuo to give tert-butyl 2-methyl-4-{4-[({2-[4-(trifluoromethyl)phenyl]-1-cyclohexen-1-yl}carbonyl)amino]-phenyl}-1-piperazinecarboxylate (258 mg) as faintly purple crystals.

¹H-NMR(CDCl₃): δ 1.23(3H, d, J=6.6 Hz), 1.49(9H, s), 3.13(1H, dt, J=4.0 and 10.6 Hz), 3.36(1H, dt, J=3.3 and 10.2 Hz), 3.61(1H, d, J=12.9 Hz), 3.74(1H, brd, J=12.2 Hz), 3.94(1H, dt, J=3.9 and 7.6 Hz), 4.34(1H, br s), 6.76(2H, d, J=9.6 Hz), 8.13(2H, d, J=9.3 Hz)

(+)ESI-MS(m/z): 566(M+Na)⁺

Preparation 181

To a solution of tert-butyl 2-methyl-4-{4-[({2-[4-(trifluoromethyl)phenyl]-1-cyclohexen-1-yl}carbonyl)amino]phenyl}-1-piperazinecarboxylate (238 mg) in dichloromethane (10 ml) was added trifluoroacetic acid (1.11 g). The reaction mixture was stirred for 13 hours, quenched with 10% aqueous potassium carbonate solution, and extracted with dichloromethane. The organic layer was washed with brine, dried over magnesium-sulfate, filtered, and concentrated in vacuo to give N-[4-(3-methyl-1-piperazinyl)phenyl]-2-[4-(trifluoromethyl)phenyl]-1-cyclohexene-1-carboxamide (187 mg) as pale brown crystals.

¹H-NMR(CDCl₃): δ 1.49(3H, d, J=6.3 Hz), 1.80(4H, br s), 2.44(2H, br s), 2.54(2H, br s), 2.96(1H, brt, J=10.2 Hz), 3.10-3.28(2H, m), 3.35-3.52(4H, m), 6.46(1H, s), 6.73(2H, d, J=8.9 Hz), 6.87(2H, d, J=8.9 Hz), 7.41(2H, d, J=8.3 Hz), 7.59(2H, d, J=8.2 Hz)

(+)ESI-MS(m/z): 444(M+H)⁺

### EXAMPLE 392

To a solution of N-[4-(3-methyl-1-piperazinyl)phenyl]-2-[4-(trifluoromethyl)phenyl]-1-cyclohexene-1-carboxamide (181 mg) and 3-formylbenzonitrile (111 mg) in dichloromethane (10 ml) was added sodium triacetoxyborohydride (268 mg) at ambient temperature. The reaction mixture was stirred for 17 hours, quenched with 10% aqueous potassium carbonate solution, and extracted with dichloromethane twice. The combined organic layers were washed with brine, dried over magnesium sulfate, and concentrated in vacuo. The residue was purified by column chromatography on silica gel by eluting with hexane:ethyl acetate (2:1 to 1:1) to give N-{4-[4-(3-cyanobenzyl)-3-methyl-1-piperazinyl]phenyl}-2-[4-(trifluoromethyl)phenyl]-1-cyclohexene-1-carboxamide (107 mg) as colorless crystals.

¹H-NMR(CDCl₃): δ 1.17(3H, d, J=4.6 Hz), 1.80(4H, br s), 2.22-2.38(1H, m), 2.43(2H, br s), 2.54(2H, br s), 2.60-2.89(4H, m), 3.15-3.38(3H, m), 4.07(1H, d, J=14.2 Hz), 6.41(1H, br s), 6.73(2H, d, J=9.2 Hz), 6.83(2H, d, J=9.2 Hz), 7.35-7.46(3H, m), 7.51-7.62(4H, m), 7.68(1H, s)

(+)ESI-MS(m/z) :559(M+H)⁺

Preparation 182

To a solution 2-{2-[(methylsulfonyl)oxy]ethyl}-4-nitrobenzyl methanesulfonate (1.5 g) in tetrahydrofuran (3 ml) was added ammonia (13.1 ml) at −78° C. in the glass autoclave. The mixture was warmed at 24° C. for 28 hours (120 psi). Ammonia was distilled off, and the mixture was poured into water and extracted with ethyl acetate. The organic layer was washed with brine, dried over magnesium sulfate, and evaporated in vacuo to give 6-nitro-1,2,3,4-tetrahydroisoquinoline (756 mg) as brown oil.

¹H-NMR(CDCl₃): δ 2.90(2H, t, J=5.7 Hz), 3.17(2H, t, J=5.9 Hz), 4.09(2H, s), 7.15(1H, d, J=9.2 Hz), 7.94-7.98(2H, m)

ESI-MS(m/z): 179(M+H)⁺

Preparation 183

To a solution of 6-nitro-1,2,3,4-tetrahydroisoquinoline (756 mg) in tetrahydrofuran (13.4 ml) was added di-t-butyl dicarbonate (1.02 g) and the mixture was stirred at ambient temperature for 15 hours. The mixture was poured into water and extracted with ethyl acetate. The organic layer was washed with brine, dried over magnesium sulfate, and evaporated in vacuo. The residue was purified by column chromatography on silica gel by eluting with hexane:ethyl acetate (6:1) to give tert-butyl 6-nitro-3,4-dihydro-2(1H)-isoquinolinecarboxylate (906 mg) as a pale yellow powder.

¹H-NMR(CDCl₃): δ 1.50(9H, s), 2.94(2H, t, J=5.7 Hz), 3.69(2H, t, J=5.9 Hz), 4.66(2H, s), 7.26(1H, d, J=8.9 Hz), 8.02-8.06(2H, m)

Preparation 184

A solution of tert-butyl 6-nitro-3,4-dihydro-2(1H)-isoquinolinecarboxylate (906 mg) in methanol (9 ml) was hydrogenated over 10% palladium on carbon (453 mg, 50% wet) at ambient temperature under atmospheric pressure of hydrogen for 2 hours. The reaction mixture was filtered through a short pad of celite, and the filtrate was concentrated in vacuo to give tert-butyl 6-amino-3,4-dihydro-2(1H)-isoquinolinecarboxylate (808 mg) as a pale brown oil.

¹H-NMR(CDCl₃): δ 1.48(9H, s), 2.73(2H, t, J=5.4 Hz), 3.40-3.80(4H, m), 4.54(2H, s), 6.47(1H, d, J=2.2 Hz), 6.54(1H, dd, J=8.1, 2.2 Hz), 6.89(1H, d, J=7.8 Hz)

ESI-MS(m/z): 271(M+Na)⁺

Preparation 185

To a solution of 2-[4-(trifluoromethyl)phenyl]-1-cyclohexene-1-carboxylic acid (1.14 g) in toluene (11.4 ml) were added thionyl chloride (0.617 ml) and N,N-dimethylformamide (3 drops) and the mixture was stirred at 80° C. for an hour. The mixture was evaporated in vacuo and the residue was dissolved in tetrahydrofuran (3 ml). The acid chloride in tetrahydrofuran was added to a solution of tert-butyl 6-amino-3,4-dihydro-2(1H)-isoquinolinecarboxylate (808 mg) and triethylamine (0.68 ml) in tetrahydrofuran (5 ml) at ambient temperature and the mixture was stirred at the same temperature for 2 hours. The mixture was poured into water and extracted with ethyl acetate. The organic layer was washed with brine, dried over magnesium sulfate, and evaporated in vacuo. The residue was recrystallized from ethyl acetate and hexane to give tert-butyl 6-[({2-[4-(trifluoromethyl)phenyl]-1-cyclohexen-1-yl}carbonyl)amino]-3,4-dihydro-2(1H)-isoquinolinecarboxylate (1.382 g) as a white powder.

¹H-NMR(CDCl₃): δ 1.47(9H, s), 1.80(4H, br s), 2.44(2H, br s), 2.55(2H, br s), 2.69(2H, t, J=5.9 Hz), 3.56(2H, t, J=5.9 Hz), 4.59(2H, s), 6.45(1H, s), 6.67-6.80(1H, m), 6.90(1H, d, J=8.1 Hz), 7.41(2H, d, J=8.1 Hz), 7.59(2H, d, J=8.4 Hz)

ESI-MS(m/z): 523(M+Na)⁺

Preparation 186

To a solution of tert-butyl 6-[({2-[4-(trifluoromethyl)phenyl]-1-cyclohexen-1-yl)carbonyl)amino]-3,4-dihydro-2(1H)-isoquinolinecarboxylate (1.3 g) in dichloromethane (6.5 ml) was added trifluoroacetic acid (1 ml). The reaction mixture was stirred at ambient temperature for 22.5 hours, quenched with 10% aqueous potassium carbonate. solution, and extracted with tetrahydrofuran and ethyl acetate. The organic layer was washed with brine, dried over magnesium sulfate, filtered, and concentrated in vacuo. The residue was recrystallized from ethyl acetate-hexane to give N-(1,2,3,4-tetrahydro-6-isoquinolinyl)-2-[4-(trifluoromethyl)phenyl]-1-cyclohexene-1-carboxamide (1.016 g) as a white powder.

¹H-NMR(DMSO-d₆): δ 1.75(4H, br s), 2.39(4H, br s), 2.67(2H, t, J=5.8 Hz), 3.03(2H, t, J=5.4 Hz), 3.88(2H, s), 6.89(1H, d, J=8.6 Hz), 7.04(1H, d, J=8.4 Hz), 7.14(1H, s), 7.47(2H, d, J=8.4 Hz), 7.62(2H, d, J=8.4 Hz), 9.54(1H, s)

ESI-MS(m/z): 401(M+H)⁺

### EXAMPLE 393

To a solution N-(1,2,3,4-tetrahydro-6-isoquinolinyl)-2-[4-(trifluoromethyl)phenyl]-1-cyclohexene-1-carboxamide (165.6 mg) in dichloromethane (2.32 ml) was added 3-formylbenzonitrile (108 mg) and sodium triacetoxyborohydride (263 mg). The mixture was stirred at ambient temperature for 3 hours. The reaction mixture was quenched with 10% aqueous potassium carbonate solution, and extracted with tetrahydrofuran and ethyl acetate. The organic layer was washed with brine, dried over magnesium sulfate, filtered, and concentrated in vacuo. The residue was recrystallized from ethyl acetate-hexane to give N-[2-(3-cyanobenzyl)-1,2,3,4-tetrahydro-6-isoquinolinyl]-2-[4-(trifluoromethyl)phenyl]-1-cyclohexene-1-carboxamide (194 mg) as a white powder.

¹H-NMR(DMSO-d₆): δ 1.74(4H, br s), 2.39(4H, br s), 2.61-2.70(4H, m), 3.43(2H, s), 3.66(2H, s), 6.82(1H, d, J=8.4 Hz), 7.00(1H, dd, J=8.1, 1.9 Hz), 7.12(1H, d, J=2.2 Hz), 7.47(2H, d, J=8.4 Hz), 7.55(1H, t, J=7.6 Hz), 7.62(2H, d, J=8.4 Hz), 7.67-7.77(3H, m), 9.51(1H, s)

ESI-MS(m/z): 516(M+H)⁺

### EXAMPLE 394

N-[2-(2-cyanobenzyl)-1,2,3,4-tetrahydro-6-isoquinolinyl]-2-[4-(trifluoromethyl)phenyl]-1-cyclohexene-1-carboxamide was obtained in the same manner as in Example 393 as a pale yellow powder.

¹H-NMR(DMSO-d₆): δ 1.74(4H, br s), 2.38(4H, br s), 2.68(4H, br s), 3.49(2H, s), 3.78(2H, s), 6.83(1H, d, J=8.4 Hz), 7.00(1H, dd, J=8.4, 1.9 Hz), 7.13(1H, d, J=1.6 Hz), 7.45-7.50(3H, m), 7.60-7.71(4H, m), 7.82(1H, d, J=7.6 Hz), 9.52(1H, s)

ESI-MS(m/z): 516(M+H)⁺

### EXAMPLE 395

N-[2-(4-Cyanobenzyl)-1,2,3,4-tetrahydro-6-isoquinolinyl]-2-[4-(trifluoromethyl)phenyl]-1-cyclohexene-1-carboxamide was obtained in the same manner as in Example 393 as a white powder.

¹H-NMR(DMSO-d₆): δ 1.74(4H, br s), 2.38(4H, br s), 2.61-2.70(4H, m), 3.43(2H, s), 3.69(2H, s), 6.82(1H, d, J=8.6 Hz), 7.00(1H, br d, J=8.4 Hz), 7.13(1H, br s), 7.47(2H, d, J=8.4 Hz), 7.54(2H, d, J=8.4 Hz), 7.62(2H, d, J=8.4 Hz), 7.79(2H, d, J=8.1 Hz), 9.51(1H, s)

ESI-MS(m/z): 516(M+H)⁺

### EXAMPLE 396

N-[2-(1,3-Thiazol-2-ylmethyl)-1,2,3,4-tetrahydro-6-isoquinolinyl]-2-[4-(trifluoromethyl)phenyl]-1-cyclohexene-1-carboxamide was obtained in the same manner as in Example 393 as pale yellow powder.

¹H-NMR(DMSO-d₆): δ 1.73(4H, br s), 2.39(4H, br s), 2.72(4H, br s), 3.59(2H, s), 3.96(2H, s), 6.85(1H, d, J=8.1 Hz), 7.02(1H, dd, J=8.4, 2.2 Hz), 7.14(1H, d, J=1.9 Hz), 7.47(2H, d, J=8.4 Hz), 7.62(2H, d, J=8.1 Hz), 7.66(1H, d, ′J=3.2 Hz), 7.73(1H, d, J=3.2 Hz), 9.54(1H, s)

ESI-MS(m/z): 498(M+H)⁺

### EXAMPLE 397

To a solution of N-(1,2,3,4-tetrahydro-6-isoquinolinyl)-2-[4-(trifluoromethyl)phenyl]-1-cyclohexene-1-carboxamide (193.3 mg) in tetrahydrofuran (3.87 ml) were added triethylamine (80.7 μl) and (1-trityl-1H-1,2,4-triazol-3-yl)methyl methanesulfonate (243 mg). The mixture was stirred at ambient temperature for 4.5 hours. The reaction mixture was poured into water and extracted with ethyl acetate. The organic layer was washed with brine, dried over magnesium sulfate, and evaporated in vacuo. The residue was purified by column chromatography on silica gel by eluting with hexane:ethyl acetate (1:2) to give 2-[4-(trifluoromethyl)phenyl]-N-{2-[(1-trityl-1H-1,2,4-triazol-3-yl)methyl]-1,2,3,4-tetrahydro-6-isoquinolinyl}-1-cyclohexene-1-carboxamide (274 mg) as a pale yellow solid.

¹H-NMR(CDCl₃): δ 1.80(4H, br s), 2.43-2.54(4H, m), 2.74(4H, s), 3.57(2H, s), 3.85(2H, s), 6.41(

¹H, s), 6.61-6.76(3H, m), 7.12-7.16(6H, m), 7.28-7.34(9H, m), 7.40(2H, d, J=8.1 Hz), 7.59(2H, d, J=8.1 Hz), 7.92(1H, s)

ESI-MS(m/z): 746(M+Na)⁺

### EXAMPLE 398

To a solution of 2-[4-(trifluoromethyl)phenyl]-N-{2-[(1-trityl-1H-1,2,4-triazol-3-yl)methyl]-1,2,3,4-tetrahydro-6-isoquinolinyl}-1-cyclohexene-1-carboxamide (262.5 mg) in methanol (2.6 ml) was added 35% hydrochloric acid (0.15 ml). The mixture was stirred at ambient temperature for 24 hours. The mixture was poured into a mixture of water and saturated aqueous sodium bicarbonate solution, and extracted with ethyl acetate and tetrahydrofuran. The organic layer was washed with brine, dried over magnesium sulfate, and evaporated in vacuo. The residue was recrystallized from ethyl acetate-hexane to give N-[2-(1H-1,2,4-triazol-3-ylmethyl)-1,2,3,4-tetrahydro-6-isoquinolinyl]-2-[4-(trifluoromethyl)phenyl]-1-cyclohexene-1-carboxamide (143 mg) as a pale yellow powder.

¹H-NMR(DMSO-d₆): δ 1.73(4H, br s), 2.38(4H, br s), 2.67(4H, br s), 3.49(2H, s), 3.74(2H, s), 6.83(1H, d, J=8.6 Hz), 7.01(1H, dd, J=8.4, 2.2 Hz), 7.11(1H, s), 7.47(2H, d, J=8.1 Hz), 7.62(2H, d, J=8.4 Hz), 7.87(1/2H, br s), 8.46(½H, br s), 9.51(1H, s), 13.86(1H, s)

ESI-MS(m/z): 482(M+Na)⁺

### EXAMPLE 399

To a solution N-(1,2,3,4-tetrahydro-6-isoquinolinyl)-2-[4-(trifluoromethyl)phenyl]-1-cyclohexene-1-carboxamide (90 mg) in dichloromethane (1.3 ml) were added 3-methylbenzaldehyde (54 mg) and sodium triacetoxyborohydride (143 mg). The mixture was stirred at ambient temperature for 3.5 hours. The reaction mixture was quenched with 10% aqueous potassium carbonate solution, and extracted with ethyl acetate. The organic layer was washed with brine, dried over magnesium sulfate, filtered, and concentrated in vacuo. The residue was recrystallized from ethyl acetate-hexane to give N-[2-(3-methylbenzyl)-1,2,3,4-tetrahydro-6-isoquinolinyl]-2-[4-(trifluoromethyl)phenyl]-1-cyclohexene-1-carboxamide (90.2 mg) as a white powder.

¹H-NMR(DMSO-d₆): δ 1.73(4H, br s), 2.29(3H, s), 2.38(4H, br s), 2.59-2.68(4H, m), 3.38(2H, s), 3.55(2H, s), 6.81(1H, d, J=8.1 Hz), 6.97-7.23(6H, m), 7.47(2H, d, J=7.8. Hz), 7.61(2H, d, J=8.4 Hz), 9.48(1H, s)

ESI-MS(m/z): 505(M+H)⁺

### EXAMPLE 400

N-[2-(3-Methoxybenzyl)-1,2,3,4-tetrahydro-6-isoquinolinyl]-2-[4-(trifluoromethyl)phenyl]-1-cyclohexene-1-carboxamide was obtained in the same′manner as in Example 399 as a pale yellow powder.

¹H-NMR(DMSO-d₆): δ 1.23(4H, br s), 2.38(4H, br s), 2.59-2.68(4H, m), 3.42(2H, s), 3.60(2H, s), 3.73(3H, s), 6.82(1H, d, J=8.1 Hz), 6.88(1H, s), 6.90(1H, d, J=7.3 Hz), 7.00(1H, d, J=8.1 Hz), 7.11(1H, s), 7.23(1H, t, J=7.8 Hz), 7.31-7.38(1H, m), 7.47(2H, d, J=7.8 Hz), 7.61(2H, d, J=7.8 Hz), 9.49(1H, s)

ESI-MS(m/z): 521(M+H)⁺

### EXAMPLE 401

N-[2-(3-Chlorobenzyl)-1,2,3,4-tetrahydro-6-isoquinolinyl]-2-[4-(trifluoromethyl)phenyl]-1-cyclohexene-1-carboxamide was obtained in the same manner as in Example 399 as a white powder.

¹H-NMR(DMSO-d₆): δ 1.73(4H, br s), 2.38(4H, br s), 2.61-2.69(4H, m), 3.42(2H, s), 3.61(2H, s), 6.81(1H, d, J=8.1 Hz), 7.00(1H, d, J=8.4 Hz), 7.12(1H, s), 7.25-7.38(4H, m), 7.47(2H, d, J=7.8 Hz), 7.62(2H, d, J=8.4 Hz), 9.49(1H, s)

ESI-MS(m/z): 526(M+H)⁺

### EXAMPLE 402

N-[2-(1H-Imidazol-5-ylmethyl)-1,2,3,4-tetrahydro-6-isoquinolinyl]-2-[4-(trifluoromethyl)phenyl]-1-cyclohexene-1-carboxamide was obtained in the same manner as in

### EXAMPLE 399 as a pale yellow powder.

¹H-NMR(DMSO-d₆): δ 1.24(4H, br s), 2.38(4H, br s), 2.62-2.65(4H, m), 3.42(2H, s), 3.54(2H, s), 6.82(1H, d, J=8.6 Hz), 6.87(1H, s), 7.00(1H, d, J=8.4 Hz), 7.09(1H, s), 7.46(2H, d, J=8.4 Hz), 7.54(1H, s), 7.61(2H, d, J=8.1 Hz), 9.48(1H, s)

ESI-MS(m/z): 481(M+H)⁺

### EXAMPLE 403

N-[2-(1H-Imidazol-2-ylmethyl)-1,2,3,4-tetrahydro-6-isoquinolinyl]-2-[4-(trifluoromethyl)phenyl]-1-cyclohexene-1-carboxamide was obtained in the same manner as in Example 399 as a pale yellow powder.

¹H-NMR(DMSO-d₆): δ 1.73(4H, br s), 2.38(4H, br s), 2.60-2.68(4H, m), 3.44(2H, s), 3.63(2H, s), 6.53(1H, s), 6.83(1H, d, J=8.4 Hz), 6.91(2H, s), 7.01(1H, d, J=8.1 Hz), 7.11(1H, s), 7.46(2H, d, J=8.4 Hz), 7.60(2H, d, J=8.4 Hz), 9.49(1H, s)

ESI-MS(m/z): 516(M+H)⁺

### EXAMPLE 404

N-[2-(1H-Pyrrol-2-ylmethyl)-1,2,3,4-tetrahydro-6-isoquinolinyl]-2-[4-(trifluoromethyl)phenyl]-1-cyclohexene-1-carboxamide was obtained in the same manner as in Example 399 as a pale brown powder.

¹H-NMR(DMSO-d₆): δ 1.73(4H, br s), 2.38(4H, br s), 2.56-2.66(4H, m), 3.38(2H, s), 3.52,(2H, s), 5.91(2H, m), 6.23(1H, s), 6.81(1H, d, J=8.4 Hz), 7.00(1H, d, J=8.4 Hz), 7.10(1H, s), 7.46(2H, d, J=8.4 Hz), 7.61(2H, d, J=8.4 Hz), 9.48(1H, s), 10.67(1H, s)

ESI-MS(m/z): 480(M+H)⁺

### EXAMPLE 405

To a solution of 1-(2-pyridinylacetyl)-5-indolinamine (760 mg), 2-[4-(trifluoromethyl)phenyl]-1-cyclopentene-1-carboxylic acid (846 mg) and benzotriazol-1-yl-oxytripyrrolidinophosphonium hexafluorophosphate (PyBOP) (2.92 g) in N,N-dimethylformamide (30 ml) was added dropwise diisopropylethylamine (776 mg) at ambient temperature and the mixture was stirred at the same temperature for 16 hours. The mixture was poured into a mixture of ethyl acetate, water and 6N hydrochloric acid, and the separated organic layer was washed with water and brine, dried over magnesium sulfate, and evaporated in vacuo. The residue was purified by column chromatography on silica gel eluting with ethyl acetate and crystallized from ethyl acetate to give N-[1-(2-pyridinylacetyl)-2,3-dihydro-1H-indol-5-yl]-2-[4-(trifluoromethyl)phenyl]-1-cyclopentene-1-carboxamide (754 mg) as white crystals.

¹H-NMR(DMSO-d₆): δ 1.9-2.1(2H, m), 2.8-2.95(4H, m), 3.12(2H, t, J=8.3 Hz), 3.99(2H, s), 4.19(2H, t, J=8.3 Hz), 7.2-7.4(3H, m), 7.5-7.8(6H, m), 7.2-7.4(5H, m), 7.92(1H, d, J=8.7 Hz), 8.45-8.5(1H, m), 9.91(1H, s)

ESI-MS(m/z): 514(M+Na)⁺, 492(M+H)⁺

### EXAMPLE 406

N-[1-(2-Pyridinylacetyl)-2,3-dihydro-1H-indol-5-yl]-2-[4-(trifluoromethyl)phenyl]-1-cycloheptene-1-carboxamide was obtained in the same manner as in Example 405 as white crystals.

¹H-NMR(DMSO-d₆): δ 1.6-1.9(6H, m), 2.21(3H, s), 2.4-2.5(4H, m), 2.85(2H, t, J=7.7 Hz), 3.99(2H, t, J=7.7 Hz), 7.0-7.3(8H, m), 7.37(2H, d, J=8.7 Hz), 7.6-7.7(1H, m), 8.25(1H, s), 8.45(1H, d, J=3.9 Hz), 9.42(1H, s)

ESI-MS(m/z): 488(M+Na)⁺, 466(M+H)⁺

### EXAMPLE 407

N-[1-(2-Pyridinylacetyl)-2,3-dihydro-1H-indol-5-yl]-2-[4-(trifluoromethyl)phenyl]-1-cyclooctene-1-carboxamide was obtained in the same manner as in Example 405 as white crystals.

¹H-NMR(DMSO-d₆): δ 1.6-1.9(6H, m), 2.21(3H, s), 2.4-2.5(4H, m), 2.85(2H, t, J=7.7 Hz), 3.99(2H, t, J=7.7 Hz), 7.0-7.3(8H, m), 7.37(2H, d, J=8.7 Hz), 7.6-7.7(1H, m), 8.25(1H, s), 8.45(1H, d, J=3.9 Hz), 9.42(1H, s)

ESI-MS(m/z): 488(M+Na)⁺, 466(M+H)⁺

### EXAMPLE 408

2-(4-Methylphenyl)-N-[1-(2-pyridinylacetyl)-2,3-dihydro-1H-indol-5-yl]-1-cyclooctene-1-carboxamide was obtained in the same manner as in Example 405 as white crystals.

¹H-NMR(DMSO-d₆): δ 1.6-1.9(6H, m), 2.21(3H, s), 2.4-2.5(4H, m), 2.85(2H, t, J=7.7 Hz), 3.99(2H, t, J=7.7 Hz), 7.0-7.3(8H, m), 7.37(2H, d, J=8.7 Hz), 7.6-7.7(1H, m), 8.25(1H, s), 8.45(1H, d, J=3.9 Hz), 9.42(1H, s)

ESI-MS(m/z): 488(M+Na)⁺, 466(M+H)⁺

### EXAMPLE 409

2-(4-Methylphenyl)-N-[1-(2-pyridinylacetyl)-2,3-dihydro-1H-indol-5-yl]-1-cyclopentene-1-carboxamide was obtained in the same manner as in Example 405 as white crystals.

¹H-NMR(DMSO-d₆): δ 1.85-2.05(2H, m), 2.25(3H, s), 2.7-2.9(4H, m), 3.12(2H, t, J=8.5 Hz), 3.99(2H, s), 4.19(2H, t, J=8.5 Hz), 7.10(2H, d, J=8.0 Hz), 7.2-7.4(5H, m), 7.56(1H, s), 7.65-7.8(1H, m), 7.92(1H, d, J=8.7 Hz), 8.49(1H, d, J=4.1 Hz), 9.85(1H, s)

negative ESI-MS(m/z): 436(M−H)⁻

### EXAMPLE 410

To a suspension of 2-(4-methylphenyl)-1-cyclohexene-1-carboxylic acid (2.38 g) in toluene (23 ml) were added thionyl chloride (1.78 g) and N,N-dimethylformamide (3 drops) and the mixture was stirred at 70° C. for 3 hours. The mixture was evaporated to dryness and the crude acid chloride was dissolved in tetrahydrofuran (15 ml). To a solution of 4-aminophenyl (2-(2-pyridinyl) ethyl) formamide (2.413 g) in tetrahydrofuran (40 ml) and triethylamine (2.02 g) was added dropwise the above acid chloride solution at ambient temperature and the mixture was stirred at the same temperature for 16 hours. The mixture was poured into a mixture of ethyl acetate, water, and 6N hydrochloric acid and the separated organic layer was washed with water and brine, dried over magnesium sulfate, and evaporated in vacuo. The residue was purified by column chromatography on silica gel eluting with ethyl acetate to give N-(4-{formyl[2-(2-pyridinyl)ethyl]amino}phenyl)-2-(4-methylphenyl)-1-cyclohexene-1-carboxamide (3.58 g) as a brown powder.

¹H-NMR(DMSO-d₆): δ 1.6-1.8(4H, m), 2.3-2.45(4H, m), 2.85(2H, t, J=7.2 Hz), 4.04(2H, t, J=7.2 Hz), 7.0-7.25(8H, m), 7.42(2H, d, J=8.8 Hz), 7.6-7.75(1H, m), 8.25(1H, s), 8.45-8.5(1H, m), 9.65(1H, s)

ESI-MS(m/z): 462(M+Na)⁺, 440(M+H)⁺

### EXAMPLE 411

To a suspension of N-(4-{formyl[2-(2-pyridinyl)ethyl]amino}phenyl)-2-(4-methylphenyl)-1-cyclohexene-1-carboxamide (3.56 g) in methanol (20 ml) was added dropwise concentrated hydrochloric acid (3.8 ml) at ambient temperature and the mixture was stirred at 35° C. for 5 hours. The mixture was poured into a mixture of ethyl acetate and water, and adjusted to pH 8 with 50% aqueous potassium carbonate solution. The separated organic layer was washed with water and brine, dried over magnesium sulfate, and evaporated in vacuo. The residue was purified by column chromatography on silica gel eluting with ethyl acetate and recrystallized from ethyl acetate and diisopropyl ether to give 2-(4-methylphenyl)-N-(4-{[2-(2-pyridinyl)ethyl]amino}-phenyl)-1-cyclohexene-1-carboxamide (1.29 g) as white crystals.

¹H-NMR(DMSO-d₆): δ 1.5-2.0(4H, m), 2.2-2.35(4H, m), 2.94(2H, t, J=7.0 Hz), 3.32(2H, td, J=7.0 and 5.7 Hz), 5.46(1H, t, J=5.7 Hz), 6.48(2H, d, J=8.8 Hz), 7.07(2H, d, J=8.0 Hz), 7.15-7.35(6H, m), 7.65-7.8(1H, m), 8.50(1H, d, J=4.4 Hz), 9.55(1H, s)

ESI-MS(m/z): 434(M+Na)⁺, 412 (M+H)⁺

Preparation 187

To a solution of 1,4-benzenediamine (1.298 g) and triethylamine (1.52 g) in acetonitrile (50 ml) was added dropwise a solution of 2-(4-methylphenyl)-1-cyclohexene-1-carboxylic acid chloride (3;52 g) in acetonitrile (20 ml) at 5° C. under a nitrogen atmosphere and the mixture was stirred at the same temperature for 4 hours. Methanol (4 ml) was added and the mixture was stirred for 10 minutes. The mixture was extracted with ethyl acetate and the separated organic layer was washed with water and brine, dried over magnesium sulfate, and evaporated in vacuo. The residue was dissolved in ethyl acetate (80 ml) and methanesulfonic acid (1.15 g) was added to the solution. The resulting precipitates were collected by filtration and washed with ethyl acetate to give N-(4-aminophenyl)-2-(4-methylphenyl)-1-cyclohexene-1-carboxamide methanesulfonate (4.28 g) as a pale brown powder.

¹H-NMR(DMSO-d₆): δ 1.6-1.8(4H, m), 2.20(3H, s), 2.31(3H, s), 2.25-2.4(4H, m), 6.95-7.3(6H, m), 7.4-7.5(2H, m), 9.66(1H, s)

ESI-MS(m/z): 329(M+Na)⁺, 307(M+H)⁺

### EXAMPLE 412

To a suspension of N-(4-aminophenyl)-2-(4-methylphenyl)-1-cyclohexene-1-carboxamide methanesulfonate (3.06 g) in 2-propanol (30 ml) was added 2-vinylpyridine (961 mg) and the mixture was refluxed for 16 hours. The reaction mixture was evaporated in vacuo. The residue was purified by column chromatography on silica gel eluting with ethyl acetate and recrystallized from ethyl acetate:diisopropyl ether (1:1) to give 2-(4-methylphenyl)-N-(4-{[2-(2-pyridinyl)ethyl]amino}-phenyl)-1-cyclohexene-1-carboxamide (2.15 g) as pale brown crystals.

¹H-NMR(DMSO-d₆): δ 1.7-1.9(4H, m), 2.22(3H, s), 2.25-2.4(4H, m), 2.94(2H, t, J=7.0 Hz), 3.30(2H, td, J=7.0 and 5.6 Hz), 5.43(1H, t, J=5.6 Hz), 6.43(2H, d, J=8.9 Hz), 6.95-7.3(10H, m), 7.6-7.75(1H, m), 8.45-8.5(1H, m), 9.05(1H, s)

ESI-MS(m/z): 434(M+Na)⁺, 412(M+H)⁺

### EXAMPLE 413

To a solution of 2-(phenylacetyl)-5-isoindolinamine in N,N-dimethylformamide (0.5 mol/L, 20 μl) were added a solution of 1-hydroxybenzotriazole hydrate in N,N-dimethylformamide (1 mol/L, 15 μl) and a solution of 2-[4-(trifluoromethyl)phenyl]-1-cyclopentene-1-carboxylic acid in N,N-dimethylformamide (0.1 mol/L, 150 μl) at ambient temperature. To the mixture was added a solution of 1-[3-(dimethylamino)propyl]-3-ethylcarbodiimide (1 mol/L, 15 μl) and the mixture was stirred at 50° C. for 6 hours. The reaction mixture was diluted with ethyl acetate (10 ml), washed with water, saturated aqueous sodium hydrogencarbonate solution and brine, and evaporated in vacuo to give N-[2-(phenylacetyl)-2,3-dihydro-1H-isoindol-5-yl]-2-[4-(trifluoromethyl)phenyl]-1-cyclopentene-1-carboxamide as a solid.

(+)ESI-MS(m/z): 513(M+Na)⁺

### EXAMPLES 414-419

The compounds of Examples 414-419 shown in Table 4 were obtained in the same manner as in Example 413 as a solid.

### EXAMPLE 420

To a solution of 2-(4-bromophenyl)-N-(2,3-dihydro-1H-isoindol-5-yl)-1-cyclohexene-1-carboxamide in N,N-dimethylformamide (0.5 mol/L, 20 μl) were added a solution of 1-hydroxybenzotriazole hydrate in N,N-dimethylformamide (1 mol/L, 15 μl) and a solution of 2-phenoxypropanoic acid in N,N-dimethylformamide (0.1 mol/L, 150 μl) at ambient temperature. To the mixture was added a solution of 1-[3-(dimethylamino)propyl]-3-ethylcarbodiimide (1 mol/L, 15 μl) and the mixture was stirred at 50° C. for 6 hours. The reaction mixture was diluted with ethyl acetate (10 ml), washed with water, saturated aqueous sodium hydrogencarbonate solution and brine, and evaporated in vacuo to give 2-(4-bromophenyl)-N-[2-(2-phenoxypropanoyl)-2,3-dihydro-1H-isoindol-5-yl]-1-cyclohexene-1-carboxamide as a solid.

(+)ESI-MS(m/z): 568(M+Na)⁺

### EXAMPLES 421-449

The compounds of Examples 421-449 shown in Table 4 were obtained in the same manner as in Example 420 as a solid.

TABLE 4

| | | (+) ESI- |
|---|---|---|
| Example | | MS |
| No. | IUPAC name | M + Na |
| 413 | N-[2-(phenylacetyl)-2,3-dihydro-1H-isoindol-5- | 513 |
| | yl]-2-[4-(trifluoromethyl)phenyl]-1- | |
| | cyclopentene-1-carboxamide | |
| 414 | N-[2-(phenylacetyl)-2,3-dihydro-1H-isoindol-5- | 527 |
| | yl]-2-[4-(trifluoromethyl)phenyl]-1- | |
| | cyclohexene-1-carboxamide | |
| 415 | N-[2-(phenylacetyl)-2,3-dihydro-1H-isoindol-5- | 515 |
| | yl]-4-[4-(trifluoromethyl)phenyl]-2,5-dihydro- | |
| | 3-furancarboxamide | |
| 416 | 2-(4-fluorophenyl)-N-[2-(phenylacetyl)-2,3- | 477 |
| | dihydro-1H-isoindol-5-yl]-1-cyclohexene-1- | |
| | carboxamide | |
| 417 | 2-(4-chlorophenyl)-N-[2-(phenylacetyl)-2,3- | 494 |
| | dihydro-1H-isoindol-5-yl]-1-cyclohexene-1- | |
| | carboxamide | |
| 418 | 2-(4-bromophenyl)-N-[2-(phenylacetyl)-2,3- | 538 |
| | dihydro-1H-isoindol-5-yl]-1-cyclohexene-1- | |
| | carboxamide | |
| 419 | 2-phenyl-N-[2-(phenylacetyl)-2,3-dihydro-1H- | 445 |
| | isoindol-5-yl]-1-cyclopentene-1-carboxamide | |
| 420 | 2-(4-bromophenyl)-N-[2-(2-phenoxypropanoyl)- | 568 |
| | 2,3-dihydro-1H-isoindol-5-yl]-1-cyclohexene-1- | |
| | carboxamide | |
| 421 | 2-(4-bromophenyl)-N-[2-(1-naphthylacetyl)-2,3- | 588 |
| | dihydro-1H-isoindol-5-yl]-1-cyclohexene-1- | |
| | carboxamide | |
| 422 | 2-(4-bromophenyl)-N-{2-[2-(4-chlorophenoxy)-2- | 617 |
| | methylpropanoyl]-2,3-dihydro-1H-isoindol-5- | |
| | yl}-1-cyclohexene-1-carboxamide | |
| 423 | 2-(4-bromophenyl)-N-{2-[(4- | 589 |
| | chlorophenoxy)acetyl]-2,3-dihydro-1H-isoindol- | |
| | 5-yl}-1-cyclohexene-1-carboxamide | |
| 424 | 2-(4-bromophenyl)-N-{2-[(2- | 573 |
| | chlorophenyl)acetyl]-2,3-dihydro-1H-isoindol- | |
| | 5-yl}-1-cyclohexene-1-carboxamide | |
| 425 | 2-(4-bromophenyl)-N-{2-[(3,4- | 598 |
| | dimethoxyphenyl)acetyl]-2,3-dihydro-1H- | |
| | isoindol-5-yl}-1-cyclohexene-1-carboxamide | |
| 426 | 2-(4-bromophenyl)-N-{2-[(4- | 556 |
| | fluorophenyl)acetyl]-2,3-dihydro-1H-isoindol- | |
| | 5-yl}-1-cyclohexene-1-carboxamide | |
| 427 | 2-(4-bromophenyl)-N-[2-((2E)-3-phenyl-2- | 550 |
| | propenoyl)-2,3-dihydro-1H-isoindol-5-yl]-1- | |
| | cyclohexene-1-carboxamide | |
| 428 | 2-(4-bromophenyl)-N-{2-[(2E)-3-(3,4- | 610 |
| | dimethoxyphenyl)-2-propenoyl]-2,3-dihydro-1H- | |
| | isoindol-5-yl}-1-cyclohexene-1-carboxamide | |
| 429 | 2-(4-bromophenyl)-N-[2-(4-phenylbutanoyl)-2,3- | 566 |
| | dihydro-1H-isoindol-5-yl]-1-cyclohexene-1- | |
| | carboxamide | |
| 430 | 2-(4-bromophenyl)-N-[2-(2-thienylacetyl)-2,3- | 544 |
| | dihydro-1H-isoindol-5-yl]-1-cyclohexene-1- | |
| | carboxamide | |
| 431 | 2-(4-bromophenyl)-N-[2-(1H-indol-3-ylacetyl)- | 577 |
| | 2,3-dihydro-1H-isoindol-5-yl]-1-cyclohexene-1- | |
| | carboxamide | |
| 432 | 2-(4-bromophenyl)-N-[2-(cyclopentylacetyl)- | 530 |
| | 2,3-dihydro-1H-isoindol-5-yl]-1-cyclohexene-1- | |
| | carboxamide | |
| 433 | 2-(4-bromophenyl)-N-[2-(cyclohexylacetyl)-2,3- | 544 |
| | dihydro-1H-isoindol-5-yl]-1-cyclohexene-1- | |
| | carboxamide | |
| 434 | 2-(4-bromophenyl)-N-[2-(3-phenoxybenzoyl)-2,3- | 616 |
| | dihydro-1H-isoindol-5-yl]-1-cyclohexene-1- | |
| | carboxamide | |
| 435 | 2-(4-bromophenyl)-N-[2-(3-phenylpropanoyl)- | 552 |
| | 2,3-dihydro-1H-isoindol-5-yl]-1-cyclohexene-1- | |
| | carboxamide | |
| 436 | 2-(4-bromophenyl)-N-{2-[(1- | 604 |
| | naphthyloxy)acetyl]-2,3-dihydro-1H-isoindol-5- | |
| | yl}-1-cyclohexene-1-carboxamide | |
| 437 | 2-(4-bromophenyl)-N-[2-(2-naphthylacetyl)-2,3- | 588 |
| | dihydro-1H-isoindol-5-yl]-1-cyclohexene-1- | |
| | carboxamide | |
| 438 | 2-(4-bromophenyl)-N-[2-(diphenylacetyl)-2,3- | 614 |
| | dihydro-1H-isoindol-5-yl]-1-cyclohexene-1- | |
| | carboxamide | |
| 439 | 2-(4-bromophenyl)-N-[2-(phenoxyacetyl)-2,3- | 554 |
| | dihydro-1H-isoindol-5-yl]-1-cyclohexene-1- | |
| | carboxamide | |
| 440 | 2-(4-bromophenyl)-N-{2-[(2- | 617 |
| | bromophenyl)acetyl]-2,3-dihydro-1H-isoindol-5- | |
| | yl}-1-cyclohexene-1-carboxamide | |
| 441 | 2-(4-bromophenyl)-N-(2-{[2- | 606 |
| | (trifluoromethyl)phenyl]acetyl}-2,3-dihydro- | |
| | 1H-isoindol-5-yl)-1-cyclohexene-1-carboxamide | |
| 442 | 2-(4-bromophenyl)-N-(2-{[3- | 606 |
| | (trifluoromethyl)phenyl]acetyl}-2,3-dihydro- | |
| | 1H-isoindol-5-yl)-1-cyclohexene-1-carboxamide | |
| 443 | 2-(4-bromophenyl)-N-{2-[(4-bromophenyl)acety]- | 617 |
| | 2,3-dihydro-1H-isoindol-5-yl}-1-cyclohexene-1- | |
| | carboxamide | |
| 444 | 2-(4-bromophenyl)-N-{2-[(4- | 573 |
| | chlorophenyl)acety]-2,3-dihydro-1H-isoindol-5- | |
| | yl}-1-cyclohexene-1-carboxamide | |
| 445 | 2-(4-bromophenyl)-N-{2-[(4- | 568 |
| | methoxyphenyl)acetyl]-2,3-dihydro-1H-isoindol- | |
| | 5-yl}-1-cyclohexene-1-carboxamide | |
| 446 | N-(2-{[3,5-bis(trifluoromethyl)phenyl]acetyl}- | 674 |
| | 2,3-dihydro-1H-isoindol-5-yl)-2-(4- | |
| | bromophenyl)-1-cyclohexene-1-carboxamide | |
| 447 | 2-(4-bromophenyl)-N-{2-[3- | 616 |
| | (phenylsulfonyl)propanoyl]-2,3-dihydro-1H- | |
| | isoindol-5-yl}-1-cyclohexene-1-carboxamide | |
| 448 | N-[2-(1,3-benzodioxol-5-ylacetyl)-2,3-dihydro- | 582 |
| | 1H-isoindol-5-yl]-2-(4-bromophenyl)-1- | |
| | cyclohexene-1-carboxamide | |
| 449 | 2-(4-bromophenyl)-N-{2-[(3-oxo-2,3-dihydro-1H- | 578 |
| | inden-1-yl)carbonyl]-2,3-dihydro-1H-isoindol- | |
| | 5-yl}-1-cyclohexene-1-carboxamide | |

This application is based on application No. PR 9164 filed in Australia, application No. PS 0443 filed in Australia, application No. 91106855 filed in Republic of China (Taiwan), and PCT application No. PCT/JP02/03529, the content of which is incorporated hereinto by reference.

1. A compound of the formula (I) 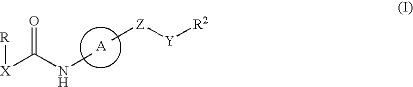
wherein
R¹ is aryl optionally substituted by substituent(s);
R² is aryl, heteroaryl, lower cycloalkyl, aryloxy, arylsulfonyl, vinyl, carbamoyl, protected carboxy or protected amino, each of said aryl, heteroaryl, lower cycloalkyl, aryloxy and arylsulfonyl is optionally substituted by substituent(s); 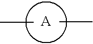
is bivalent residue derived from aryl or heteroaryl, each of which is optionally substituted by nitro, oxo or optionally protected amino;
X is bivalent residue derived from the group consisting of cycloalkene, naphthalene, unsaturated 5 or 6-membered heteromonocyclic group, each of which is optionally substituted by substituent(s), and benzene which is substituted by substituent(s);
Y is -(A¹)ₘ₁-(A²)ₘ₂-
wherein A¹ is —NH—, —N (R³)—, —CO—, —NH—CO—, —CO—NH—, —CO—CH═CH—, —O—, —CH₂—O—, —CH₂—NH—CO—, —CH₂—CO—NH or —CH(OH)—, wherein R³ is amino protective group,
A² is lower alkylene optionally substituted by aryl, and
m1 and m2 are independently 0 or 1; and
Z is direct bond or bivalent residue derived from piperazine or piperazine substituted by lower alkyl;
provided that when Z is direct bond, then R² is aryl, heteroaryl, lower cycloalkyl, aryloxy, arylsulfonyl or protected amino, each of said aryl, heteroaryl, lower cycloalkyl, aryloxy and arylsulfonyl is optionally substituted by substituent(s),
or a salt thereof.

2. The compound of claim 1, wherein
R¹ is aryl optionally substituted by substituent(s);
R² is aryl, heteroaryl, lower cycloalkyl, aryloxy, arylsulfonyl, vinyl, carbamoyl, protected carboxy or protected amino, each of said aryl, heteroaryl, lower cycloalkyl, aryloxy and arylsulfonyl is optionally substituted by substituent(s) selected from the group consisting of lower alkyl, trihalo(lower)alkyl, optionally protected amino, optionally substituted heteroaryl, cyano, lower alkoxy, halogen, aryloxy, lower alkylenedioxy, oxo, lower alkanoylamino and amino protective group; 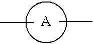
is bivalent residue derived from aryl or heteroaryl, each of which is optionally substituted by nitro, oxo or optionally protected amino;
X is bivalent residue derived from the group consisting of cycloalkene, naphthalene, unsaturated 5 or 6-membered heteromonocyclic group, each of which is optionally substituted by substituent(s), and benzene which is substituted by substituent(s);
Y is -(A¹)ₘ₁-(A²)ₘ₂-
wherein A¹ is —NH—, —N(R³)—, —CO—, —NH—CO—, —CO—NH—, —CO—CH═CH—, —O—, —CH₂—O—, —CH₂—NH—CO—, —CH₂—CO—NH or —CH(OH)—, wherein R³ is amino protective group,
A² is lower alkylene optionally substituted by aryl, and
m1 and m2 are independently 0 or 1; and
Z is direct bond or bivalent residue derived from piperazine or piperazine substituted by lower alkyl;
provided that when Z is direct bond, then R² is aryl, heteroaryl, lower cycloalkyl, aryloxy, arylsulfonyl or. protected amino, each of said aryl, heteroaryl, lower cycloalkyl, aryloxy and arylsulfonyl is optionally substituted by substituent(s) selected from the group consisting of lower alkyl, trihalo(lower)alkyl, optionally protected amino, optionally substituted heteroaryl, cyano, lower alkoxy, halogen, aryloxy, lower alkylenedioxy, oxo, lower alkanoylamino and amino protective group,
or a salt thereof.

3. The compound of claim 2, wherein
R¹ is phenyl optionally substituted by substituent(s) selected from the group consisting of lower alkyl, lower alkoxy, halogen, trihalo(lower)alkyl, trihalo(lower)alkoxy, lower alkanoyl, di(lower)alkylamino and lower alkylthio;
R² is phenyl, naphthyl, indanyl, pyridinyl, pyrimidinyl, pyrazinyl, thiazolyl, pyrrolyl, imidazolyl, triazolyl, thienyl, indolyl, lower cycloalkyl, phenoxy, naphthyloxy, phenylsulfonyl or protected amino, each of said phenyl, naphthyl, indanyl, pyridinyl, pyrimidinyl, pyrazinyl, thiazolyl, pyrrolyl, imidazolyl, triazolyl, thienyl, indolyl, lower cycloalkyl, phenoxy, naphthyloxy and phenylsulfonyl is optionally substituted by substituent(s) selected from the group consisting of lower alkyl, trihalo(lower)alkyl, optionally protected amino, optionally substituted pyrrolyl, cyano, lower alkoxy, halogen, aryloxy, lower alkylenedioxy, oxo, lower alkanoylamino and amino protective group; 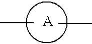
is bivalent residue derived from phenyl optionally substituted by nitro or optionally protected amino, indanyl, pyridinyl, indolinyl, tetrahydroisoquinolyl or isoindolinyl each of which is optionally substituted by oxo or amino;
X is bivalent residue derived from the group consisting of cycloalkene, naphthalene, unsaturated 5 or 6-membered heteromonocyclic group, each of which is optionally substituted by substituent(s), and benzene which is substituted by substituent(s), wherein the substituent is selected from the group consisiting of lower alkyl, lower alkoxy, halogen, lower alkanoyl, lower alkoxy(lower)alkyl and hydroxy(lower)alkyl; Y is -(A¹)ₘ₁-(A²)ₘ₂-
wherein A¹ is —NH—, —N(R³)—, —CO—, —NH—CO—, —CO—NH—, —CO—CH═CH—, —O—, —CH₂—O—, —CH₂—NH—CO—, —CH₂—CO—NH— or —CH(OH)—, wherein R³ is amino protective group,
A² is lower alkylene optionally substituted by aryl, and
m1 and m2 are independently 0 or 1; and
Z is direct bond,
or a salt thereof.

4. The compound of claim 3, wherein
R¹ is phenyl optionally substituted by substituent(s) selected from the group consisting of methyl, ethyl, isopropyl, methoxy, chloro, fluoro, bromo, trifluoromethyl, trifluoromethoxy, acetyl, dimethylamino and methylthio;
R² is pyridinyl, pyrimidinyl, pyrazinyl or thiazolyl, each of said pyridinyl, pyrimidinyl, pyrazinyl and thiazolyl is optionally substituted by substituent(s) selected from the group consisting of methyl, amino, acetylamino or tert-butoxycarbonylamino, optionally substituted pyrrolyl, cyano and methoxy; 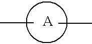
is bivalent residue derived from phenyl or pyridinyl;
X is 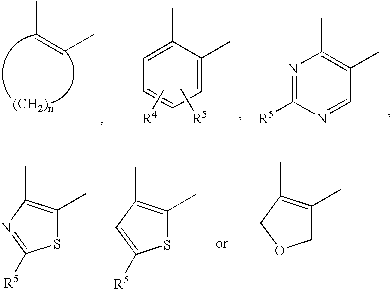
wherein R⁴ is lower alkyl, lower alkoxy, lower alkanoyl, hydroxy(lower)alkyl, lower alkoxy(lower)alkyl or halogen,
R⁵ is hydrogen or lower alkyl, and
n is 3, 4, 5 or 6;
Y is direct bond or bivalent residue selected from the group consisting of 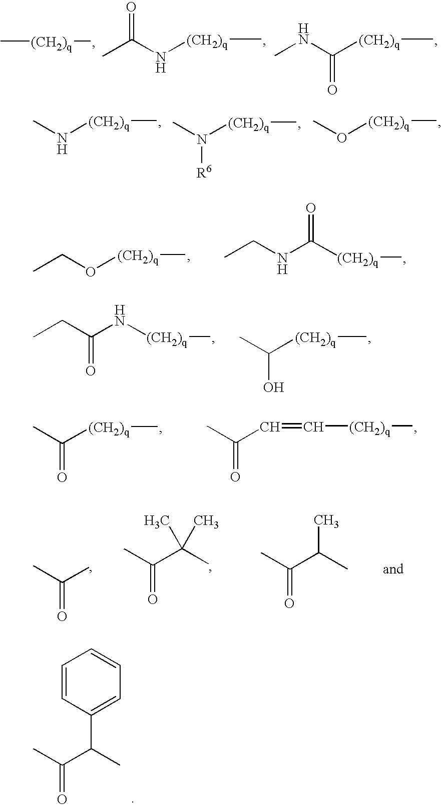
wherein q is an integer of 0 to 3, and R⁶ is amino protective group,
or a salt thereof.

5. The compound of claim 1, wherein X is 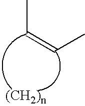
wherein n is 3, 4, 5 or 6,
or a salt thereof.

6. The compound of claim 5, wherein
R¹ is phenyl optionally substituted by substituent(s) selected from the group consisting of lower alkyl, lower alkoxy, halogen, trihalo(lower)alkyl, trihalo(lower)alkoxy, lower alkanoyl, di(lower)alkylamino and lower alkylthio;
R² is aryl, heteroaryl, lower cycloalkyl, aryloxy, arylsulfonyl, vinyl, carbamoyl, protected carboxy or protected amino, each of said aryl, heteroaryl, lower cycloalkyl, aryloxy and arylsulfonyl is optionally substituted by substituent(s) selected from the group consisting of lower alkyl, trihalo(lower)alkyl, optionally protected amino, optionally substituted heteroaryl, cyano, lower alkoxy, halogen, aryloxy, lower alkylenedioxy, oxo, lower alkanoylamino and amino protective group; 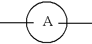
is bivalent residue derived from aryl or heteroaryl;
X is 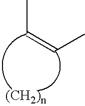
wherein n is 3, 4, 5 or 6;
Y is -(A¹)ₘ₁-(A²)ₘ₂-
wherein A¹ is —NH—, —N(R³)—, —CO—, —NH—CO—, —CO—NH—, —CO—CH═CH—, —O, —CH₂—O—, —CH₂—NH—CO—, —CH₂—CO—NH— or —CH(OH)—, wherein R³ is amino protective group,
A² is lower alkylene optionally substituted by aryl, and
m1 and m2 are independently 0 or 1; and.
Z is direct bond or bivalent residue derived from piperazine or piperazine substituted by lower alkyl;
provided that when Z is direct bond, then R² is aryl, heteroaryl, lower cycloalkyl, aryloxy, arylsulfonyl or protected amino, each of said aryl, heteroaryl, lower cycloalkyl, aryloxy and arylsulfonyl is optionally substituted by substituent(s) selected from the group consisting of lower alkyl, trihalo(lower)alkyl, optionally protected amino, optionally substituted heteroaryl, cyano, lower alkoxy, halogen, aryloxy, lower alkylenedioxy, oxo, lower alkanoylamino and amino protective group,
or a salt thereof.

7. The compound of claim 6, wherein
R² is phenyl, naphthyl, indanyl, pyridinyl, pyrimidinyl, thiazolyl, pyrrolyl, imidazolyl, triazolyl, thienyl, indolyl, lower cycloalkyl, phenoxy, naphthyloxy, phenylsulfonyl, vinyl, carbamoyl, protected carboxy or protected amino, each of said phenyl, naphthyl, indanyl, pyridinyl, pyrimidinyl, thiazolyl, pyrrolyl, imidazolyl, triazolyl, thienyl, indolyl, lower cycloalkyl, phenoxy, naphthyloxy and phenylsulfonyl is optionally substituted by substituent(s) selected from the group consisting of lower alkyl, trihalo(lower)alkyl, optionally protected amino, optionally substituted pyrrolyl, cyano, lower alkoxy, halogen, aryloxy, lower alkylenedioxy, oxo, lower alkanoylamino and amino protective group; 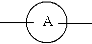
is bivalent residue derived from phenyl, indanyl, pyridinyl, indolinyl, isoindolinyl or 1,2,3,4-tetrahydroisoquinolinyl;
Y is direct bond or bivalent residue selected from the group consisting of 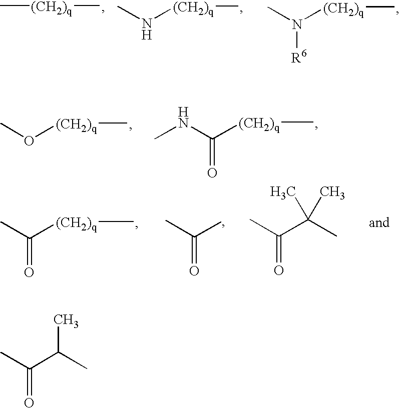
wherein q is an integer of 0 to 3, and R⁶ is amino protective group;
provided that when Z is direct bond, then R² is phenyl, naphthyl, indanyl, pyridinyl, pyrimidinyl, thiazolyl, pyrrolyl, imidazolyl, triazolyl, thienyl, indolyl, lower cycloalkyl, phenoxy, naphthyloxy, phenylsulfonyl or protected amino, each of said phenyl, naphthyl, indanyl, pyridinyl, pyrimidinyl, thiazolyl, pyrrolyl, imidazolyl, triazolyl, thienyl, indolyl, lower cycloalkyl, phenoxy, naphthyloxy and phenylsulfonyl is optionally substituted by substituent(s) selected from the group consisting of lower alkyl, trihalo(lower)alkyl, optionally protected amino, optionally substituted pyrrolyl, cyano, lower alkoxy, halogen, aryloxy, lower alkylenedioxy, oxo, lower alkanoylamino and amino protective group,
or a salt thereof.

8. The compound of claim 5 having the following formula: 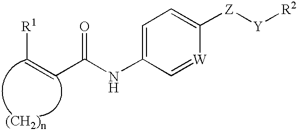
wherein
R¹ is phenyl optionally substituted by substituent(s) selected from the group consisting of lower alkyl, lower alkoxy, halogen, trihalo(lower)alkyl, trihalo(lower)alkoxy, lower alkanoyl and di(lower)alkylamino;
R² is aryl or heteroaryl, each of said aryl and heteroaryl is optionally substituted by substituent(s) selected from the group consisting of lower alkyl, trihalo(lower)alkyl, optionally protected amino, optionally substituted heteroaryl, cyano, lower alkoxy, lower alkanoylamino and amino protective group;
W is CH or N;
Y is -(A¹)ₘ₁-(A²)ₘ₂-
wherein A¹ is —NH—, —N(R³)—, —CO—, —NH—CO—, —CO—NH—, —CO—CH═CH—, —O—, —CH₂—O—, —CH₂—NH—CO—, —CH₂—CO—NH— or —CH(OH)—, wherein R³ is amino protective group,
A² is lower alkylene optionally substituted by aryl, and
m1 and m2 are independently 0 or 1;
Z is direct bond; and
n is 3, 4, 5 or 6,
or a salt thereof.

9. The compound of claim 8 having the following formula: 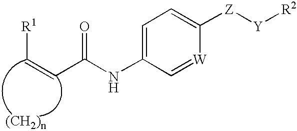
wherein
R¹ is phenyl optionally substituted by substituent(s) selected from the group consisting of lower alkyl and trihalo(lower)alkyl;
R² is pyridinyl or thiazolyl, each of said pyridinyl and thiazolyl is optionally substituted by optionally protected amino;
W is CH or N;
Y is -(A¹)ₘ₁-(A²)ₘ₂-
wherein A¹ is —NH—, —N(R³)— or —O—, wherein R³ is amino protective group,
A² is lower alkylene optionally substituted by aryl, and
m1 and m2 are independently 0 or 1;
Z is direct bond; and
n is 4,
or a salt thereof.

10. The compound of claim 5 having the following formula: 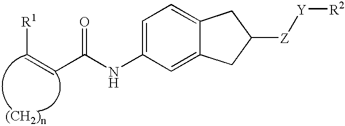
wherein
R¹ is phenyl optionally substituted by substituent(s) selected from the group consisting of lower alkyl, lower alkoxy, halogen, trihalo(lower)alkyl, trihalo(lower)alkoxy, lower alkanoyl, di(lower)alkylamino and lower alkylthio;
R² is aryl, heteroaryl or protected amino, each of said aryl and heteroaryl is optionally substituted by substituent(s) selected from the group consisting of lower alkyl, trihalo(lower)alkyl, optionally protected amino, optionally substituted heteroaryl, cyano, lower alkoxy, halogen, aryloxy, lower alkylenedioxy, lower alkanoylamino and amino protective group;
Y is -(A¹)ₘ₁-(A²)ₘ₂-
wherein A¹ is —NH—, —N(R³)—, —CO—, —NH—CO—, —CO—CH═CH— or —O—, wherein R³ is amino protective group,
A² is lower alkylene optionally substituted by aryl, and
m1 and m2 are independently 0 or 1;
Z is direct bond; and
n is 3, 4, 5 or 6,
or a salt thereof.

11. The compound of claim 5 having the following formula: 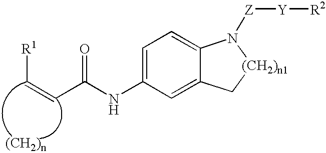
wherein
R¹ is phenyl optionally substituted by substituent(s) selected from the group consisting of lower alkyl, lower alkoxy, halogen, trihalo(lower)alkyl, trihalo(lower)alkoxy, lower alkanoyl, di(lower)alkylamino and lower alkylthio;
R² is aryl or heteroaryl, each of said aryl and heteroaryl is optionally substituted by substituent(s) selected from the group consisting of lower alkyl, trihalo(lower)alkyl, optionally protected amino, optionally substituted heteroaryl, cyano, lower alkoxy, halogen, aryloxy, lower alkylenedioxy, oxo, lower alkanoylamino and amino protective group;
Y is -(A¹)ₘ₁-(A²)ₘ₂-
wherein A¹ is —NH—, —N(R³)—, —CO—, —NH—CO—, —CO—CH═CH— or —O—, wherein R³ is amino protective group,
A² is lower alkylene optionally substituted by aryl, and
m1 and m2 are independently 0 or 1;
Z is direct bond;
n is 3, 4, 5 or 6; and
n1 is 1 or 2,
or a salt thereof.

12. The compound of claim 5 having the following formula: 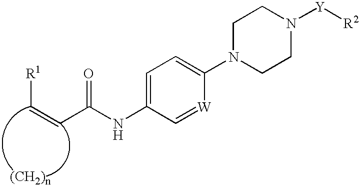
wherein
R¹ is phenyl optionally substituted by substituent(s) selected from the group consisting of lower alkyl, lower alkoxy, halogen, trihalo (lower) alkyl, trihalo(lower)alkoxy, lower alkanoyl, di(lower)alkylamino and lower alkylthio;
R² is aryl, heteroaryl, vinyl, carbamoyl, protected carboxy or protected amino, each of said aryl and heteroaryl is optionally substituted by substituent(s) selected from the group consisting of lower alkyl, trihalo(lower)alkyl, optionally protected amino, optionally substituted heteroaryl, cyano, lower alkoxy, halogen, aryloxy, lower alkylenedioxy, oxo, lower alkanoylamino and amino protective group;
W is CH or N;
Y is -(A¹)ₘ₁-(A²)ₘ₂-
wherein A¹ is —NH—, —N(R³)—, —CO—, —NH—CO—, —CO—CH═CH— or —O—, wherein R³ is amino protective group,
A² is lower alkylene optionally substituted by aryl, and
m1 and m2 are independently 0 or 1;
n is 3, 4, 5 or 6,
or a salt thereof.

13. The compound of claim 12 having the following formula: 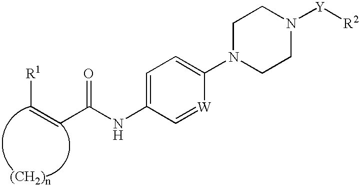
wherein
R¹ is phenyl optionally substituted by substituent(s) selected from the group consisting of lower alkyl and trihalo(lower)alkyl;
R² is aryl optionally substituted by cyano;
W is CH or N;
Y is -(A²)ₘ₂-
wherein A² is lower alkylene, and
m2 is 1;
n is 4,
or a salt thereof.

14. The compound of claim 5 having the following formula: 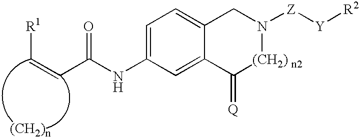
wherein
R¹ is phenyl optionally substituted by substituent(s) selected from the group consisting of lower alkyl, lower alkoxy, halogen, trihalo(lower)alkyl, trihalo(lower)alkoxy, lower alkanoyl, di(lower)alkylamino and lower alkylthio;
R² is aryl, heteroaryl or protected amino, each of said aryl and heteroaryl is optionally substituted by substituent(s) selected from the group consisting of lower alkyl, trihalo(lower)alkyl, optionally protected amino, optionally substituted heteroaryl, cyano, lower alkoxy, halogen, aryloxy, lower alkylenedioxy, oxo, lower alkanoylamino and amino protective group;
Y is -(A¹)ₘ₁-(A²)ₘ₂-
wherein A¹ is —NH—, —N(R³)—, —CO—, —NH—CO—, —CO—CH═CH— or —O—, wherein R³ is amino protective group,
A² is lower alkylene optionally substituted by aryl, and
m1 and m2 are independently 0 or 1;
Z is direct bond;
Q is 0 or a pair of hydrogen atoms;
n is 3, 4, 5 or 6; and
n2 is 0 or 1,
or a salt thereof.

15. The compound of claim 1, wherein X is 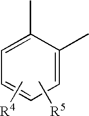
wherein R⁴ is lower alkyl, lower alkoxy, lower alkanoyl, hydroxy(lower)alkyl, lower alkoxy(lower)alkyl or halogen, and
R⁵ is hydrogen or lower alkyl,
or a salt thereof.

16. The compound of claim 15, wherein
R¹ is phenyl optionally substituted by substituent(s) selected from the group consisting of lower alkyl and trihalo(lower)alkyl;
R² is heteroaryl optionally substituted by optionally protected amino; 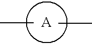
is bivalent residue derived from aryl or pyridinyl;
X is 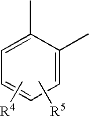
wherein R⁴ is lower alkyl, and R⁵ is hydrogen;
Y is -(A¹)ₘ₁-(A²)ₘ₂-
wherein A¹ is —NH—, —N(R³)—, —O—, wherein R³ is amino protective group,
A² is lower alkylene optionally substituted by aryl, and
m1 and m2 are independently 0 or 1; and
Z is direct bond,
or a salt thereof.

17. The compound of claim 1 selected from the group consisting of
4′,5-dimethyl-N-(4-([2-(2-pyridinyl)ethyl]amino}phenyl)-1,1′-biphenyl-2-carboxamide,
5-methyl-N-(4-{[2-(2-pyridinyl)ethyl]amino}phenyl)-4′-(trifluoromethyl)-1,1′-biphenyl-2-carboxamide,
N-{4-[2-(6-amino-2-pyridinyl)ethoxy]phenyl}-5-methyl-4′-(trifluoromethyl)-1,1′-biphenyl-2-carboxamide,
2-(4-methylphenyl)-N-(4-{[2-(2-pyridinyl)ethyl]amino}phenyl)-1-cyclohexene-1-carboxamide,
N-(4-{[2-(2-pyridinyl)ethyl]amino}phenyl)-2-[4-(trifluoromethyl)phenyl]-1-cyclohexene-1-carboxamide,
N-(4-{[2-(6-amino-2-pyridinyl)ethyl]amino}phenyl)-2-[4-(trifluoromethyl)phenyl]-1-cyclohexene-1-carboxamide,
N-(4-{[2-(2-amino-1,3-thiazol-4-yl)ethyl]amino}phenyl)-2-(4-methylphenyl)-1-cyclohexene-1-carboxamide,
N-{4-[4-(3-cyanobenzyl)-1-piperazinyl]phenyl}-2-[4-(trifluoromethyl)phenyl]-1-cyclohexene-1-carboxamide,
N-{6-[4-(3-cyanobenzyl)-1-piperazinyl]-3-pyridinyl}-2-[4-(trifluoromethyl)phenyl]-1-cyclohexene-1-carboxamide,
N-(6-{[2-(6-amino-2-pyridinyl)ethyl]amino}-3-pyridinyl)-2-[4-(trifluoromethyl)phenyl]-1-cyclohexene-1-carboxamide, or a salt thereof.

18. A compound of the following formula: 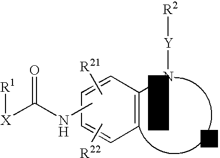
wherein
R¹ is 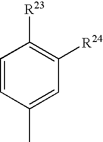
wherein R²³ and R²⁴ are independently hydrogen or a substituent;
R²¹ and R²² are independently hydrogen or a substituent;
R² is unsaturated 5 to 6-membered heteromonocyclic group, which is optionally substituted by one or more substituent(s);
X is cycloalkenylene optionally substituted by one or more substituent (s);
y¹ is bivalent group selected from the group consisting of ethylene, trimethylene and vinylene, wherein CH₂ is optionally replaced by NH or O, and CH is optionally replaced by N, and said bivalent group is optionally substituted by one or more substituent(s);
and
Y is —(CH₂)ᵣ—, —CO—(CH₂)ₛ— or —CO—NH—, wherein r is 1, 2 or 3 and s is 1 or 2,
or a salt thereof.

19. A compound of the formula: 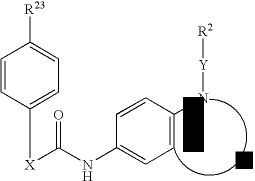
wherein
R²³ is hydrogen, lower alkyl, lower alkoxy, halogen, trihalo(lower)alkyl or di(lower)alkylamino;
R² is 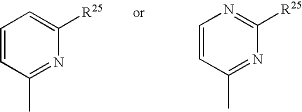
wherein R²⁵ is hydrogen, amino or 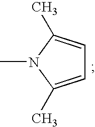
X is 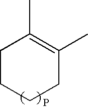
wherein p is 1 or 2;
Y¹ is —CH₂—CH₂—; and
Y is —CO—CH₂—,
or a salt thereof.

20. The compound of claim 1 or a pharmaceutically acceptable salt thereof for use as a medicament.

21. A process for preparing a compound of the formula (I) 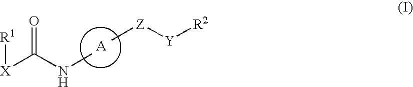
wherein
R¹ is aryl optionally substituted by substituent(s);
R² is aryl, heteroaryl, lower cycloalkyl, aryloxy, arylsulfonyl, vinyl, carbamoyl, protected carboxy or protected amino, each of said aryl, heteroaryl, lower cycloalkyl, aryloxy and arylsulfonyl is optionally substituted by substituent(s); 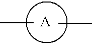
is bivalent residue derived from aryl or heteroaryl, each of which is optionally substituted by nitro, oxo or optionally protected amino;
X is bivalent residue derived from the group consisting of cycloalkene, naphthalene, unsaturated 5 or 6-membered heteromonocyclic group, each of which is optionally substituted by substituent(s), and benzene which is substituted by substituent(s);
Y is -(A¹)ₘ₁-(A²)ₘ₂-
wherein A¹ is —NH—, —N(R³)—, —CO—, —NH—CO—, —CO—NH—, —CO—CH═CH—, —O—, —CH₂—O—, —CH₂—NH—CO—, —CH₂—CO—NH or —CH(OH)—, wherein R³ is amino protective group,
A² is lower alkylene optionally substituted by aryl, and
m1 and m2 are independently 0 or 1; and
Z is direct bond or bivalent residue derived from piperazine or piperazine substituted by lower alkyl;
provided that when Z is direct bond, then R² is aryl, heteroaryl, lower cycloalkyl, aryloxy, arylsulfonyl or protected amino, each of said aryl, heteroaryl, lower cycloalkyl, aryloxy and arylsulfonyl is optionally substituted by substituent(s),
or a salt thereof, which comprises
(a) reacting a compound of the formula (II) 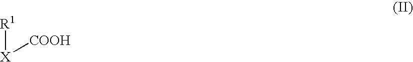
wherein R¹ and X are each as defined above, or its reactive derivative at the carboxy group, or a salt thereof with a compound of the formula (III) 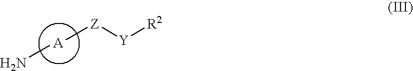
wherein R², Y, Z and ring A are each as defined above, or its reactive derivative at the amino group, or a salt thereof to give a compound of the formula (I) 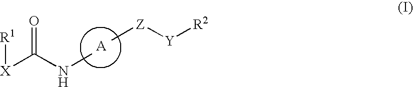
wherein R¹, R², X, Y, Z and ring A are each as defined above, or
(b) reacting a compound of the formula (II) 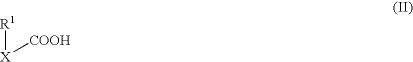
wherein R¹ and X are each as defined above, or its reactive derivative at the carboxy group, or a salt thereof with a compound of the formula (XVI) 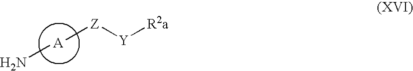
wherein R²a is aryl, heteroaryl, lower cycloalkyl, aryloxy or arylsulfonyl, each of which is substituted by protected amino, and Y, Z and ring A are each as defined above, or its reactive derivative at the amino group, or a salt thereof to give a compound of the formula (I)-14 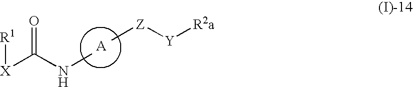
wherein R¹, R²a, X, Y, Z and ring A are each as defined above, or a salt thereof, or
(c) subjecting a compound of the formula (I)-14 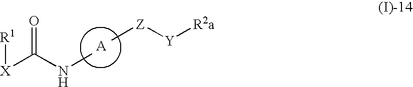
wherein R¹, R²a, X, Y, Z and ring A are each as defined above, or a salt thereof to elimination reaction of the amino protective group to give a compound of the formula (I)-15 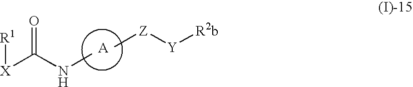
wherein R²b is aryl, heteroaryl, lower cycloalkyl, aryloxy or arylsulfonyl, each of which is substituted by amino, and R¹, X, Y, Z and ring A are each as defined above, or salt thereof, or
(d) reacting a compound of the formula (II) 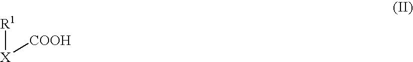
wherein R¹ and X are each as defined above, or its reactive derivative at the carboxy group, or a salt thereof with a compound of the formula (XVIII) 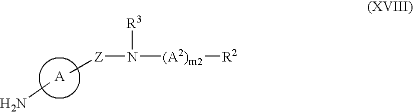
wherein R², R³, Z, ring A, A² and m2 are each as defined above, or its reactive derivative at the amino group, or a salt thereof to give a compound of the formula (I)-18 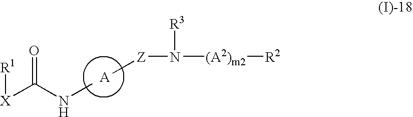
wherein R¹, R², R³, X, Z, ring A, A² and m2 are each as defined above, or a salt thereof, or
(e) subjecting a compound of the formula (I)-18 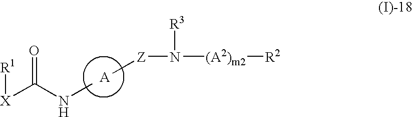
wherein R¹, R², R³, X, Z, ring A, A² and m2 are each as defined above, or a salt thereof to elimination reaction of the amino protective group to give a compound of the formula (I)-19 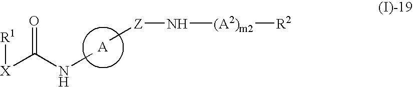
wherein R¹, R², X, Z, ring A, A² and m2 are each as defined above, or a salt thereof, or
(f) reacting a compound of the formula (XXVIII) 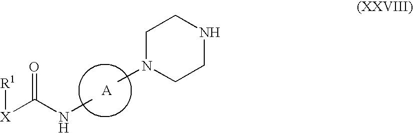
wherein R¹, X and ring A are each as defined above, or a salt thereof with a compound of the formula (XXIX)
OHC—R²c (XXIX)
wherein R²c is aryl, heteroaryl or lower cycloalkyl, each of which is optionally substituted by substituent(s) in the presence of a reducing agent to give a compound of the formula (I)-20 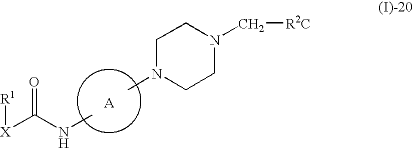
wherein R¹, R²c, X and ring A are each as defined above, or a salt thereof, or
(g) reacting a compound of the formula (XXVIII) 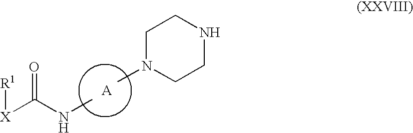
wherein R¹, X and ring A are each as defined above, or a salt thereof with a compound of the formula (XXX)
X²—Y—R² (XXX)
wherein R² and Y are each as defined above, and X² is leaving group in the presence of a base to give a compound of the formula (I)-21 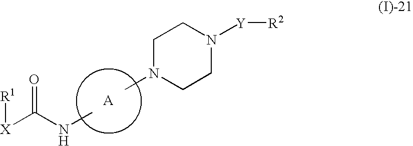
wherein R¹, R², X, Y and ring A are each as defined above, or a salt thereof.

22. A pharmaceutical composition comprising a compound of claim 1 or a pharmaceutically acceptable salt thereof in admixture with a pharmaceutically acceptable carrier.

23. Use of a compound of claim 1 or a pharmaceutically acceptable salt thereof for preparing a medicament as an apolipoprotein B (Apo B) secretion inhibitor.

24. Use of a compound of claim 1 or a pharmaceutically acceptable salt thereof for preparing a medicament for the prophylaxis or treatment of a disease or condition resulting from elevated circulating levels of Apo B.

25. Use of a compound of claim 1 or a pharmaceutically acceptable salt thereof for preparing a medicament for the prophylaxis or treatment of hyperlipemia, hyperlipidemia, hyperlipoproteinemia, hypoalphalipoproteinemia, hypercholesterolemia, hypertriglyceridemia, atherosclerosis, pancreatitis, non-insulin dependent diabetes mellitus (NIDDM), obesity, coronary heart diseases, myocardial infarction, stroke, restenosis or Syndrome X.

26. A method for inhibiting or decreasing Apo B secretion in a mammal, which comprises administering an Apo B secretion inhibiting or decreasing amount of a compound of claim 1 or a pharmaceutically acceptable salt thereof to the mammal.

27. A method for preventing or treating a disease or condition resulting from elevated circulating levels of Apo B in a mammal, which comprises administering an effective amount of a compound of claim 1 or a pharmaceutically acceptable salt thereof to the mammal.

28. The method of claim 27 wherein the disease or condition resulting from the elevated circulating levels of Apo B is selected from the group consisting of hyperlipemia, hyperlipidemia, hyperlipoproteinemia, hypoalphalipoproteinemia, hypercholesterolemia, hypertriglyceridemia, atherosclerosis, pancreatitis, non-insulin dependent diabetes mellitus (NIDDM), obesity, coronary heart diseases, myocardial infarction, stroke, restenosis and Syndrome X.
